(19) United States
(12) Patent Application Publication
(10) Pub. No.: US 2023/0192679 A1
(43) Pub. Date: Jun. 22, 2023

(54) CRYSTALLINE FORMS, PHARMACEUTICAL COMPOSITIONS AND METHODS OF USE THEREOF

(71) Applicant: Tango Therapeutics, Inc., Boston, MA (US)

(72) Inventors: Kevin M. Cottrell, Arlington, MA (US); Sapna Makhija Garad, Malden, MA (US); Magnus Ronn, Melrose, MA (US); Kimberly Jane Briggs, Framingham, MA (US); Erik William Wilker, Lexington, MA (US); Matthew Robert Tonini, Cambridge, MA (US); Minjie Zhang, Wayland, MA (US); Alice Wanjung Tsai-Marie, Arlington, MA (US); Jianglin Liang, Needham, MA (US); Hongming Li, Newton, MA (US)

(21) Appl. No.: 18/083,222

(22) Filed: Dec. 16, 2022

Related U.S. Application Data
(60) Provisional application No. 63/291,007, filed on Dec. 17, 2021.
(60) Provisional application No. 63/419,221, filed on Oct. 25, 2022.

(51) Int. Cl. C07D 417/14

(52) U.S. Cl. CPC C07D 417/14; C07B 2200/13

(57) ABSTRACT

Crystalline forms and pharmaceutical composition of a PRMT5 inhibitor of formula (I), methods of making PRMT5 inhibitor of formula (I) and crystalline forms thereof, methods of making the pharmaceutical compositions of the PRMT5 inhibitor of formula (I) and methods of using the PRMT5 inhibitor of formula (I) or crystalline solid forms and pharmaceutically acceptable compositions thereof.

### CROSS-REFERENCE TO RELATED APPLICATIONS

This application claims the benefit of and priority to U.S. Provisional Patent Application No. 63/291,007 filed on Dec. 17, 2021 and U.S. Provisional Patent Application No. 63/419,221 filed on Oct. 25, 2022, each of which is herein incorporated by reference in their entirety.

### BACKGROUND

Development of pharmaceutical compositions comprising one or more novel active ingredients requires a variety of considerations, such as route of administration (e.g., enteral, parenteral, topical, etc.), dosage form (e.g., solid—tablet, capsule, etc.; liquid—solution, suspension, syrup, etc.), strength of active ingredient(s) (e.g., 1 mg-1,000 mg), non-therapeutic component(s) (e.g., excipients) and their respective amounts, and each of these considerations may involve additional considerations such as stability, degradation, sensitivity to light, solubility, taste if administered enterally, palatability, pH, skin irritability, microbial growth, etc. Advancing a novel active ingredient (e.g., a PRMT5 inhibitor) through rigorous regulatory entities requires discovering and developing a pharmaceutical composition that addresses these, or other, considerations.

Accordingly, there is a need for pharmaceutical compositions comprising compounds (e.g., PRMT5 inhibitors) that exhibit desirable properties treat diseases or disorders (e.g., cancers) in human patients.

### SUMMARY

In some embodiments, provided is a crystalline form of N-(6-amino-5-methylpyridin-3-yl)-2-((2R,5 S)-2-(benzo[d]thiazol-5-yl)-5-methylpiperidin-1-yl)-2-oxoacetamide (a compound of formula (I))

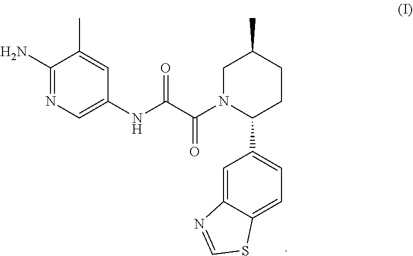 [SMILES: Cc1cc(NC(=O)C(=O)N2C[C@@H](C)CC[C@@H]2c2ccc3scnc3c2)cnc1N]

In some embodiments, provided is a crystalline form of N-(6-amino-5-methylpyridin-3-yl)-2-((2R,5 S)-2-(benzo[d]thiazol-5-yl)-5-methylpiperidin-1-yl)-2-oxoacetamide (a compound of formula (I))

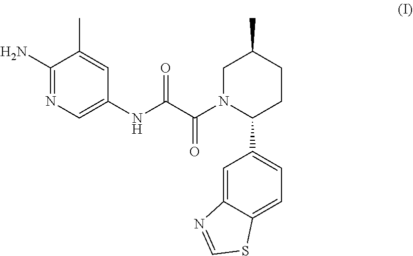 [SMILES: Cc1cc(NC(=O)C(=O)N2C[C@@H](C)CC[C@@H]2c2ccc3scnc3c2)cnc1N]

wherein the X-ray powder diffraction (XRPD) pattern of the crystalline form comprises one or more peaks at 20 angles selected from 6.4±0.2, 8.9±0.2, 12.7±0.2, 14.0±0.2, 19.1±0.2, 19.9±0.2, 22.6±0.2 degrees (Form A).

In some embodiments, provided are pharmaceutical compositions comprising a compound of formula (I), or a pharmaceutically acceptable salt thereof, and at least one pharmaceutically acceptable excipient.

In certain embodiments, the pharmaceutical compositions comprise crystalline form A of the compound of formula (I).

In some embodiments, provided is a pharmaceutical composition comprising:

(a) a compound of formula (I)

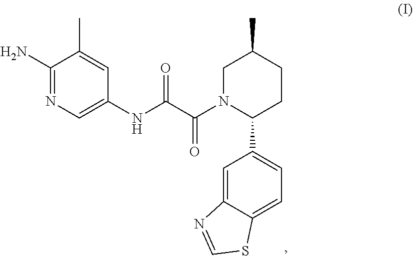 [SMILES: Cc1cc(NC(=O)C(=O)N2C[C@@H](C)CC[C@@H]2c2ccc3scnc3c2)cnc1N]

or a pharmaceutically acceptable salt thereof;
(b) a filler (e.g., microcrystalline cellulose);
(c) a glidant (e.g., colloidal silicon dioxide);
(d) a disintegrant (e.g., croscarmellose sodium); and
(e) a lubricant (e.g., magnesium stearate).

In some embodiments, the composition comprises a crystalline form of the compound of formula (I) described herein (e.g., Form A).

In some embodiments, provided is a dosage form containing a pharmaceutical composition as described herein.

In some embodiments, provided is a method for treating an MTAP-deficient and/or an MTA-accumulating disease in a subject in need thereof comprising administering to the subject a therapeutically effective amount of a crystalline form as described herein (e.g., Form A of a compound of formula (I).

In some embodiments, provided is a method for treating an MTAP-deficient and/or an MTA-accumulating disease in a subject in need thereof comprising administering to the subject a pharmaceutical composition as described herein, containing a therapeutically effective amount of the compound of formula (I).

In some embodiments, provided is a process for preparing N-(6-amino-5-methylpyridin-3-yl)-2-((2R,5S)-2-(benzo[d]thiazol-5-yl)-5-methylpiperidin-1-yl)-2-oxoacetamide (a compound of formula (I)), or a salt thereof:

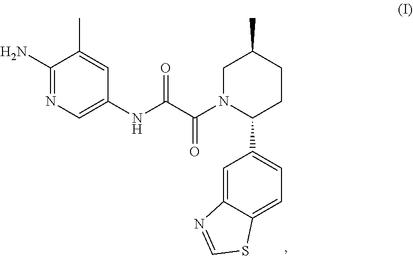 [SMILES: Cc1cc(NC(=O)C(=O)N2C[C@@H](C)CC[C@@H]2c2ccc3scnc3c2)cnc1N]

comprising:

hydrogenating a compound of formula (II):

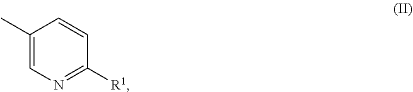

thereby producing a compound of formula (III-a):

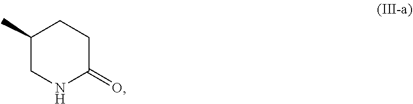

wherein R¹ is a chiral auxiliary.

In some embodiments, provided is a process for preparing N-(6-amino-5-methylpyridin-3-yl)-2-((2R,5S)-2-(benzo[d]thiazol-5-yl)-5-methylpiperidin-1-yl)-2-oxoacetamide (a compound of formula (I)), or a salt thereof:

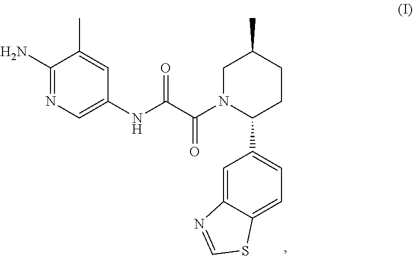 [SMILES: Cc1cc(NC(=O)C(=O)N2C[C@@H](C)CC[C@@H]2c2ccc3scnc3c2)cnc1N]

comprising:

coupling a compound of formula (VI) with a compound of formula (VII):

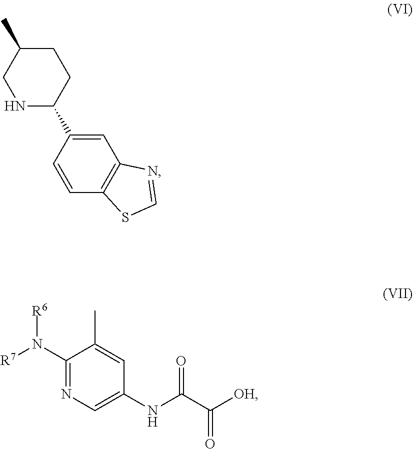

thereby producing a compound of formula (I-a):

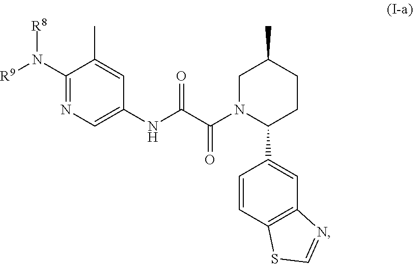

wherein each of R⁶, R⁷, R⁸, and R⁹ is, independently, H or a nitrogen protecting group; and

optionally, if R⁸, R⁹, or both R⁸ and R⁹ are a nitrogen protecting group, the process further comprises a deprotection step to remove the nitrogen protecting group from the compound of formula (I-a), thereby producing the compound of formula (I) or a salt thereof.

### BRIEF DESCRIPTION OF THE DRAWINGS

FIG. 1A is an exemplary X-ray powder diffraction pattern of crystalline Form A of the compound of formula (I).

FIG. 2A shows an exemplary thermogravimetric analysis (TGA) thermogram of the compound of formula (I).

FIG. 2B shows a differential scanning calorimetry (DSC) thermogram for crystalline Form A the compound of formula (I), under a heat-cool-heat protocol, showing a melt peak at about 168° C.

FIG. 2C shows a differential scanning calorimetry (DSC) thermogram for crystalline Form A the compound of formula (I) under a heat-cool-heat protocol, showing a glass transition temperature (T_{g}) of 94.6° C.

FIG. 3A shows the change of water content (thick curve) and relative humidity (thin curve) as a function of time in an exemplary DVS experiment for the compound of formula (I) (Crystalline form A).

FIG. 3B shows the change of water content as a function of relative humidity in an exemplary DVS experiment for the compound of formula (I) (Crystalline form A).

FIG. 4A depicts a viability curve at different concentrations of the PRMT5 inhibitor of formula (I) in a pair of MTAP-isogenic HAP1 (CIVIL-chronic myelogenous leukemia) cell lines, one with intact MTAP and one engineered by CRISPR-mediated MTAP gene knockout. As determined by CellTiter-Glo assay. Data are represented as mean±SD.

FIG. 4B shows a plot of the pharmacodynamic activity of PRMT5 inhibitor of formula (I) to inhibit PRMT5 in the HAP1 MTAP-isogenic cell line pair. The normalized SDMA levels are shown for the MTAP WT and MTAP-null cell line. The data are normalized to a DMSO control for each cell line and presented as mean±SD.

FIG. 4C depicts a viability curve at different concentrations of the PRMT5 inhibitor of formula (I) in a pair of MTAP-isogenic SW1573 (NSCLC-non small cell lung cancer) cell lines, one that is endogenous MTAP deleted and one where exogenous MTAP is reconstituted. As determined by CellTiter-Glo assay. Data are represented as mean±SD.

FIG. 5A depicts a viability curve at different concentrations of the PRMT5 inhibitor of formula (I) in a pair of MTAP-isogenic LN18 (GBM-glioblastoma) cell lines, one endogenous MTAP-deleted and one engineered by reconstituting exogenous MTAP, as determined by CellTiter-Glo assay. Data are represented as mean±SD.

FIG. 5B shows a plot of the EC₅₀ values for 12 glioblastoma cancer cell lines (5 MTAP WT and 7 MTAP-null) that were treated for 7-days with a 9-point dose titration of PRMT5 inhibitor of formula (I) and antiproliferative activity was determined by CellTiter-Glo assay. For each cell line a variable-slope (four parameter) curve was fit and the concentration at which half-maximal efficacy was determined and plotted on the y-axis.

FIG. 6A shows graphs of tumor volumes for an MTAP-null HCT116 xenograft model dosed with PRMT5 inhibitor of formula (I) as a function of time.

FIG. 6B shows graphs of tumor volumes for an MTAP wild type HCT116 xenograft model dosed with PRMT5 inhibitor of formula (I) as a function of time.

FIG. 7 shows a graph of a normalized single SDMA-modified protein level at 30 mpk and 90 mpk BID dosing of PRMT5 inhibitor of formula (I) in HCT116 MTAP-isogenic xenograft models. A single SDMA-modified substrate was quantified by immunoblot and normalized to a loading control from tumors processed 8 hrs post-last dose; n=4 tumors per group. Data are represented as mean±SEM.

FIG. 8 shows waterfall plots of 199 cancer cell lines representing multiple cancer lineages including NSCLC, PDAC, bladder, CNS, and heme malignancies, which were profiled with PRMT5 inhibitor of formula (I) in a 7-day CellTiter-Glo assay.

FIG. 9A shows lineage agnostic responses to PRMT5 inhibitor of formula (I) treatment. FIG. 9A shows waterfall plot for PRMT5 inhibitor of formula (I) activity dosed at 120 mpk BID in MTAP-deleted CDX and PDX models representing the indicated tumor histologies. −% TGI is reported for tumors with Tumor Volume_{final}≥Tumor Volumeᵢₙᵢₜᵢₐₗ (values −100 to 0). % Tumor Volumeᵢₙᵢₜᵢₐₗ −100 is reported for models with Tumor Volume_{final}<Tumor Volumeᵢₙᵢₜᵢₐₗ (values −200 to −100). “Stasis” is defined as 100% TGI and “Complete response” is defined as % Tumor Volumeᵢₙᵢₜᵢₐₗ equal to −100%.

FIG. 9B shows a western blot (probed for SDMA and ACTB as control) as a representative terminal PD analysis of a PRMT5 inhibitor of formula (I)-treated PDX tumor dosed at the indicated levels BID.

FIG. 10 shows a graph of the free concentration of PRMT5 inhibitor of formula (I) in cynomolgus monkeys (N=3/group), in serial samples of cerebrospinal fluid (CSF, a surrogate for free brain concentration) and plasma.

FIG. 11A shows a plot of 111 glioblastoma samples from TCGA Firehouse Legacy that were profiled for MGMT methylation (HM27 and HM450) and expression status (z-scores relative to diploid samples; RNA Seq V2 RSEM). MGMT methylation threshold was defined as >0.2 for further analyses.

FIG. 11B shows the MGMT methylation levels in GBM samples from FIG. 11A segregated by MTAP-status.

FIG. 11C shows the MGMT status from GBM samples in GBM samples from FIG. 11A segregated by MTAP status.

FIG. 11D shows an exemplary 7-day antiproliferative assay and western blot from MTAP-deleted GBM cell lines from FIG. 11A. Cell lines are color-coded by MGMT status according to MGMT immunoblot.

FIG. 11E shows an exemplary western blot from MTAP-deleted GBM cell lines from FIG. 11A.

FIG. 12A shows a graph of tumor volumes for subcutaneous LN18 MTAP-null GBM CDX mouse model dosed with PRMT5 inhibitor of formula (I) at 10 mg/kg, 30 mg/kg and 60 mg/kg BID; data are plotted as mean±SEM; n=8 mice per group.

FIG. 12B shows a PK/PD plot of plasma concentration and single SDMA-modified protein normalized to ACTB (% of vehicle) for subcutaneous LN18 MTAP-null GBM CDX mouse model dosed with PRMT5 inhibitor of formula (I) at 3 mg/kg, 10 mg/kg, 30 mg/kg and 60 mg/kg BID; data are plotted as mean±SEM; n=8 mice per group.

FIG. 12C shows a graph of tumor volumes for subcutaneous U87MG MTAP-null GBM CDX mouse model dosed with PRMT5 inhibitor of formula (I) at 30 mg/kg and 120 mg/kg BID; data are plotted as mean±SEM; n=5 mice per group.

FIG. 12D shows a graph of tumor volumes for subcutaneous MTAP-null GBM PDX mouse model dosed with PRMT5 inhibitor of formula (I) at 30 mg/kg and 120 mg/kg BID; data are plotted as mean±SEM; n=5 mice per group.

FIG. 12E shows a graph of the tumor size as measured by bioluminescence (BLI) for the orthotopic U87MG MTAP-null GBM CDX mouse model treated with 120 mpk BID PRMT5 inhibitor of formula (I) or vehicle.

FIG. 12F shows a Kaplan Meier curve for the probability of survival for the orthotopic U87MG MTAP-null GBM CDX mouse model treated with 120 mpk BID PRMT5 inhibitor of formula (I) or vehicle.

FIG. 12G shows exemplary weekly bioluminescent images at the indicated timepoints for the orthotopic U87MG MTAP-null GBM CDX mouse model treated with 120 mpk BID PRMT5 inhibitor of formula (I) or vehicle until day 34.

FIG. 13A shows a graph of the tumor volume for cholangiocarcinoma PDX tumor-bearing mice that were dosed with 30 mpk and 120 mpk BID of PRMT5 inhibitor of formula (I) for the indicated time period; n=3 mice per group, and data are presented as mean±SEM.

FIG. 13B shows a graph of the tumor volume for NSCLC (adeno) PDX tumor-bearing mice that were dosed with 30 mpk and 120 mpk BID of PRMT5 inhibitor of formula (I) for the indicated time period; n=3 mice per group, and data are presented as mean±SEM.

FIG. 13C shows a graph of the tumor volume for bladder cancer PDX tumor-bearing mice that were dosed with 30 mpk and 120 mpk BID of PRMT5 inhibitor of formula (I) for the indicated time period; n=3 mice per group, and data are presented as mean±SEM.

FIG. 13D shows a graph of the tumor volume for DLBCL OCI-Ly19 CDX tumor-bearing mice that were dosed with 30 mpk and 120 mpk BID of PRMT5 inhibitor of formula (I) for the indicated time period; n=3 mice per group, and data are presented as mean±SEM.

FIG. 13E shows a graph of the tumor volume for NSCLC (squamous) PDX tumor-bearing mice that were dosed with 30 mpk and 120 mpk BID of PRMT5 inhibitor of formula (I) for the indicated time period; n=3 mice per group, and data are presented as mean±SEM.

FIG. 14A. shows a graph of the tumor volume for LU99 (lung cancer) MTAP-null/KRAS G12C CDX tumor-bearing mice that were dosed with 30 mpk and 90 mpk BID of the compound of formula (I) or with 18 mpk QD of sotorasib, and with a combination of 30 mpk and 90 mpk BID of the compound of formula (I) and 18 mpk of sotorasib QD for the indicated time period; n=8 mice per group, and data are presented as mean±SEM.

FIG. 14B. shows a graph of the tumor volume for LU99 (lung cancer) MTAP-null/KRAS G12C CDX tumor-bearing mice that were dosed with 30 mpk and 90 mpk BID of the compound of formula (I) or with 100 mpk QD of sotorasib, and with a combination of 30 mpk and 90 mpk BID of the compound of formula (I) and 100 mpk of sotorasib QD for the indicated time period; n=8 mice per group, and data are presented as mean±SEM.

FIG. 15. shows a graph of the tumor volume for LU99 (lung cancer) MTAP-null subcutaneous CDX tumor-bearing mice that were dosed with: 80 mpk BID of the compound of formula (I); 50 mpk QD of palbociclib; 50 mpk QD of abemaciclib; a combination of 80 mpk BID of the compound of formula (I) and 50mpk QD of palbociclib; a combination of 80 mpk BID of the compound of formula (I) and 50mpk QD of abemaciclib, respectively; for the indicated time period; n=8 mice per group.

FIG. 16A. Combination of AG-270, a MAT2A inhibitor with the compound of formula (I) in a 7-day viability assay in the MTAP-null SW1573 (NSCLC) cancer cell line demonstrates enhanced cellular viability defects.

FIG. 16B. Combination of AG-270, a MAT2A inhibitor with the compound of formula (I) in a 7-day viability assay in the MTAP-null LN18 (GBM) cancer cell line demonstrates enhanced cellular viability defects.

FIG. 16C. Combination of AG-270, a MAT2A inhibitor with the compound of formula (I) in a 7-day viability assay in the MTAP-null RT112/84 (bladder) cancer cell line demonstrates enhanced cellular viability defects.

### DETAILED DESCRIPTION

As generally described herein, the present disclosure provides pharmaceutical compositions containing a PRMT5 inhibitor (e.g., an MTA-uncompetitive PRMT5 inhibitor) e.g., a compound of formula (I):

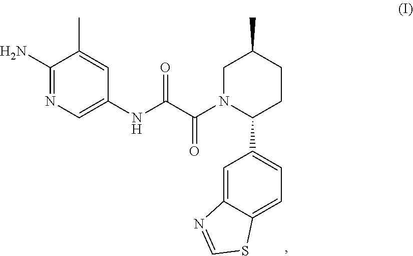 [SMILES: Cc1cc(NC(=O)C(=O)N2C[C@@H](C)CC[C@@H]2c2ccc3scnc3c2)cnc1N]

and crystalline forms thereof, methods of making the pharmaceutical compositions, and methods of using the pharmaceutical compositions to treat medical conditions, diseases, and disorders e.g., proliferation diseases such as cancers.

### Definitions

As used in the present disclosure, the following words and phrases are generally intended to have the meanings as set forth below unless expressly indicated otherwise or the context in which they are used indicates otherwise.

Unless defined otherwise, all technical and scientific terms used herein have the same meaning as commonly understood by one of ordinary skill in the art to which this invention belongs. The abbreviations used herein have their conventional meaning within the chemical and biological arts. The chemical structures and formulae set forth herein are constructed according to the standard rules of chemical valency known in the chemical arts.

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. Where elements are presented as lists, e.g., in Markush group format, each subgroup of the elements is also disclosed, and any element(s) can be removed from the group. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

The articles “a” and “an” are used in this disclosure to refer to one or more than one (i.e., to at least one) of the grammatical object of the article, unless the context is inappropriate. By way of example, in certain contexts, “an element” means one element and/or in certain contexts more than one element. By way of another example, in certain contexts “a filler” means one filler and/or in certain contexts more than one filler (e.g., a mixture of two or more fillers).

The term “and/or” is used in this disclosure to mean either “and” or “or” unless indicated otherwise.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred from the context.

At various places in the present specification, variables or parameters are disclosed in groups or in ranges. It is specifically intended that the description include each and every individual subcombination of the members of such groups and ranges. For example, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

As a general matter, compositions specifying a percentage are by weight unless otherwise specified. Further, if a variable is not accompanied by a definition, then the previous definition of the variable controls.

As used herein, “XRPD” refers to X-ray powder diffraction. An XRPD pattern is an x-y graph with 20 (diffraction angle) plotted on the x-axis and intensity plotted on the y-axis. These are the diffraction peaks which may be used to characterize a crystalline material. The diffraction peaks are usually represented and referred to by their position on the x-axis rather than the intensity of the diffraction peaks on the y-axis because diffraction peak intensity can be particularly sensitive to sample orientation (see Pharmaceutical Analysis, Lee & Web, pp. 255-257 (2003)). Thus, intensity is not typically used by those of skill in the art to characterize a crystalline material. As with any data measurement, there may be variability in XRPD data. In addition to the variability in diffraction peak intensity, there may also be variability in the position of the diffraction peaks on the x-axis. This variability can, however, typically be accounted for when reporting the positions of diffraction peaks for purposes of characterization. Such variability in the position of diffraction peaks along the x-axis may be derived from several sources. One such source can be sample preparation. Samples of the same crystalline material prepared under different conditions may yield slightly different diffractograms. Factors such as particle size, moisture content, solvent content, temperature, and orientation may all affect how a sample diffracts X-rays. Another source of variability comes from instrument parameters. Different X-ray powder diffractometers operate using different parameters and may lead to slightly different diffraction patterns from the same crystalline material. Likewise, different software packages process XRPD data differently and this may also lead to variability. These and other sources of variability are known to those of ordinary skill in the art. Due to such sources of variability, the values of each X-ray diffraction peak may be preceded with the term “about” or proceeded with an appropriate range defining the experimental variability (e.g., ±0.1°, ±0.2°, ±0.3°, ±0.4°, ±0.5°, etc.).

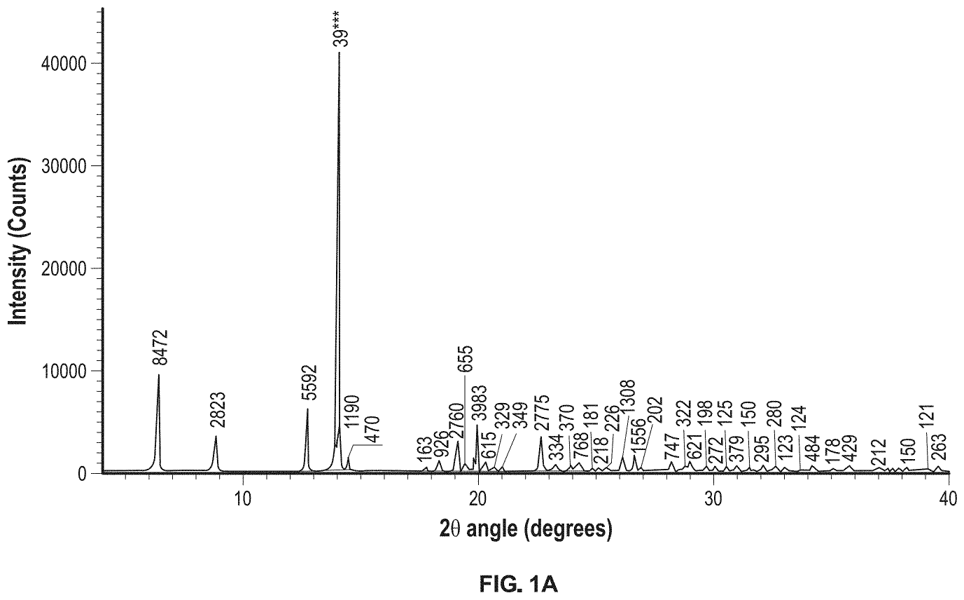

Crystalline forms, such as crystalline forms of a compound of formula (I), are readily analyzed by XRPD. The data from x-ray powder diffraction may be used in multiple ways to characterize crystalline forms. For example, the entire x-ray powder diffraction pattern output from a diffractometer may be used to characterize a crystalline form (e.g., of a compound of formula (I). A smaller subset of such data, however, may also be suitable and used for characterizing such crystalline forms. Indeed, often even a single x-ray powder diffraction peak may be used to characterize such a crystalline form. With respect to crystalline forms of a compound of formula (I), any one or more of the peaks in the x-ray powder diffraction pattern of FIG. 1A may be used to characterize the crystalline form of a compound of formula (I) disclosed herein.

The term “characteristic peaks” when referring to the peaks in an XRPD pattern of a crystalline form of a given chemical entity (e.g., a crystalline form of a compound of formula (I)) refers to a collection of specific diffraction peaks whose values span a range of 20 values (e.g., 0°-40° that are, as a whole, unique to that specific crystalline form.

As used herein, “crystalline” refers to a solid phase of a given chemical entity having well-defined 3-dimensional structural order. The atoms, ions, and/or molecules are arranged in a regular, periodic manner within a repeating 3-dimensional lattice. In various embodiments, a crystalline material may comprise one or more discreet crystalline forms.

As used herein, the terms “crystalline form”, “crystalline solid form,” “crystal form,” “solid form,” and related terms herein refer to crystalline modifications comprising a given substance (e.g., the compound of formula (I)), including single-component crystal forms and multiple-component crystal forms, and including, but not limited to, polymorphs, solvates, hydrates, and salts.

The term “substantially crystalline” refers to solid forms that may be at least a particular weight percent crystalline. Particular weight percentages may include 70%, 75%, 80%, 85%, 87%, 88%, 89%, 90%, 91%, 92%, 93%, 94%, 95%, 96%, 97%, 98%, 99%, 99.5%, 99.9%, or any percentage between 70% and 100%. In certain embodiments, the particular weight percent of crystallinity is at least 90%. In certain other embodiments, the particular weight percent of crystallinity is at least 95%. In some embodiments, the compound of formula (I) can be a substantially crystalline sample of any of the crystalline solid forms described herein (e.g., a crystalline form with the XRPD pattern shown in FIG. 1A).

The term “substantially pure” relates to the composition of a specific crystalline solid form (e.g., a crystalline form of the compound of formula (I)) that may be at least a particular weight percent free of impurities and/or other solid forms. Particular weight percentages may include 70%, 75%, 80%, 85%, 90%, 95%, 99%, or any percentage between 70% and 100%. In certain embodiments, the compound of formula (I) can be a substantially pure sample of any of the crystalline solid forms described herein, (e.g., a crystalline form with the XRPD pattern shown in FIG. 1A (Form A)). In certain embodiments, the compound of formula (I) can be a substantially pure crystalline form with the XRPD pattern shown in FIG. 1A (Form A).

As used herein, the term “anhydrous” or “anhydrate” when referring to a crystalline form (e.g., a crystalline form of the compound of formula (I)) means that no water molecules form a portion of the unit cell of the crystalline form. An anhydrous crystalline form may nonetheless contain water molecules that do not form part of the unit cell of the anhydrous crystalline form (e.g., as residual solvent molecule left behind from the production of the crystalline form). In a preferred embodiment, water can make up about 0.5% by weight of the total composition of a sample of an anhydrous form. In a more preferred embodiment, water can make up about 0.2% by weight of the total composition of a sample of an anhydrous form. In some embodiments, a sample of an anhydrous crystalline form of the compound of formula (I) contains no water molecules, e.g., no detectable amount of water.

As used herein, the term “desolvated” or “unsolvated” when referring to a crystalline form (e.g., a crystalline form of the compound of formula (I)) means that no solvent molecules form a portion of the unit cell of the crystalline form. An unsolvated crystalline form may nonetheless contain solvent molecules that do not form part of the unit cell of the unsolvated crystalline form (e.g., as residual solvent molecule left behind from the production of the crystalline form). In a preferred embodiment, the solvent can make up 0.5% by weight of the total composition of a sample of an unsolvated form. In a more preferred embodiment, solvent can make up 0.2% by weight of the total composition of a sample of an unsolvated form. In some embodiments, a sample of an unsolvated crystalline form of the compound of formula (I) contains no solvent molecules, e.g., no detectable amount of solvent.

As used herein, the terms “polymorph,” “polymorphic form,” “polymorphs,” “polymorphic forms” and related terms herein refer to two or more crystal forms that consist essentially of the same molecule, molecules, or ions (e.g., the compound of formula (I)). Different polymorphs may exhibit different physicochemical properties including, but not limited to, melting temperatures, solubilities, dissolution rates, and physical stabilities as a result of differences in the arrangement or conformation of the molecules or ions in the crystal lattice.

The term “solvate” when referring to a crystalline form of the compound of formula (I) means that solvent molecules (e.g., organic solvents and water), form a portion of the unit cell of the crystalline form. Solvates that contain water as the solvent are also referred to herein as “hydrates.”

As used herein, “dissolution profile” refers to dissolution testing of a drug substance or drug product at multiple time points. Dissolution profiles for drug substances (e.g., the compound of formula (I)) or drug products (e.g., the pharmaceutical compositions described herein) may be performed for characterization and quality control to ensure the drug is released at a defined range of rates in a well-defined dissolution aqueous media that is at least sink conditions for that drug, or in biorelevant media such as simulated gastric or intestinal fluids representing either the fasted or fed states. In certain cases, but not others, dissolution testing may be predictive of or give insight into in vivo bioavailability of the drug substance. Dissolution testing may be performed using USP testing protocols and dissolution apparatus.

As used herein, “granulation” refers to a process of forming granules from a powdered or particulate material. As used herein, “Dry granulation” refers to a process in which granules are formed without the presence of a liquid solution and may be useful in the preparation of granules of materials sensitive to heat, moisture, or solvents. For example, roller compaction is a dry granulation process. As used herein, “Wet granulation” refers to the formation of granules wherein the particles are bound together using a binder or a liquid solution. Examples of wet granulation are high shear granulation and fluid bed granulation.

As used herein, “pharmaceutical composition” or “pharmaceutical formulation” refer to the combination of a therapeutically active agent with a pharmaceutically acceptable excipient, inert or active, making the composition especially suitable for diagnostic or therapeutic use in vivo or ex vivo.

“Pharmaceutically acceptable” refers to compounds, molecular entities, compositions, materials and/or dosage forms that do not produce an adverse, allergic or other untoward reaction when administered to an animal, or human, as appropriate; or means approved or approvable by a regulatory agency of the federal or a state government or the corresponding agency in countries other than the United States, or that is listed in the U.S. Pharmacopoeia or other generally recognized pharmacopoeia for use in animals, and more particularly, in humans.

As used herein, “pharmaceutically acceptable salt” refers to any salt of an acidic or a basic group that may be present in a compound of the present disclosure (e.g., the compound of formula (I)), which salt is compatible with pharmaceutical administration.

As is known to those of skill in the art, “salts” of compounds may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic and benzenesulfonic acid. Other acids, such as oxalic, while not in themselves pharmaceutically acceptable, may be employed in the preparation of salts useful as intermediates in obtaining the compounds described herein and their pharmaceutically acceptable acid addition salts.

Examples of bases include, but are not limited to, alkali metal (e.g., sodium and potassium) hydroxides, alkaline earth metal (e.g., magnesium and calcium) hydroxides, ammonia, and compounds of formula NW⁴⁺, wherein W is C₁₋₄ alkyl, and the like.

Examples of salts include, but are not limited, to acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present disclosure compounded with a suitable cation such as Na⁺, K⁺, Ca²⁺, NH⁴⁺ and NW⁴⁺ (where W can be a C₁₋₄ alkyl group), and the like.

For therapeutic use, salts of the compounds of the present disclosure are contemplated as being pharmaceutically acceptable. However, salts of acids and bases that are non-pharmaceutically acceptable may also find use, for example, in the preparation or purification of a pharmaceutically acceptable compound.

As used herein, “pharmaceutically acceptable excipient” refers to a substance that aids the administration of an active agent to and/or absorption by a subject and can be included in the compositions of the present disclosure without causing a significant adverse toxicological effect on the patient. Non-limiting examples of pharmaceutically acceptable excipients include binders, diluents, carriers, adjuvants, fillers (e.g., brittle diluents or fillers and ductile diluents or fillers), disintegrants, lubricants, coatings, sweeteners, flavors, gelatins, carbohydrates such as lactose, amylose or starch, fatty acid esters, hydroxypropylmethylcellulose, polyvinyl pyrrolidine, and colors, and the like. For examples of excipients, see Gennaro, Remington's Pharmaceutical Sciences, 18^{th} Ed., Mack Publ. Co., Easton, Pa. (1990) or Shesky, Hancock, Moss and Goldfarb, Handbook of Pharmaceutical Excipients, 9^{th} Ed. Pharmaceutical Press, London, UK (2020).

Examples of diluents or fillers include, but are not limited to, a sugar (e.g., mannitol, lactose, sorbitol, lactitol, erythritol, sucrose, fructose, glucose, agarose, maltose, isomalt, polydextrose, and combinations thereof), an inorganic material (e.g., dibasic calcium phosphate, hydroxyapatite, sodium carbonate, sodium bicarbonate, calcium carbonate, calcium sulfate, magnesium carbonate, magnesium oxide, bentonite, kaolin), calcium lactate, a starch (e.g., a pregelatinized starch), a microcrystalline cellulose, a silicified microcrystalline cellulose, a polysaccharide, a cellulose (e.g., a hydroxypropylcellulose, a hypromellose, a carboxymethylcellulose, a methylcellulose, a hydroxypropylmethylcellulose, a hydroxyethylcellulose), a dextrin, a maltodextrin, an alginate, a collagen, a polyvinylpyrrolidone, a polyvinylacrylate, polyethylene oxide, and polyethylene glycol. Sugar is defined herein to include sugar alcohols.

Examples of disintegrants include, but are not limited to, alginic acid, an alginate, primogel, a cellulose (e.g., hydroxypropylcellulose), polacrillin potassium, sodium starch glycolate, sodium croscarmellose, a polyplasdone (e.g., a crospovidone), and a starch (e.g., corn starch, pregelatinized starch, hydroxypropyl starch, and carboxymethyl starch).

Examples of binders include, but are not limited to, a hydroxypropylcellulose, hydroxyethylcellulose, a hydroxypropylmethycellulose (e.g., a low viscosity hydroxypropylmethycellulose), a sugar, a polyvinylpyrrolidone, a polyvinyl alcohol, a polyvinyl acetate, a polydextrose, a chitosan, a carrageenan, carbophil, a microcrystalline cellulose, gum tragacanth, guar gum, gellan gum, gelatin, and a starch (e.g., corn starch).

Examples of wetting agents include, but are not limited to, a poloxamer (e.g., poloxamer 407), sodium dodecyl sulfate, sodium lauryl sulfate (SLS), sodium stearyl fumarate (SSF), a polydimethylsiloxane, a polysorbate (e.g., polyoxyethylene 20 sorbitan mono-oleate (Tween® 20)), sorbitan monooleate, sorbitan trioleate, sorbitan laurate, sorbitan stearate, sorbitan monopalmitate, lecithin, sodium taurocholate, ursodeoxycholate, polyethoxylated castor oil, cetyl trimethylammonium bromide, nonoxynol, \acute{α}-tocopherol polyethylene glycol 1000 succinate, and docusate sodium.

Examples of lubricants and glidants include, but are not limited to, a wax, a glyceride, a light mineral oil, a polyethylene glycol, sodium stearyl fumarate, magnesium stearate, stearic acid, hydrogenated oil (e.g., hydrogenated vegetable oil), an alkyl sulfate, sodium benzoate, sodium acetate, glyceryl behenate, palmitic acid, and coconut oil.

Examples of glidants include, but are not limited to, colloidal silicon dioxide, colloidal silicon dioxide, talc, kaolin, bentonite, and activated carbon/charcoal.

Examples of colorants include, but are not limited to, titanium dioxide, aluminum lakes, iron oxides and carbon black.

Examples of coatings include but are not limited to, a film forming polymer (e.g., a hypromellose, a methyl cellulose, an ethylcellulose, cellulose acetate, a hydroxypropylmethyl cellulose, a hydroxypropyl cellulose, hydroxypropylmethyl cellulose acetate succinate, cellulose acetate phthalate, a polyvinylpyrrolidone, polyvinyl alcohol, a Eudragit/acrylate) and a plasticizer (e.g., triacetin, polyethylene glycol, propylene glycol).

Pharmaceutical compositions for oral administration (e.g., pharmaceutical compositions of the compound of formula (I) described herein) can take the form of bulk liquid solutions or suspensions or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include pills, tablets, capsules or the like in the case of solid compositions.

A “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal.

As used herein, “solid dosage form” means a pharmaceutical dose(s) in solid form, e.g., tablets, capsules, granules, powders, minitabs, sachets, stickpacks, reconstitutable powders, dry powder inhalers, lozenges, and chewables.

As used herein, “administering” means oral administration, administration as a pulmonary, suppository, intramuscular administration, intrathecal administration, intranasal administration or subcutaneous administration, or the implantation of a slow-release device, e.g., a mini-osmotic pump, to a subject. Administration is by any route, including transmucosal (e.g., buccal, sublingual, palatal, gingival, nasal, vaginal, rectal, or). Parenteral administration includes, e.g., intramuscular and subcutaneous. Other modes of delivery include, but are not limited to, the use of liposomal formulations, etc. By “co-administer” it is meant that a composition described herein is administered at the same time, just prior to, or just after the administration of one or more additional therapies (e.g., anti-cancer agent, chemotherapeutic, or treatment for a neurodegenerative disease). The compound of formula (I) can be administered alone or can be co-administered to the patient. Co-administration is meant to include simultaneous or sequential administration of the compound individually or in combination (more than one compound or agent). Thus, the preparations can also be combined, when desired, with other active substances (e.g., to reduce metabolic degradation).

The terms “disease,” “disorder,” and “condition” are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition (“therapeutic treatment”), and also contemplates an action that occurs before a subject begins to suffer from the specified disease, disorder or condition (“prophylactic treatment”). In one embodiment, the compounds provided herein are contemplated to be used in methods of therapeutic treatment wherein the action occurs while a subject is suffering from the specified disease, disorder or condition and results in a reduction in the severity of the disease, disorder or condition, or retardation or slowing of the progression of the disease, disorder or condition. In an alternate embodiment, the compounds provided herein are contemplated to be used in methods of prophylactic treatment wherein the action occurs before a subject begins to suffer from the specified disease, disorder or condition and results in preventing a disease, disorder or condition, or one or more symptoms associated with the disease, disorder or condition, or preventing the recurrence of the disease, disorder or condition.

In general, the “effective amount” of a compound refers to an amount sufficient to elicit the desired biological response e.g., to treat a disease or disorder described herein. As will be appreciated by those of ordinary skill in this art, the effective amount of a compound of the disclosure may vary depending on such factors as the desired biological endpoint, the pharmacokinetics of the compound, the disease being treated, the mode of administration, and the age, health, and condition of the subject. An effective amount encompasses therapeutic and prophylactic treatment (i.e., encompasses a “therapeutically effective amount” and a “prophylactically effective amount”).

As used herein, and unless otherwise specified, a “therapeutically effective amount” of a compound is an amount sufficient to provide a therapeutic benefit in the therapeutic treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the therapeutic treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

As used herein, and unless otherwise specified, a “prophylactically effective amount” of a compound is an amount sufficient to prevent a disease, disorder or condition, or one or more symptoms associated with the disease, disorder or condition, or prevent its recurrence. A prophylactically effective amount of a compound means an amount of a therapeutic agent, alone or in combination with other agents, which provides a prophylactic benefit in the prevention of the disease, disorder or condition. The term “prophylactically effective amount” can encompass an amount that improves overall prophylaxis or enhances the prophylactic efficacy of another prophylactic agent.

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high-pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, N Y, 1962); and Wilen, Tables of Resolving Agents and Optical Resolutions p. 268 (E. L. Eliel, Ed., Univ. of Notre Dame Press, Notre Dame, Ind. 1972). Additionally encompassed are compounds described herein as individual isomers substantially free of other isomers, and alternatively, as mixtures of various isomers.

The “enantiomeric excess” (“e.e.”) or “% enantiomeric excess” (“% e.e.”) of a composition as used herein refers to an excess of one enantiomer relative to the other enantiomer present in the composition. For example, a composition can contain 90% of one enantiomer, e.g., the S enantiomer, and 10% of the other enantiomer, i.e., the R enantiomer.

e.e. =(90−10)/100=80%.

Thus, a composition containing 90% of one enantiomer and 10% of the other enantiomer is said to have an enantiomeric excess of 80%.

The “diastereomeric excess” (“d.e.”) or “% diastereomeric excess” (“% d.e.”) of a composition as used herein refers to an excess of one diastereomer relative to one or more different diastereomers present in the composition. For example, a composition can contain 90% of one diastereomer, and 10% of one or more different diastereomers.

d.e. =(90−10)/100=80%.

Thus, a composition containing 90% of one diastereomers and 10% of one or more different diastereomers is said to have a diastereomeric excess of 80%.

When a range of values is listed, it is intended to encompass each value and sub-range within the range. For example, “C₁₋₆ alkyl” is intended to encompass, C₁, C₂, C₃, C₄, C₅, C₆, C₁-6, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl. It should also be understood that when described herein any of the moieties defined forth below may be substituted with a variety of substituents, and that the respective definitions are intended to include such substituted moieties within their scope as set out below. Unless otherwise stated, the term “substituted” is to be defined as set out below. It should be further understood that the terms “groups” and “radicals” can be considered interchangeable when used herein. The articles “a” and “an” may be used herein to refer to one or to more than one (i.e. at least one) of the grammatical objects of the article. By way of example “an analogue” means one analogue or more than one analogue.

The term “unsaturated bond” refers to a double or triple bond.

The term “unsaturated” or “partially unsaturated” refers to a moiety that includes at least one double or triple bond.

The term “saturated” refers to a moiety that does not contain a double or triple bond, i.e., the moiety only contains single bonds.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

“Aliphatic” refers to an alkyl, alkenyl, alkynyl, or carbocyclyl group, as defined herein.

“Cycloalkylalkyl” refers to an alkyl radical in which the alkyl group is substituted with a cycloalkyl group. Typical cycloalkylalkyl groups include, but are not limited to, cyclopropylmethyl, cyclobutylmethyl, cyclopentylmethyl, cyclohexylmethyl, cycloheptylmethyl, cyclooctylmethyl, cyclopropylethyl, cyclobutylethyl, cyclopentylethyl, cyclohexylethyl, cycloheptylethyl, and cyclooctylethyl, and the like.

“Heterocyclylalkyl” refers to an alkyl radical in which the alkyl group is substituted with a heterocyclyl group. Typical heterocyclylalkyl groups include, but are not limited to, pyrrolidinylmethyl, piperidinylmethyl, piperazinylmethyl, morpholinylmethyl, pyrrolidinylethyl, piperidinylethyl, piperazinylethyl, morpholinylethyl, and the like.

“Aralkyl” or “arylalkyl” is a subset of alkyl and aryl, as defined herein, and refers to an optionally substituted alkyl group substituted by an optionally substituted aryl group.

“Alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”, also referred to herein as “lower alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₅) and the like. Unless otherwise specified, each instance of an alkyl group is independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In certain embodiments, the alkyl group is unsubstituted C₁₋₁₀ alkyl (e.g., —CH₃). In certain embodiments, the alkyl group is substituted C₁₋₁₀ alkyl. Common alkyl abbreviations include Me (—CH₃), Et (—CH₂CH₃), ⁱPr (—CH(CH₃)₂), ⁿPr (—CH₂CH₂CH₃), ⁿBu (—CH₂CH₂CH₂CH₃), or ⁱBu (—CH₂CH(CH₃)₂).

“Alkylene” refers to an alkyl group wherein two hydrogens are removed to provide a divalent radical, and which may be substituted or unsubstituted. Unsubstituted alkylene groups include, but are not limited to, methylene (—CH₂—), ethylene (—CH₂CH₂—), propylene (—CH₂CH₂CH₂—), butylene (—CH₂CH₂CH₂CH₂—), pentylene (—CH₂CH₂CH₂CH₂CH₂—), hexylene (—CH₂CH₂CH₂CH₂CH₂CH₂—), and the like. Exemplary substituted alkylene groups, e.g., substituted with one or more alkyl (methyl) groups, include but are not limited to, substituted methylene (—CH(CH₃)—, (—C(CH₃)₂—), substituted ethylene (—CH(CH₃)CH₂—, —CH₂CH(CH₃)—, —C(CH₃)₂CH₂—, —CH₂C(CH₃)₂—), substituted propylene (—CH(CH₃)CH₂CH₂—, —CH₂CH(CH₃)CH₂—, —CH₂CH₂CH(CH₃)—, —C(CH₃)₂CH₂CH₂—, —CH₂C(CH₃)₂CH₂—, —CH₂CH₂C(CH₃)₂—), and the like. When a range or number of carbons is provided for a particular alkylene group, it is understood that the range or number refers to the range or number of carbons in the linear carbon divalent chain. Alkylene groups may be substituted or unsubstituted with one or more substituents as described herein.

“Alkenyl” refers to a radical of a straight-chain or branched hydrocarbon group having from 2 to 20 carbon atoms, one or more carbon-carbon double bonds (e.g., 1, 2, 3, or 4 carbon-carbon double bonds), and optionally one or more carbon-carbon triple bonds (e.g., 1, 2, 3, or 4 carbon-carbon triple bonds) (“C₂₋₂₀ alkenyl”). In certain embodiments, alkenyl does not contain any triple bonds. In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent. In certain embodiments, the alkenyl group is unsubstituted C₂₋₁₀ alkenyl. In certain embodiments, the alkenyl group is substituted C₂₋₁₀ alkenyl.

“Alkynyl” refers to a radical of a straight-chain or branched hydrocarbon group having from 2 to 20 carbon atoms, one or more carbon-carbon triple bonds (e.g., 1, 2, 3, or 4 carbon-carbon triple bonds), and optionally one or more carbon-carbon double bonds (e.g., 1, 2, 3, or 4 carbon-carbon double bonds) (“C₂₋₂₀ alkynyl”). In certain embodiments, alkynyl does not contain any double bonds. In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently optionally substituted, i.e., unsubstituted (an “unsubstituted alkynyl”) or substituted (a “substituted alkynyl”) with one or more substituents; e.g., for instance from 1 to 5 substituents, 1 to 3 substituents, or 1 substituent. In certain embodiments, the alkynyl group is unsubstituted C₂₋₁₀ alkynyl. In certain embodiments, the alkynyl group is substituted C₂₋₁₀ alkynyl.

The term “heteroalkyl,” as used herein, refers to an alkyl group, as defined herein, which further comprises 1 or more (e.g., 1, 2, 3, or 4) heteroatoms (e.g., oxygen, sulfur, nitrogen, boron, silicon, phosphorus) within the parent chain, wherein the one or more heteroatoms is inserted between adjacent carbon atoms within the parent carbon chain and/or one or more heteroatoms is inserted between a carbon atom and the parent molecule, i.e., between the point of attachment. In certain embodiments, a heteroalkyl group refers to a saturated group having from 1 to 10 carbon atoms and 1, 2, 3, or 4 heteroatoms (“heteroC₁₋₁₀ alkyl”). In some embodiments, a heteroalkyl group is a saturated group having 1 to 9 carbon atoms and 1, 2, 3, or 4 heteroatoms (“heteroC₁₋₉ alkyl”). In some embodiments, a heteroalkyl group is a saturated group having 1 to 8 carbon atoms and 1, 2, 3, or 4 heteroatoms (“heteroC₁₋₈ alkyl”). In some embodiments, a heteroalkyl group is a saturated group having 1 to 7 carbon atoms and 1, 2, 3, or 4 heteroatoms (“heteroC₁₋₇ alkyl”). In some embodiments, a heteroalkyl group is a group having 1 to 6 carbon atoms and 1, 2, or 3 heteroatoms (“heteroC₁₋₆ alkyl”). In some embodiments, a heteroalkyl group is a saturated group having 1 to 5 carbon atoms and 1 or 2 heteroatoms (“heteroC₁₋₅ alkyl”). In some embodiments, a heteroalkyl group is a saturated group having 1 to 4 carbon atoms and 1 or 2 heteroatoms (“heteroC₁₋₄ alkyl”). In some embodiments, a heteroalkyl group is a saturated group having 1 to 3 carbon atoms and 1 heteroatom (“heteroC₁₋₃ alkyl”). In some embodiments, a heteroalkyl group is a saturated group having 1 to 2 carbon atoms and 1 heteroatom (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms (“heteroC₂₋₆alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₀ alkyl. In certain embodiments, the heteroalkyl group is a substituted heteroC₁-10 alkyl. Exemplary heteroalkyl groups include: —CH₂OH, —CH₂OCH₃, —CH₂NH₂, —CH₂NH(CH₃), —CH₂N(CH₃)₂, —CH₂CH₂OH, —CH₂CH₂OCH₃, —CH₂CH₂NH₂, —CH₂CH₂NH(CH₃), —CH₂CH₂N(CH₃)₂.

“Aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Particularly aryl groups include phenyl, naphthyl, indenyl, and tetrahydronaphthyl. Unless otherwise specified, each instance of an aryl group is independently optionally substituted, i.e., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl.

In certain embodiments, an aryl group is substituted with one or more of groups selected from halo, C₁-C₈ alkyl, C₁-C₈ haloalkyl, cyano, hydroxy, C₁-C₈ alkoxy, and amino.

Examples of representative substituted aryls include the following

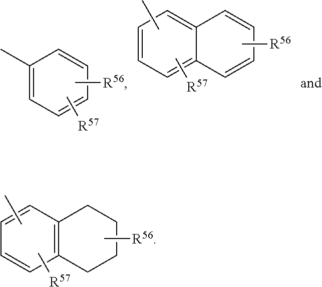

wherein one of R⁵⁶ and R⁵⁷ may be hydrogen and at least one of R⁵⁶ and R⁵⁷ is each independently selected from C₁-C₈ alkyl, C₁-C₈ haloalkyl, 4-10 membered heterocyclyl, alkanoyl, C₁-C₈ alkoxy, heteroaryloxy, alkylamino, acylamino, heteroarylamino, NR⁵⁸COR⁵⁹, NR⁵⁸SOR⁵⁹NR⁵⁸SO₂R⁵⁹, COOalkyl, COOaryl, CONR⁵⁸R⁵⁹, CONR⁵⁸OR⁵⁹, NR⁵⁸R⁵⁹, SO₂NR⁵⁸R⁵⁹, S-alkyl, Soalkyl, SO₂alkyl, Saryl, Soaryl, SO₂aryl; or R⁵⁶ and R⁵⁷ may be joined to form a cyclic ring (saturated or unsaturated) from 5 to 8 atoms, optionally containing one or more heteroatoms selected from the group consisting of N, O, or S. R⁶⁰ and R⁶¹ are independently hydrogen, C₁-C₈ alkyl, C₁-C₄ haloalkyl, C₃-C₁₀ cycloalkyl, 4-10 membered heterocyclyl, C₆-C₁₀ aryl, substituted C₆-C₁₀ aryl, 5-10 membered heteroaryl, or substituted 5-10 membered heteroaryl.

Fused aryl” refers to an aryl having two of its ring carbons in common with a second aryl or heteroaryl ring or with a carbocyclyl or heterocyclyl ring.

“Heteroaryl” refers to a radical of a 5-10 membered monocyclic or bicyclic 4n+2 aromatic ring system (e.g., having 6 or 10 π electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring. In such instances, unless otherwise specified, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently optionally substituted, i.e., unsubstituted (an “unsubstituted heteroaryl”) or substituted (a “substituted heteroaryl”) with one or more substituents. In certain embodiments, the heteroaryl group is unsubstituted 5-14 membered heteroaryl. In certain embodiments, the heteroaryl group is substituted 5-14 membered heteroaryl. In some embodiments, a heteroaryl group is a bicyclic 8-12 membered aromatic ring system having ring carbon atoms and 1-6 ring heteroatoms provided in the aromatic ring system, wherein each heteroatom is independently selected from nitrogen, oxygen, and sulfur (“8-12 membered bicyclic heteroaryl”). In some embodiments, a heteroaryl group is an 8-10 membered bicyclic aromatic ring system having ring carbon atoms and 1-6 ring heteroatoms provided in the aromatic ring system, wherein each heteroatom is independently selected from nitrogen, oxygen, and sulfur (“8-10 membered bicyclic heteroaryl”). In some embodiments, a heteroaryl group is a 9-10 membered bicyclic aromatic ring system having ring carbon atoms and 1-6 ring heteroatoms provided in the aromatic ring system, wherein each heteroatom is independently selected from nitrogen, oxygen, and sulfur (“9-10 membered bicyclic heteroaryl”). Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

Examples of representative heteroaryls include the following:

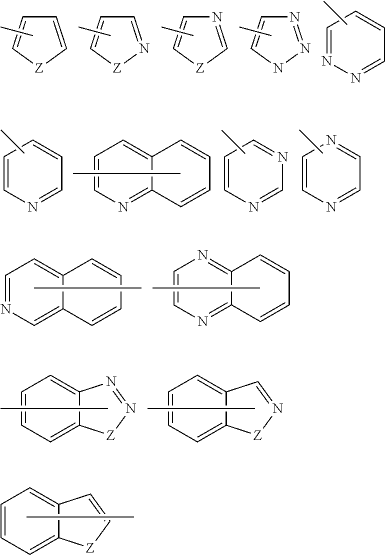

wherein each Z is selected from carbonyl, N, NR⁶⁵, O, and S; and R⁶⁵ is independently hydrogen, C₁-C₈ alkyl, C₃-C₁₀ cycloalkyl, 4-10 membered heterocyclyl, C₆-C₁₀ aryl, and 5-10 membered heteroaryl.

In the structures described herein, a substituent attached to a polycyclic (e.g., bicyclic or tricyclic) cycloalkyl, heterocyclyl, aryl or heteroaryl with a bond that spans two or more rings is understood to mean that the substituent can be attached at any position in each of the rings.

“Heteroaralkyl” or “heteroarylalkyl” is a subset of “alkyl” and refers to an alkyl group substituted by a heteroaryl group, wherein the point of attachment is on the alkyl moiety.

The term “carbocyclyl” or “carbocyclic” refers to a radical of a non-aromatic monocyclic, bicyclic, or tricyclic or polycyclic hydrocarbon ring system having from 3 to 14 ring carbon atoms (“C₃₋₁₄ carbocyclyl”) and zero heteroatoms in the non-aromatic ring system. Carbocyclyl groups include fully saturated ring systems (e.g., cycloalkyls), and partially saturated ring systems. In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like.

As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

The term “cycloalkyl” as employed herein includes saturated cyclic, bicyclic, tricyclic, or polycyclic hydrocarbon groups having 3 to 14 carbons containing the indicated number of rings and carbon atoms (for example a C₃-C₁₄ monocyclic, C₄-C₁₄ bicyclic, C₅-C₁₄ tricyclic, or C₆-C₁₄ polycyclic cycloalkyl). In some embodiments “cycloalkyl” is a monocyclic cycloalkyl. In some embodiments, a monocyclic cycloalkyl has 3-14 ring carbon atoms. (“C₃₋₁₄ monocyclic cycloalkyl”). In some embodiments, a monocyclic cycloalkyl group has 3 to 10 ring carbon atoms (“C₃₋₁₀ monocyclic cycloalkyl”). In some embodiments, a monocyclic cycloalkyl group has 3 to 8 ring carbon atoms (“C₃₋₈ monocyclic cycloalkyl”). In some embodiments, a monocyclic cycloalkyl group has 3 to 6 ring carbon atoms (“C₃₋₆ monocyclic cycloalkyl”). In some embodiments, a monocyclic cycloalkyl group has 4 to 6 ring carbon atoms (“C₄₋₆ monocyclic cycloalkyl”). In some embodiments, a monocyclic cycloalkyl group has 5 to 6 ring carbon atoms (“C₅₋₆ monocyclic cycloalkyl”). In some embodiments, a monocyclic cycloalkyl group has 5 to 10 ring carbon atoms (“C₅₋₁₀ monocyclic cycloalkyl”). Examples of monocyclic C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈).

In some embodiments “cycloalkyl” is a bicyclic cycloalkyl. In some embodiments, a bicyclic cycloalkyl has 4-14 ring carbon atoms. (“C₄₋₁₄ bicyclic cycloalkyl”). In some embodiments, a bicyclic cycloalkyl group has 4 to 12 ring carbon atoms (“C₄₋₁₂ bicyclic cycloalkyl”). In some embodiments, a bicyclic cycloalkyl group has 4 to 10 ring carbon atoms (“C₄₋₁₀ bicyclic cycloalkyl”). In some embodiments, a bicyclic cycloalkyl group has 5 to 10 ring carbon atoms (“C₅₋₁₀ bicyclic cycloalkyl”). In some embodiments, a bicyclic cycloalkyl group has 6 to 10 ring carbon atoms (“C₆₋₁₀ bicyclic cycloalkyl”). In some embodiments, a bicyclic cycloalkyl group has 8 to 10 ring carbon atoms (“C₅₋₁₀ bicyclic cycloalkyl”). In some embodiments, a bicyclic cycloalkyl group has 7 to 9 ring carbon atoms (“C₇₋₉ bicyclic cycloalkyl”). Examples of bicyclic cycloalkyls include bicyclo[1.1.0]butane (C₄), bicyclo[1.1.1]pentane (C₅), spiro[2.2] pentane (C₅), bicyclo[2.1.0]pentane (C₅), bicyclo[2.1.1]hexane (C₆), bicyclo[3.1.0]hexane (C₆), spiro[2.3] hexane (C₆), bicyclo[2.2.1]heptane (norbornane) (C₇), bicyclo[3.2.0]heptane (C₇), bicyclo[3.1.1]heptane (C₇), bicyclo[3.1.1]heptane (C₇), bicyclo[4.1.0]heptane (C₇), spiro[2.4] heptane (C₇), spiro [3.3] heptane (C₇), bicyclo[2.2.2]octane (C₈), bicyclo[4.1.1]octane (C₈)octahydropentalene (C₈), bicyclo[3.2.1]octane (C₈), bicyclo[4.2.0]octane (C₈), spiro[2.5]octane (C₈), spiro[3.4]octane (C₈), bicyclo[3.3.1]nonane (C₉), octahydro-1H-indene (C₉), bicyclo[4.2.1]nonane (C₉), spiro[3.5]nonane (C₉), spiro[4.4]nonane (C₉), bicyclo[3.3.2]decane (C₁₀), bicyclo[4.3.1]decane (C₁₀), spiro[4.5]decane (C₁₀), bicyclo[3.3.3]undecane (C₁₁), decahydronaphthalene (C₁₀), bicyclo[4.3.2]undecane spiro[5.5]undecane (C₁₁) and bicyclo[4.3.3]dodecane (C₁₂). In some embodiments “cycloalkyl” is a tricyclic cycloalkyl. In some embodiments, a tricyclic cycloalkyl has 6-14 ring carbon atoms. (“C₆₋₁₄ tricyclic cycloalkyl”). In some embodiments, a tricyclic cycloalkyl group has 8 to 12 ring carbon atoms (“C₈₋₁₂ tricyclic cycloalkyl”). In some embodiments, a tricyclic cycloalkyl group has 10 to 12 ring carbon atoms (“C₁₀₋₁₂ tricyclic cycloalkyl. Examples of tricyclic cycloalkyls include adamantine (C₁₂).

Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl.

“Heterocyclyl” or “heterocyclic” refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, sulfur, boron, phosphorus, and silicon (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, aziridinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like. “Nitrogen-containing heterocyclyl” group means a 4- to 7-membered non-aromatic cyclic group containing at least one nitrogen atom, for example, but without limitation, morpholine, piperidine (e.g., 2-piperidinyl, 3-piperidinyl and 4-piperidinyl), pyrrolidine (e.g., 2-pyrrolidinyl and 3-pyrrolidinyl), azetidine, pyrrolidone, imidazoline, imidazolidinone, 2-pyrazoline, pyrazolidine, piperazine, and N-alkyl piperazines such as N-methyl piperazine. Particular examples include azetidine, piperidone and piperazone.

“Hetero” when used to describe a compound or a group present on a compound means that one or more carbon atoms in the compound or group have been replaced by a nitrogen, oxygen, or sulfur heteroatom. Hetero may be applied to any of the hydrocarbyl groups described above such as alkyl, e.g., heteroalkyl, cycloalkyl, e.g., heterocyclyl, aryl, e.g., heteroaryl, cycloalkenyl, e.g., cycloheteroalkenyl, and the like having from 1 to 5, and particularly from 1 to 3 heteroatoms.

“Acyl” refers to a radical —C(═O)R²⁰, where R²⁰ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, as defined herein. “Alkanoyl” is an acyl group wherein R²⁰ is a group other than hydrogen. Representative acyl groups include, but are not limited to, formyl (—CHO), acetyl (—C(═O)CH₃), cyclohexylcarbonyl, cyclohexylmethylcarbonyl, benzoyl (—C(═O)Ph), benzylcarbonyl (—C(═O)CH₂Ph), —C(═O)—C₁-C₈ alkyl, —C(═O)—(CH₂)ₜ(C₆-C₁₀ aryl), —C(═O)—(CH₂)ₜ(5-10 membered heteroaryl), —C(═O)—(CH₂)ₜ(C₃-C₁₀ cycloalkyl), and —C(═O)—(CH₂)ₜ(4-10 membered heterocyclyl), wherein t is an integer from 0 to 4. In certain embodiments, R²¹ is C₁-C₈ alkyl, substituted with halo or hydroxy; or C₃-C₁₀ cycloalkyl, 4-10 membered heterocyclyl, C₆-C₁₀ aryl, arylalkyl, 5-10 membered heteroaryl or heteroarylalkyl, each of which is substituted with unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy.

The term aminoalkyl refers to a substituted alkyl group wherein one or more of the hydrogen atoms are independently replaced by an —NH₂ group.

The term hydroxyalkyl refers to a substituted alkyl group wherein one or more of the hydrogen atoms are independently replaced by an —OH group.

The terms “alkylamino” and “dialkylamino” refer to —NH(alkyl) and —N(alkyl)₂ radicals respectively. In some embodiments the alkylamino is a —NH(C₁-C₄ alkyl). In some embodiments the alkylamino is methylamino, ethylamino, propylamino, isopropylamino, n-butylamino, iso-butylamino, sec-butylamino or tert-butylamino. In some embodiments the dialkylamino is —N(C₁-C₆ alkyl)₂. In some embodiments the dialkylamino is a dimethylamino, a methylethylamino, a diethylamino, a methylpropylamino, a methylisopropylamino, a methylbutylamino, a methylisobutylamino or a methyltertbutylamino.

The term “aryloxy” refers to an —O-aryl radical. In some embodiments the aryloxy group is phenoxy.

The term “haloalkoxy” refers to alkoxy structures that are substituted with one or more halo groups or with combinations thereof. For example, the term “fluoroalkoxy” includes haloalkoxy groups, in which the halo is fluorine. In some embodiments haloalkoxy groups are difluoromethoxy and trifluoromethoxy.

“Alkoxy” refers to the group —OR²⁹ where R²⁹ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl. Particular alkoxy groups are methoxy, ethoxy, n-propoxy, isopropoxy, n-butoxy, tert-butoxy, sec-butoxy, n-pentoxy, n-hexoxy, and 1,2-dimethylbutoxy. Particular alkoxy groups are lower alkoxy, i.e. with between 1 and 6 carbon atoms. Further particular alkoxy groups have between 1 and 4 carbon atoms.

In certain embodiments, R²⁹ is a group that has 1 or more substituents, for instance from 1 to 5 substituents, and particularly from 1 to 3 substituents, in particular 1 substituent, selected from the group consisting of amino, substituted amino, C₆-C₁₀ aryl, aryloxy, carboxyl, cyano, C₃-C₁₀ cycloalkyl, 4-10 membered heterocyclyl, halogen, 5-10 membered heteroaryl, hydroxyl, nitro, thioalkoxy, thioaryloxy, thiol, alkyl-S(O)—, aryl-S(O)—, alkyl-S(O)₂— and aryl-S(O)₂—. Exemplary ‘substituted alkoxy’ groups include, but are not limited to, —O—(CH₂)ₜ(C₆-C₁₀ aryl), —O—(CH₂)ₜ(5-10 membered heteroaryl), —O—(CH₂)ₜ(C₃-C₁₀ cycloalkyl), and —O—(CH₂)ₜ(4-10 membered heterocyclyl), wherein t is an integer from 0 to 4 and any aryl, heteroaryl, cycloalkyl or heterocyclyl groups present, may themselves be substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy. Particular exemplary ‘substituted alkoxy’ groups are —OCF₃, —OCH₂CF₃, —OCH₂Ph, —OCH₂-cyclopropyl, —OCH₂CH₂OH, and —OCH₂CH₂N(CH₃)₂.

“Amino” refers to the radical —NH₂.

“Oxo group” refers to —C(═O)—.

“Substituted amino” refers to an amino group of the formula —N(R³⁸)₂ wherein R³⁸ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, or an amino protecting group, wherein at least one of R³⁸ is not a hydrogen. In certain embodiments, each R³⁸ is independently selected from hydrogen, C₁-C₈ alkyl, C₃-C₈ alkenyl, C₃-C₈ alkynyl, C₆-C₁₀ aryl, 5-10 membered heteroaryl, 4-10 membered heterocyclyl, or C₃-C₁₀ cycloalkyl; or C₁-C₈ alkyl, substituted with halo or hydroxy; C₃-C₈ alkenyl, substituted with halo or hydroxy; C₃-C₈ alkynyl, substituted with halo or hydroxy, or —(CH₂)ₜ(C₆-C₁₀ aryl), —(CH₂)ₜ(5-10 membered heteroaryl), —(CH₂)ₜ(C₃-C₁₀ cycloalkyl), or —(CH₂)ₜ(4-10 membered heterocyclyl), wherein t is an integer between 0 and 8, each of which is substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy; or both R³⁸ groups are joined to form an alkylene group.

Exemplary “substituted amino” groups include, but are not limited to, —NR³⁹—C₁-C₈ alkyl, —NR³⁹—(CH₂)ₜ(C₆-C₁₀ aryl), —NR³⁹—(CH₂)ₜ(5-10 membered heteroaryl), —NR³⁹—(CH₂)ₜ(C₃-C₁₀ and —NR³⁹—(CH₂)ₜ(4-10 membered heterocyclyl), wherein t is an integer from 0 to 4, for instance 1 or 2, each R³⁹ independently represents H or C₁-C₈ alkyl; and any alkyl groups present, may themselves be substituted by halo, substituted or unsubstituted amino, or hydroxy; and any aryl, heteroaryl, cycloalkyl, or heterocyclyl groups present, may themselves be substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy. For the avoidance of doubt the term ‘substituted amino’ includes the groups alkylamino, substituted alkylamino, alkylarylamino, substituted alkylarylamino, arylamino, substituted arylamino, dialkylamino, and substituted dialkylamino as defined below. Substituted amino encompasses both monosubstituted amino and disubstituted amino groups.

In certain embodiments, the substituent present on the nitrogen atom is a nitrogen protecting group (also referred to herein as an “amino protecting group”). Nitrogen protecting groups include, but are not limited to, —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc}), —SO₂R^{cc}, —SO₂OR^{cc}, —SOR^{aa}, —C(═S)N(R^{cc})₂, —C(═O)SR^{cc}, —C(═S)SR^{cc}, —C₁₋₁₀ alkyl (e.g., aralkyl, heteroaralkyl), —C₂₋₁₀ alkenyl, —C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Each instance of R^{aa} is, independently, selected from —C₁₋₁₀ alkyl, —C₁₋₁₀ perhaloalkyl, —C₂₋₁₀ alkenyl, —C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)NR^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, —C₁₋₁₀ alkyl, —C₁₋₁₀ perhaloalkyl, —C₂₋₁₀ alkenyl, —C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion.

Each instance of R^{cc} is, independently, selected from hydrogen, —C₁₋₁₀ alkyl, —C₁₋₁₀ perhaloalkyl, —C₂₋₁₀ alkenyl, —C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{T})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —Osi(R^{ee})₃, —C(═S)N(R^{ff})₂, —C(═O)SR^{ee}, —C(═S)SR^{ee}, —SC(═S)SR^{ee}, —P(═O)(OR^{ee})₂—P(═O)(R^{ee})₂_OP(═O)(R^{ee})₂, —OP(═O)(OR^{ee})₂, —C₁₋₆ alkyl, —C₁₋₆ perhaloalkyl, —C₂₋₆ alkenyl, —C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S; wherein X⁻ is a counterion;

each instance of R^{ee} is, independently, selected from —C₁₋₆ alkyl, —C₁₋₆ perhaloalkyl, —C₂₋₆ alkenyl, —C₂₋₆ alkynyl, heteroC₁₋₆ alkyl, heteroC₂₋₆alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;

each instance of R^{ff} is, independently, selected from hydrogen, —C₁₋₆ alkyl, —C₁₋₆ perhaloalkyl, —C₂₋₆ alkenyl, —C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and

each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)₂, (C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —Osi(C₁₋₆ alkyl)₃ —C(═S)N(C₁₋₆ alkyl)₂, —C(═S)NH(C₁₋₆ alkyl), —C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, —C₁₋₆ alkyl, —C₁₋₆ perhaloalkyl, —C₂₋₆ alkenyl, —C₂₋₆ alkynyl, heteroC₁₋₆alkyl, heteroC₂₋₆alkenyl, heteroC₂₋₆alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

For example, nitrogen protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitrophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide and o-(benzoyloxymethyl)benzamide.

Nitrogen protecting groups such as carbamate groups (e.g., —C(═O)OR^{aa}) include, but are not limited to, methyl carbamate, ethyl carbamate, 9-fluorenylmethyl carbamate (Fmoc), 9-(2-sulfo)fluorenylmethyl carbamate, 9-(2,7-dibromo)fluorenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl (o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isobornyl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., —S(═O)₂R^{aa}) include, but are not limited to, p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mb s), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-S-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N-(N′,N′-dimethylaminomethylene)amine, N,N′-isopropylidenediamine, N-p-nitrobenzylideneamine, N-salicylideneamine, N-S-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, the substituent present on an oxygen atom is an oxygen protecting group (also referred to herein as an “hydroxyl protecting group”). Oxygen protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein R^{aa}, R^{bb} and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include, but are not limited to, methyl, methoxymethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethyl silyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, 2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

The term “leaving group” is given its ordinary meaning in the art of synthetic organic chemistry and refers to an atom or a group capable of being displaced by a nucleophile. Examples of suitable leaving groups include, but are not limited to, halogen (such as F, Cl, Br, or I (iodine)), alkoxycarbonyloxy, aryloxycarbonyloxy, alkanesulfonyloxy, arenesulfonyloxy, alkyl-carbonyloxy (e.g., acetoxy), arylcarbonyloxy, aryloxy, methoxy, N,O-dimethylhydroxylamino, pixyl, and haloformates. In certain embodiments, the leaving group is halogen, alkanesulfonyloxy, arenesulfonyloxy, diazonium, alkyl diazenes, aryl diazenes, alkyl triazenes, aryl triazenes, nitro, alkyl nitrate, aryl nitrate, alkyl phosphate, aryl phosphate, alkyl carbonyl oxy, aryl carbonyl oxy, alkoxcarbonyl oxy, aryoxcarbonyl oxy ammonia, alkyl amines, aryl amines, hydroxyl group, alkyloxy group, or aryloxy. In some cases, the leaving group is a sulfonic acid ester, such as toluenesulfonate (tosylate, -OTs), methanesulfonate (mesylate, -OMs), p-bromobenzenesulfonyloxy (brosylate, -OBs), —OS(═O)₂(CF₂)₃CF₃ (nonaflate, -ONf), or trifluoromethanesulfonate (triflate, -OTf). In some cases, the leaving group is a brosylate, such as p-bromobenzenesulfonyloxy. In some cases, the leaving group is a nosylate, such as 2-nitrobenzenesulfonyloxy. In some embodiments, the leaving group is a sulfonate-containing group. In some embodiments, the leaving group is a tosylate group. The leaving group may also be a phosphineoxide (e.g., formed during a Mitsunobu reaction) or an internal leaving group such as an epoxide or cyclic sulfate. Other non-limiting examples of leaving groups are water, ammonia, alcohols, ether moieties, thioether moieties, zinc halides, magnesium moieties, diazonium salts, and copper moieties.

“Carboxy” refers to the radical —C(═O)OH.

“Cyano” refers to the radical —CN.

“Halo” or “halogen” refers to fluoro (F), chloro (Cl), bromo (Br), and iodo (I). In certain embodiments, the halo group is either fluoro or chloro.

“Haloalkyl” refers to an alkyl radical in which the alkyl group is substituted with one or more halogens. Typical haloalkyl groups include, but are not limited to, trifluoromethyl (—CF₃), difluoromethyl (—CHF₂), fluoromethyl (—CH₂F), chloromethyl (—CH₂Cl), dichloromethyl (—CHCl₂), tribromomethyl (—CH₂Br), and the like.

“Hydroxy” refers to the radical —OH.

“Nitro” refers to the radical —NO₂.

Alkyl, alkenyl, alkynyl, carbocyclyl, heterocyclyl, aryl, and heteroaryl groups, as defined herein, are optionally substituted (e.g., “substituted” or “unsubstituted” alkyl, “substituted” or “unsubstituted” alkenyl, “substituted” or “unsubstituted” alkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described herein that results in the formation of a stable compound. Any and all such combinations are contemplated in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}. —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —S(═O)(═NR^{bb})R^{aa}, —OS(═O)R^{aa}, —Si(R^{aa})₃, —OSi(R^{aa})₃ —C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)₂R^{aa}, —OP(═O)₂R^{aa}, —P(═O)(R^{aa})₂, —OP(═O)(R^{aa})₂, —OP(═O)(OR^{cc})₂, —P(═O)₂N(R^{bb})₂, —OP(═O)₂N(R^{bb})₂, —P(═O)(NR^{bb})₂, —OP(═O)(NR^{bb})₂, —NR^{bb}P(═O)(OR^{cc})₂, —NR^{bb}P(═O)(NR^{bb})₂, —P(R^{cc})₂, —P(R^{cc})₃, —OP(R^{cc})₂, —OP(R^{cc})₃, —B(R^{aa})₂, —B(OR^{cc})₂, —BR^{aa}(OR^{cc}), C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, carbocyclyl, heterocyclyl, aryl, and heteroaryl is independently substituted with 0, 1, 2, 3, 4, or 5 R^{dd} groups; or two geminal hydrogens on a carbon atom are replaced with the group ═O, ═S, ═NN(R^{bb})₂═NNR^{bb}C(═O)R^{aa}, ═NNR^{bb}C(═O)OR^{aa}, ═NNR^{bb}S(═O)₂R^{aa}═NR^{bb}, or ═NOR^{cc};

each instance of R^{aa} is, independently, selected from C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;

each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;

each instance of R^{ee} is, independently, selected from hydrogen, C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;

each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})_{R}ee, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)₂R^{ee}, —P(═O)(R^{ee})₂, —OP(═O)(R^{ee})₂, —OP(═O)(OR^{ee})₂, C₁₋₆ alkyl, C₁₋₆ haloalkyl, C₂₋₆ alkenyl, C₂₋₆ alkynyl, C₃₋₁₀ carbocyclyl, 3-10 membered heterocyclyl, C₆₋₁₀ aryl, 5-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S;

each instance of R^{ee} is, independently, selected from C₁₋₆ alkyl, C₁₋₆ haloalkyl, C₂₋₆ alkenyl, C₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups;

each instance of R^{ff} is, independently, selected from hydrogen, C₁₋₆ alkyl, C₁₋₆ haloalkyl, C₂₋₆ alkenyl, C₂₋₆ alkynyl, C₃₋₁₀ carbocyclyl, 3-10 membered heterocyclyl, C₆₋₁₀ aryl and 5-10 membered heteroaryl, or two R^{ff} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{gg} groups; and

each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃ —C(═S)N(C₁₋₆ alkyl)₂, —C(═S)NH(C₁₋₆ alkyl), —C(═S)NH₂, —C(═O)S(C₁₋₆ alkyl), —C(═S)SC₁₋₆ alkyl, —SC(═S)SC₁₋₆ alkyl, —P(═O)₂(C₁₋₆ alkyl), —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ haloalkyl, C₂₋₆ alkenyl, C₂₋₆ alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

A “counterion” or “anionic counterion” is a negatively charged group associated with a cationic quaternary amino group in order to maintain electronic neutrality. Exemplary counterions include halide ions (e.g., F⁻, Cl⁻, Br⁻, I⁻), NO₃⁻, ClO₄⁻, OH⁻, H₂PO₄, HSO₄, SO₄⁻²sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), and carboxylate ions (e.g., acetate, ethanoate, propanoate, benzoate, glycerate, lactate, tartrate, glycolate, and the like).

Nitrogen atoms can be substituted or unsubstituted as valency permits, and include primary, secondary, tertiary, and quarternary nitrogen atoms. Exemplary nitrogen atom substitutents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{cc} groups attached to a nitrogen atom are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

“Boronic ester group” refers to

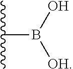 [SMILES: *B(O)O]

“Boronic acid group” refers to

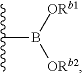

wherein each of R^{b1} and R^{b2} is, independently, selected from substituted or unsubstituted alky substituted or unsubstituted alkenyl, substituted or unsubstituted alkynyl, substituted or unsubstituted carbocyclyl, substituted or unsubstituted heterocyclyl, substituted or unsubstituted aryl, and substituted or unsubstituted heteroaryl, wherein R^{b1} and R^{b2} are optionally joined together with their intervening atoms to form a substituted or unsubstituted ring.

As used herein, “chiral auxiliary” refers to a substituent having one or more asymmetric atoms (e.g., asymmetric carbon atoms) that biases a chemical reaction to favor selective formation of one isomer (e.g., stereoisomer) over another and is covalently attached to a substrate. Examples of chiral auxiliaries include but are not limited to chiral oxazolidones.

These and other exemplary substituents are described in more detail in the Detailed Description, Examples, and Claims. The invention is not intended to be limited in any manner by the above exemplary listing of substituents.

### Compound

In some embodiments, provided herein is N-(6-amino-5-methylpyridin-3-yl)-2-((2R,5S)-2-(benzo[d]thiazol-5-yl)-5-methylpiperidin-1-yl)-2-oxoacetamide, a PRMT5 inhibitor (e.g., an MTA-uncompetitive PRMT5 inhibitor) compound of formula (I).

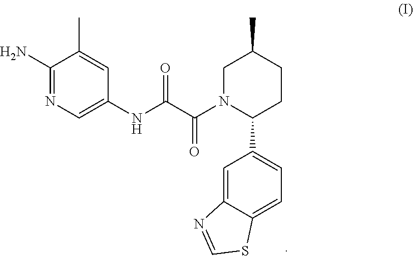 [SMILES: Cc1cc(NC(=O)C(=O)N2C[C@@H](C)CC[C@@H]2c2ccc3scnc3c2)cnc1N]

In certain embodiments, the compound of formula (I) is a crystalline form of the compound of formula (I). The compound of formula (I) can also be referred to as “Compound I.”

In certain embodiments, the crystalline form of the compound of formula (I) is crystalline Form A. In certain embodiments, Form A has an XRPD pattern with one or more (e.g., one, two, three, four or five) characteristic peaks between and including the following values of 20 in degrees: 6.2 to 6.6 (e.g., 6.4±0.2), 8.7 to 9.1 (e.g., 8.9±0.2), 12.5 to 12.9 (e.g., 12.7±0.2), 13.8 to 14.2 (e.g., 14.0±0.2), 18.9 to 19.3 (e.g., 19.1±0.2), 19.7 to 20.1 (e.g., 19.9±0.2) and 22.4 to 22.8 (e.g., 22.6±0.2). In certain embodiments, Form A has an XRPD pattern with one or more (e.g., one, two, three, four, five, six, seven, eight, nine or ten) characteristic peaks between and including the following values of 20 in degrees: 6.2 to 6.6 (e.g., 6.4±0.2), 8.7 to 9.1 (e.g., 8.9±0.2), 12.5 to 12.9 (e.g., 12.7±0.2), 13.6 to 14.0 (e.g., 13.8±0.2), 13.8 to 14.2 (e.g., 14.0±0.2), 18.1 to 18.5 (e.g., 18.3±0.2), 18.9 to 19.3 (e.g., 19.1±0.2), 19.7 to 20.1 (e.g., 19.9±0.2), 22.4 to 22.8 (e.g., 22.6±0.2), 24.1 to 24.5 (e.g., 24.3±0.2), 26.0 to 26.4 (e.g., 26.2±0.2), 26.5 to 26.9 (e.g., 26.7±0.2) and 28.0 to 28.4 (e.g., 28.2±0.2).

In certain embodiments, the X-ray powder diffraction pattern for Form A may comprise one or more (e.g., one, two, three, four or five) characteristic peaks, in terms of 20, selected from the peaks at 6.4±0.2, 8.9±0.2, 12.7±0.2, 14.0±0.2, 19.1±0.2, 19.9±0.2, 22.6±0.2.

In certain embodiments, the X-ray powder diffraction pattern for Form A may comprise one or more (e.g., one, two, three, four, five, six, seven, eight, nine, or ten) characteristic peaks, in terms of 20, selected from the peaks at 6.4±0.2, 8.9±0.2, 12.7±0.2, 13.8±0.2, 14.0±0.2, 18.3±0.2, 19.1±0.2, 19.9±0.2, 22.6±0.2, 24.3±0.2, 26.2±0.2 and 26.7±0.2, 28.2±0.2.

In certain embodiments, the X-ray powder diffraction pattern for Form A comprises at least one characteristic peak, in terms of 20, selected from the peaks at 6.4±0.2, 8.9±0.2, 12.7±0.2, 13.8±0.2, 14.0±0.2, 18.3±0.2, 19.1±0.2, 19.9±0.2, 22.6±0.2, 24.3±0.2, 26.2±0.2 and 26.7±0.2, 28.2±0.2. In certain embodiments, the X-ray powder diffraction pattern for Form A comprises at least two characteristic peaks, in terms of 20, selected from the peaks at 6.4±0.2, 8.9±0.2, 12.7±0.2, 13.8±0.2, 14.0±0.2, 18.3±0.2, 19.1±0.2, 19.9±0.2, 22.6±0.2, 24.3±0.2, 26.2±0.2 and 26.7±0.2, 28.2±0.2. In certain embodiments, the X-ray powder diffraction pattern for Form A comprises at least three characteristic peaks, in terms of 20, selected from the peaks at 6.4±0.2, 8.9±0.2, 12.7±0.2, 13.8±0.2, 14.0±0.2, 18.3±0.2, 19.1±0.2, 19.9±0.2, 22.6±0.2, 24.3±0.2, 26.2±0.2 and 26.7±0.2, 28.2±0.2. In certain embodiments, the X-ray powder diffraction pattern for Form A comprises at least four characteristic peaks, in terms of 2θ, selected from the peaks at 6.4±0.2, 8.9±0.2, 12.7±0.2, 13.8±0.2, 14.0±0.2, 18.3±0.2, 19.1±0.2, 19.9±0.2, 22.6±0.2, 24.3±0.2, 26.2±0.2 and 26.7±0.2, 28.2±0.2. In certain embodiments, the X-ray powder diffraction pattern for Form A comprises at least five characteristic peaks, in terms of 2θ, selected from the peaks at 6.4±0.2, 8.9±0.2, 12.7±0.2, 13.8±0.2, 14.0±0.2, 18.3±0.2, 19.1±0.2, 19.9±0.2, 22.6±0.2, 24.3±0.2, 26.2±0.2 and 26.7±0.2, 28.2±0.2. In certain embodiments, the X-ray powder diffraction pattern for Form A comprises at least six characteristic peaks, in terms of 2θ, selected from the peaks at 6.4±0.2, 8.9±0.2, 12.7±0.2, 13.8±0.2, 14.0±0.2, 18.3±0.2, 19.1±0.2, 19.9±0.2, 22.6±0.2, 24.3±0.2, 26.2±0.2 and 26.7±0.2, 28.2±0.2. In certain embodiments, the X-ray powder diffraction pattern for Form A comprises at least seven characteristic peaks, in terms of 2θ, selected from the peaks at 6.4±0.2, 8.9±0.2, 12.7±0.2, 13.8±0.2, 14.0±0.2, 18.3±0.2, 19.1±0.2, 19.9±0.2, 22.6±0.2, 24.3±0.2, 26.2±0.2 and 26.7±0.2, 28.2±0.2. In certain embodiments, the X-ray powder diffraction pattern for Form A comprises at least eight characteristic peaks, in terms of 2θ, selected from the peaks at 6.4±0.2, 8.9±0.2, 12.7±0.2, 13.8±0.2, 14.0±0.2, 18.3±0.2, 19.1±0.2, 19.9±0.2, 22.6±0.2, 24.3±0.2, 26.2±0.2 and 26.7±0.2, 28.2±0.2. In certain embodiments, the X-ray powder diffraction pattern for Form A comprises at least nine characteristic peaks, in terms of 2θ, selected from the peaks at 6.4±0.2, 8.9±0.2, 12.7±0.2, 13.8±0.2, 14.0±0.2, 18.3±0.2, 19.1±0.2, 19.9±0.2, 22.6±0.2, 24.3±0.2, 26.2±0.2 and 26.7±0.2, 28.2±0.2. In certain embodiments, the X-ray powder diffraction pattern for Form A comprises at least ten characteristic peaks, in terms of 2θ, selected from the peaks at 6.4±0.2, 8.9±0.2, 12.7±0.2, 14.0±0.2, 19.1±0.2, 19.9±0.2 and 22.6±0.2.

In certain embodiments, Form A has an XRPD pattern with characteristic peaks at the following values of 2θ in degrees: 6.4±0.2, 8.9±0.2, 12.7±0.2, 14.0±0.2, 19.1±0.2, 19.9±0.2, 22.6±0.2.

In certain embodiments, Form A has an XRPD pattern with characteristic peaks at the following values of 2θ in degrees: 6.4±0.2, 8.9±0.2, 12.7±0.2, 13.8±0.2, 14.0±0.2, 18.3±0.2, 19.1±0.2, 19.9±0.2, 22.6±0.2, 24.3±0.2, 26.2±0.2, 26.7±0.2 and 28.2±0.2.

In some embodiments, Form A has an XRPD pattern (obtained using CuKa radiation) with characteristic peaks comprising one, two, three, four, five, six, seven, eight, nine, or ten characteristic peaks, in terms of 20 values in degrees shown in Table 1 (±0.2 degrees).

TABLE 1
XRPD peaks for Form A of a compound of formula (I)

| Peak | 2θ Angle | Relative intensity |
|---|---|---|
| 1 | 6.39 | 21.4% |
| 2 | 8.86 | 7.1% |
| 3 | 12.74 | 14.1% |
| 4 | 13.85 | 3.0% |
| 5 | 14.04 | 100.0% |
| 6 | 14.41 | 1.2% |
| 7 | 17.80 | 0.4% |
| 8 | 18.34 | 2.3% |
| 9 | 19.14 | 7.0% |
| 10 | 19.45 | 1.7% |
| 11 | 19.95 | 10.0% |
| 12 | 20.28 | 1.6% |
| 13 | 20.67 | 0.8% |
| 14 | 21.00 | 0.9% |
| 15 | 22.64 | 7.0% |
| 16 | 23.25 | 0.8% |
| 17 | 23.92 | 0.9% |
| 18 | 24.29 | 1.9% |
| 19 | 24.82 | 0.5% |
| 20 | 25.11 | 0.6% |
| 21 | 25.46 | 0.6% |
| 22 | 26.16 | 3.3% |
| 23 | 26.65 | 3.9% |
| 24 | 26.91 | 0.5% |
| 25 | 28.22 | 1.9% |
| 26 | 28.26 | 0.8% |
| 27 | 29.00 | 1.6% |
| 28 | 29.69 | 0.5% |
| 29 | 30.87 | 0.7% |
| 30 | 30.53 | 0.3% |
| 31 | 31.01 | 1.0% |
| 32 | 31.52 | 0.4% |
| 33 | 32.10 | 0.7% |
| 34 | 32.64 | 0.7% |
| 35 | 33.04 | 0.3% |
| 36 | 33.84 | 0.3% |
| 37 | 34.19 | 1.2% |
| 38 | 35.07 | 0.4% |
| 39 | 35.77 | 1.1% |
| 40 | 36.97 | 0.5% |
| 41 | 38.21 | 0.4% |
| 42 | 38.68 | 0.3% |
| 43 | 39.53 | 0.7% |

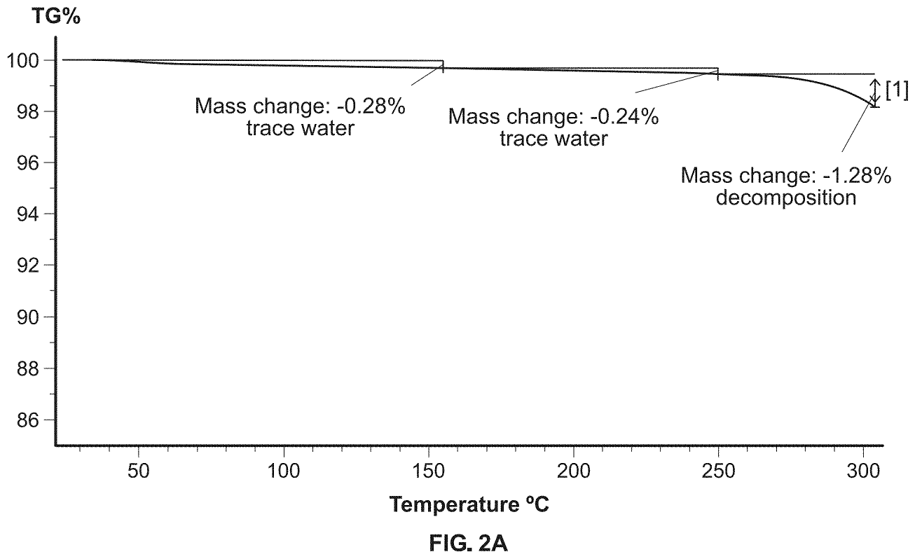

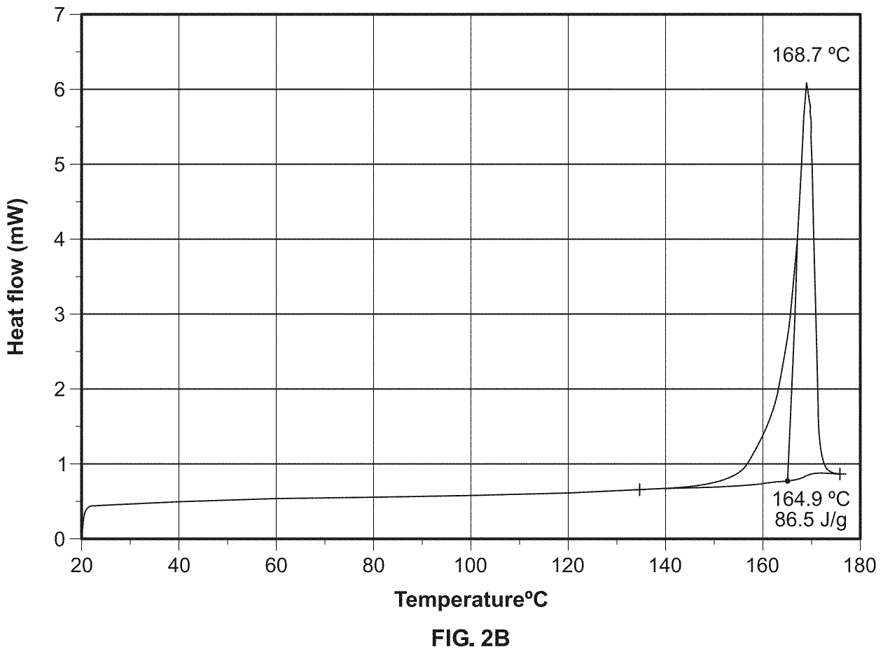

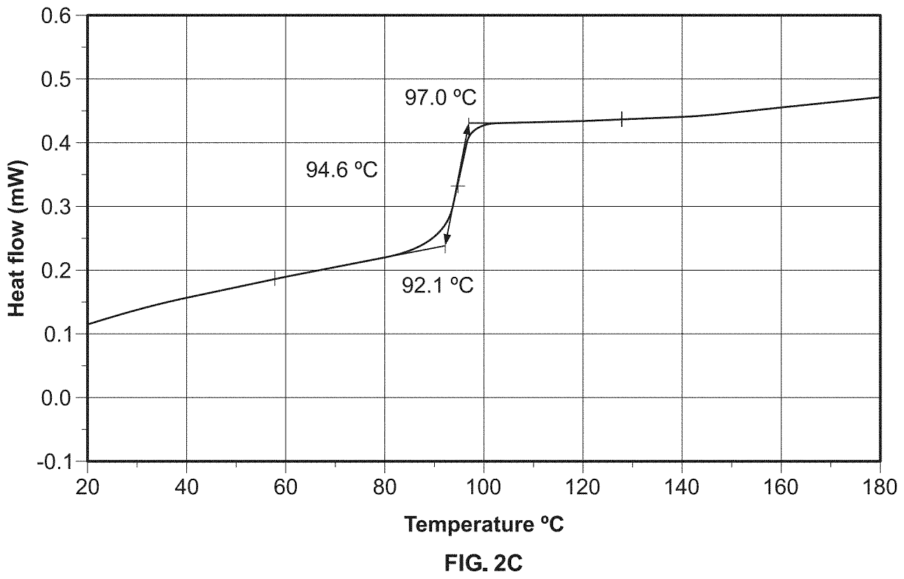

In certain embodiments, Form A is substantially characterized by the thermal gravimetric analysis (TGA) as shown in FIG. 2A. In certain embodiments, Form A can be characterized by the thermal gravimetric analysis (TGA) shown in FIG. 2A, which shows two small loses in mass below 250° C., totaling to about 0.5% loss of water, and showing decomposition at temperatures above 250° C.

In certain embodiments, Form A is substantially characterized by the differential scanning calorimetry profile (DSC) shown in FIG. 2B. In some embodiments, Form A can be characterized by the differential scanning calorimetry profile (DSC) shown in FIG. 2B, showing a melt peak at about 168.7° C.

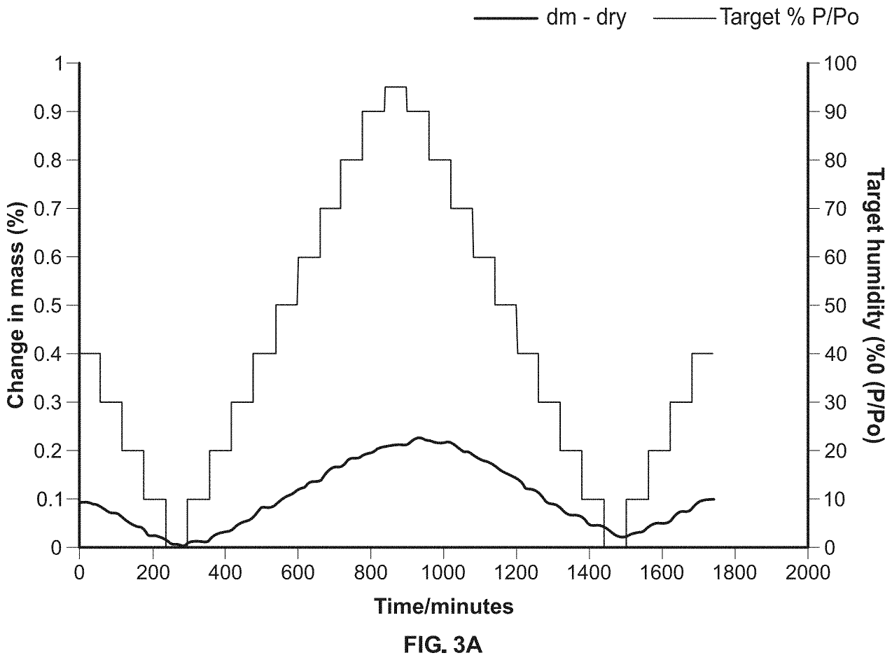

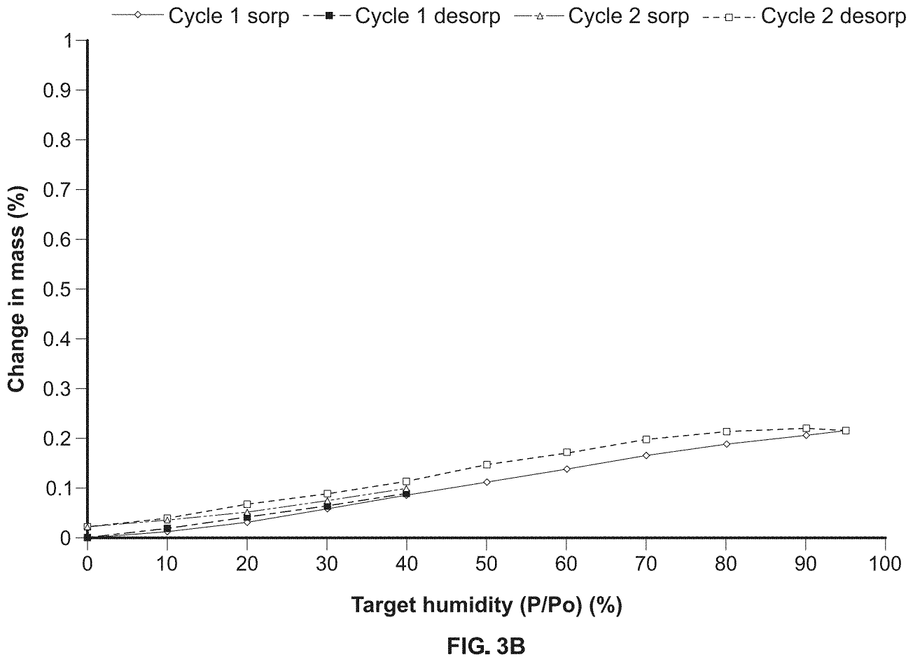

In certain embodiments, Form A is substantially characterized by the DVS profile as shown in FIG. 3A and FIG. 3B.

In certain embodiments, Form A has a unit cell as determined by crystal X-ray crystallography of the following dimensions: a=5.0705(2) Å; b=13.9249(7) Å; c=13.9877(7) α=87.522(2)°; β=85.730(2°) and γ=81.474(2)°. In some embodiments, Form A has a triclinic crystal system. In some embodiments, Form A has a P1 space group. In some embodiments, form A has a volume of 973.47(8) Å³. In some embodiments, form A has a Z value of 2. In some embodiments, Form A has a density of 1.397 Mg/m³. In certain embodiments, the crystalline form of the compound of formula (I) comprises a mixture of two or more crystalline forms. In certain embodiments, the crystalline form of the compound of formula (I) is substantially pure crystalline Form A.

### Methods of Preparing Compound

In some embodiments, provided herein is a process for preparing N-(6-amino-5-methylpyridin-3-yl)-2-((2R,5 S)-2-(benzo[d]thiazol-5-yl)-5-methylpiperidin-1-yl)-2-oxoacetamide (a compound of formula (I)) or a salt thereof:

comprising:

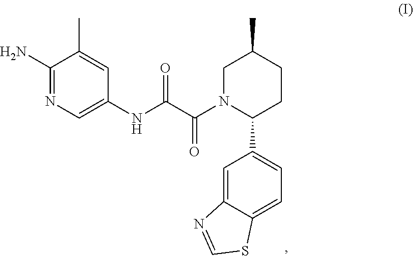 [SMILES: Cc1cc(NC(=O)C(=O)N2C[C@@H](C)CC[C@@H]2c2ccc3scnc3c2)cnc1N]

hydrogenating a compound of formula (II):

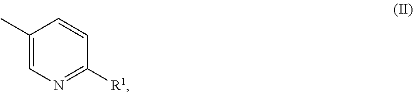

thereby producing a compound of formula (III-a):

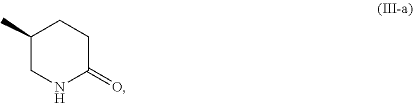

wherein R¹ is a chiral auxiliary.

In some embodiments, the process further comprises protecting the nitrogen group of the compound of formula (III-a), thereby forming a compound of formula (III):

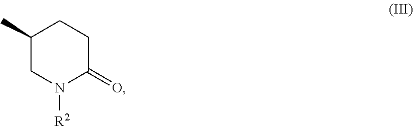

wherein R² is a nitrogen protecting group.

In some embodiments, the process further comprises:

cross-coupling a compound of formula (III) with a compound of formula (IV):

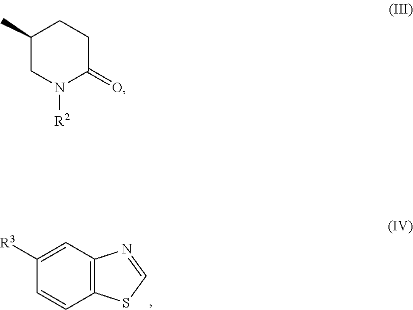

thereby producing a compound of formula (V):

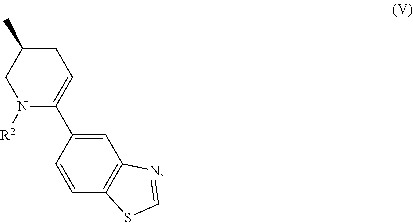

wherein R² is a nitrogen protecting group; and

R³ is a boronic acid or a boronic ester.

In some embodiments, the process further comprises removing the nitrogen protective group from the compound of formula (V), thereby forming a compound of formula (V-a):

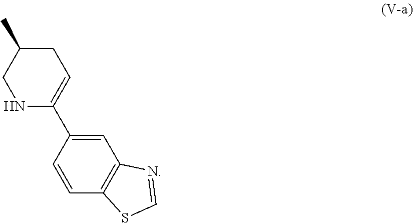 [SMILES: C[C@H]1CC=C(c2ccc3scnc3c2)NC1]

In some embodiments, the process further comprises:

reducing the compound of formula (V-a):

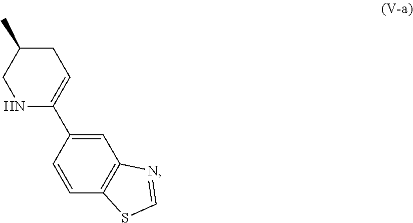 [SMILES: C[C@H]1CC=C(c2ccc3scnc3c2)NC1]

thereby producing a compound of formula (VI):

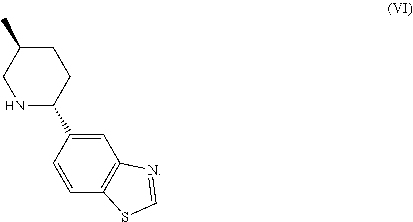 [SMILES: C[C@H]1CC[C@H](c2ccc3scnc3c2)NC1]

In some embodiments, the process further comprises:

coupling the compound of formula (VI) with a compound of formula (VII):

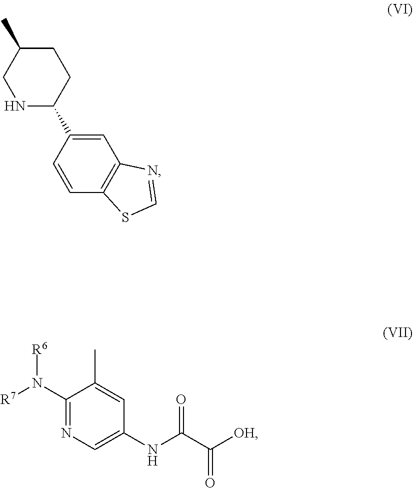

thereby producing a compound of formula (I-a):

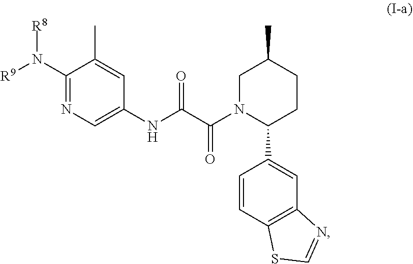

wherein each of R⁶, R⁷, R⁸, and R⁹ is, independently, H or a nitrogen protecting group.

In some embodiments, if R⁸, R⁹ or both R⁸ and R⁹ are a nitrogen protecting group, the process further comprises a deprotection step to remove the nitrogen protecting group from the compound of formula (I-a), thereby producing the compound of formula (I) or a salt thereof.

In some embodiments, the process further comprises converting a salt of the compound of formula (I) to the free base of the compound of formula (I).

In some embodiments, the process further comprises a crystallization step to produce a crystalline form of the compound of formula (I)(e.g., the free base of the compound of formula (I).

In some embodiments, provided is a process for producing a crystalline form of a compound of formula (I), wherein the process comprises crystallizing a compound of Formula (I)

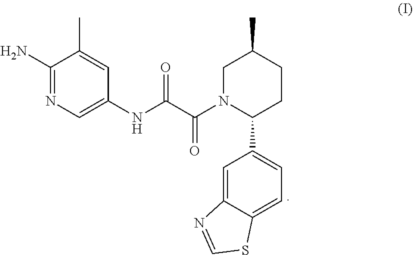 [SMILES: Cc1cc(NC(=O)C(=O)N2C[C@@H](C)CC[C@@H]2c2ccc3scnc3c2)cnc1N]

In some embodiments, provided herein is a process for preparing N-(6-amino-5-methylpyridin-3-yl)-2-((2R,5 S)-2-(benzo[d]thiazol-5-yl)-5-methylpiperidin-1-yl)-2-oxoacetamide (a compound of formula (I)) or a salt thereof:

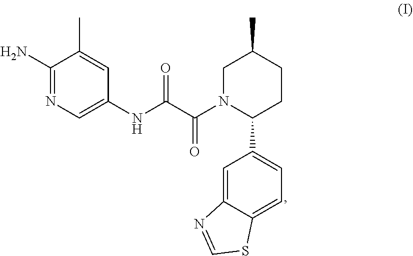 [SMILES: Cc1cc(NC(=O)C(=O)N2C[C@@H](C)CC[C@@H]2c2ccc3scnc3c2)cnc1N]

comprising:

(a) hydrogenating a compound of formula (II):

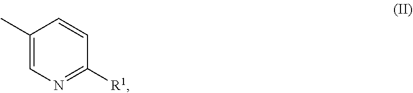

thereby producing a compound of formula (III-a):

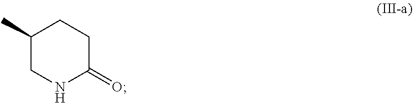

(b) protecting the nitrogen of the compound of formula (III-a) with a nitrogen protecting group, thereby providing a compound of formula (III)

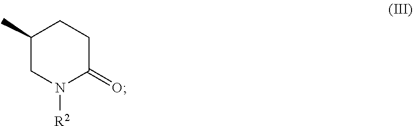

(c) cross-coupling the compound of formula (III) with a compound of formula (IV):

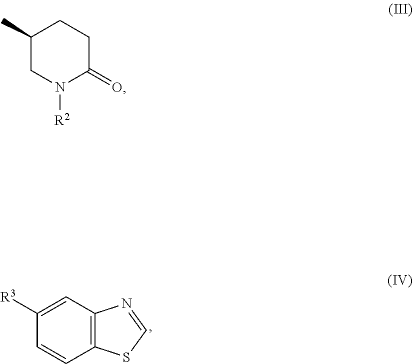

thereby producing a compound of formula (V):

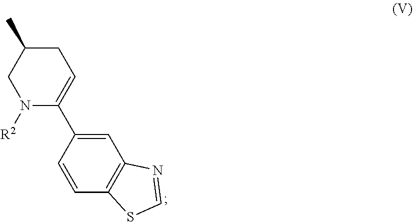

(d) removing nitrogen protecting group from the compound of formula (V), thereby producing a compound of formula (V-a);

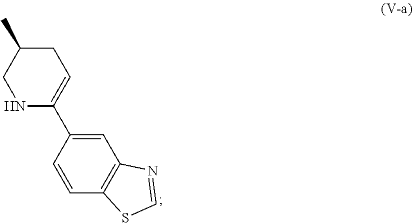 [SMILES: C[C@H]1CC=C(c2ccc3scnc3c2)NC1]

(e) reducing the compound of formula (V-a), thereby producing a compound of formula (VI):

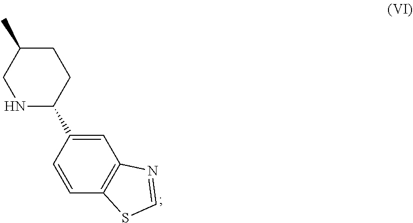 [SMILES: C[C@H]1CC[C@H](c2ccc3scnc3c2)NC1]

(f) coupling the compound of formula (VI) with a compound of formula (VII):

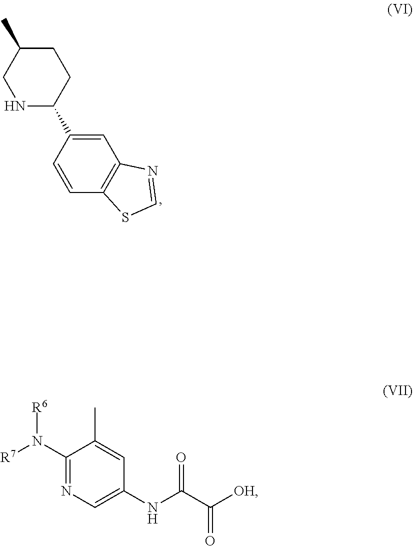

thereby producing a compound of formula (I-a):

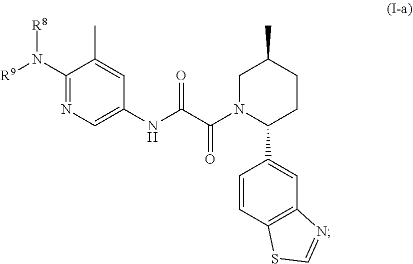

and

(g) optionally, if R⁸, R⁹, or both R⁸ and R⁹ are nitrogen protecting groups, deprotecting the compound of formula (I-a), thereby providing the compound of formula (I) or a salt thereof;

wherein R¹ is a chiral auxiliary;

R² is a nitrogen protecting group;

each of R⁶, R⁷, R⁸, and R⁹ is, independently, H or a nitrogen protecting group; and

R³ is a boronic acid group or a boronic ester group.

In some embodiments, provided herein is a process for preparing N-(6-amino-5-methylpyridin-3-yl)-2-((2R,5 S)-2-(benzo[d]thiazol-5-yl)-5-methylpiperidin-1-yl)-2-oxoacetamide (a compound of formula (I)) or a salt thereof:

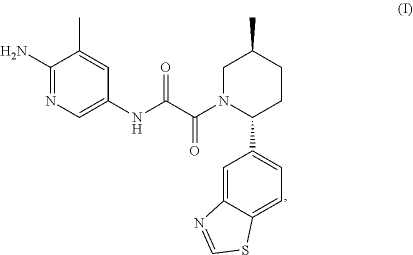 [SMILES: Cc1cc(NC(=O)C(=O)N2C[C@@H](C)CC[C@@H]2c2ccc3scnc3c2)cnc1N]

comprising:

coupling a compound of formula (VI) with a compound of formula (VII):

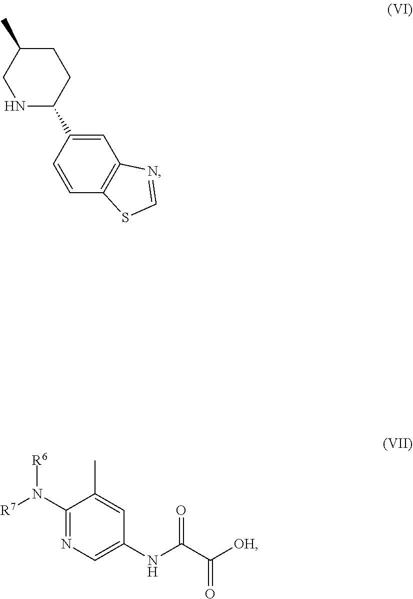

thereby producing the compound of formula (I-a):

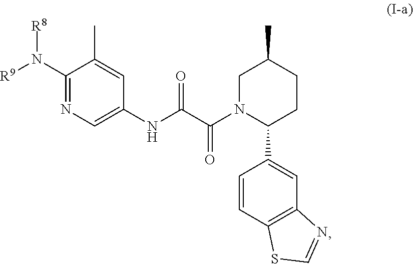

wherein each of R⁶, R⁷, R⁸, and R⁹ is, independently, H or a nitrogen protecting group; and

optionally, if R⁸, R⁹ or both R⁸ and R⁹ are nitrogen protecting groups, deprotecting the compound of formula (I-a), thereby providing the compound of formula (I) or a salt thereof.

In some embodiments, the process further comprises:

reducing a compound of formula (V-a):

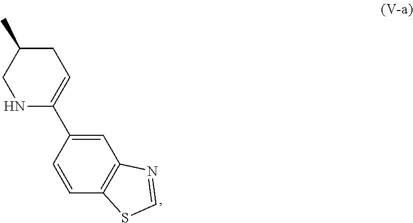 [SMILES: C[C@H]1CC=C(c2ccc3scnc3c2)NC1]

thereby producing a compound of formula (VI):

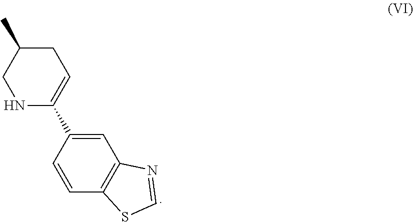

In some embodiments, the process further comprises removing the nitrogen protecting group from a compound of formula (V),

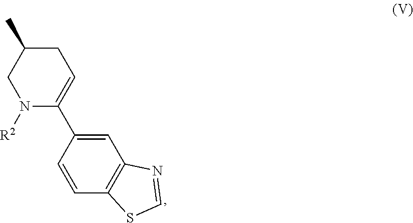

thereby producing the compound of formula (V-a):

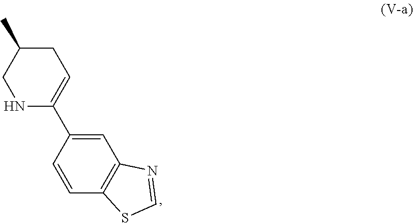

wherein R² is a nitrogen protecting group.

In some embodiments, the process further comprises:

cross-coupling a compound of formula (III) with a compound of formula (IV):

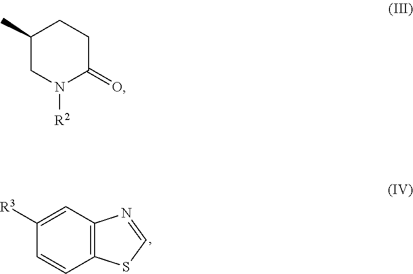

thereby producing the compound of formula (V):

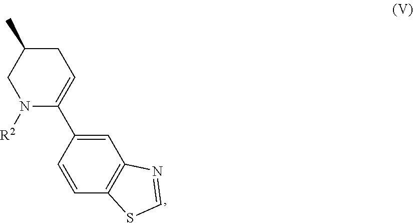

wherein R² is a nitrogen protecting group;

R³ is a boronic acid group or a boronic ester group.

In some embodiments, the process further comprises protecting the nitrogen group of compound of Formula (III-a) with a nitrogen protecting group, thereby producing a compound of formula (III). In some embodiments, R² is a nitrogen protecting group. In some embodiments, R² is a carbamate group. In some embodiments, R² is

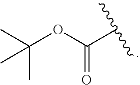 [SMILES: *C(=O)OC(C)(C)C]

In some embodiments, protecting the nitrogen group of formula (III-a) takes place in a sixth solvent. In some embodiments, the sixth solvent is an aprotic solvent. In some embodiments, the sixth solvent is acetonitrile. In some embodiments, protecting the nitrogen group of formula (III-a) takes place in the presence of a fourth base. In some embodiments, the fourth base is a pyridine base. In some embodiments, the fourth base is dimethylaminopyridine (DMAP).

In some embodiments, the process further comprises:

hydrogenating a compound of formula (II):

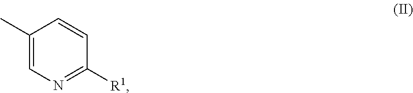

thereby producing a compound of formula (III-a):

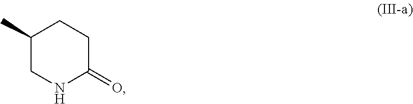

wherein R¹ is a chiral auxiliary.

In some embodiments, the chiral auxiliary is an optionally substituted oxazolidinone.

In some embodiments, the chiral auxiliary is

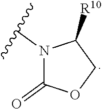

wherein R¹⁰ is C₁₋₆ alkyl, benzyl (Bn), or phenyl (Ph). In some embodiments, R¹⁰ is C₁₋₆ alkyl. In some embodiments, R¹⁰ is benzyl (Bn). In some embodiments, R¹⁰ is phenyl (Ph).

In some embodiments, the chiral auxiliary is

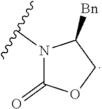 [SMILES: *N1C(=O)OC[C@@H]1Cc1ccccc1]

In some embodiments, hydrogenating the compound of formula (II) comprises contacting the compound of formula (II) with a first catalyst.

In some embodiments, hydrogenating the compound of formula (II) comprises contacting the compound of formula (II) with less than about 10 mol % (e.g., less than about 9 mol %; less than about 8 mol %; less than about 7 mol %; less than about 6 mol %; less than about 5 mol %; less than about 4 mol %; less than about 3 mol %; less than about 2.75 mol %; less than about 2.5 mol %; less than about 2.25 mol %; less than about 2 mol %; less than about 1.95 mol %; less than about 1.85 mol %; less than about 1.75 mol %; less than about 1.65 mol %; less than about 1.55 mol %; less than about 1.45 mol %; less than about 1.35 mol %; less than about 1.25 mol %; less than about 1.15 mol %; less than about 1.05 mol %; less than about 1 mol %; less than about 0.9 mol %; less than about 0.8 mol %; less than about 0.7 mol %; less than about 0.6 mol %; less than about 0.5 mol %; less than about 0.4 mol %; less than about 0.3 mol %; less than about 0.2 mol %; or less than about 0.1 mol %) of a first catalyst. In some embodiments, hydrogenating the compound of formula (II) comprises contacting the compound of formula (II) with less than about 10 mol % of the first catalyst. In some embodiments, hydrogenating the compound of formula (II) comprises contacting the compound of formula (II) with less than about 9 mol % of the first catalyst. In some embodiments, hydrogenating the compound of formula (II) comprises contacting the compound of formula (II) with less than about 8 mol % of the first catalyst. In some embodiments, hydrogenating the compound of formula (II) comprises contacting the compound of formula (II) with less than about 7 mol % of the first catalyst. In some embodiments, hydrogenating the compound of formula (II) comprises contacting the compound of formula (II) with less than about 6 mol % of the first catalyst. In some embodiments, hydrogenating the compound of formula (II) comprises contacting the compound of formula (II) with less than about 5 mol % of the first catalyst. In some embodiments, hydrogenating the compound of formula (II) comprises contacting the compound of formula (II) with less than about 4 mol % of the first catalyst. In some embodiments, hydrogenating the compound of formula (II) comprises contacting the compound of formula (II) with less than about 3 mol % of the first catalyst. In some embodiments, hydrogenating the compound of formula (II) comprises contacting the compound of formula (II) with less than about 2.75 mol % of the first catalyst. In some embodiments, hydrogenating the compound of formula (II) comprises contacting the compound of formula (II) with less than about 2.5 mol % of the first catalyst. In some embodiments, hydrogenating the compound of formula (II) comprises contacting the compound of formula (II) with less than about 2.25 mol % of the first catalyst. In some embodiments, hydrogenating the compound of formula (II) comprises contacting the compound of formula (II) with less than about 2 mol % of the first catalyst. In some embodiments, hydrogenating the compound of formula (II) comprises contacting the compound of formula (II) with less than about 1.95 mol % of the first catalyst. In some embodiments, hydrogenating the compound of formula (II) comprises contacting the compound of formula (II) with less than about 1.85 mol % of the first catalyst. In some embodiments, hydrogenating the compound of formula (II) comprises contacting the compound of formula (II) with less than about 1.75 mol % of the first catalyst. In some embodiments, hydrogenating the compound of formula (II) comprises contacting the compound of formula (II) with less than about 1.65 mol % of the first catalyst. In some embodiments, hydrogenating the compound of formula (II) comprises contacting the compound of formula (II) with less than about 1.55 mol % of the first catalyst. In some embodiments, hydrogenating the compound of formula (II) comprises contacting the compound of formula (II) with less than about 1.45 mol % of the first catalyst. In some embodiments, hydrogenating the compound of formula (II) comprises contacting the compound of formula (II) with less than about 1.35 mol % of the first catalyst. In some embodiments, hydrogenating the compound of formula (II) comprises contacting the compound of formula (II) with less than about 1.25 mol % of the first catalyst. In some embodiments, hydrogenating the compound of formula (II) comprises contacting the compound of formula (II) with less than about 1.15 mol % of the first catalyst. In some embodiments, hydrogenating the compound of formula (II) comprises contacting the compound of formula (II) with less than about 1.05 mol % of the first catalyst. In some embodiments, hydrogenating the compound of formula (II) comprises contacting the compound of formula (II) with less than about 1 mol % of the first catalyst. In some embodiments, hydrogenating the compound of formula (II) comprises contacting the compound of formula (II) with less than about 0.9 mol %; less than about 0.8 mol % of the first catalyst. In some embodiments, hydrogenating the compound of formula (II) comprises contacting the compound of formula (II) with less than about 0.7 mol % of the first catalyst. In some embodiments, hydrogenating the compound of formula (II) comprises contacting the compound of formula (II) with less than about 0.6 mol % of the first catalyst. In some embodiments, hydrogenating the compound of formula (II) comprises contacting the compound of formula (II) with less than about 0.5 mol % of the first catalyst. In some embodiments, hydrogenating the compound of formula (II) comprises contacting the compound of formula (II) with less than about 0.4 mol % of the first catalyst. In some embodiments, hydrogenating the compound of formula (II) comprises contacting the compound of formula (II) with less than about 0.3 mol % of the first catalyst. In some embodiments, hydrogenating the compound of formula (II) comprises contacting the compound of formula (II) with less than about 0.2 mol % of the first catalyst. In some embodiments, hydrogenating the compound of formula (II) comprises contacting the compound of formula (II) with less than about 0.1 mol % of the first catalyst. In some embodiments, hydrogenating the compound of formula (II) comprises contacting the compound of formula (II) with about 0.1 mol % to about 10 mol % (e.g., about 0.5 mol % to about 1 mol %; about 1 mol % to about 1.5 mol %; about 1 mol % to about 2 mol %; about 1.5 mol % to about 2 mol %; or about 1.5 mol % to about 2.5 mol %) of a first catalyst.

In some embodiments, hydrogenating the compound of formula (II) comprises contacting the compound of formula (II) with about 0.1 mol % to about 10 mol % of the first catalyst. In some embodiments, hydrogenating the compound of formula (II) comprises contacting the compound of formula (II) with about 0.5 mol % to about 1 mol % of the first catalyst. In some embodiments, hydrogenating the compound of formula (II) comprises contacting the compound of formula (II) with about 1 mol % to about 1.5 mol % of the first catalyst. In some embodiments, hydrogenating the compound of formula (II) comprises contacting the compound of formula (II) with about 1 mol % to about 2 mol % of the first catalyst. In some embodiments, hydrogenating the compound of formula (II) comprises contacting the compound of formula (II) with about 1.5 mol % to about 2 mol % of the first catalyst. In some embodiments, hydrogenating the compound of formula (II) comprises contacting the compound of formula (II) with about 1.5 mol % to about 2.5 mol % of the first catalyst.

In some embodiments, hydrogenating the compound of formula (II) comprises contacting the compound of formula (II) with about 1.0 mol % of the first catalyst. In some embodiments, hydrogenating the compound of formula (II) comprises contacting the compound of formula (II) with about 1.2 mol % of the first catalyst. In some embodiments, hydrogenating the compound of formula (II) comprises contacting the compound of formula (II) with about 1.4 mol % of the first catalyst. In some embodiments, hydrogenating the compound of formula (II) comprises contacting the compound of formula (II) with about 1.6 mol % of the first catalyst. In some embodiments, hydrogenating the compound of formula (II) comprises contacting the compound of formula (II) with about 1.7 mol % of the first catalyst. In some embodiments, hydrogenating the compound of formula (II) comprises contacting the compound of formula (II) with about 1.8 mol % of the first catalyst. In some embodiments, hydrogenating the compound of formula (II) comprises contacting the compound of formula (II) with about 2.0 mol % of the first catalyst. In some embodiments, hydrogenating the compound of formula (II) comprises contacting the compound of formula (II) with about 2.2 mol % of the first catalyst. In some embodiments, hydrogenating the compound of formula (II) comprises contacting the compound of formula (II) with about 2.5 mol % of the first catalyst. In some embodiments, hydrogenating the compound of formula (II) comprises contacting the compound of formula (II) with about 3.0 mol % of the first catalyst. In some embodiments, hydrogenating the compound of formula (II) comprises contacting the compound of formula (II) with about 4.0 mol % of the first catalyst. In some embodiments, hydrogenating the compound of formula (II) comprises contacting the compound of formula (II) with about 5.0 mol % of the first catalyst.

In some embodiments, the first catalyst is a palladium catalyst. In some embodiments, the first catalyst is a palladium (0) catalyst. In some embodiments, the first catalyst is a palladium on carbon. In some embodiments, the first catalyst is about 5% by weight palladium on carbon. In some embodiments, the first catalyst is about 10% by weight palladium on carbon.

In some embodiments, hydrogenating the compound of formula (II) takes place in the presence of a third acid. In some embodiments, the third acid is an inorganic acid. In some embodiments, the third acid is hydrochloric acid. In some embodiments, hydrogenating the compound of formula (II) takes place in a fifth solvent. In some embodiments, the fifth solvent is a mixture of solvents. In some embodiments, the fifth solvent is a mixture of tetrahydrofuran and water.

In some embodiments, some embodiments, hydrogenating the compound of formula (II) takes place at a temperature between about 20° C. and about 100° C. (e.g., between about 30° C. and about 90° C.; between about 40° C. and about 80° C.; between about 50° C. and about 70° C.; between about 60° C. and about 70° C.). In some embodiments, hydrogenating the compound of formula (II) takes place at a temperature between about 60° C. and about 70° C.

In some embodiments, hydrogenating the compound of formula (II) comprises the use of a flow system. In some embodiments, hydrogenating the compound of formula (II) comprises the use of a continuous flow system. In some embodiments, hydrogenating the compound of formula (II) comprises the use of a micropacked bed reactor. In some embodiments, hydrogenating the compound of formula (II) is performed under flow hydrogenation conditions. In some embodiments, hydrogenating the compound of formula (II) is performed under continuous flow hydrogenation conditions.

In some embodiments, hydrogenating the compound of formula (II) comprises contacting the compound of formula (II) with hydrogen gas. In some embodiments, hydrogenating the compound of formula (II) comprises contacting the compound of formula (II) with hydrogen gas at a pressure of at least about 1.0 megapascal (MPa) (e.g., at least about 2.0 megapascal (MPa); at least about 3.0 megapascal (MPa); or at least about 4.0 megapascal (MPa); or at least about 5.0 megapascal (MPa)). In some embodiments, hydrogenating the compound of formula (II) comprises contacting the compound of formula (II) with hydrogen gas at a pressure of about 1.0 MPa to about 5.0 MPa. In some embodiments, hydrogenating the compound of formula (II) comprises contacting the compound of formula (II) with hydrogen gas at a pressure of about 2.0 MPa to about 4.0 MPa. In some embodiments, hydrogenating the compound of formula (II) comprises contacting the compound of formula (II) with hydrogen gas at a pressure of about 1.0 MPa. In some embodiments, hydrogenating the compound of formula (II) comprises contacting the compound of formula (II) with hydrogen gas at a pressure of about 2.0 MPa. In some embodiments, hydrogenating the compound of formula (II) comprises contacting the compound of formula (II) with hydrogen gas at a pressure of about 3.0 MPa. In some embodiments, hydrogenating the compound of formula (II) comprises contacting the compound of formula (II) with hydrogen gas at a pressure of about 4.0 MPa. In some embodiments, hydrogenating the compound of formula (II) comprises contacting the compound of formula (II) with hydrogen gas at a pressure of about 5.0 MPa.

In some embodiments, R³ is a boronic acid group. In some embodiments, R³ is a boronic ester group. In some embodiments, R³ is

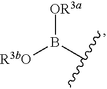

wherein each of R^{3a} and R^{3b} is, independently, H or C₁₋₆ alkyl, wherein R^{3a} and R^{3b} are optionally joined together with their intervening atoms to form a 5-10 membered ring that is optionally substituted with 0, 1, 2, 3, 4, 5, or 6 instances of R^{3c}, wherein each R^{3c} is, independently, C₁₋₆ alkyl.

In some embodiments, each of R^{3a} and R^{3b} is, independently, C₁₋₆ alkyl, wherein R^{3a} and R^{3b} are joined together with their intervening atoms to form a 5-10 membered ring that is optionally substituted with 0, 1, 2, 3, 4, 5, or 6 instances of R^{3c}, wherein each R^{3c} is, independently, C₁₋₆ alkyl. In some embodiments, each of R^{3a} and R^{3b} is, independently, C₁₋₆ alkyl, wherein R^{3a} and R^{3b} are joined together with their intervening atoms to form a 5-6 membered ring that is optionally substituted with 0, 1, 2, 3, 4, 5, or 6 instances of R^{3c}, wherein each R^{3c} is -Me. In some embodiments, R³ is

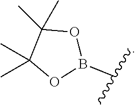 [SMILES: *B1OC(C)(C)C(C)(C)O1]

In some embodiments, cross-coupling the compound of formula (III) with the compound of formula (IV) comprises:

(a) converting the compound of formula (III):

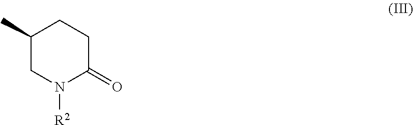

to a compound of formula (III-b):

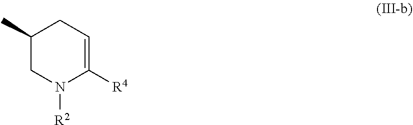

by contacting the compound of formula (III) with a sulfonylating/dehydrating agent, thereby providing the compound of formula (III-b),

wherein R² is a nitrogen protecting group as defined herein and R⁴ is an alkyl, haloalkyl or aryl sulfonate (e.g., methanesulfonate (—OS(═O)₂CH₃, trifluoromethanesulfonate (—OS(═O)₂CF₃), phenyl sulfonate (—OS(═O)₂Ph), toluenesulfonate (—OS(═O)₂C₆H₄—CH₃); and

(b) contacting the compound of formula (III-b) with a compound of formula (IV):

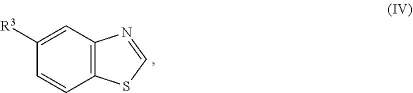

thereby producing a compound of formula (V):

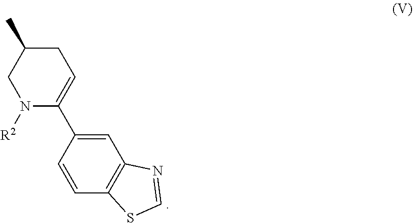

In some embodiments, R⁴ is trifluoromethanesulfonate and the compound of formula (III-b) is

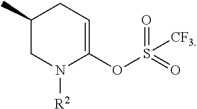

In some embodiments, cross-coupling the compound of formula (III) with the compound of formula (IV) comprises contacting the compound of formula (III) with a sulfonylating/dehydrating agent in the presence of a first base (e.g., thereby converting the compound of formula (III) into a compound of formula (III-b).

In some embodiments, the dehydrating agent is a sulfonimide. In some embodiments, the sulfonylating/dehydrating agent is 1,1,1-trifluoro-N-phenyl-N-(trifluoromethanesulfonyl)methanesulfonamide (PHNTf₂).

In some embodiments, the first base is an inorganic base. In some embodiments, the first base is an organic base.

In some embodiments, the first base is a lithium base. In some embodiments, the first base is an amine base. In some embodiments, the first base is lithium bis(trimethylsilyl)amide (LiHMDS).

In some embodiments, contacting the compound of formula (III) with a sulfonylating/dehydrating agent to provide a compound of formula (III-b) takes place in a fourth solvent. In some embodiments, the fourth solvent is an aprotic solvent. In some embodiments (e.g., an ether solvent). In some embodiments, the solvent is tetrahydrofuran.

In some embodiments, contacting the compound of formula (III) with a sulfonylating/dehydrating agent to provide a compound of formula (III-b) takes place at a temperature below about 0° C. (e.g., below about −10° C.; below about −20° C.; below about −30° C.; below about −40° C.; below about −60° C.; below about −70° C.; below about −80° C.). In some embodiments, contacting the compound of formula (III) with a sulfonylating/dehydrating agent to provide a compound of formula (III-b) takes place at a temperature below about −60° C. In some embodiments, contacting the compound of formula (III) with a sulfonylating/dehydrating agent to provide a compound of formula (III-b) takes place at a temperature between about −50° C. and −80° C. In some embodiments, contacting the compound of formula (III) with a sulfonylating/dehydrating agent to provide a compound of formula (III-b) takes place at a temperature of about −70° C.

In some embodiments, cross-coupling the compound of formula (III) with the compound of formula (IV) comprises contacting the compound of formula (III-b) with the compound of formula (IV) in the presence of a second catalyst.

In some embodiments, cross-coupling the compound of formula (III) with the compound of formula (IV) comprises contacting the compound of formula (III-b) with the compound of formula (IV) in the presence of less than about 10 mol % (e.g., less than about 9 mol %; less than about 8 mol %; less than about 7 mol %; less than about 6 mol %; less than about 5 mol %; less than about 4 mol %; less than about 3 mol %; less than about 2.75 mol %; less than about 2.5 mol %; less than about 2.25 mol %; less than about 2 mol %; less than about 1.95 mol %; less than about 1.85 mol %; less than about 1.75 mol %; less than about 1.65 mol %; less than about 1.55 mol %; less than about 1.45 mol %; less than about 1.35 mol %; less than about 1.25 mol %; less than about 1.15 mol %; less than about 1.05 mol %; less than about 1 mol %; less than about 0.9 mol %; less than about 0.8 mol %; less than about 0.7 mol %; less than about 0.6 mol %; less than about 0.5 mol %; less than about 0.4 mol %; less than about 0.3 mol %; less than about 0.2 mol %; or less than about 0.1 mol %) of a second catalyst. In some embodiments, cross-coupling the compound of formula (III) with the compound of formula (IV) comprises contacting the compound of formula (III-b) with the compound of formula (IV) in the presence of about 0.1 mol % to about 10 mol % (e.g., about 0.5 mol % to about 7 mol %; about 1 mol % to about 5 mol %; about 1 mol % to about 4 mol %; about 2 mol % to about 5 mol %; or about 2 mol % to about 4 mol %) of a second catalyst.

In some embodiments, cross-coupling the compound of formula (III) with the compound of formula (IV) comprises contacting the compound of formula (III-b) with the compound of formula (IV) in the presence of about 0.1 mol % to about 10 mol % of the second catalyst. In some embodiments, cross-coupling the compound of formula (III) with the compound of formula (IV) comprises contacting the compound of formula (III-b) with the compound of formula (IV) in the presence of about 0.5 mol % to about 7 mol % of the second catalyst. In some embodiments, cross-coupling the compound of formula (III) with the compound of formula (IV) comprises contacting the compound of formula (III-b) with the compound of formula (IV) in the presence of about 1 mol % to about 5 mol % of the second catalyst. In some embodiments, cross-coupling the compound of formula (III) with the compound of formula (IV) comprises contacting the compound of formula (III-b) with the compound of formula (IV) in the presence of about 1 mol % to about 4 mol % of the second catalyst. In some embodiments, cross-coupling the compound of formula (III) with the compound of formula (IV) comprises contacting the compound of formula (III-b) with the compound of formula (IV) in the presence of about 2 mol % to about 5 mol % of the second catalyst. In some embodiments, cross-coupling the compound of formula (III) with the compound of formula (IV) comprises contacting the compound of formula (III-b) with the compound of formula (IV) in the presence of about 2 mol % to about 4 mol %) of a second catalyst.

In some embodiments, cross-coupling the compound of formula (III) with the compound of formula (IV) comprises contacting the compound of formula (III-b) with the compound of formula (IV) in the presence of about 1 mol % of the second catalyst. In some embodiments, cross-coupling the compound of formula (III) with the compound of formula (IV) comprises contacting the compound of formula (III-b) with the compound of formula (IV) in the presence of about 2 mol % of the second catalyst. In some embodiments, cross-coupling the compound of formula (III) with the compound of formula (IV) comprises contacting the compound of formula (III-b) with the compound of formula (IV) in the presence of about 3 mol % of the second catalyst. In some embodiments, cross-coupling the compound of formula (III) with the compound of formula (IV) comprises contacting the compound of formula (III-b) with the compound of formula (IV) in the presence of about 4 mol % of the second catalyst. In some embodiments, cross-coupling the compound of formula (III) with the compound of formula (IV) comprises contacting the compound of formula (III-b) with the compound of formula (IV) in the presence of about 5 mol % of the second catalyst.

In some embodiments, the second catalyst is a palladium catalyst. In some embodiments, the second catalyst is a palladium (II) catalyst. In some embodiments, the second catalyst is bis(triphenylphosphine)palladium(II) dichloride.

In some embodiments, cross-coupling the compound of formula (III) with the compound of formula (IV) comprises contacting the compound of formula (III-b) with the compound of formula (IV) in the presence of a second base.

In some embodiments, the second base is an inorganic base. In some embodiments, the second base is a carbonate base. In some embodiments, the second base is tripotassium phosphate (K₃PO₄).

In some embodiments, contacting the compound of formula (III-b) with the compound of formula (IV) takes place in a seventh solvent. In some embodiments, the seventh solvent is a mixture of solvents. In some embodiments, the seventh solvent is a mixture of tetrahydrofuran and water.

In some embodiments, contacting the compound of formula (III-b) with the compound of formula (IV) takes place at a temperature between about 20° C. and about 120° C. (e.g., between about 30° C. and about 110° C.; between about 40° C. and about 100° C.; between about 50° C. and about 90° C.; between about 60° C. and about 80° C.). In some embodiments, hydrogenating the compound of formula (II) takes place at a temperature between about 60° C. and about 80° C.

In some embodiments, the process further comprises removing the nitrogen protecting group of the compound of formula (V) to provide a compound of formula (V-a). In some embodiments, removing the nitrogen protecting group of the compound of formula (V) comprises contacting the compound of formula (V) with a first acid.

In some embodiments, the first acid is an inorganic acid. In some embodiments, the first acid is hydrochloric acid (HCl) or phosphoric acid (H₃PO₄). In some embodiments, the first acid is hydrochloric acid (HCl).

In some embodiments, the first acid is an organic acid. In some embodiments, the first acid is trifluoromethanesulfonic acid (TfOH), trifluoroacetic acid (TFA), or p-toluenesulfonic acid (PTSA).

In some embodiments, removing the nitrogen protecting group of the compound of formula (V) takes place in an eight solvent. In some embodiments, the eight solvent is a protic solvent. In some embodiments, the eight solvent is methanol.

In some embodiments, reducing the compound of formula (V-a) comprises contacting the compound of formula (V-a) with a reducing agent. In some embodiments, the reducing agent is a hydride reducing agent. In some embodiments, the reducing agent is a borohydride reducing agent. In some embodiments, the reducing agent is sodium borohydride (NaBH₄).

In some embodiments, reducing the compound of formula (V-a) comprises contacting the compound of formula (V-a) with a first solvent at a temperature below about 0° C. (e.g., below about −1° C.; below about −2° C.; below about −3° C.; below about −4° C.; below about −5° C.; below about −6° C.; below about −7° C.; below about −8° C.; or below about −10° C.). In some embodiments, reducing the compound of formula (V-a) comprises contacting the compound of formula (V-a) with a first solvent at a temperature between about −20° C. and 20° C. In some embodiments, reducing the compound of formula (V-a) comprises contacting the compound of formula (V-a) with a first solvent at a temperature between about −15° C. and 10° C. In some embodiments, reducing the compound of formula (V-a) comprises contacting the compound of formula (V-a) with a first solvent at a temperature between about −10° C. and 0° C. In some embodiments, reducing the compound of formula (V-a) comprises contacting the compound of formula (V-a) with a first solvent at a temperature between about −15° C. and −5° C. In some embodiments, reducing the compound of formula (V-a) comprises contacting the compound of formula (V-a) with a first solvent at a temperature of about −20° C. In some embodiments, reducing the compound of formula (V-a) comprises contacting the compound of formula (V-a) with a first solvent at a temperature of about −10° C. In some embodiments, reducing the compound of formula (V-a) comprises contacting the compound of formula (V-a) with a first solvent at a temperature of about −0° C. In some embodiments, reducing the compound of formula (V-a) comprises contacting the compound of formula (V-a) with a first solvent at a temperature of about 10° C. In some embodiments, reducing the compound of formula (V-a) comprises contacting the compound of formula (V-a) with a first solvent at a temperature of about 20° C.

In some embodiments, the first solvent is a protic solvent. In some embodiments, the first solvent is methanol.

In some embodiments, R⁶ is a nitrogen protecting group and R⁷ is a nitrogen protecting group. In some embodiments, R⁶ is a nitrogen protecting group and R⁷ is H. In some embodiments, R⁶ is a carbamate group and R⁷ is a carbamate group. In some embodiments, R⁶ is a carbamate group and R⁷ is H. In some embodiments, R⁶ is

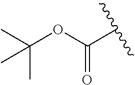 [SMILES: *C(=O)OC(C)(C)C]

### and R⁷ is,

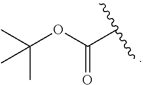 [SMILES: *C(=O)OC(C)(C)C]

In some embodiments, R⁶ is

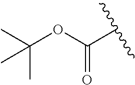 [SMILES: *C(=O)OC(C)(C)C]

and R⁷ is H. In some embodiments, coupling the compound of formula (VI) with the compound of formula (VII) comprises contacting the compound of formula (VI) with the compound of formula (VII) having a molar ratio of about 1.5:1 to about 1:1.5. In some embodiments, coupling the compound of formula (VI) with the compound of formula (VII) comprises contacting the compound of formula (VI) with the compound of formula (VII) having a molar ratio of about 1:1 to about 1:1.4. In some embodiments, coupling the compound of formula (VI) with the compound of formula (VII) comprises contacting the compound of formula (VI) with the compound of formula (VII) having a molar ratio of about 1:1.1 to about 1:1.4. In some embodiments, coupling the compound of formula (VI) with the compound of formula (VII) comprises contacting the compound of formula (VI) with the compound of formula (VII) having a molar ratio of about 1:1.3. In some embodiments, coupling the compound of formula (VIa) with the compound of formula (VII) comprises contacting the compound of formula (VI) with the compound of formula (VII) in the presence of a coupling reagent.

In some embodiments, the coupling reagent is an anhydride coupling reagent; a triazole-based coupling reagent; a carbodiimide coupling reagent; an imidazolium coupling reagent; a phosphonium salt coupling reagent; or a pyridinium salt coupling reagent.

In some embodiments, the coupling reagent is an anhydride coupling reagent. Exemplary anhydride coupling reagents include but are not limited to propylphosphonic anhydride (T3P®).

In some embodiments, the coupling reagent is a triazole-based coupling reagent. Exemplary triazole-based coupling reagents include but are not limited to 1-[bis(dimethylamino)methylene]-1H-1,2,3-triazolo[4,5-b]pyridinium 3-oxide hexafluorophosphate (HATU); N,N,N′,N′-tetramethyl-O-(1H-benzotriazol-1-yl)uronium hexafluorophosphate (HBTU); O-(1H-6-chlorobenzotriazole-1-yl)-1,1,3,3-tetramethyluronium hexafluorophosphate (HCTU); and 2-(1H-benzotriazole-1-yl)-1,1,3,3-tetramethylaminium tetrafluoroborate (TBTU). In some embodiments, the coupling reagent is 1-[bis(dimethylamino)methylene]-1H-1,2,3-triazolo[4,5-b]pyridinium 3-oxide hexafluorophosphate (HATU) or 2-(1H-benzotriazole-1-yl)-1,1,3,3-tetramethylaminium tetrafluoroborate (TBTU). In some embodiments, the coupling reagent is 1-[bis(dimethylamino)methylene]-1H-1,2,3-triazolo[4,5-b]pyridinium 3-oxide hexafluorophosphate (HATU); N,N,N′,N′-tetramethyl-O-(1H-benzotriazol-1-yl)uronium hexafluorophosphate (HBTU); O-(1H-6-chlorobenzotriazole-1-yl)-1,1,3,3-tetramethyluronium hexafluorophosphate (HCTU); or 2-(1H-benzotriazole-1-yl)-1,1,3,3-tetramethylaminium tetrafluoroborate (TBTU). In some embodiments, the coupling reagent is 1-[bis(dimethylamino)methylene]-1H-1,2,3-triazolo[4,5-b]pyridinium 3-oxid hexafluorophosphate (HATU) or 2-(1H-benzotriazole-1-yl)-1,1,3,3-tetramethylaminium tetrafluoroborate (TBTU). In some embodiments, the coupling reagent is 2-(1H-benzotriazole-1-yl)-1,1,3,3-tetramethylaminium tetrafluoroborate (TBTU).

In some embodiments, the coupling reagent is a carbodiimide coupling reagent. Exemplary carbodiimide coupling reagents include but are not limited to dicyclohexylcarbodiimide (DCC); diisopropylcarbodiimide (DIC); and ethyl-(N′,N′-dimethylamino)propylcarbodiimide hydrochloride (EDC).

In some embodiments, the coupling reagent is an imidazolium coupling reagent. Exemplary imidazolium coupling reagents include but are not limited to carbonyldiimidazole (CDI); chloro-1,3-dimethylimidazolidinium hexafluorophosphate; 2-chloro-1,3-dimethylimidazolinium chloride; and 1,1′-oxalyldiimidazole.

In some embodiments, the coupling reagent is a phosphonium salt coupling reagent. Exemplary phosphonium salt coupling reagents include but are not limited to benzotriazole-1-yl-oxy-tris-(dimethylamino)-phosphonium hexafluorophosphate (BOP); benzotriazole-1-yl-oxy-tris-pyrrolidino-phosphonium hexafluorophosphate (PyBOP); (7-Azabenzotriazol-1-yloxy)trispyrrolidinophosphonium hexafluorophosphate (PyAOP); bromotripyrrolidinophosphonium hexafluorophosphate (PyBrOP); and bis(2-oxo-3-oxazolidinyl)phosphinic chloride (BOP-Cl).

In some embodiments, the coupling reagent is a pyridinium salt coupling reagent. Exemplary pyridinium salt coupling reagents include but are not limited to 2-chloro-1-methylpyridinium iodide (Mukaiyama reagent).

In some embodiments, the compound of formula (VI) and the coupling reagent have a molar ratio of about 1.5:1 to about 1:1.5. In some embodiments, the compound of formula (VI) and the coupling reagent have a molar ratio of about 1:1 to about 1:1.5. In some embodiments, the coupling reagent have a molar ratio of about 1:1.1 to about 1:1.4.

In some embodiments, the compound of formula (VI) and the coupling reagent have a molar ratio of about 1:1.2 to about 1:1.4. In some embodiments, the compound of formula (VI) and the coupling reagent have a molar ratio of about 1:1.3.

In some embodiments, coupling the compound of formula (VI) with the compound of formula (VII) comprises contacting the compound of formula (VI) with the compound of formula (VII) in the presence of a third base.

In some embodiments, the third base is an organic base. In some embodiments, the third base is an amine base. In some embodiments, the third base is triethylamine (TEA), N,N-dii sopropylethylamine (DIPEA), or pyridine.

In some embodiments, the third base is triethylamine (TEA) or N,N-diisopropylethylamine (DIPEA).

In some embodiments, coupling the compound of formula (VI) with the compound of formula (VII) comprises contacting the compound of formula (VI) with the compound of formula (VII) in a ninth solvent. In some embodiments, the ninth solvent is an aprotic solvent. In some embodiments, the ninth solvent is tetrahydrofuran.

In some embodiments, R⁸ is a nitrogen protecting group and R⁹ is a nitrogen protecting group. In some embodiments, R⁸ is a nitrogen protecting group and R⁹ is H. In some embodiments, R⁸ is a carbamate group and R⁹ is a carbamate group. In some embodiments, R⁸ is a carbamate group and R⁹ is a H. In some embodiments, R⁸ is

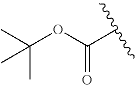 [SMILES: *C(=O)OC(C)(C)C]

### and R⁹ is

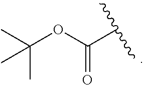 [SMILES: *C(=O)OC(C)(C)C]

In some embodiments, R⁸ is

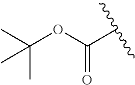 [SMILES: *C(=O)OC(C)(C)C]

### and R⁹ is H.

In some embodiments, the process further comprises removing each nitrogen protecting group from the compound of formula (I-a).

In some embodiments, removing each nitrogen protecting group from the compound of formula (I-a) comprises contacting the compound of formula (I-a) with a second acid.

In some embodiments, removing each nitrogen protecting group from the compound of formula (I-a) comprises contacting the compound of formula (I-a) with a second acid at a temperature of about 0° C. to about 100° C. (e.g., about 15° C. to about 65° C.; about 20° C. to about 50° C.; about 25° C. to about 45° C.; or about 35° C. to about 55° C.). In some embodiments, removing each nitrogen protecting group from the compound of formula (I-a) comprises contacting the compound of formula (I-a) with a second acid at a temperature of about 15° C. to about 65° C. In some embodiments, removing each nitrogen protecting group from the compound of formula (I-a) comprises contacting the compound of formula (I-a) with a second acid at a temperature of about 20° C. to about 50° C. In some embodiments, removing each nitrogen protecting group from the compound of formula (I-a) comprises contacting the compound of formula (I-a) with a second acid at a temperature of about 25° C. to about 45° C. In some embodiments, removing each nitrogen protecting group from the compound of formula (I-a) comprises contacting the compound of formula (I-a) with a second acid at a temperature of about 35° C. to about 55° C. In some embodiments, removing each nitrogen protecting group from the compound of formula (I-a) comprises contacting the compound of formula (I-a) with a second acid at a temperature of about 70° C. In some embodiments, removing each nitrogen protecting group from the compound of formula (I-a) comprises contacting the compound of formula (I-a) with a second acid at a temperature of about 60° C. In some embodiments, removing each nitrogen protecting group from the compound of formula (I-a) comprises contacting the compound of formula (I-a) with a second acid at a temperature of about 50° C. In some embodiments, removing each nitrogen protecting group from the compound of formula (I-a) comprises contacting the compound of formula (I-a) with a second acid at a temperature of about 45° C. In some embodiments, removing each nitrogen protecting group from the compound of formula (I-a) comprises contacting the compound of formula (I-a) with a second acid at a temperature of about 40° C. In some embodiments, removing each nitrogen protecting group from the compound of formula (I-a) comprises contacting the compound of formula (I-a) with a second acid at a temperature of about 30° C. In some embodiments, removing each nitrogen protecting group from the compound of formula (I-a) comprises contacting the compound of formula (I-a) with a second acid at a temperature of about 20° C.

In some embodiments, the compound of formula (I-a) and the second acid have a molar ratio of about 1:1 to about 1:10. In some embodiments, the compound of formula (I-a) and the second acid have a molar ratio of about 1:2 to about 1:7. In some embodiments, the compound of formula (I-a) and the second acid have a molar ratio of about 1:3 to about 1:6. In some embodiments, the compound of formula (I-a) and the second acid have a molar ratio of about 1:3 to about 1:5. In some embodiments, the compound of formula (I-a) and the second acid have a molar ratio of about 1:4.

In some embodiments, the second acid is an inorganic acid. In some embodiments, the second acid is hydrochloric acid (HCl) or phosphoric acid (H₃PO₄).

In some embodiments, the second acid is an organic acid. In some embodiments, the second acid is trifluoromethanesulfonic acid (TfOH), trifluoroacetic acid (TFA), or p-toluenesulfonic acid (PTSA). In some embodiments, the second acid is trifluoromethanesulfonic acid (TfOH).

In some embodiments, removal of each nitrogen protecting group from the compound of formula (I-a) results in a salt of the compound of formula (I). In some embodiments, the salt of the compound of formula (I) is a hydrochloride salt. In some embodiments, the salt of the compound of formula (I) is a trifluoroacetate salt. In some embodiments, the salt of the compound of formula (I) is a trifluoromethanesulfonate salt. In some embodiments, the salt of the compound of formula (I) is a paratoluenesulfonate salt.

In some embodiments, the compound of formula (I) is not purified by chromatography. In some embodiments, the entire process of preparing the compound of formula (I) does not include purification by chromatography.

In some embodiments, the process further comprises converting a salt of the compound of formula (I) into the free base of the compound of formula (I). In some embodiments, converting the salt of the compound of formula (I) into the free base of the compound of formula (I) comprises contacting the salt of the compound of formula (I) with a fifth base in a in a tenth solvent. In some embodiments, the fifth base is an inorganic base. In some embodiments, the fifth base is a carbonate base. In some embodiments, the fifth base is sodium carbonate. In some embodiments, the tenth solvent is a mixture of water and an organic solvent. In some embodiments, the tenth solvent is a mixture of water, ethanol and tetrahydrofuran.

In some embodiments, the process further comprises producing a crystalline form of the compound of formula (I) (e.g., the free base of the compound of formula (I)).

In some embodiments, producing a crystalline form of the compound of formula (I) comprises subjecting a solution of the compound of formula (I) to conditions that result in crystallization of the compound of formula (I).

In some embodiments, producing a crystalline form of the compound of formula (I) comprises: dissolving the compound of formula (I) in a first solvent; and partially evaporating the first solvent. In some embodiments, the process further comprises seeding the solution resulting from dissolving the compound of formula (I) in the first solvent with a small amount of crystalline compound of formula (I) (e.g., less than about 5% of the amount of compound of formula (I) present in the solution).

In some embodiments, the first solvent is a protic solvent. In some embodiments, the first solvent is a polar solvent.

In some embodiments, the first solvent is MeCN, methanol, ethanol, isopropyl alcohol, n-propanol, n-BuOH, water, or a mixture thereof. In some embodiments, the first solvent is MeCN, ethanol, methanol, water, or a mixture thereof. In some embodiments, the first solvent is a mixture of water and methanol (e.g., a 9:1 v/v mixture). In some embodiments, the process further comprises adding water to a mixture obtained after partially evaporating the first solvent. In some embodiments, the process further comprises stirring a suspension resulting from the previous steps for at least 1 hr (e.g., for about 1-10 hrs, about 2-8 hours, about 3-6 hours, about 5 hours). In some embodiments, producing a crystalline form of the compound of formula (I) further comprises a filtration step to collect a crude crystalline form of the compound of formula (I).

In some embodiments, producing a crystalline form of the compound of formula (I) further comprises: slurrying the crude crystalline form of the compound of formula (I) in a second solvent; and filtering by centrifugation.

In some embodiments, the second solvent is a protic solvent. In some embodiments, the second solvent is a polar solvent. In some embodiments, the second solvent is MeCN, methanol, ethanol, isopropyl alcohol, n-propanol, n-BuOH, water, or a mixture thereof.

In some embodiments, the second solvent is MeCN, ethanol, methanol, water, or a mixture thereof. In some embodiments, the second solvent is a mixture of methanol and water.

### Pharmaceutical Compositions

In some embodiments, provided are pharmaceutical compositions comprising a compound of formula (I)

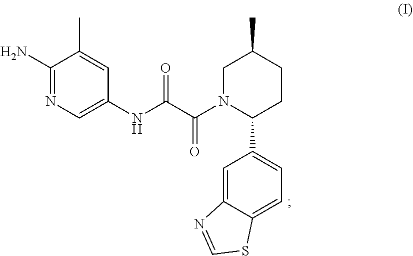 [SMILES: Cc1cc(NC(=O)C(=O)N2C[C@@H](C)CC[C@@H]2c2ccc3scnc3c2)cnc1N]

or a pharmaceutically acceptable salt thereof and at least one pharmaceutically acceptable excipient.

In some embodiments, provided are pharmaceutical compositions comprising a compound of formula (I)

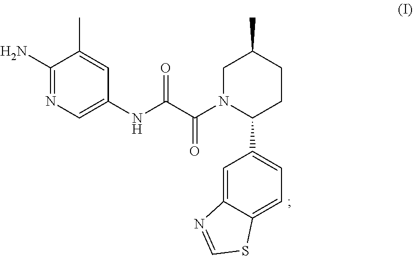 [SMILES: Cc1cc(NC(=O)C(=O)N2C[C@@H](C)CC[C@@H]2c2ccc3scnc3c2)cnc1N]

as the free base and at least one pharmaceutically acceptable excipient.

In some embodiments, provided are pharmaceutical compositions comprising as the pharmaceutically active ingredient a compound of formula (I)

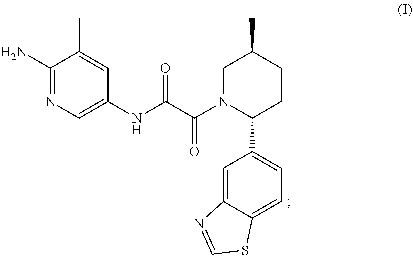 [SMILES: Cc1cc(NC(=O)C(=O)N2C[C@@H](C)CC[C@@H]2c2ccc3scnc3c2)cnc1N]

or a pharmaceutically acceptable salt thereof and at least one pharmaceutically acceptable excipient.

In some embodiments, the pharmaceutically active ingredient is the free base of the compound of formula (I).

In some embodiments, the composition comprises a crystalline form of the compound of formula (I). In some embodiments, the composition comprises crystalline form A of the compound of formula (I) as described herein and at least one pharmaceutically acceptable excipient.

The amounts and ranges described below as pertaining to “a compound of formula (I)” can refer to the compound of formula (I) generally, to a crystalline form of a compound of formula (I), or to the crystalline form A of the compound of formula (I) described herein. For compositions and dosage forms comprising a pharmaceutically acceptable salt of a compound of formula (I), the amounts and ranges described below as pertaining to “a compound of formula (I)” refer to the equivalent dose of the free base of the compound contained in the composition or dosage form.

In some embodiments, the pharmaceutical composition comprises about 5% (w/w) to about 50% (w/w) of the compound of formula (I). In some embodiments, the pharmaceutical composition comprises about 10% (w/w) to about 45% (w/w) of a compound of formula (I).

In some embodiments, the pharmaceutical composition comprises about 8% (w/w) to about 17% (w/w), about 25% (w/w) to about 35% (w/w) or about 35% (w/w) to about 45% (w/w) of a compound of formula (I).

In some embodiments, the pharmaceutical composition comprises about 8% (w/w) to about 17% (w/w) of a compound of formula (I). In some embodiments, the pharmaceutical composition comprises about 10% (w/w) to about 15% (w/w) of a compound of formula (I).

In some embodiments, the pharmaceutical composition comprises about 12% (w/w) to about 13% (w/w) of a compound of formula (I).

In some embodiments, the pharmaceutical composition comprises about 12.5% (w/w) of a compound of formula (I).

In some embodiments, the pharmaceutical composition comprises about 25% (w/w) to about 35% (w/w) of a compound of formula (I). In some embodiments, the pharmaceutical composition comprises about 27% (w/w) to about 33% (w/w) of a compound of formula (I). In some embodiments, the pharmaceutical composition comprises about 29% (w/w) to about 31% (w/w) of a compound of formula (I).

In some embodiments, the pharmaceutical composition comprises about 29.2% (w/w) to about 29.7% (w/w) of a compound of formula (I).

In some embodiments, the pharmaceutical composition comprises about 29.4% (w/w) of a compound of formula (I).

In some embodiments, the pharmaceutical composition comprises about 35% (w/w) to about 45% (w/w) of a compound of formula (I). In some embodiments, the pharmaceutical composition comprises about 37% (w/w) to about 43% (w/w) of a compound of formula (I).

In some embodiments, the pharmaceutical composition comprises about 39% (w/w) to about 31% (w/w) of a compound of formula (I).

In some embodiments, the pharmaceutical composition comprises about 40% (w/w) of a compound of formula (I).

In certain embodiments, the pharmaceutical composition comprises about 12.5% (w/w), about 29.4% (w/w) or about 40% (w/w) of a compound of formula (I).

In some embodiments, the pharmaceutical composition comprises a filler. In certain embodiments, the filler is selected from the group consisting of a sugar, an inorganic material, a microcrystalline cellulose, a starch, a polysaccharide, a cellulose, a polyvinylpyrrolidone, a polyvinyl acrylate and combinations thereof.

In certain embodiments, the filler is selected from the group consisting of a sugar, an inorganic material, and combinations thereof. In certain embodiments, the sugar is selected from the group consisting of mannitol, lactose, sucrose, fructose, glucose, maltose, and combinations thereof. In certain embodiments, the inorganic material is selected from the group consisting of dibasic calcium phosphate, hydroxyapatite, sodium carbonate, sodium bicarbonate, calcium carbonate, bentonite, kaolin, and combinations thereof.

In certain embodiments, the filler is selected from the group consisting of a microcrystalline cellulose, a starch, a polysaccharide, a cellulose, a polyvinylpyrrolidone, a polyvinyl acrylate, and combinations thereof. In certain embodiments, the cellulose is selected from the group consisting of a hydroxypropylcellulose, a hypromellose, a carboxymethylcellulose, a methylcellulose, a hydroxypropylmethylcellulose, and combinations thereof.

In some embodiments, the filler is microcrystalline cellulose (e.g., Avicel®). The microcrystalline cellulose filler can be of different grades. In some embodiments, the filler is microcrystalline cellulose PH 102, PH 200 or a mixture thereof. In some embodiments, the filler is microcrystalline cellulose PH 102 (e.g., Avicel® PH 102). In some embodiments, the filler is microcrystalline cellulose PH 200 (e.g., Avicel® PH 200). In some embodiments, the filler is a mixture of microcrystalline cellulose PH 102 (e.g., Avicel® PH 102) and microcrystalline cellulose PH 200 (e.g., Avicel® PH 200). In further embodiments, the filler contains equal amounts of the two grades of microcrystalline cellulose.

In certain embodiments, the pharmaceutical composition comprises about 50% (w/w) to about 90% (w/w) filler. In certain embodiments, the pharmaceutical composition comprises about 50% (w/w) to about 60% (w/w) filler. In certain embodiments, the pharmaceutical composition comprises about 52% (w/w) to about 58% (w/w) filler. In certain embodiments, the pharmaceutical composition comprises about 52% (w/w) to about 56% (w/w) filler.

In certain embodiments, the pharmaceutical composition comprises about 50% (w/w), about 51% (w/w), about 52% (w/w), about 53% (w/w), about 54% (w/w), about 55% (w/w), about 56% (w/w), about 57% (w/w) or about 58% (w/w) filler.

In certain embodiments, the pharmaceutical composition comprises about 54% (w/w) filler.

In certain embodiments, the pharmaceutical composition comprises about 61% (w/w) to about 70% (w/w) filler. In certain embodiments, the pharmaceutical composition comprises about 63% (w/w) to about 68% (w/w) filler. In certain embodiments, the pharmaceutical composition comprises about 65% (w/w) to about 66% (w/w) filler.

In certain embodiments, the pharmaceutical composition comprises about 61% (w/w), about 62% (w/w), about 63% (w/w), about 64% (w/w), about 65% (w/w), about 65.4% (w/w), about 66% (w/w), about 67% (w/w), about 68% (w/w), about 69% (w/w) or about 70% (w/w) filler. In certain embodiments, the pharmaceutical composition comprises about 64% (w/w), about 65% (w/w), about 65.4% (w/w), about 66% (w/w) or about 67% (w/w) filler.

In certain embodiments, the pharmaceutical composition comprises about 65.4% (w/w) filler.

In certain embodiments, the pharmaceutical composition comprises about 75% (w/w) to about 85% (w/w) filler. In certain embodiments, the pharmaceutical composition comprises about 77% (w/w) to about 83% (w/w) filler. In certain embodiments, the pharmaceutical composition comprises about 79% (w/w) to about 82% (w/w) filler. In certain embodiments, the pharmaceutical composition comprises about 80% (w/w) to about 82% (w/w) filler.

In certain embodiments, the pharmaceutical composition comprises about 75% (w/w), about 76% (w/w), about 77% (w/w), about 78% (w/w), about 79% (w/w), about 80% (w/w), about 81% (w/w), about 81.5% (w/w), about 82% (w/w), about 83% (w/w), about 84% (w/w) or about 85% (w/w) filler. In certain embodiments, the pharmaceutical composition comprises about 80% (w/w), about 81% (w/w), about 81.5% (w/w), about 82% (w/w) or about 83% (w/w) filler.

In certain embodiments, the pharmaceutical composition comprises about 81.5% (w/w) filler.

In some embodiments, the pharmaceutical composition comprises a glidant. In certain embodiments, the glidant is selected from the group consisting of colloidal silicon dioxide, talc, kaolin, bentonite, or combinations thereof.

In further embodiments, the glidant is colloidal silicon dioxide. In some embodiments, the colloidal silicon dioxide is prepared through a process involving flame hydrolysis of silicon tetrachloride in an oxy-hydrogen flame and is referred to as “fumed silica” or “untreated fumed silica” (e.g., Aerosil® 200, CAB-O-SIL® M-5P).

In certain embodiments, the pharmaceutical composition comprises about 0.5% (w/w) to about 1.5% (w/w) glidant. In certain embodiments, the pharmaceutical composition comprises about 0.75% (w/w) to about 1.25% (w/w) glidant. In certain embodiments, the pharmaceutical composition comprises about 0.8% (w/w) to about 1% (w/w) glidant. In certain embodiments, the pharmaceutical composition comprises about 0.75% (w/w) to about 0.95% (w/w) glidant. In certain embodiments, the pharmaceutical composition comprises about 0.85% (w/w) to about 0.9% (w/w) glidant. In certain embodiments, the pharmaceutical composition comprises about 0.9% (w/w) to about 1.1% (w/w) glidant. In certain embodiments, the pharmaceutical composition comprises about 0.95% (w/w) to about 1.05% (w/w) glidant.

In certain embodiments, the pharmaceutical composition comprises about 0.8% (w/w), about 0.82% (w/w), about 0.84% (w/w), about 0.86% (w/w), about 0.87% (w/w), about 0.88% (w/w), about 0.9% (w/w), about 0.92% (w/w), about 0.94% (w/w), about 0.96% (w/w), about 0.98% (w/w), about 1.0% (w/w), about 1.02% (w/w), about 1.04% (w/w), about 1.06% (w/w), about 1.08% (w/w) or about 1.1% (w/w) glidant. In certain embodiments, the pharmaceutical composition comprises about 0.86% (w/w), about 0.87% (w/w), about 0.88% (w/w), about 0.98% (w/w), about 1.00% (w/w) or about 1.02% (w/w) glidant.

In certain embodiments, the pharmaceutical composition comprises about 0.87% (w/w) glidant.

In certain embodiments, the pharmaceutical composition comprises about 1% (w/w) glidant.

In some embodiments, the pharmaceutical composition comprises a disintegrant. In certain embodiments, the disintegrant is selected from the group consisting of sodium starch glycolate, a crospovidone, croscarmellose sodium, and combinations thereof. In further embodiments, the disintegrant is croscarmellose sodium (e.g., Ac-Di-Sol®).

In certain embodiments, the pharmaceutical composition comprises about 2% (w/w) to about 6% (w/w) disintegrant. In certain embodiments, the pharmaceutical composition comprises about 3% (w/w) to about 5% (w/w) disintegrant. In certain embodiments, the pharmaceutical composition comprises about 3.2% (w/w) to about 4% (w/w) disintegrant. In certain embodiments, the pharmaceutical composition comprises about 3% (w/w) to about 3.8% (w/w) disintegrant. In certain embodiments, the pharmaceutical composition comprises about 3.4% (w/w) to about 3.6% (w/w) disintegrant. In certain embodiments, the pharmaceutical composition comprises about 3.6% (w/w) to about 4.4% (w/w) disintegrant. In certain embodiments, the pharmaceutical composition comprises about 3.8% (w/w) to about 4.2% (w/w) disintegrant.

In certain embodiments, the pharmaceutical composition comprises about 3.2% (w/w), about 3.28% (w/w), about 3.36% (w/w), about 3.44% (w/w), about 3.47% (w/w), about 3.52% (w/w), about 3.6% (w/w), about 3.68% (w/w), about 3.76% (w/w), about 3.84% (w/w), about 3.92% (w/w), about 4% (w/w), about 4.08% (w/w), about 4.16% (w/w), about 4.24% (w/w), about 4.32% (w/w) or about 4.4% (w/w) disintegrant. In certain embodiments, the pharmaceutical composition comprises about 3.44% (w/w), about 3.47% (w/w), about 3.52% (w/w), about 3.92% (w/w), about 4.00% (w/w) or about 4.08% (w/w) disintegrant.

In certain embodiments, the pharmaceutical composition comprises about 3.47% (w/w) disintegrant.

In certain embodiments, the pharmaceutical composition comprises about 4% (w/w) disintegrant.

In some embodiments, the pharmaceutical composition comprises a lubricant. In certain embodiments, the lubricant is selected from the group consisting of sodium stearyl fumarate, magnesium stearate, stearic acid, glyceryl behenate, and combinations thereof.

In further embodiments, the lubricant is magnesium stearate.

In some embodiments, the pharmaceutical composition comprises about 0.5% (w/w) to about 1.5% (w/w) lubricant. In some embodiments, the pharmaceutical composition comprises about 0.75% (w/w) to about 1.25% (w/w) lubricant. In some embodiments, the pharmaceutical composition comprises about 0.8% (w/w) to about 1% (w/w) lubricant. In some embodiments, the pharmaceutical composition comprises about 0.75% (w/w) to about 0.95% (w/w) lubricant.

In some embodiments, the pharmaceutical composition comprises about 0.85% (w/w) to about 0.9% (w/w) lubricant. In some embodiments, the pharmaceutical composition comprises about 0.9% (w/w) to about 1.1% (w/w) lubricant. In some embodiments, the pharmaceutical composition comprises about 0.95% (w/w) to about 1.05% (w/w) lubricant.

In some embodiments, the pharmaceutical composition comprises about 0.8% (w/w), about 0.82% (w/w), about 0.84% (w/w), about 0.86% (w/w), about 0.87% (w/w), about 0.88% (w/w), about 0.9% (w/w), about 0.92% (w/w), about 0.94% (w/w), about 0.96% (w/w), about 0.98% (w/w), about 1.0% (w/w), about 1.02% (w/w), about 1.04% (w/w), about 1.06% (w/w), about 1.08% (w/w) or about 1.1% (w/w) lubricant. In some embodiments, the pharmaceutical composition comprises about 0.86% (w/w), about 0.87% (w/w), about 0.88% (w/w), about 0.98% (w/w), about 1.00% (w/w) or about 1.02% (w/w) lubricant.

In some embodiments, the pharmaceutical composition comprises about 0.87% (w/w) lubricant.

In some embodiments, the pharmaceutical composition comprises about 1% (w/w) lubricant.

In certain embodiments, provided is a pharmaceutical composition comprising: (a) a compound of formula (I)

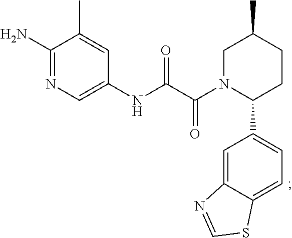 [SMILES: Cc1cc(NC(=O)C(=O)N2C[C@@H](C)CC[C@@H]2c2ccc3scnc3c2)cnc1N]

(b) a filler (e.g., microcrystalline cellulose);
(c) a glidant (e.g., colloidal silicon dioxide);
(d) a disintegrant (e.g., croscarmellose sodium); and
(e) a lubricant (e.g., magnesium stearate).

In some embodiments, the composition comprises a crystalline form of the compound of formula (I) described herein (e.g., Form A).

In some embodiments, the composition comprises:

(a) about 5% (w/w) to about 50% (w/w) of the compound of formula (I);
(b) about 50% (w/w) to about 90% (w/w) of a filler (e.g., microcrystalline cellulose);
(c) about 0.5% (w/w) to about 1.5% (w/w) of a glidant (e.g., colloidal silicon dioxide);
(d) about 2% (w/w) to about 6% (w/w) of a disintegrant (e.g., croscarmellose sodium);
(e) about 0.5% (w/w) to about 1.5% (w/w) of a lubricant (e.g., magnesium stearate);
thereby totaling 100% (w/w) of the composition.

In some embodiments, the composition comprises:

(a) about 10% (w/w) to about 45% (w/w) of the compound of formula (I);
(b) about 50% (w/w) to about 90% (w/w) of a filler (e.g., microcrystalline cellulose);
(c) about 0.8% (w/w) to about 1% (w/w) of a glidant (e.g., colloidal silicon dioxide);
(d) about 3.2% (w/w) to about 4% (w/w) of a disintegrant (e.g., croscarmellose sodium);
(e) about 0.8% (w/w) to about 1% (w/w) of a lubricant (e.g., magnesium stearate);
thereby totaling 100% (w/w) of the composition.

In some embodiments, the composition comprises:

(a) about 10% (w/w) to about 15% (w/w) of the compound of formula (I);
(b) about 77% (w/w) to about 83% (w/w) of a filler (e.g., microcrystalline cellulose);
(c) about 0.95% (w/w) to about 1.05% (w/w) of a glidant (e.g., colloidal silicon dioxide);
(d) about 3.8% (w/w) to about 4.2% (w/w) of a disintegrant (e.g., croscarmellose sodium);
(e) about 0.95% (w/w) to about 1.05% (w/w) of a lubricant (e.g., magnesium stearate);
thereby totaling 100% (w/w) of the composition.

In some embodiments, the composition comprises:

(a) about 12.5% (w/w) of the compound of formula (I);
(b) about 81.5% (w/w) of a filler (e.g., microcrystalline cellulose);
(c) about 1% (w/w) of a glidant (e.g., colloidal silicon dioxide);
(d) about 4% (w/w) of a disintegrant (e.g., croscarmellose sodium);
(e) about 1% (w/w) of a lubricant (e.g., magnesium stearate);
thereby totaling 100% (w/w) of the composition.

In some embodiments, the composition comprises:

(a) about 27% (w/w) to about 33% (w/w) of the compound of formula (I);
(b) about 63% (w/w) to about 68% (w/w) of a filler (e.g., microcrystalline cellulose);
(c) about 0.85% (w/w) to about 0.9% (w/w) of a glidant (e.g., colloidal silicon dioxide);
(d) about 3.4% (w/w) to about 3.6% (w/w) of a disintegrant (e.g., croscarmellose sodium);
(e) about 0.85% (w/w) to about 0.9% (w/w) of a lubricant (e.g., magnesium stearate);
thereby totaling 100% (w/w) of the composition.

In some embodiments, the composition comprises:

(a) about 29.4% (w/w) of the compound of formula (I);
(b) about 65.4% (w/w) of a filler (e.g., microcrystalline cellulose);
(c) about 0.87% (w/w) of a glidant (e.g., colloidal silicon dioxide);
(d) about 3.47% (w/w) of a disintegrant (e.g., croscarmellose sodium);
(e) about 0.87% (w/w) of a lubricant (e.g., magnesium stearate);
thereby totaling 100% (w/w) of the composition.

In some embodiments, the composition comprises:

(a) about 37% (w/w) to about 43% (w/w) of the compound of formula (I);
(b) about 52% (w/w) to about 58% (w/w) of a filler (e.g., microcrystalline cellulose);
(c) about 0.95% (w/w) to about 1.05% (w/w) of a glidant (e.g., colloidal silicon dioxide);
(d) about 3.8% (w/w) to about 4.2% (w/w) of a disintegrant (e.g., croscarmellose sodium);
(e) about 0.95% (w/w) to about 1.05% (w/w) of a lubricant (e.g., magnesium stearate);
thereby totaling 100% (w/w) of the composition.

In some embodiments, the composition comprises:

(a) about 40% (w/w) of the compound of formula (I);
(b) about 54% (w/w) of a filler (e.g., microcrystalline cellulose);
(c) about 1% (w/w) of a glidant (e.g., colloidal silicon dioxide);
(d) about 4% (w/w) of a disintegrant (e.g., croscarmellose sodium);
(e) about 1% (w/w) of a lubricant (e.g., magnesium stearate);
thereby totaling 100% (w/w) of the composition.

The pharmaceutically acceptable excipients can be present in either the intragranular or the extragranular components of the pharmaceutical composition. In some embodiments, one or more pharmaceutically acceptable excipients are present in both the intragranular and the extragranular components.

In some embodiments, the pharmaceutical composition contains an intragranular filler selected from the fillers described herein. In certain embodiments, the intragranular filler is a microcrystalline cellulose (e.g., Avicel®). In further embodiments, the intragranular filler is a microcrystalline cellulose PH 102 (e.g., Avicel® PH 102).

In certain embodiments, the pharmaceutical composition comprises about 30% (w/w) to about 70% (w/w) intragranular filler. In certain embodiments, the pharmaceutical composition comprises about 35% (w/w) to about 60% (w/w) intragranular filler.

In certain embodiments, the pharmaceutical composition comprises about 50% (w/w) to about 60% (w/w) intragranular filler. In certain embodiments, the pharmaceutical composition comprises about 52% (w/w) to about 58% (w/w) intragranular filler. In certain embodiments, the pharmaceutical composition comprises about 54% (w/w) to about 57% (w/w) intragranular filler.

In certain embodiments, the pharmaceutical composition comprises about 50% (w/w), about 51% (w/w), about 52% (w/w), about 53% (w/w), about 54% (w/w), about 55% (w/w), about 56% (w/w), about 56.5% (w/w), about 57% (w/w), about 58% (w/w), about 59% (w/w), or about 60% (w/w) intragranular filler. In some embodiments, the pharmaceutical composition comprises about 53% (w/w), about 54% (w/w), about 55% (w/w), about 56% (w/w), about 56.5% (w/w), about 57% (w/w) or about 58% (w/w) intragranular filler.

In some embodiments, the pharmaceutical composition comprises about 54% (w/w) intragranular filler.

In some embodiments, the pharmaceutical composition comprises about 56.5% (w/w) intragranular filler.

In some embodiments, the pharmaceutical composition comprises about 35% (w/w) to about 45% (w/w) intragranular filler. In some embodiments, the pharmaceutical composition comprises about 37% (w/w) to about 43% (w/w) intragranular filler. In some embodiments, the pharmaceutical composition comprises about 37% (w/w) to about 41% (w/w) intragranular filler.

In some embodiments, the pharmaceutical composition comprises about 35% (w/w), about 36% (w/w), about 37% (w/w), about 38% (w/w), about 39% (w/w), about 39.7% (w/w), about 40% (w/w), about 41% (w/w), about 42% (w/w), or about 43% (w/w) intragranular filler. In some embodiments, the pharmaceutical composition comprises about 38% (w/w), about 39% (w/w), about 39.7% (w/w), about 40% (w/w) or about 41% (w/w) intragranular filler.

In some embodiments, the pharmaceutical composition comprises about 39.7% (w/w) intragranular filler.

In some embodiments, the pharmaceutical composition contains an extragranular filler selected from the fillers described herein. In certain embodiments, the extragranular filler is a microcrystalline cellulose (e.g., Avicel®). In some embodiments, the extragranular filler is selected from microcrystalline cellulose PH 102 (e.g., Avicel® PH 102), PH 200 (e.g., Avicel® PH 200) or a mixture thereof. In some embodiments, the extragranular filler is a single grade of microcrystalline cellulose. In further embodiments, the intragranular filler is a microcrystalline cellulose PH 200 (e.g., Avicel® PH 200). In some embodiments, the extragranular filler is a mixture of grades of microcrystalline cellulose. In some embodiments, the extragranular filler is a mixture of microcrystalline cellulose PH 200 (e.g., Avicel® PH 200) and PH102 (e.g., Avicel® PH 102). In some embodiments, the mixture contains equal percentages by weight of microcrystalline cellulose PH 200 and PH102. (e.g., “25.68% w/w of filler” can comprise 12.84% w/w PH 200 and 12.84% w/w PH 102).

In some embodiments, the pharmaceutical composition comprises about 0% (w/w) to about 40% (w/w) extragranular filler. In some embodiments, the pharmaceutical composition comprises about 0% (w/w) to about 30% (w/w) extragranular filler. In some embodiments, the pharmaceutical composition comprises about 0% (w/w) to about 25% (w/w) extragranular filler.

In some embodiments, the pharmaceutical composition comprises 0% (w/w) extragranular filler.

In some embodiments, the pharmaceutical composition comprises about 15% (w/w) to about 35% (w/w) extragranular filler. In some embodiments, the pharmaceutical composition comprises about 20% (w/w) to about 30% (w/w) extragranular filler. In some embodiments, the pharmaceutical composition comprises about 22% (w/w) to about 27% (w/w) extragranular filler. In some embodiments, the pharmaceutical composition comprises about 15% (w/w) to about 35% (w/w) extragranular filler wherein the extragranular filler is microcrystalline cellulose PH 200 (e.g., Avicel® PH 200). In some embodiments, the pharmaceutical composition comprises about 20% (w/w) to about 30% (w/w) extragranular filler wherein the extragranular filler is microcrystalline cellulose PH 200 (e.g., Avicel® PH 200). In some embodiments, the pharmaceutical composition comprises about 22% (w/w) to about 27% (w/w) extragranular filler wherein the extragranular filler is microcrystalline cellulose PH 200 (e.g., Avicel® PH 200).

In some embodiments, the pharmaceutical composition comprises about 20% (w/w), about 21% (w/w), about 22% (w/w), about 23% (w/w), about 24% (w/w), about 25% (w/w), about 25.7% (w/w), about 26% (w/w), about 27% (w/w), about 28% (w/w), about 29% (w/w) or about 30% (w/w) extragranular filler. In some embodiments, the pharmaceutical composition comprises about 20% (w/w), about 21% (w/w), about 22% (w/w), about 23% (w/w), about 24% (w/w), about 25% (w/w), about 25.7% (w/w), about 26% (w/w), about 27% (w/w), about 28% (w/w), about 29% (w/w) or about 30% (w/w) extragranular filler wherein the extragranular filler is microcrystalline cellulose PH 200 (e.g., Avicel® PH 200).

In some embodiments, the pharmaceutical composition comprises about 25% (w/w) extragranular filler. In some embodiments, the pharmaceutical composition comprises about 25% (w/w) extragranular filler wherein the extragranular filler is microcrystalline cellulose PH 200 (e.g., Avicel® PH 200). In some embodiments, the pharmaceutical composition comprises about 25% (w/w) extragranular filler comprising about 12.5% w/w extragranular microcrystalline cellulose PH 200 and about 12.5% w/w extragranular microcrystalline cellulose PH 102.

In some embodiments, the pharmaceutical composition comprises about 25.7% (w/w) extragranular filler comprising about 12.85% w/w extragranular microcrystalline cellulose PH 200 and about 12.85% w/w extragranular microcrystalline cellulose PH 102.

In some embodiments, the pharmaceutical composition comprises an intragranular glidant selected from the glidants described herein. In some embodiments, the intragranular glidant is colloidal silicon dioxide (e.g., Aerosil® 200, CAB-O-SIL® M-5P).

In some embodiments, the pharmaceutical composition comprises about 0.25% (w/w) to about 0.75% (w/w) intragranular glidant. In some embodiments, the pharmaceutical composition comprises about 0.3% (w/w) to about 0.6% (w/w) intragranular glidant. In some embodiments, the pharmaceutical composition comprises about 0.35% (w/w) to about 0.55% (w/w) intragranular glidant. In some embodiments, the pharmaceutical composition comprises about 0.3% (w/w) to about 0.45% (w/w) intragranular glidant. In some embodiments, the pharmaceutical composition comprises about 0.35% (w/w) to about 0.4% (w/w) intragranular glidant. In some embodiments, the pharmaceutical composition comprises about 0.45% (w/w) to about 0.55% (w/w) intragranular glidant. In some embodiments, the pharmaceutical composition comprises about 0.48% (w/w) to about 0.52% (w/w) intragranular glidant.

In some embodiments, the pharmaceutical composition comprises about 0.30% (w/w), about 0.31% (w/w), about 0.32% (w/w), about 0.33% (w/w), about 0.34% (w/w), about 0.35% (w/w), about 0.36% (w/w), about 0.37% (w/w), about 0.38% (w/w), about 0.39% (w/w), about 0.4% (w/w), about 0.41% (w/w), about 0.42% (w/w), about 0.43% (w/w), about 0.44% (w/w), about 0.45% (w/w), about 0.46% (w/w), about 0.47% (w/w), about 0.48% (w/w), about 0.49% (w/w), about 0.5% (w/w), about 0.51% (w/w), about 0.52% (w/w), about 0.53% (w/w), about 0.54% (w/w) or about 0.55% (w/w) intragranular glidant. In some embodiments, the pharmaceutical composition comprises about 0.35% (w/w), about 0.36% (w/w), about 0.37% (w/w), about 0.38% (w/w), about 0.39% (w/w), about 0.48% (w/w), about 0.49% (w/w), about 0.5% (w/w), about 0.51% (w/w) or about 0.52% (w/w) intragranular glidant.

In some embodiments, the pharmaceutical composition comprises about 0.37% (w/w) intragranular glidant.

In some embodiments, the pharmaceutical composition comprises about 0.5% (w/w) intragranular glidant.

In some embodiments, the pharmaceutical composition comprises an extragranular glidant selected from the glidants described herein. In some embodiments, the extragranular glidant is colloidal silicon dioxide (e.g., Aerosil® 200, CAB-O-SIL® M-5P).

In some embodiments, the pharmaceutical composition comprises about 0.25% (w/w) to about 0.75% (w/w) extragranular glidant. In some embodiments, the pharmaceutical composition comprises about 0.3% (w/w) to about 0.7% (w/w) extragranular glidant. In some embodiments, the pharmaceutical composition comprises about 0.4% (w/w) to about 0.6% (w/w) extragranular glidant. In some embodiments, the pharmaceutical composition comprises about 0.45% (w/w) to about 0.55% (w/w) extragranular glidant.

In some embodiments, the pharmaceutical composition comprises about 0.45% (w/w), about 0.46% (w/w), about 0.47% (w/w), about 0.48% (w/w), about 0.49% (w/w), about 0.5% (w/w), about 0.51% (w/w), about 0.52% (w/w), about 0.53% (w/w), about 0.54% (w/w) or about 0.55% (w/w) extragranular glidant. In some embodiments, the pharmaceutical composition comprises about 0.48% (w/w), about 0.49% (w/w), about 0.5% (w/w), about 0.51% (w/w) or about 0.52% (w/w) extragranular glidant.

In some embodiments, the pharmaceutical composition comprises about 0.5% (w/w) extragranular glidant.

In some embodiments, the pharmaceutical composition comprises an intragranular disintegrant selected from the disintegrants described herein. In certain embodiments, the intragranular disintegrant is croscarmellose sodium.

In some embodiments, the pharmaceutical composition comprises about 1% (w/w) to about 3% (w/w) intragranular disintegrant. In some embodiments, the pharmaceutical composition comprises about 1.2% (w/w) to about 2.4% (w/w) intragranular disintegrant.

In some embodiments, the pharmaceutical composition comprises about 1.4% (w/w) to about 2.2% (w/w) intragranular disintegrant. In some embodiments, the pharmaceutical composition comprises about 1.2% (w/w) to about 1.8% (w/w) intragranular disintegrant.

In some embodiments, the pharmaceutical composition comprises about 1.4% (w/w) to about 1.6% (w/w) intragranular disintegrant. In some embodiments, the pharmaceutical composition comprises about 1.8% (w/w) to about 2.2% (w/w) intragranular disintegrant.

In some embodiments, the pharmaceutical composition comprises about 1.92% (w/w) to about 2.08% (w/w) intragranular disintegrant.

In some embodiments, the pharmaceutical composition comprises about 1.2% (w/w), about 1.24% (w/w), about 1.28% (w/w), about 1.32% (w/w), about 1.36% (w/w), about 1.4% (w/w), about 1.44% (w/w), about 1.47% (w/w), about 1.52% (w/w), about 1.56% (w/w), about 1.6% (w/w), about 1.64% (w/w), about 1.68% (w/w), about 1.72% (w/w), about 1.76% (w/w), about 1.8% (w/w), about 1.84% (w/w), about 1.88% (w/w), about 1.92% (w/w), about 1.96% (w/w), about 2% (w/w), about 2.04% (w/w), about 2.08% (w/w), about 2.12% (w/w), about 2.16% (w/w) or about 2.2% (w/w) intragranular disintegrant. In some embodiments, the pharmaceutical composition comprises about 1.4% (w/w), about 1.44% (w/w), about 1.47% (w/w), about 1.52% (w/w), about 1.56% (w/w), about 1.92% (w/w), about 1.96% (w/w), about 2% (w/w), about 2.04% (w/w) or about 2.08% (w/w) intragranular disintegrant.

In some embodiments, the pharmaceutical composition comprises about 1.47% (w/w) intragranular disintegrant.

In some embodiments, the pharmaceutical composition comprises about 2% (w/w) intragranular disintegrant.

In some embodiments, the pharmaceutical composition comprises an extragranular disintegrant selected from the disintegrants described herein. In certain embodiments, the extragranular disintegrant is croscarmellose sodium.

In some embodiments, the pharmaceutical composition comprises about 2% (w/w) to about 3% (w/w) extragranular disintegrant. In some embodiments, the pharmaceutical composition comprises about 1.2% (w/w) to about 2.8% (w/w) extragranular disintegrant.

In some embodiments, the pharmaceutical composition comprises about 1.6% (w/w) to about 2.4% (w/w) extragranular disintegrant. In some embodiments, the pharmaceutical composition comprises about 1.8% (w/w) to about 2.2% (w/w) extragranular disintegrant.

In some embodiments, the pharmaceutical composition comprises about 1.8% (w/w), about 1.84% (w/w), about 1.88% (w/w), about 1.92% (w/w), about 1.96% (w/w), about 2% (w/w), about 2.04% (w/w), about 2.08% (w/w), about 2.12% (w/w), about 2.16% (w/w) or about 2.2% (w/w) extragranular disintegrant. In some embodiments, the pharmaceutical composition comprises about 1.92% (w/w), about 1.96% (w/w), about 2% (w/w), about 2.04% (w/w) or about 2.08% (w/w) extragranular disintegrant.

In some embodiments, the pharmaceutical composition comprises about 2% (w/w) extragranular disintegrant.

In some embodiments, the pharmaceutical composition comprises an intragranular lubricant selected from the lubricants described herein. In certain embodiments, the intragranular lubricant is magnesium stearate.

In some embodiments, the pharmaceutical composition comprises about 0.25% (w/w) to about 0.75% (w/w) intragranular lubricant. In some embodiments, the pharmaceutical composition comprises about 0.3% (w/w) to about 0.6% (w/w) intragranular lubricant. In some embodiments, the pharmaceutical composition comprises about 0.35% (w/w) to about 0.55% (w/w) intragranular lubricant. In some embodiments, the pharmaceutical composition comprises about 0.3% (w/w) to about 0.45% (w/w) intragranular lubricant. In some embodiments, the pharmaceutical composition comprises about 0.35% (w/w) to about 0.4% (w/w) intragranular lubricant. In some embodiments, the pharmaceutical composition comprises about 0.45% (w/w) to about 0.55% (w/w) intragranular lubricant. In some embodiments, the pharmaceutical composition comprises about 0.48% (w/w) to about 0.52% (w/w) intragranular lubricant.

In some embodiments, the pharmaceutical composition comprises about 0.30% (w/w), about 0.31% (w/w), about 0.32% (w/w), about 0.33% (w/w), about 0.34% (w/w), about 0.35% (w/w), about 0.36% (w/w), about 0.37% (w/w), about 0.38% (w/w), about 0.39% (w/w), about 0.4% (w/w), about 0.41% (w/w), about 0.42% (w/w), about 0.43% (w/w), about 0.44% (w/w), about 0.45% (w/w), about 0.46% (w/w), about 0.47% (w/w), about 0.48% (w/w), about 0.49% (w/w), about 0.5% (w/w), about 0.51% (w/w), about 0.52% (w/w), about 0.53% (w/w), about 0.54% (w/w) or about 0.55% (w/w) intragranular lubricant. In some embodiments, the pharmaceutical composition comprises about 0.35% (w/w), about 0.36% (w/w), about 0.37% (w/w), about 0.38% (w/w), about 0.39% (w/w), about 0.48% (w/w), about 0.49% (w/w), about 0.5% (w/w), about 0.51% (w/w) or about 0.52% (w/w) intragranular lubricant.

In some embodiments, the pharmaceutical composition comprises about 0.37% (w/w) intragranular lubricant.

In some embodiments, the pharmaceutical composition comprises about 0.5% (w/w) intragranular lubricant.

In some embodiments, the pharmaceutical composition comprises an extragranular lubricant selected from the lubricants described herein. In certain embodiments, the extragranular lubricant is magnesium stearate.

In some embodiments, the pharmaceutical composition comprises about 0.25% (w/w) to about 0.75% (w/w) extragranular lubricant. In some embodiments, the pharmaceutical composition comprises about 0.3% (w/w) to about 0.7% (w/w) extragranular lubricant.

In some embodiments, the pharmaceutical composition comprises about 0.4% (w/w) to about 0.6% (w/w) extragranular lubricant. In some embodiments, the pharmaceutical composition comprises about 0.45% (w/w) to about 0.55% (w/w) extragranular lubricant.

In some embodiments, the pharmaceutical composition comprises about 0.45% (w/w), about 0.46% (w/w), about 0.47% (w/w), about 0.48% (w/w), about 0.49% (w/w), about 0.5% (w/w), about 0.51% (w/w), about 0.52% (w/w), about 0.53% (w/w), about 0.54% (w/w) or about 0.55% (w/w) extragranular lubricant. In some embodiments, the pharmaceutical composition comprises about 0.48% (w/w), about 0.49% (w/w), about 0.5% (w/w), about 0.51% (w/w) or about 0.52% (w/w) extragranular lubricant.

In some embodiments, the pharmaceutical composition comprises about 0.5% (w/w) extragranular lubricant.

In some embodiments, provided is a pharmaceutical composition comprising:

(a) a compound of formula (I)

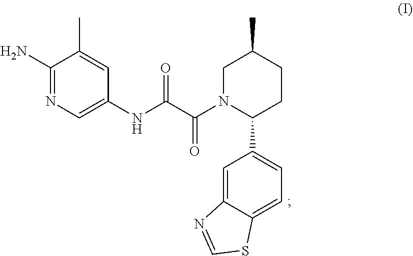 [SMILES: Cc1cc(NC(=O)C(=O)N2C[C@@H](C)CC[C@@H]2c2ccc3scnc3c2)cnc1N]

(b) an intragranular filler (e.g., microcrystalline cellulose);
(c) an intragranular glidant (e.g., colloidal silicon dioxide);
(d) an intragranular disintegrant (e.g., croscarmellose sodium);
(e) an extragranular lubricant (e.g., magnesium stearate);
(f) an extragranular filler (e.g., microcrystalline cellulose);
(g) an extragranular glidant (e.g., colloidal silicon dioxide);
(h) an extragranular disintegrant (e.g., croscarmellose sodium); and
(i) an extragranular lubricant (e.g., magnesium stearate).

In some embodiments, provided is a pharmaceutical composition comprising:

(a) a crystalline form of a compound of formula (I) (e.g., crystalline form A as described herein)

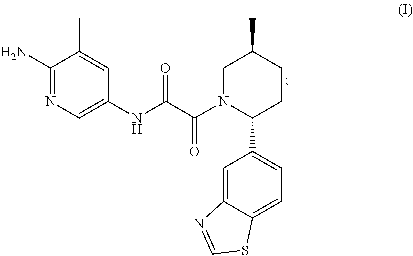 [SMILES: Cc1cc(NC(=O)C(=O)N2C[C@@H](C)CC[C@@H]2c2ccc3scnc3c2)cnc1N]

(b) an intragranular filler (e.g., microcrystalline cellulose);
(c) an intragranular glidant (e.g., colloidal silicon dioxide);
(d) an intragranular disintegrant (e.g., croscarmellose sodium);
(e) an extragranular lubricant (e.g., magnesium stearate);
(f) an extragranular filler (e.g., microcrystalline cellulose);
(g) an extragranular glidant (e.g., colloidal silicon dioxide);
(h) an extragranular disintegrant (e.g., croscarmellose sodium); and
(i) an extragranular lubricant (e.g., magnesium stearate).

In some embodiments, the pharmaceutical composition comprises:

(a) about 5% (w/w) to about 50% (w/w) of the compound of formula (I) (e.g., crystalline form A);
(b) about 30% (w/w) to about 70% (w/w) of an intragranular filler (e.g., microcrystalline cellulose);
(c) about 0.25% (w/w) to about 0.75% (w/w) of an intragranular glidant (e.g., colloidal silicon dioxide);
(d) about 1% (w/w) to about 3% (w/w) of an intragranular disintegrant (e.g., croscarmellose sodium);
(e) about 0.25% (w/w) to about 0.75% (w/w) of an intragranular lubricant (e.g., magnesium stearate);
(f) about 0% (w/w) to about 40% (w/w) of an extragranular filler (e.g., microcrystalline cellulose);
(g) about 0.25% (w/w) to about 0.75% (w/w) of an extragranular glidant (e.g., colloidal silicon dioxide);
(h) about 1% (w/w) to about 3% (w/w) of an extragranular disintegrant (e.g., croscarmellose sodium); and
(i) about 0.25% (w/w) to about 0.75% (w/w) of an extragranular lubricant (e.g., magnesium stearate);
thereby totaling 100% (w/w) of the composition.

In some embodiments, the pharmaceutical composition comprises:

(a) about 10% (w/w) to about 45% (w/w) of the compound of formula (I) (e.g., crystalline form A);
(b) about 35% (w/w) to about 60% (w/w) of an intragranular filler (e.g., microcrystalline cellulose);
(c) about 0.35% (w/w) to about 0.55% (w/w) of an intragranular glidant (e.g., colloidal silicon dioxide);
(d) about 1.4% (w/w) to about 2.2% (w/w) of an intragranular disintegrant (e.g., croscarmellose sodium);
(e) about 0.35% (w/w) to about 0.55% (w/w) of an intragranular lubricant (e.g., magnesium stearate);
(f) about 0% (w/w) to about 30% (w/w) of an extragranular filler (e.g., microcrystalline cellulose);
(g) about 0.35% (w/w) to about 0.55% (w/w) of an extragranular glidant (e.g., colloidal silicon dioxide);
(h) about 1.8% (w/w) to about 2.2% (w/w) of an extragranular disintegrant (e.g., croscarmellose sodium);
(i) about 0.35% (w/w) to about 0.55% (w/w) of an extragranular lubricant (e.g., magnesium stearate); and
thereby totaling 100% (w/w) of the composition.

In some embodiments, the pharmaceutical composition comprises:

(a) about 10% (w/w) to about 15% (w/w) of the compound of formula (I) (e.g., crystalline form A);
(b) about 52% (w/w) to about 58% (w/w) of an intragranular filler (e.g., microcrystalline cellulose);
(c) about 0.95% (w/w) to about 1.05% (w/w) of an intragranular glidant (e.g., colloidal silicon dioxide);
(d) about 1.92% (w/w) to about 2.08% (w/w) of an intragranular disintegrant (e.g., croscarmellose sodium);
(e) about 0.95% (w/w) to about 1.05% (w/w) of an intragranular lubricant (e.g., magnesium stearate);
(f) about 22% (w/w) to about 27% (w/w) of an extragranular filler (e.g., microcrystalline cellulose);
(g) about 0.45% (w/w) to about 0.55% (w/w) of an extragranular glidant (e.g., colloidal silicon dioxide);
(h) about 1.8% (w/w) to about 2.2% (w/w) of an extragranular disintegrant (e.g., croscarmellose sodium); and
(i) about 0.45% (w/w) to about 0.55% (w/w) of an extragranular lubricant (e.g., magnesium stearate);
thereby totaling 100% (w/w) of the composition.

In some embodiments, the pharmaceutical composition comprises:

(a) about 12.5% (w/w) of the compound of formula (I) (e.g., crystalline form A);
(b) about 56.5% (w/w) of an intragranular filler (e.g., microcrystalline cellulose);
(c) about 0.5% (w/w) of an intragranular glidant (e.g., colloidal silicon dioxide);
(d) about 2% (w/w) of an intragranular disintegrant (e.g., croscarmellose sodium);
(e) about 0.5% (w/w) of an intragranular lubricant (e.g., magnesium stearate);
(f) about 25% (w/w) of an extragranular filler (e.g., microcrystalline cellulose);
(g) about 0.5% (w/w) of an extragranular glidant (e.g., colloidal silicon dioxide);
(h) about 2% (w/w) of an extragranular disintegrant (e.g., croscarmellose sodium); and
(i) about 0.5% (w/w) of an extragranular lubricant (e.g., magnesium stearate);
thereby totaling 100% (w/w) of the composition.

In some embodiments, the pharmaceutical composition comprises:

(a) about 27% (w/w) to about 33% (w/w) of the compound of formula (I) (e.g., crystalline form A);
(b) about 37% (w/w) to about 43% (w/w) of an intragranular filler (e.g., microcrystalline cellulose);
(c) about 0.85% (w/w) to about 0.9% (w/w) of an intragranular glidant (e.g., colloidal silicon dioxide);
(d) about 1.4% (w/w) to about 1.6% (w/w) of an intragranular disintegrant (e.g., croscarmellose sodium);
(e) about 0.85% (w/w) to about 0.9% (w/w) of an intragranular lubricant (e.g., magnesium stearate);
(f) about 0% (w/w) to about 30% (w/w) of an extragranular filler (e.g., microcrystalline cellulose);
(g) about 0.45% (w/w) to about 0.55% (w/w) of an extragranular glidant (e.g., colloidal silicon dioxide);
(h) about 1.8% (w/w) to about 2.2% (w/w) of an extragranular disintegrant (e.g., croscarmellose sodium); and
(i) about 0.45% (w/w) to about 0.55% (w/w) of an extragranular lubricant (e.g., magnesium stearate);
thereby totaling 100% (w/w) of the composition.

In some embodiments, the pharmaceutical composition comprises:

(a) about 29.4% (w/w) of the compound of formula (I) (e.g., crystalline form A);
(b) about 39.7% (w/w) of an intragranular filler (e.g., microcrystalline cellulose);
(c) about 0.37% (w/w) of an intragranular glidant (e.g., colloidal silicon dioxide);
(d) about 1.47% (w/w) of an intragranular disintegrant (e.g., croscarmellose sodium);
(e) about 0.37% (w/w) of an intragranular lubricant (e.g., magnesium stearate);
(f) about 25.7% (w/w) of an extragranular filler (e.g., microcrystalline cellulose);
(g) about 0.5% (w/w) of an extragranular glidant (e.g., colloidal silicon dioxide);
(h) about 2% (w/w) of an extragranular disintegrant (e.g., croscarmellose sodium); and
(i) about 0.5% (w/w) of an extragranular lubricant (e.g., magnesium stearate);
thereby totaling 100% (w/w) of the composition.

In some embodiments, the pharmaceutical composition comprises:

(a) about 37% (w/w) to about 43% (w/w) of the compound of formula (I) (e.g., crystalline form A);
(b) about 52% (w/w) to about 58% (w/w) of an intragranular filler (e.g., microcrystalline cellulose);
(c) about 0.95% (w/w) to about 1.05% (w/w) of an intragranular glidant (e.g., colloidal silicon dioxide);
(d) about 1.92% (w/w) to about 2.08% (w/w) of an intragranular disintegrant (e.g., croscarmellose sodium);
(e) about 0.95% (w/w) to about 1.05% (w/w) of an intragranular lubricant (e.g., magnesium stearate);
(f) about 0% (w/w) to about 30% (w/w) of an extragranular filler (e.g., microcrystalline cellulose);
(g) about 0.45% (w/w) to about 0.55% (w/w) of an extragranular glidant (e.g., colloidal silicon dioxide);
(h) about 1.8% (w/w) to about 2.2% (w/w) of an extragranular disintegrant (e.g., croscarmellose sodium); and
(i) about 0.45% (w/w) to about 0.55% (w/w) of an extragranular lubricant (e.g., magnesium stearate);
thereby totaling 100% (w/w) of the composition.

In some embodiments, the pharmaceutical composition comprises:

(a) about 40% (w/w) of the compound of formula (I) (e.g., crystalline form A);
(b) about 54% (w/w) of an intragranular filler (e.g., microcrystalline cellulose);
(c) about 0.5% (w/w) of an intragranular glidant (e.g., colloidal silicon dioxide);
(d) about 2% (w/w) of an intragranular disintegrant (e.g., croscarmellose sodium);
(e) about 0.5% (w/w) of an intragranular lubricant (e.g., magnesium stearate);
(f) about 0% (w/w) of an extragranular filler (e.g., microcrystalline cellulose);
(g) about 0.5% (w/w) of an extragranular glidant (e.g., colloidal silicon dioxide);
(h) about 2% (w/w) of an extragranular disintegrant (e.g., croscarmellose sodium); and
(i) about 0.5% (w/w) of an extragranular lubricant (e.g., magnesium stearate);
thereby totaling 100% (w/w) of the composition.

In some embodiments, the pharmaceutical composition comprises:

(a) about 5% (w/w) to about 50% (w/w) of the compound of formula (I) (e.g., crystalline form A);
(b) about 30% (w/w) to about 70% (w/w) of intragranular microcrystalline cellulose (e.g., microcrystalline cellulose PH 102, e.g., Avicel® PH 102);
(c) about 0.25% (w/w) to about 0.75% (w/w) of intragranular colloidal silicon dioxide (e.g., Aerosil® 200, CAB-O-SIL® M-5P);
(d) about 1% (w/w) to about 3% (w/w) of intragranular croscarmellose sodium (Ac-Di-Sol®);
(e) about 0.25% (w/w) to about 0.75% (w/w) of intragranular magnesium stearate;
(f) about 0% (w/w) to about 40% (w/w) of extragranular microcrystalline cellulose;
(g) about 0.25% (w/w) to about 0.75% (w/w) of extragranular colloidal silicon dioxide (e.g., Aerosil® 200, CAB-O-SIL® M-5P);
(h) about 1% (w/w) to about 3% (w/w) of extragranular croscarmellose sodium (Ac-Di-Sol®); and
(i) about 0.25% (w/w) to about 0.75% (w/w) of extragranular magnesium stearate;
thereby totaling 100% (w/w) of the composition.

In some embodiments, the pharmaceutical composition comprises:

(a) about 10% (w/w) to about 45% (w/w) of the compound of formula (I) (e.g., crystalline form A);
(b) about 35% (w/w) to about 60% (w/w) of intragranular microcrystalline cellulose (e.g., microcrystalline cellulose PH 102, e.g., Avicel® PH 102);
(c) about 0.35% (w/w) to about 0.55% (w/w) of intragranular colloidal silicon dioxide (e.g., Aerosil® 200, CAB-O-SIL® M-5P);
(d) about 1.4% (w/w) to about 2.2% (w/w) of intragranular croscarmellose sodium (Ac-Di-Sol®);
(e) about 0.35% (w/w) to about 0.55% (w/w) of intragranular magnesium stearate;
(f) about 0% (w/w) to about 30% (w/w) of extragranular microcrystalline cellulose;
(g) about 0.35% (w/w) to about 0.55% (w/w) of extragranular colloidal silicon dioxide (e.g., Aerosil® 200, CAB-O-SIL® M-5P);
(h) about 1.8% (w/w) to about 2.2% (w/w) of extragranular croscarmellose sodium (Ac-Di-Sol®); and
(i) about 0.35% (w/w) to about 0.55% (w/w) of extragranular magnesium stearate;
thereby totaling 100% (w/w) of the composition.

In some embodiments, the pharmaceutical composition comprises:

(a) about 10% (w/w) to about 15% (w/w) of the compound of formula (I) (e.g., crystalline form A);
(b) about 52% (w/w) to about 58% (w/w) of intragranular microcrystalline cellulose (e.g., microcrystalline cellulose PH 102, e.g., Avicel® PH 102);
(c) about 0.95% (w/w) to about 1.05% (w/w) of intragranular colloidal silicon dioxide (e.g., Aerosil® 200, CAB-O-SIL® M-5P);
(d) about 1.92% (w/w) to about 2.08% (w/w) of intragranular croscarmellose sodium (Ac-Di-Sol®);
(e) about 0.95% (w/w) to about 1.05% (w/w) of intragranular magnesium stearate;
(f) about 22% (w/w) to about 27% (w/w) of extragranular microcrystalline cellulose (e.g., microcrystalline cellulose PH 200, e.g., Avicel® PH 200);
(g) about 0.45% (w/w) to about 0.55% (w/w) of extragranular colloidal silicon dioxide (e.g., Aerosil® 200, CAB-O-SIL® M-5P);
(h) about 1.8% (w/w) to about 2.2% (w/w) of extragranular croscarmellose sodium (Ac-Di-Sol®); and
(i) about 0.45% (w/w) to about 0.55% (w/w) of extragranular magnesium stearate;
thereby totaling 100% (w/w) of the composition.

In some embodiments, the pharmaceutical composition comprises:

(a) about 12.5% (w/w) of the compound of formula (I) (e.g., crystalline form A);
(b) about 56.5% (w/w) of intragranular microcrystalline cellulose (e.g., microcrystalline cellulose PH 102, e.g., Avicel® PH 102);
(c) about 0.5% (w/w) of intragranular colloidal silicon dioxide (e.g., Aerosil® 200, CAB-O-SIL® M-5P);
(d) about 2% (w/w) of intragranular croscarmellose sodium (Ac-Di-Sol®);
(e) about 0.5% (w/w) of intragranular magnesium stearate;
(f) about 25% (w/w) of extragranular microcrystalline cellulose (e.g., microcrystalline cellulose PH 200, e.g., Avicel® PH 200);
(g) about 0.5% (w/w) of extragranular colloidal silicon dioxide (e.g., Aerosil® 200, CAB-O-SIL® M-5P);
(h) about 2% (w/w) of extragranular croscarmellose sodium (Ac-Di-Sol®); and
(i) about 0.5% (w/w) of extragranular magnesium stearate;
thereby totaling 100% (w/w) of the composition.

In some embodiments, the pharmaceutical composition comprises:

(a) about 27% (w/w) to about 33% (w/w) of the compound of formula (I) (e.g., crystalline form A);
(b) about 37% (w/w) to about 43% (w/w) of intragranular microcrystalline cellulose (e.g., microcrystalline cellulose PH 102, e.g., Avicel® PH 102);
(c) about 0.85% (w/w) to about 0.9% (w/w) of intragranular colloidal silicon dioxide (e.g., Aerosil® 200, CAB-O-SIL® M-5P);
(d) about 1.4% (w/w) to about 1.6% (w/w) of intragranular croscarmellose sodium (Ac-Di-Sol®);
(e) about 0.85% (w/w) to about 0.9% (w/w) of intragranular magnesium stearate;
(f) about 0% (w/w) to about 30% (w/w) of extragranular microcrystalline cellulose (e.g., 1:1 ratio of microcrystalline cellulose PH 200 (e.g., Avicel® PH 200 and microcrystalline cellulose PH 102 (e.g., Avicel® PH 102));
(g) about 0.45% (w/w) to about 0.55% (w/w) of extragranular colloidal silicon dioxide (e.g., Aerosil® 200, CAB-O-SIL® M-5P);
(h) about 1.8% (w/w) to about 2.2% (w/w) of extragranular croscarmellose sodium (Ac-Di-Sol®; and
(i) about 0.45% (w/w) to about 0.55% (w/w) of extragranular magnesium stearate;
thereby totaling 100% (w/w) of the composition.

In some embodiments, the pharmaceutical composition comprises:

(a) about 29.4% (w/w) of the compound of formula (I) (e.g., crystalline form A);
(b) about 39.7% (w/w) of intragranular microcrystalline cellulose (e.g., microcrystalline cellulose PH 102, e.g., Avicel® PH 102);
(c) about 0.37% (w/w) of intragranular colloidal silicon dioxide (e.g., Aerosil® 200, CAB-O-SIL® M-5P);
(d) about 1.47% (w/w) of intragranular croscarmellose sodium (Ac-Di-Sol®);
(e) about 0.37% (w/w) of intragranular magnesium stearate;
(f) about 25.7% (w/w) of extragranular microcrystalline cellulose ((e.g., 1:1 ratio of microcrystalline cellulose PH 200 (e.g., Avicel® PH 200) (about 12.85% w/w) and microcrystalline cellulose PH 102 (e.g., Avicel® PH 102) (about 12.85% w/w));
(g) about 0.5% (w/w) of extragranular colloidal silicon dioxide (e.g., Aerosil® 200, CAB-O-SIL® M-5P);
(h) about 2% (w/w) of extragranular croscarmellose sodium (Ac-Di-Sol®); and
(i) about 0.5% (w/w) of extragranular magnesium stearate;
thereby totaling 100% (w/w) of the composition.

In some embodiments, the pharmaceutical composition comprises:

(a) about 37% (w/w) to about 43% (w/w) of the compound of formula (I) (e.g., crystalline form A);
(b) about 52% (w/w) to about 58% (w/w) of intragranular microcrystalline cellulose (e.g., microcrystalline cellulose PH 102, e.g., Avicel® PH 102);
(c) about 0.95% (w/w) to about 1.05% (w/w) of intragranular colloidal silicon dioxide (e.g., Aerosil® 200, CAB-O-SIL® M-5P);
(d) about 1.92% (w/w) to about 2.08% (w/w) of intragranular croscarmellose sodium (Ac-Di-Sol®);
(e) about 0.95% (w/w) to about 1.05% (w/w) of intragranular magnesium stearate;
(f) about 0% (w/w) to about 30% (w/w) of extragranular microcrystalline cellulose;
(g) about 0.45% (w/w) to about 0.55% (w/w) of extragranular colloidal silicon dioxide (e.g., Aerosil® 200, CAB-O-SIL® M-5P);
(h) about 1.8% (w/w) to about 2.2% (w/w) of extragranular croscarmellose sodium (Ac-Di-Sol®); and
(i) about 0.45% (w/w) to about 0.55% (w/w) of extragranular magnesium stearate;
thereby totaling 100% (w/w) of the composition.

In some embodiments, the pharmaceutical composition comprises:

(a) about 40% (w/w) of the compound of formula (I) (e.g., crystalline form A);
(b) about 54% (w/w) of intragranular microcrystalline cellulose (e.g., microcrystalline cellulose PH 102, e.g., Avicel® PH 102);
(c) about 0.5% (w/w) of intragranular colloidal silicon dioxide (e.g., Aerosil® 200, CAB-O-SIL® M-5P);
(d) about 2% (w/w) of intragranular croscarmellose sodium (Ac-Di-Sol®);
(e) about 0.5% (w/w) of intragranular magnesium stearate;
(f) about 0% (w/w) of extragranular microcrystalline cellulose;
(g) about 0.5% (w/w) of extragranular colloidal silicon dioxide (e.g., Aerosil® 200, CAB-O-SIL® M-5P);
(h) about 2% (w/w) of extragranular croscarmellose sodium (Ac-Di-Sol®); and
(i) about 0.5% (w/w) of extragranular magnesium stearate;
thereby totaling 100% (w/w) of the composition.

### Dosage Forms

In some embodiments, provided are dosage forms comprising a pharmaceutical composition described herein.

In some embodiments, provided are dosage forms intended for oral administration comprising a pharmaceutical composition described herein.

In certain embodiments, the dosage form is selected from the group consisting of a powder, a sachet, a stickpack, a capsule, a minitab, and a tablet.

In certain embodiments, the dosage form is a tablet.

In some embodiments, the total weight of the pharmaceutical composition in the dosage form is about 50 mg to 1000 mg.

In some embodiments, the total weight of the pharmaceutical composition in the dosage form is about 100 mg to 750 mg.

In some embodiments, the total weight of the pharmaceutical composition in the dosage form is about 50 mg to 150 mg.

In some embodiments, the total weight of the pharmaceutical composition in the dosage form is about 50 mg, about 60 mg, about 70 mg, about 80 mg, about 90 mg, about 100 mg, about 110 mg, about 120 mg, about 130 mg, about 140 mg or about 150 mg.

In some embodiments, the total weight of the pharmaceutical composition in the dosage form is about 80 mg, about 90 mg, about 100 mg, about 110 mg, or about 120 mg.

In some embodiments, the total weight of the pharmaceutical composition in the dosage form is about 100 mg.

In some embodiments, the total weight of the pharmaceutical composition in the dosage form is about 300 mg to 500 mg.

In some embodiments, the total weight of the pharmaceutical composition in the dosage form is about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg or about 500 mg.

In some embodiments, the total weight of the pharmaceutical composition in the dosage form is about 320 mg, about 330 mg, about 340 mg, about 350 mg or about 360 mg.

In some embodiments, the total weight of the pharmaceutical composition in the dosage form is about 340 mg.

In some embodiments, the total weight of the pharmaceutical composition in the dosage form is about 380 mg, about 390 mg, about 400 mg, about 410 mg or about 420 mg.

In some embodiments, the total weight of the pharmaceutical composition in the dosage form is about 400 mg.

In some embodiments, the total weight of the pharmaceutical composition in the dosage form is about 600 mg to 900 mg.

In some embodiments, the total weight of the pharmaceutical composition in the dosage form is about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, about 800 mg, about 810 mg, about 820 mg, about 830 mg, about 840 mg, about 850 mg, about 860 mg, about 870 mg, about 880 mg, about 890 mg or about 900 mg.

In some embodiments, the total weight of the pharmaceutical composition in the dosage form is about 700 mg, about 710 mg, about 720 mg, about 730 mg, about 740 mg, about 750 mg, about 760 mg, about 770 mg, about 780 mg, about 790 mg or about 800 mg.

In some embodiments, the total weight of the pharmaceutical composition in the dosage form is about 750 mg.

In some embodiments, the total weight of the pharmaceutical composition in the dosage form is about 100 mg, about 340 mg, about 400 mg or about 750 mg.

In some embodiments, the composition comprises about 5 mg to about 400 mg of a compound of formula (I). In some embodiments, the dosage form comprises about 10 mg to about 350 mg of the compound of formula (I).

In some embodiments, the dosage form comprises about 8 mg to about 17 mg, about 40 mg to about 60 mg, about 80 mg to about 120 mg or about 250 to about 350 mg of the compound of formula (I). In some embodiments, the dosage form comprises about 8 mg to about 17 mg of the compound of formula (I). In some embodiments, the dosage form comprises about 10 mg to about 15 mg of the compound of formula (I). In some embodiments, the dosage form comprises about 12 mg to about 13 mg of the compound of formula (I).

In some embodiments, the dosage form comprises about 8 mg, about 9 mg, about 10 mg, about 11 mg, about 12 mg, about 12.5 mg, about 13 mg, about 14 mg, about 15 mg, about 16 mg or about 17 mg of the compound of formula (I).

In some embodiments, the dosage form comprises about 12.5 mg of the compound of formula (I).

In some embodiments, the dosage form comprises about 40 mg to about 60 mg of the compound of formula (I). In some embodiments, the dosage form comprises about 45 mg to about 55 mg of the compound of formula (I). In some embodiments, the dosage form comprises about 47 mg to about 53 mg of the compound of formula (I).

In some embodiments, the dosage form comprises about 49 mg to about 51 mg of the compound of formula (I). In some embodiments, the dosage form comprises about 45 mg, about 46 mg, about 47 mg, about 48 mg, about 49 mg, about 50 mg, about 51 mg, about 52 mg, about 53 mg, about 54 mg or about 55 mg of the compound of formula (I).

In some embodiments, the dosage form comprises about 50 mg of the compound of formula (I).

In some embodiments, the dosage form comprises about 80 mg to about 120 mg of the compound of formula (I). In some embodiments, the dosage form comprises about 90 mg to about 110 mg of the compound of formula (I). In some embodiments, the dosage form comprises about 95 mg to about 105 mg of the compound of formula (I). In some embodiments, the dosage form comprises about 98 mg to about 102 mg of the compound of formula (I).

In some embodiments, the dosage form comprises about 90 mg, about 91 mg, about 92 mg, about 93 mg, about 94 mg, about 95 mg, about 96 mg, about 97 mg, about 98 mg, about 99 mg, about 100 mg, about 101 mg, about 102 mg, about 103 mg, about 104 mg, about 105 mg about 106 mg, about 107 mg, about 108 mg, about 109 mg or about 110 mg of the compound of formula (I).

In some embodiments, the dosage form comprises about 100 mg of the compound of formula (I).

In some embodiments, the dosage form comprises about 250 to about 350 mg of the compound of formula (I). In some embodiments, the dosage form comprises about 260 to about 340 mg of the compound of formula (I). In some embodiments, the dosage form comprises about 270 to about 330 mg of the compound of formula (I). In some embodiments, the dosage form comprises about 280 to about 320 mg of the compound of formula (I). In some embodiments, the dosage form comprises about 290 to about 310 mg of the compound of formula (I). In some embodiments, the dosage form comprises about 295 to about 305 mg of the compound of formula (I).

In some embodiments, the dosage form comprises about 290 mg, about 291 mg, about 292 mg, about 293 mg, about 294 mg, about 295 mg, about 296 mg, about 297 mg, about 298 mg, about 299 mg, about 300 mg, about 301 mg, about 302 mg, about 303 mg, about 304 mg, about 305 mg about 306 mg, about 307 mg, about 308 mg, about 309 mg or about 310 mg of the compound of formula (I).

In particular embodiments, the dosage form comprises about 300 mg of the compound of formula (I).

In some embodiments, the dosage form comprises about 8 mg, about 9 mg, about 10 mg, about 11 mg, about 12 mg, about 12.5 mg, about 13 mg, about 14 mg, about 15 mg, about 16 mg, about 17 mg, about 45 mg, about 46 mg, about 47 mg, about 48 mg, about 49 mg, about 50 mg, about 51 mg, about 52 mg, about 53 mg, about 54 mg, about 55 mg, about 90 mg, about 91 mg, about 92 mg, about 93 mg, about 94 mg, about 95 mg, about 96 mg, about 97 mg, about 98 mg, about 99 mg, about 100 mg, about 101 mg, about 102 mg, about 103 mg, about 104 mg, about 105 mg about 106 mg, about 107 mg, about 108 mg, about 109 mg, about 110 mg, about 290 mg, about 291 mg, about 292 mg, about 293 mg, about 294 mg, about 295 mg, about 296 mg, about 297 mg, about 298 mg, about 299 mg, about 300 mg, about 301 mg, about 302 mg, about 303 mg, about 304 mg, about 305 mg about 306 mg, about 307 mg, about 308 mg, about 309 mg or about 310 mg of the compound of formula (I).

In some embodiments, the dosage form comprises about 10 mg, about 11 mg, about 12 mg, about 12.5 mg, about 13 mg, about 14 mg, about 15 mg, about 48 mg, about 49 mg, about 50 mg, about 51 mg, about 52 mg, about 53 mg, about 96 mg, about 97 mg, about 98 mg, about 99 mg, about 100 mg, about 101 mg, about 102 mg, about 103 mg, about 104 mg, about 295 mg, about 296 mg, about 297 mg, about 298 mg, about 299 mg, about 300 mg, about 301 mg, about 302 mg, about 303 mg, about 304 mg or about 305 mg of the compound of formula (I).

In certain embodiments, the dosage form comprises about 12.5 mg, about 50 mg, about 100 mg or about 300 mg of the compound of formula (I).

In some embodiments, the dosage form comprises 12.5 mg Compound of formula (I), 56.5 mg intragranular microcrystalline cellulose, 0.5 mg intragranular colloidal silicon dioxide (e.g., Aerosil® 200, CAB-O-SIL® M-5P), 2 mg intragranular croscarmellose sodium (Ac-Di-Sol®), 0.5 mg intragranular magnesium stearate, 25 mg extragranular microcrystalline cellulose, 0.5 mg extragranular colloidal silicon dioxide (e.g., Aerosil® 200, CAB-O-SIL® M-5P), 2 mg extragranular croscarmellose sodium (Ac-Di-Sol®) and 0.5 mg extragranular magnesium stearate.

In some embodiments, the dosage form comprises 50 mg Compound of formula (I), 226 mg intragranular microcrystalline cellulose, 2 mg intragranular colloidal silicon dioxide (e.g., Aerosil® 200, CAB-O-SIL® M-5P), 8 mg intragranular croscarmellose sodium (Ac-Di-Sol®), 2 mg intragranular magnesium stearate, 100 mg extragranular microcrystalline cellulose, 2 mg extragranular colloidal silicon dioxide (e.g., Aerosil® 200, CAB-O-SIL® M-5P), 8 mg extragranular croscarmellose sodium (Ac-Di-Sol®) and 2 mg extragranular magnesium stearate.

In some embodiments, the dosage form comprises 100 mg Compound of formula (I), 135 mg intragranular microcrystalline cellulose, 1.25 mg intragranular colloidal silicon dioxide (e.g., Aerosil® 200, CAB-O-SIL® M-5P), 5 mg intragranular croscarmellose sodium (Ac-Di-Sol®), 1.25 mg intragranular magnesium stearate, 87.3 mg extragranular microcrystalline cellulose (e.g., 43.65 mg PH 200 and 43.65 mg PH 102), 1.7 mg extragranular colloidal silicon dioxide (e.g., Aerosil® 200, CAB-O-SIL® M-5P), 6.8 mg extragranular croscarmellose sodium (Ac-Di-Sol®) and 1.7 mg extragranular magnesium stearate.

In some embodiments, the dosage form comprises 300 mg Compound of formula (I), 405 mg intragranular microcrystalline cellulose, 3.75 mg intragranular colloidal silicon dioxide (e.g., Aerosil® 200, CAB-O-SIL® M-5P), 15 mg intragranular croscarmellose sodium (Ac-Di-Sol®), 3.75 mg intragranular magnesium stearate, 3.75 mg extragranular colloidal silicon dioxide (e.g., Aerosil® 200, CAB-O-SIL® M-5P), 15 mg extragranular croscarmellose sodium (Ac-Di-Sol®) and 3.75 mg extragranular magnesium stearate.

In certain embodiments, the tablet further comprises a coating. In certain embodiments, the coating is selected from the group consisting of a film forming polymer, a plasticizer, and combinations thereof. In certain embodiments, the film forming polymer is selected from the group consisting of a hypromellose, an ethylcellulose, cellulose acetate, a polyvinylpyrrolidone, a polyvinyl alcohol, a polyacrylate, and combinations thereof. In certain embodiments, the plasticizer is selected from the group consisting of triacetin, polyethylene glycol, propylene glycol, and combinations thereof. In certain embodiments, the coating comprises polyvinyl alcohol.

In certain embodiments, the coating comprises a colorant selected from the group consisting of titanium dioxide, an aluminum lake, an iron oxide, carbon black, and combinations thereof. In some embodiments, the colorant is titanium dioxide.

### Methods of Making

In some embodiments, provided are processes for preparing the pharmaceutical compositions described herein, for example, comprising:
(a) blending the compound of formula (I) with one or more pharmaceutically acceptable excipients to obtain a blend;
(b) granulating the blend to obtain granules;
(c) milling the granules to obtain an intragranular phase; and
(d) blending the intragranular phase with one or more extragranular pharmaceutical excipients to obtain the pharmaceutical composition.

In another aspect, provided are processes for preparing the dosage forms described herein, for example, comprising:
(e) blending the compound of formula (I) with one or more pharmaceutically acceptable excipients to obtain a blend;
(f) granulating the blend to obtain granules;
(g) milling the granules to obtain an intragranular phase; and
(h) blending the intragranular phase with one or more extragranular pharmaceutical excipients to obtain the pharmaceutical composition (pharmaceutical blend).
(i) compressing the pharmaceutical blend into a tablet.

In certain embodiments, in step (a), the one or more pharmaceutically acceptable excipients is selected from the group consisting of a filler, a disintegrant, a binder, a wetting agent, a lubricant, a glidant, and combinations thereof.

In certain embodiments, in step (a), the compound of formula (I) is blended with a filler, a disintegrant, a lubricant, and a glidant.

In certain embodiments, in step (a), the filler is microcrystalline cellulose.

In some embodiments, in step (a), the disintegrant is croscarmellose sodium.

In certain embodiments, in step (a), the glidant is colloidal silicon dioxide.

In certain embodiments, in step (a), the lubricant is magnesium stearate.

In certain embodiments, in step (d), the one or more extragranular excipients is selected from the group consisting of a filler, a disintegrant, a lubricant, a glidant, and combinations thereof.

In some embodiments, in step (d), the intragranular phase is blended with a filler, a disintegrant, a lubricant, and a glidant.

In certain embodiments, in step (d), the disintegrant is croscarmellose sodium.

In some embodiments, in step (d), the glidant is colloidal silicon dioxide.

In certain embodiments, in step (d), the lubricant is magnesium stearate.

In certain embodiments, granulating the blend to obtain granules comprises a dry granulation process step. In certain embodiments, granulating the blend to obtain granules comprises a wet granulation process step.

In certain embodiments, the tablet comprises a coating. In certain embodiments, the coating comprises one or more film-forming polymers selected from the group consisting of a hypromellose, an ethylcellulose, a polyvinylpyrrolidone, a polyacrylate, a plasticizer, and combinations thereof. In further embodiments, the coating comprises a polyvinyl alcohol.

In certain embodiments, the coating comprises a colorant selected from the group consisting of titanium dioxide, an aluminum lake, an iron oxide, carbon black, and combinations thereof. In some embodiments, the colorant is titanium dioxide.

In some embodiments, provided is a pharmaceutical composition described herein prepared by the processes described herein.

### Methods of Use and Treatment

Treatment of MTAP-Deficient and/or MTA-Accumulating Proliferation Disorders

5-Methylthioadenosine phosphorylase (MTAP) catalyzes the reversible phosphorylation of S-methyl-5′-thioadenosine (MTA) to adenine and 5-methylthioribose-1-phosphate. MTAP-deletion is a common genetic event in human cancer. MTAP deletion frequency in a subset of human cancers is described in Cerami et al., Cancer Discov. (2012); 2(5):401-4; Gao et al., Sci Signal. (2013); 6(269):pl1; and Lee et al., Nat. Gen. (2014) 46(11):1227-32. For example, more than 50% of malignant peripheral nerve sheath tumor (MPNST) have deletions in MTAP (Lee et al., Nat. Gen. (2014)). Other cancers with high MTAP deletion frequencies are glioblastoma (GBM), mesothelioma, bladder cancer, pancreatic cancer, esophageal cancer, squamous lung cancer, melanoma, diffuse large B cell lymphoma (DLBCL), head and neck cancer, cholangiocarcinoma, lung adenoma, sarcoma, stomach cancer, glioma, adrenal carcinoma, thymoma, breast cancer, liver cancer, ovarian cancer, renal papillary cancer, uterine cancer, prostate cancer, and renal clear cell cancer. MTAP deletion in cells is one of the mechanisms that leads to MTAP-deficiency, increased intracellular MTA accumulation, and confers enhanced dependency on the protein arginine methyltransferase 5 (PRMT5) in cancer cells. Other mechanisms leading to MTAP deficiency include, inter alia, MTAP translocations and MTAP epigenetic silencing which could also lead to MTAP-null and/or MTAP deficient tumors. PRMT5 mediates the formation of symmetric dimethylarginine (SDMA); thus, the PRMT5 activity can be assessed by measuring the SDMA levels using the antibody against an SDMA or SDMA modified polypeptide.

In some embodiments, provided are methods of treating human or animal subjects having or having been diagnosed with an MTAP-deficiency-related and/or MTA-accumulating proliferative disorder (e.g., cancer) comprising administering to the subject in need thereof a therapeutically effective amount of a compound of the present disclosure (e.g., a crystalline form of a compound of formula (I), crystalline Form A of the compound of formula (I)), a pharmaceutical composition comprising a compound of formula (I) or a pharmaceutically acceptable salt thereof as described herein, or a dosage form as described herein.

In some embodiments, provided is a compound of the present disclosure (e.g., a crystalline form of a compound of formula (I), crystalline Form A of the compound of formula (I)), or a pharmaceutical composition comprising a compound of formula (I) of the present disclosure for use in a method of treating human or animal subjects having or having been diagnosed with an MTAP-deficiency-related and/or MTA-accumulating proliferative disorder (e.g., cancer). In some embodiments, the compound or composition is provided in a therapeutically effective amount.

In some embodiments, provided is a compound of the present disclosure (e.g., a crystalline form of a compound of formula (I), crystalline Form A of the compound of formula (I)), or a pharmaceutical composition comprising a compound of formula (I) of the present disclosure for use in the manufacturing of a medicament for treating human or animal subjects having or having been diagnosed with an MTAP-deficiency-related and/or MTA-accumulating proliferative disorder (e.g., cancer). In some embodiments, the compound or composition is provided in a therapeutically effective amount.

In some embodiments, provided is a use of a compound of the present disclosure (e.g., a crystalline form of a compound of formula (I), crystalline Form A of the compound of formula (I)), or of a pharmaceutical composition comprising a compound of formula (I) of the present disclosure in a method of treating human or animal subjects having or having been diagnosed with an MTAP-deficiency-related and/or MTA-accumulating proliferative disorder (e.g., cancer). In some embodiments, the use is of a therapeutically effective amount of the compound or composition.

In some embodiments, provided is use of a compound of the present disclosure (e.g., a crystalline form of a compound of formula (I), crystalline Form A of the compound of formula (I)), or of a pharmaceutical composition comprising a compound of formula (I) of the present disclosure in the manufacturing of a medicament for treating human or animal subjects having or having been diagnosed with an MTAP-deficiency-related and/or MTA-accumulating proliferative disorder (e.g., cancer). In some embodiments, the use is of a therapeutically effective amount of the compound or composition.

In some embodiments, provided are methods for treating an MTAP-deficiency-related and/or MTA-accumulating proliferative disorder (e.g., cancer) in a subject in need thereof comprising administering to the subject an effective amount (e.g., a therapeutically effective amount) of a compound of the present disclosure (e.g., a crystalline form of a compound of formula (I), crystalline Form A of the compound of formula (I)), a pharmaceutical composition comprising a compound of formula (I) or a pharmaceutically acceptable salt thereof as described herein, or a dosage form as described herein.

In some embodiments, provided are methods of treating human or animal subjects having or having been diagnosed with an MTAP-deficiency-related and/or MTA-accumulating proliferative disorder (e.g., cancer) comprising administering to the subject in need thereof a therapeutically effective amount of a pharmaceutical composition of the present disclosure. In some embodiments, the method comprises administering to the subject a dosage form of the present disclosure. In one embodiment, the compound or composition is administered in combination with a second therapeutic agent.

In some embodiments, provided are methods of treating an MTAP-deficiency-related and/or MTA-accumulating proliferative disorder (e.g., cancer) in a subject in need thereof comprising administering to the subject a therapeutically effective amount of a pharmaceutical composition of the present disclosure. In some embodiment, the method comprises administering to the subject a dosage form of the present disclosure. In one embodiment, the compound or composition is administered in combination with a second therapeutic agent.

In some embodiments, the subject is human.

In certain embodiments, the disease is an MTAP-deficient and/or MTA-accumulating cancer.

In some embodiments, the cancer is glioma, glioblastoma, malignant peripheral nerve sheath tumors (MPNST, e.g., intracranial MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

In some embodiments, the cancer is an MTAP-deficient and/or MTA-accumulating glioma, glioblastoma, malignant peripheral nerve sheath tumors (MPNST, e.g., intracranial MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine, sarcoma or a CNS metastasis from a solid tumor.

In some embodiments, the cancer is a central nervous system (CNS) malignancy. In some embodiments, the CNS malignancy is selected from glioma (e.g., low grade glioma, intermediate grade glioma), intracranial MPNST tumors, glioblastoma, glioblastoma multiforme, or CNS metastases from solid tumors. In some embodiments, the CNS malignancy is glioma. In some embodiments, the CNS malignancy is low grade glioma. In some embodiments, the CNS malignancy is intermediate grade glioma. In some embodiments, the CNS malignancy is glioblastoma or glioblastoma multiforme. In some embodiments, the CNS malignancy is glioblastoma. In some embodiments, the CNS malignancy is glioblastoma multiforme. In some embodiments, the CNS malignancy r is a MTAP-deleted glioblastoma. In some embodiments, the CNS malignancy is an intracranial MPNST tumor. In some embodiments, the CNS malignancy is CNS metastases from solid tumors.

In some embodiments, the cancer is a cancer selected from the group of cholangiocarcinoma, NSCLC (adenocarcinoma), NSCLC (squamous), bladder cancer, and DLBCL. In some embodiments, the cancer is a cancer selected from the group consisting of glioma, glioblastoma, non-small cell lung cancer (adenocarcinoma and squamous), mesothelioma, cholangiocarcinoma, urothelial carcinoma, and malignant peripheral nerve sheath tumor. In some embodiments, the cancer is cholangiocarcinoma. In some embodiments, the cancer is NSCLC (adenocarcinoma). In some embodiments, the cancer is NSCLC (squamous). In some embodiments, the cancer is bladder cancer. In some embodiments, the cancer is DLBCL.

In some embodiments, the cancer is a cancer selected from the group consisting of glioma, glioblastoma, non-small cell lung cancer (adenocarcinoma and squamous), mesothelioma, cholangiocarcinoma, urothelial carcinoma, and malignant peripheral nerve sheath tumor.

In some embodiments, the compound of formula (I), crystalline forms (e.g., crystalline form A), pharmaceutical compositions (e.g., comprising a compound of formula (I) or a pharmaceutically acceptable salt thereof) and dosage forms thereof, as described herein can be used in a method of inhibiting proliferation of MTAP-deficient cells in a subject in need thereof, the method comprising the step of administering to the subject a compound of formula (I), crystalline forms (e.g., crystalline form A), pharmaceutical compositions (e.g., comprising a compound of formula (I) or a pharmaceutically acceptable salt thereof) or dosage forms thereof, as described herein in an amount that is effective to inhibit proliferation of the MTAP-deficient cells. In one embodiment, the subject in need thereof suffers from a cancer selected from the group consisting of glioma, glioblastoma, malignant peripheral nerve sheath tumors (MPNST, e.g., intracranial MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine, sarcoma or a CNS metastasis from a solid tumor.

In some embodiments, the cancer is a central nervous system (CNS) malignancy. In some embodiments, the CNS malignancy is selected from glioma (e.g., low grade glioma, intermediate grade glioma), intracranial MPNST tumors, glioblastoma, glioblastoma multiforme, or CNS metastases from solid tumors. In some embodiments, the CNS malignancy is glioma. In some embodiments, the CNS malignancy is low grade glioma. In some embodiments, the CNS malignancy is intermediate grade glioma. In some embodiments, the CNS malignancy is glioblastoma or glioblastoma multiforme. In some embodiments, the CNS malignancy is glioblastoma. In some embodiments, the CNS malignancy is glioblastoma multiforme. In some embodiments, the CNS malignancy is a MTAP-deleted glioblastoma. In some embodiments, the CNS malignancy is an intracranial MPNST tumor. In some embodiments, the CNS malignancy is CNS metastases from solid tumors.

In some embodiments, the cancer is a cancer selected from the group of cholangiocarcinoma, NSCLC (adenocarcinoma), NSCLC (squamous), bladder cancer, and DLBCL. In some embodiments, the cancer is a cancer selected from the group consisting of glioma, glioblastoma, non-small cell lung cancer (adenocarcinoma and squamous), mesothelioma, cholangiocarcinoma, urothelial carcinoma, and malignant peripheral nerve sheath tumor. In some embodiments, the cancer is cholangiocarcinoma. In some embodiments, the cancer is NSCLC (adenocarcinoma). In some embodiments, the cancer is NSCLC (squamous). In some embodiments, the cancer is bladder cancer. In some embodiments, the cancer is DLBCL.

In some embodiments, the cancer is a cancer selected from the group consisting of glioma, glioblastoma, non-small cell lung cancer (adenocarcinoma and squamous), mesothelioma, cholangiocarcinoma, urothelial carcinoma, and malignant peripheral nerve sheath tumor.

In some embodiments, the compound of formula (I), crystalline forms (e.g., crystalline form A), pharmaceutical compositions (e.g., comprising a compound of formula (I) or a pharmaceutically acceptable salt thereof) or dosage forms thereof, as described herein can be used in a method of inhibiting proliferation of MTA-accumulating cells in a subject in need thereof, the method comprising the step of administering to the subject a compound of formula (I), crystalline forms (e.g., crystalline form A), pharmaceutical compositions (e.g., comprising a compound of formula (I) or a pharmaceutically acceptable salt thereof) or dosage forms thereof, as described herein in an amount that is effective to inhibit proliferation of the MTA-accumulating cells. In one embodiment, the subject in need thereof suffers from a cancer selected from the group consisting of glioma, glioblastoma, malignant peripheral nerve sheath tumors (MPNST, e.g., intracranial MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine, sarcoma or a CNS metastasis from a solid tumor.

In some embodiments, the cancer is a CNS malignancy. In some embodiments, the CNS malignancy is selected from glioma (e.g., low grade glioma, intermediate grade glioma), intracranial MPNST tumors, glioblastoma, glioblastoma multiforme, or CNS metastases from solid tumors. In some embodiments, the CNS malignancy is glioma. In some embodiments, the CNS malignancy is low grade glioma. In some embodiments, the CNS malignancy is intermediate grade glioma. In some embodiments, the CNS malignancy is glioblastoma or glioblastoma multiforme. In some embodiments, the CNS malignancy is glioblastoma. In some embodiments, the CNS malignancy is glioblastoma multiforme. In some embodiments, the CNS malignancy is a MTAP-deleted glioblastoma. In some embodiments, the CNS malignancy is an intracranial MPNST tumor. In some embodiments, the CNS malignancy is CNS metastases from solid tumors.

In some embodiments, the cancer is a cancer selected from the group of cholangiocarcinoma, NSCLC (adenocarcinoma), NSCLC (squamous), bladder cancer, and DLBCL. In some embodiments, the cancer is a cancer selected from the group consisting of glioma, glioblastoma, non-small cell lung cancer (adenocarcinoma and squamous), mesothelioma, cholangiocarcinoma, urothelial carcinoma, and malignant peripheral nerve sheath tumor. In some embodiments, the cancer is cholangiocarcinoma. In some embodiments, the cancer is NSCLC (adenocarcinoma). In some embodiments, the cancer is NSCLC (squamous). In some embodiments, the cancer is bladder cancer. In some embodiments, the cancer is DLBCL.

In some embodiments, the cancer is a cancer selected from the group consisting of glioma, glioblastoma, non-small cell lung cancer (adenocarcinoma and squamous), mesothelioma, cholangiocarcinoma, urothelial carcinoma, and malignant peripheral nerve sheath tumor.

In some embodiments, the compound of formula (I), crystalline forms (e.g., crystalline form A), pharmaceutical compositions (e.g., comprising a compound of formula (I) or a pharmaceutically acceptable salt thereof) or dosage forms thereof, as described herein can be used in a method of inhibiting proliferation of MTAP deficient and/or MTA-accumulating cells in a subject in need thereof, the method comprising the step of administering to the subject a compound of formula (I), crystalline forms (e.g., crystalline form A), pharmaceutical compositions (e.g., comprising a compound of formula (I) or a pharmaceutically acceptable salt thereof) or dosage forms thereof, as described herein in an amount that is effective to inhibit proliferation of the MTAP deficient and/or MTA-accumulating cells. In one embodiment, the subject in need thereof suffers from a cancer selected from the group consisting of glioma, glioblastoma, malignant peripheral nerve sheath tumors (MPNST, e.g., intracranial MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine, sarcoma or a CNS metastasis from a solid tumor.

In some embodiments, the cancer is a CNS malignancy. In some embodiments, the CNS malignancy is selected from glioma (e.g., low grade glioma, intermediate grade glioma), intracranial MPNST tumors, glioblastoma, glioblastoma multiforme, or CNS metastases from solid tumors. In some embodiments, the CNS malignancy is glioma. In some embodiments, the CNS malignancy is low grade glioma. In some embodiments, the CNS malignancy is intermediate grade glioma. In some embodiments, the CNS malignancy is glioblastoma or glioblastoma multiforme. In some embodiments, the CNS malignancy is glioblastoma. In some embodiments, the CNS malignancy is glioblastoma multiforme. In some embodiments, the CNS malignancy is a MTAP-deleted glioblastoma. In some embodiments, the CNS malignancy is an intracranial MPNST tumor. In some embodiments, the CNS malignancy is CNS metastases from solid tumors.

In some embodiments, the cancer is a cancer selected from the group of cholangiocarcinoma, NSCLC (adenocarcinoma), NSCLC (squamous), bladder cancer, and DLBCL. In some embodiments, the cancer is a cancer selected from the group consisting of glioma, glioblastoma, non-small cell lung cancer (adenocarcinoma and squamous), mesothelioma, cholangiocarcinoma, urothelial carcinoma, and malignant peripheral nerve sheath tumor. In some embodiments, the cancer is cholangiocarcinoma. In some embodiments, the cancer is NSCLC (adenocarcinoma). In some embodiments, the cancer is NSCLC (squamous). In some embodiments, the cancer is bladder cancer. In some embodiments, the cancer is DLBCL.

In some embodiments, the cancer is a cancer selected from the group consisting of glioma, glioblastoma, non-small cell lung cancer (adenocarcinoma and squamous), mesothelioma, cholangiocarcinoma, urothelial carcinoma, and malignant peripheral nerve sheath tumor.

### Combination Therapies

In some embodiments, provided are methods of treatment of MTAP-deficient and/or MTA accumulating proliferative disorders (e.g., cancers) with a compound of formula (I), crystalline forms (e.g., crystalline form A), pharmaceutical compositions (e.g., comprising a compound of formula (I) or a pharmaceutically acceptable salt thereof) or dosage forms thereof, as described herein in combination with one or more therapeutic agents.

In some embodiments, provided are methods of treatment of MTAP-deficient and/or MTA accumulating proliferative disorders (e.g., cancers) with a compound of formula (I), crystalline forms (e.g., crystalline form A), pharmaceutical compositions (e.g., comprising a compound of formula (I) or a pharmaceutically acceptable salt thereof) or dosage forms thereof, as described herein in combination with a second therapeutic agent. In some embodiments, provided are methods of treatment of MTAP-deficient and/or MTA accumulating proliferative disorders (e.g., cancers) with a compound of formula (I), crystalline forms (e.g., crystalline form A), pharmaceutical compositions (e.g., comprising a compound of formula (I) or a pharmaceutically acceptable salt thereof) or dosage forms thereof, as described herein in combination with a second therapeutic agent and a third therapeutic agent. In some embodiments, provided are methods of treatment of MTAP-deficient and/or MTA accumulating proliferative disorders (e.g., cancers) with a compound of formula (I), crystalline forms (e.g., crystalline form A), pharmaceutical compositions (e.g., comprising a compound of formula (I) or a pharmaceutically acceptable salt thereof) or dosage forms thereof, as described herein in combination with a second therapeutic agent, a third therapeutic agent, and a fourth therapeutic agent.

The term “Combination” refers to either a fixed combination in one dosage unit form, or a combined administration where a compound of formula (I), crystalline forms (e.g., crystalline form A), pharmaceutical compositions (e.g., comprising a compound of formula (I) or a pharmaceutically acceptable salt thereof) or dosage forms thereof, as described herein and a combination partner (e.g., another drug as explained below, also referred to as “therapeutic agent” or “co-agent”) may be administered independently at the same time or separately within time intervals, especially where these time intervals allow that the combination partners show a cooperative, e.g., synergistic effect. The single components may be packaged in a kit or separately. One or both of the components (e.g., powders or liquids) may be reconstituted or diluted to a desired dose prior to administration. The terms “co-administration” or “combined administration” or the like as utilized herein are meant to encompass administration of the selected combination partner to a single subject in need thereof (e.g., a patient), and are intended to include treatment regimens in which the agents are not necessarily administered by the same route of administration or at the same time. The term “pharmaceutical combination” as used herein means a product that results from the mixing or combining of more than one therapeutic agent and includes both fixed and non-fixed combinations of the therapeutic agents. The term “fixed combination” means that the therapeutic agents, e.g., a compound of formula (I), crystalline forms (e.g., crystalline form A), pharmaceutical compositions (e.g., comprising a compound of formula (I) or a pharmaceutically acceptable salt thereof) or dosage forms thereof, as described herein and a combination partner, are both administered to a patient simultaneously in the form of a single entity or dosage. The term “non-fixed combination” means that the therapeutic agents, e.g., a compound of formula (I), crystalline forms (e.g., crystalline form A), pharmaceutical compositions (e.g., comprising a compound of formula (I) or a pharmaceutically acceptable salt thereof) or dosage forms thereof, as described herein and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g., the administration of three or more therapeutic agent.

The term “combination therapy” refers to the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients. Alternatively, such administration encompasses co-administration in multiple, or in separate containers (e.g., tablets, capsules, powders, and liquids) for each active ingredient. Powders and/or liquids may be reconstituted or diluted to a desired dose prior to administration. In addition, such administration also encompasses use of each type of therapeutic agent in a sequential manner, either at approximately the same time or at different times.

In certain embodiments, a compound of formula (I), crystalline forms (e.g., crystalline form A), pharmaceutical compositions (e.g., comprising a compound of formula (I) or a pharmaceutically acceptable salt thereof) or dosage forms thereof, as described herein are combined with other therapeutic agents, including, but not limited to, other anti-cancer agents, anti-allergic agents, anti-nausea agents (or anti-emetics), pain relievers, cytoprotective agents, and combinations thereof.

In some embodiments, provided is a method of treating a disease or disorder (e.g., cancer) comprising administering or coadministering, in any order, to a patient in need thereof a compound of formula (I), crystalline forms (e.g., crystalline form A), pharmaceutical compositions (e.g., comprising a compound of formula (I) or a pharmaceutically acceptable salt thereof) or dosage forms thereof, as described herein, and a general chemotherapeutic agent selected from anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), nab-paclitaxel (Abraxane®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®).

In some embodiments, provided is a method of treating a disease or disorder (e.g., cancer) comprising administering or coadministering, in any order, to a patient in need thereof a compound of formula (I), crystalline forms (e.g., crystalline form A), pharmaceutical compositions (e.g., comprising a compound of formula (I) or a pharmaceutically acceptable salt thereof) or dosage forms thereof, as described herein, and an EGFR-inhibitor (e.g., cetuximab, panitumimab, erlotinib, gefitinib and EGFRi NOS). In some embodiments, provided is a method of treating a disease or disorder (e.g., cancer) comprising administering or coadministering, in any order, to a patient in need thereof a compound of formula (I), crystalline forms (e.g., crystalline form A), pharmaceutical compositions (e.g., comprising a compound of formula (I) or a pharmaceutically acceptable salt thereof) or dosage forms thereof, as described herein, and a MAPK-pathway inhibitor (e.g., BRAFi, panRAFi, MEKi, ERKi; PI3K-mTOR pathway inhibitors, such as alpha-specific PI3Ki, pan-class I PI3Ki and mTOR/PI3Ki, particularly everolimus and analogues thereof).

MTAP-deletion can co-occur with mutations in the KRAS gene (e.g., KRASG12C). In some embodiments, provided is a method of treating a disease or disorder (e.g., cancer) comprising administering or coadministering, in any order, to a patient in need thereof a compound of formula (I), crystalline forms (e.g., crystalline form A), pharmaceutical compositions (e.g., comprising a compound of formula (I) or a pharmaceutically acceptable salt thereof) or dosage forms thereof, as described herein, and a KRAS inhibitor (e.g., a pan-KRAS or a specific G12C, G12D, G13C inhibitor, e.g., adagrasib, sotorasib, LY3537982, RMC-6236, RMC-6291, RMC-9805, RMC-8839).

In some embodiments, provided is a method of treating a disease or disorder (e.g., cancer) comprising administering or coadministering, in any order, to a patient in need thereof a compound of formula (I), crystalline forms (e.g., crystalline form A), pharmaceutical compositions (e.g., comprising a compound of formula (I) or a pharmaceutically acceptable salt thereof) or dosage forms thereof, as described herein, and a Spliceosome inhibitor (e.g., SF3b 1 inhibitors; e.g., E7107).

In some embodiments, provided is a method of treating a disease or disorder (e.g., cancer) comprising administering or coadministering, in any order, to a patient in need thereof a compound of formula (I), crystalline forms (e.g., crystalline form A), pharmaceutical compositions (e.g., comprising a compound of formula (I) or a pharmaceutically acceptable salt thereof) or dosage forms thereof, as described herein, and an HDAC inhibitor or DNA methyltransferase inhibitor. In some embodiments, the HDAC inhibitor is Trichostatin A. In some embodiments, the DNA methyltransferase inhibitor is 5-azacytidine.

In some embodiments, provided is a method of treating a disease or disorder (e.g., cancer) comprising administering or coadministering, in any order, to a patient in need thereof a compound of formula (I), crystalline forms (e.g., crystalline form A), pharmaceutical compositions (e.g., comprising a compound of formula (I) or a pharmaceutically acceptable salt thereof) or dosage forms thereof, as described herein, and a MAT2A inhibitor (e.g., AG-270, IDE397, S95035).

In some embodiments, provided is a method of treating a disease or disorder (e.g., cancer) comprising administering or coadministering, in any order, to a patient in need thereof a compound of formula (I), crystalline forms (e.g., crystalline form A), pharmaceutical compositions (e.g., comprising a compound of formula (I) or a pharmaceutically acceptable salt thereof) or dosage forms thereof, as described herein, and an inhibitor of a protein which interacts with or is required for PRMT5 function, including, but not limited to, pICIN, WDR77 or RIOK 1.

In some embodiments, provided is a method of treating a disease or disorder (e.g., cancer) comprising administering or coadministering, in any order, to a patient in need thereof a compound of formula (I), crystalline forms (e.g., crystalline form A), pharmaceutical compositions (e.g., comprising a compound of formula (I) or a pharmaceutically acceptable salt thereof) or dosage forms thereof, as described herein, and an HDM2 inhibitor and/or 5-FU or other purine analogues (e.g., 6-thioguanine, 6-mercaptopurine).

In some embodiments, provided is a method of treating a disease or disorder (e.g., cancer) comprising administering or coadministering, in any order, to a patient in need thereof a compound of formula (I), crystalline forms (e.g., crystalline form A), pharmaceutical compositions (e.g., comprising a compound of formula (I) or a pharmaceutically acceptable salt thereof) or dosage forms thereof, as described herein, and a CDK4 inhibitor, including, but not limited to, LEE011 or a CDK 4/6 inhibitor (e.g., palbociclib (Ibrance®), ribociclib (Kisgali®), and abemaciclib (Verzenio®).

In some embodiments, provided is a method of treating a disease or disorder (e.g., cancer) comprising administering or coadministering, in any order, to a patient in need thereof a compound of formula (I), crystalline forms (e.g., crystalline form A), pharmaceutical compositions (e.g., comprising a compound of formula (I) or a pharmaceutically acceptable salt thereof) or dosage forms thereof, as described herein, and targeted treatments contingent on the dependency of individual target tumors on relevant pathways as determined by suitable predictive markers, including but not limited to: inhibitors of HDM2i, PI3K/mTOR-I, MAPKi, RTKi (EGFRi, FGFRi, METi, IGFiRi, JAKi, and WNTi.

In some embodiments, provided is a method of treating a disease or disorder (e.g., cancer) comprising administering or coadministering, in any order, to a patient in need thereof a compound of formula (I), crystalline forms (e.g., crystalline form A), pharmaceutical compositions (e.g., comprising a compound of formula (I) or a pharmaceutically acceptable salt thereof) or dosage forms thereof, as described herein) and immunotherapy.

In some embodiments, provided is a method of treating a disease or disorder (e.g., cancer) comprising administering or coadministering, in any order, to a patient in need thereof a cancer immunotherapy (e.g., a checkpoint blocking antibody) to treat a subject (e.g., a human subject), e.g., having a disease or disorder described herein (e.g., a cancer described herein)).

In some embodiments, the immunotherapeutic agent is an anti-CTLA-4 antibody (e.g., ipilimumab, tremelimumab).

In some embodiments, the immunotherapeutic agent is an anti-PD-1 antibody (e.g., anti-PD-1 or anti-PD-L1). In some embodiments, the immunotherapeutic agent is an anti-PD-1 agent (e.g., an anti-PD-1 antibody, e.g., nivolumab (i.e., MDX-1106, BMS-936558, ONO-4538); CT-011; AMP-224; pembrolizumab (MK-3475); pidilizumab; cemiplimab; dostarlimab; prolgolimab; spartalizumab; camrelizumab; sasanlimab, sintilimab; tislelizumab; toripalimab; retifanlimab; MEDI0680; budigalimab; geptanolimab). In some embodiments, the immunotherapeutic agent is an anti-PD-L1 agent (e.g., an anti-PD-L1 antibody, e.g., BMS936559 (i.e., MDX-1105); durvalumab (MEDI4736); avelumab (MSB0010718C); envafolimab; cosibelimab; sugemalimab, AUNP-12 or atezolizumab (MPDL-3280A) or an anti-PD-L1 small molecule (e.g., CA-170)).

In some embodiments, the immunotherapeutic agent is a checkpoint blocking antibody (e.g., anti-TIM3, anti-LAG3, anti-TIGIT including IMP321 and MGA271).

In some embodiments, the immunotherapeutic agent is a cell-based therapy. In some embodiments, the cell-based therapy is a CAR-T therapy.

In some embodiments, the immunotherapeutic agent is a co-stimulatory antibody (e.g., anti-4-1BB, anti-OX40, anti-GITR, anti-CD27, anti-CD40).

In some embodiments, the immunotherapeutic agent is a cancer vaccine such as a neoantigen. These vaccines can be developed using peptides or RNA.

In some embodiments, the immunotherapeutic agent is an oncolytic virus.

In some embodiments, the immunotherapeutic agent is a STING pathway agonist. Exemplary STING agonists include MK-1454 and ADU-5100.

In some embodiments, provided is a method of treating a disease or disorder (e.g., cancer) comprising administering or coadministering, in any order, to a patient in need thereof a compound of formula (I), crystalline forms (e.g., crystalline form A), pharmaceutical compositions (e.g., comprising a compound of formula (I) or a pharmaceutically acceptable salt thereof) or dosage forms thereof, as described herein, and a disease-specific huMAB (e.g., an anti-HER3 huMAB).

In some embodiments, provided is a method of treating a disease or disorder (e.g., cancer) comprising administering or coadministering, in any order, to a patient in need thereof a compound of formula (I), crystalline forms (e.g., crystalline form A), pharmaceutical compositions (e.g., comprising a compound of formula (I) or a pharmaceutically acceptable salt thereof) or dosage forms thereof, as described herein, and an ADC/ADCC contingent on the expression of relevant surface targets on target tumors of interest.

In some embodiments, provided is a method of treating a disease or disorder (e.g., cancer) comprising administering or coadministering, in any order, to a patient in need thereof a compound of formula (I), crystalline forms (e.g., crystalline form A), pharmaceutical compositions (e.g., comprising a compound of formula (I) or a pharmaceutically acceptable salt thereof) or dosage forms thereof, as described herein, and one or more DNA damage pathway inhibitor. In some embodiments, a DNA damage pathway inhibitor is selected from the group consisting of bleomycin, an ATM inhibitor (e.g., AZD1390), a USP1 inhibitor, a WEE1 inhibitor (e.g., AZD1775), and a Chk1 inhibitor (e.g., AZD7762). In some embodiments, a DNA damage pathway inhibitor is a DNA alkylating agent.

In some embodiments, provided is a method of treating a disease or disorder (e.g., cancer) comprising administering or coadministering, in any order, to a patient in need thereof a compound of formula (I), crystalline forms (e.g., crystalline form A), pharmaceutical compositions (e.g., comprising a compound of formula (I) or a pharmaceutically acceptable salt thereof) or dosage forms thereof, as described herein, and a PARP inhibitor. In some embodiments, a PARP inhibitor is selected from the group consisting of olaparib, rucaparib, niraparib, talazoparib, veliparib, pamiparib, CEP 9722, E7016, iniparib, and 3-aminobenzamide.

Some patients may experience allergic reactions to the PRMT5 inhibitors described herein and/or other anti-cancer agent(s) during or after administration; therefore, anti-allergic agents are often administered to minimize the risk of an allergic reaction. In some embodiments, provided is a method of treating a disease or disorder (e.g., cancer) comprising administering or coadministering, in any order, to a patient in need thereof a compound of formula (I), crystalline forms (e.g., crystalline form A), pharmaceutical compositions (e.g., comprising a compound of formula (I) or a pharmaceutically acceptable salt thereof) or dosage forms thereof, as described herein, and an anti-allergic agent (e.g., corticosteroids, including, but not limited to, dexamethasone (e.g., Decadron®), beclomethasone (e.g., Beclovent®), hydrocortisone (also known as cortisone, hydrocortisone sodium succinate, hydrocortisone sodium phosphate, and sold under the tradenames Ala-Cort®, hydrocortisone phosphate, Solu-Cortef®, Hydrocort Acetate® and Lanacort®), prednisolone (sold under the tradenames Delta-Cortel®, Orapred®, Pediapred® and Prelone®), prednisone (sold under the tradenames Deltasone®, Liquid Red®, Meticorten® and Orasone®), methylprednisolone (also known as 6-methylprednisolone, methylprednisolone acetate, methylprednisolone sodium succinate, sold under the tradenames Duralone®, Medralone®, Medrol®, M-Prednisol® and Solu-Medrol®); antihistamines, such as diphenhydramine (e.g., Benadryl®), hydroxyzine, and cyproheptadine; and bronchodilators, such as the beta-adrenergic receptor agonists, albuterol (e.g., Proventil®), and terbutaline (Brethine®)).

Some patients may experience nausea during and after administration of the PRMT5 inhibitors described herein and/or other anti-cancer agent(s); therefore, anti-emetics are used in preventing nausea (upper stomach) and vomiting. In some embodiments, provided is a method of treating a disease or disorder (e.g., cancer) comprising administering or coadministering, in any order, to a patient in need thereof a compound of formula (I), crystalline forms (e.g., crystalline form A), pharmaceutical compositions (e.g., comprising a compound of formula (I) or a pharmaceutically acceptable salt thereof) or dosage forms thereof, as described herein, and an anti-emetic (e.g., aprepitant (Emend®), ondansetron (Zofran®), granisetron HCl (Kytril®), lorazepam (Ativan®. dexamethasone (Decadron®), prochlorperazine (Compazine®), casopitant (Rezonic® and Zunrisa®), and combinations thereof).

Medication to alleviate the pain experienced during the treatment period is often prescribed to make the patient more comfortable. In some embodiments, provided is a method of treating a disease or disorder (e.g., cancer) comprising administering or coadministering, in any order, to a patient in need thereof a compound of formula (I), crystalline forms (e.g., crystalline form A), pharmaceutical compositions (e.g., comprising a compound of formula (I) or a pharmaceutically acceptable salt thereof) or dosage forms thereof, as described herein, and an analgesic (e.g., an over-the-counter analgesic (e.g., Tylenol °), an opioid analgesic (e.g., hydrocodone/paracetamol or hydrocodone/acetaminophen (e.g., Vicodin®), morphine (e.g., Astramorph® or Avinza®), oxycodone (e.g., OxyContin® or Percocet®), oxymorphone hydrochloride (Opana®), and fentanyl (e.g., Duragesic®)).

In an effort to protect normal cells from treatment toxicity and to limit organ toxicities, cytoprotective agents (such as neuroprotectants, free-radical scavengers, cardioprotectors, anthracycline extravasation neutralizers, nutrients and the like) may be used as an adjunct therapy. In some embodiments, provided is a method of treating a disease or disorder (e.g., cancer) comprising administering or coadministering, in any order, to a patient in need thereof a compound of formula (I), crystalline forms (e.g., crystalline form A), pharmaceutical compositions (e.g., comprising a compound of formula (I) or a pharmaceutically acceptable salt thereof) or dosage forms thereof, as described herein, and a cytoprotective agent (e.g., Amifostine (Ethyol®), glutamine, dimesna (Tavocept®), mesna (Mesnex®), dexrazoxane (Zinecard® or Totect®), xaliproden (Xaprila®), and leucovorin (also known as calcium leucovorin, citrovorum factor and folinic acid)).

The structure of the active compounds identified by code numbers, generic or trade names may be taken from the actual edition of the standard compendium “The Merck Index” or from databases, e.g., Patents International (e.g., IMS World Publications).

The above-mentioned compounds, which can be used in combination with a PRMT5 inhibitor as described herein, can be prepared and administered as described in the art, including, but not limited to, in the documents cited above.

In one embodiment, provided are pharmaceutical compositions comprising at least one compound of the present disclosure (e.g., a crystalline form of a compound of formula (I), e.g., crystalline form A) together with a pharmaceutically acceptable carrier suitable for administration to a human or animal subject, either alone or together with other anti-cancer agents.

In particular, compositions will either be formulated together as a combination therapeutic or administered separately.

In combination therapy, a PRMT5 inhibitor as described herein and other anti-cancer agent(s) may be administered either simultaneously, concurrently or sequentially with no specific time limits, wherein such administration provides therapeutically effective levels of the two compounds in the body of the patient.

In a preferred embodiment, the compound of the present disclosure (a compound of formula (I), crystalline forms (e.g., crystalline form A), pharmaceutical compositions (e.g., comprising a compound of formula (I) or a pharmaceutically acceptable salt thereof) or dosage forms thereof, as described herein) and the other anti-cancer agent(s) is generally administered sequentially in any order by infusion or orally. The dosing regimen may vary depending upon the stage of the disease, physical fitness of the patient, safety profiles of the individual drugs, and tolerance of the individual drugs, as well as other criteria well-known to the attending physician and medical practitioner(s) administering the combination. The PRMT5 inhibitor as described herein and other anti-cancer agent(s) may be administered within minutes of each other, hours, days, or even weeks apart depending upon the particular cycle being used for treatment. In addition, the cycle could include administration of one drug more often than the other during the treatment cycle and at different doses per administration of the drug.

In another aspect, provided are kits that include one or more PRMT5 inhibitor(s) as described herein (a compound of formula (I), crystalline forms (e.g., crystalline form A), pharmaceutical compositions (e.g., comprising a compound of formula (I) or a pharmaceutically acceptable salt thereof) or dosage forms thereof, as described herein) and a second therapeutic agent as disclosed herein are provided. Representative kits include (a) a PRMT5 inhibitor as described herein (a compound of formula (I), crystalline forms (e.g., crystalline form A), pharmaceutical compositions (e.g., comprising a compound of formula (I) or a pharmaceutically acceptable salt thereof) or dosage forms thereof, as described herein), (b) at least one other therapeutic agent, e.g., as indicated above, whereby such kit may comprise a package insert or other labeling including directions for administration.

A compound of formula (I), crystalline forms (e.g., crystalline form A), pharmaceutical compositions (e.g., comprising a compound of formula (I) or a pharmaceutically acceptable salt thereof) or dosage forms thereof, as described herein may also be used in combination with known therapeutic processes, for example, the administration of hormones or especially radiation. A compound of the present disclosure may in particular be used as a radiosensitizer, especially for the treatment of tumors which exhibit poor sensitivity to radiotherapy. In some embodiments, provided is a method of treating a disease or disorder (e.g., cancer) comprising administering or coadministering, in any order, to a patient in need thereof a compound of formula (I), crystalline forms (e.g., crystalline form A), pharmaceutical compositions (e.g., comprising a compound of formula (I) or a pharmaceutically acceptable salt thereof) or dosage forms thereof, as described herein, and radiation.

### Patient Selection and Monitoring

In some embodiments, provided is a method of determining if a subject having or having been diagnosed with a cancer (e.g., a cancer patient) will respond to therapeutic treatment with a PRMT5 inhibitor (e.g., an MTA-uncompetitive PRMT5 inhibitor, e.g., a compound of formula (I), crystalline forms (e.g., crystalline form A), pharmaceutical compositions (e.g., comprising a compound of formula (I) or a pharmaceutically acceptable salt thereof) or dosage forms thereof, as described herein), comprising the steps of:
a) contacting a test sample obtained from said subject with a reagent capable of detecting human cancer cells that have MTAP deficiency and/or MTA accumulation; and
b) comparing the test sample with a reference (e.g., a reference sample taken from a non cancerous or normal control subject),
wherein the presence of MTAP deficiency and/or MTA accumulation in said test sample indicates that the subject will respond to therapeutic treatment with a PRMT5 inhibitor (e.g., an MTA-uncompetitive, non-competitive, or mixed mode PRMT5 inhibitor or an MTA-cooperative binding agent, e.g., a compound of formula (I), crystalline forms (e.g., crystalline form A), pharmaceutical compositions (e.g., comprising a compound of formula (I) or a pharmaceutically acceptable salt thereof) or dosage forms thereof, as described herein).

In some embodiments, provided is a method of determining if a cancer will respond to therapeutic treatment with a PRMT5 inhibitor (e.g., an MTA-uncompetitive, non-competitive, or mixed mode PRMT5 inhibitor or an MTA-cooperative binding agent, a compound of formula (I), crystalline forms (e.g., crystalline form A), pharmaceutical compositions (e.g., comprising a compound of formula (I) or a pharmaceutically acceptable salt thereof) or dosage forms thereof, as described herein), comprising the steps of:
a) contacting a test sample obtained from a subject having or having been diagnosed with said cancer with a reagent capable of detecting human cancer cells that have MTAP deficiency and/or MTA accumulation; and
b) comparing the test sample with a reference (e.g., a reference sample taken from a non cancerous or normal control subject),
   wherein the presence of MTAP deficiency and/or MTA accumulation in said test sample indicates that the cancer will respond to therapeutic treatment with a PRMT5 inhibitor (e.g., an MTA-uncompetitive, non-competitive, or mixed mode PRMT5 inhibitor or an MTA-cooperative binding agent, e.g., a compound of formula (I), crystalline forms (e.g., crystalline form A), pharmaceutical compositions (e.g., comprising a compound of formula (I) or a pharmaceutically acceptable salt thereof) or dosage forms thereof, as described herein). In some embodiments, the cancer is glioma, glioblastoma, malignant peripheral nerve sheath tumors (MPNST, e.g., intracranial MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine, sarcoma or a CNS metastasis from a solid tumor.

In some embodiments, the cancer is a CNS malignancy. In some embodiments, the CNS malignancy is selected from glioma (e.g., low grade glioma, intermediate grade glioma), intracranial MPNST tumors, glioblastoma, glioblastoma multiforme, or CNS metastases from solid tumors. In some embodiments, the CNS malignancy is glioma. In some embodiments, the CNS malignancy is low grade glioma. In some embodiments, the CNS malignancy is intermediate grade glioma. In some embodiments, the CNS malignancy is glioblastoma or glioblastoma multiforme. In some embodiments, the CNS malignancy is glioblastoma. In some embodiments, the CNS malignancy is glioblastoma multiforme. In some embodiments, the CNS malignancy is a MTAP-deleted glioblastoma. In some embodiments, the CNS malignancy is an intracranial MPNST tumor. In some embodiments, the CNS malignancy is CNS metastases from solid tumors.

In some embodiments, the cancer is a cancer selected from the group of cholangiocarcinoma, NSCLC (adenocarcinoma), NSCLC (squamous), bladder cancer, and DLBCL. In some embodiments, the cancer is a cancer selected from the group consisting of glioma, glioblastoma, non-small cell lung cancer (adenocarcinoma and squamous), mesothelioma, cholangiocarcinoma, urothelial carcinoma, and malignant peripheral nerve sheath tumor. In some embodiments, the cancer is cholangiocarcinoma. In some embodiments, the cancer is NSCLC (adenocarcinoma). In some embodiments, the cancer is NSCLC (squamous). In some embodiments, the cancer is bladder cancer. In some embodiments, the cancer is DLBCL.

In some embodiments, the cancer is a cancer selected from the group consisting of glioma, glioblastoma, non-small cell lung cancer (adenocarcinoma and squamous), mesothelioma, cholangiocarcinoma, urothelial carcinoma, and malignant peripheral nerve sheath tumor.

In some embodiments, the method further comprises the step of determining the level of PRMT5 in the cancer cells. The level of expression of PRMT5 can be considered when determining the therapeutically effective dosage of a PRMT5 inhibitor.

In one aspect, provided is a method of determining the sensitivity of a cancer cell to PRMT5 inhibition (e.g., inhibition with an MTA-uncompetitive PRMT5 inhibitor, e.g., a compound of formula (I), crystalline forms (e.g., crystalline form A), pharmaceutical compositions (e.g., comprising a compound of formula (I) or a pharmaceutically acceptable salt thereof) or dosage forms thereof, as described herein), comprising the steps of:
a) assaying the production, level, activity, expression or presence of MTAP), in said cancer cell;
b) comparing the production, level, activity, expression or presence of MTAP in the cancer cell with the production, level, activity, expression or presence of MTAP, respectively, in a non-cancerous or normal control cell,
   wherein a decreased level, activity or expression in the cancer cell indicates MTAP deficiency and wherein MTAP deficiency indicates that said cancer cell is sensitive to the PRMT5 inhibitor.

In some embodiments, the cancer is glioma, glioblastoma, malignant peripheral nerve sheath tumors (MPNST, e.g., intracranial MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine, sarcoma or a CNS metastasis from a solid tumor.

In some embodiments, the cancer is a CNS malignancy. In some embodiments, the CNS malignancy is selected from glioma (e.g., low grade glioma, intermediate grade glioma), intracranial MPNST tumors, glioblastoma, glioblastoma multiforme, or CNS metastases from solid tumors. In some embodiments, the CNS malignancy is glioma. In some embodiments, the CNS malignancy is low grade glioma. In some embodiments, the CNS malignancy is intermediate grade glioma. In some embodiments, the CNS malignancy is glioblastoma or glioblastoma multiforme. In some embodiments, the CNS malignancy is glioblastoma. In some embodiments, the CNS malignancy is glioblastoma multiforme. In some embodiments, the CNS malignancy is a MTAP-deleted glioblastoma. In some embodiments, the CNS malignancy is an intracranial MPNST tumor. In some embodiments, the CNS malignancy is CNS metastases from solid tumors.

In some embodiments, the cancer is a cancer selected from the group of cholangiocarcinoma, NSCLC (adenocarcinoma), NSCLC (squamous), bladder cancer, and DLBCL. In some embodiments, the cancer is a cancer selected from the group consisting of glioma, glioblastoma, non-small cell lung cancer (adenocarcinoma and squamous), mesothelioma, cholangiocarcinoma, urothelial carcinoma, and malignant peripheral nerve sheath tumor. In some embodiments, the cancer is cholangiocarcinoma. In some embodiments, the cancer is NSCLC (adenocarcinoma). In some embodiments, the cancer is NSCLC (squamous). In some embodiments, the cancer is bladder cancer. In some embodiments, the cancer is DLBCL.

In some embodiments, the cancer is a cancer selected from the group consisting of glioma, glioblastoma, non-small cell lung cancer (adenocarcinoma and squamous), mesothelioma, cholangiocarcinoma, urothelial carcinoma, and malignant peripheral nerve sheath tumor.

In one embodiment, provided is a method of determining the sensitivity of a cancer cell to a PRMT5 inhibitor (e.g., an MTA-uncompetitive, non-competitive, or mixed mode PRMT5 inhibitor or an MTA-cooperative binding agent, e.g., a compound of formula (I), crystalline forms (e.g., crystalline form A), pharmaceutical compositions (e.g., comprising a compound of formula (I) or a pharmaceutically acceptable salt thereof) or dosage forms thereof, as described herein), comprising the steps of:
a) assaying for level, activity or expression of the MTAP gene or its gene product in both the cancer cell and a normal control cell, wherein a decreased level, activity or expression in the cancer cell indicates MTAP deficiency; b) assaying for PRMT5 expression in said cancer cell; c) comparing the PRMT5 expression with PRMT5 expression in the cancer cell and a normal control cell; wherein the similarity in PRMT5 expression, and the presence of said MTAP deficiency in said cancer cell, indicates said cell is sensitive to a PRMT5 inhibitor.

In some embodiments, the cancer is glioma, glioblastoma, malignant peripheral nerve sheath tumors (MPNST, e.g., intracranial MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine, sarcoma or a CNS metastasis from a solid tumor.

In some embodiments, the cancer is a CNS malignancy. In some embodiments, the CNS malignancy is selected from glioma (e.g., low grade glioma, intermediate grade glioma), intracranial MPNST tumors, glioblastoma, glioblastoma multiforme, or CNS metastases from solid tumors. In some embodiments, the CNS malignancy is glioma. In some embodiments, the CNS malignancy is low grade glioma. In some embodiments, the CNS malignancy is intermediate grade glioma. In some embodiments, the CNS malignancy is glioblastoma or glioblastoma multiforme. In some embodiments, the CNS malignancy is glioblastoma. In some embodiments, the CNS malignancy is glioblastoma multiforme. In some embodiments, the CNS malignancy is a MTAP-deleted glioblastoma. In some embodiments, the CNS malignancy is an intracranial MPNST tumor. In some embodiments, the CNS malignancy is CNS metastases from solid tumors.

In some embodiments, the cancer is a cancer selected from the group of cholangiocarcinoma, NSCLC (adenocarcinoma), NSCLC (squamous), bladder cancer, and DLBCL. In some embodiments, the cancer is a cancer selected from the group consisting of glioma, glioblastoma, non-small cell lung cancer (adenocarcinoma and squamous), mesothelioma, cholangiocarcinoma, urothelial carcinoma, and malignant peripheral nerve sheath tumor. In some embodiments, the cancer is cholangiocarcinoma. In some embodiments, the cancer is NSCLC (adenocarcinoma). In some embodiments, the cancer is NSCLC (squamous). In some embodiments, the cancer is bladder cancer. In some embodiments, the cancer is DLBCL.

In some embodiments, the cancer is a cancer selected from the group consisting of glioma, glioblastoma, non-small cell lung cancer (adenocarcinoma and squamous), mesothelioma, cholangiocarcinoma, urothelial carcinoma, and malignant peripheral nerve sheath tumor.

In one aspect the provided is a therapeutic method of treating a subject having or having been diagnosed with a cancer (e.g., a cancer associated with MTAP deficiency and/or MTA accumulation) comprising the steps of:
a) assessing the level of MTAP and/or MTA in a test sample obtained from said subject (e.g., by contacting the sample with a reagent capable of detecting human MTAP-deficient and/or MTA-accumulating cancer cells in a test sample obtained from said subject), wherein the MTA level can be assessed directly (e.g., by ELISA or LC-MS/MS) or indirectly (e.g., by SDMA-modified protein ELISA or IHC, or by RNA splicing);
   b) comparing the test sample with a reference (e.g., a reference sample taken from a non cancerous or normal control subject), wherein MTAP deficiency and/or MTA accumulation in said test sample indicates said subject will respond to therapeutic treatment with a PRMT5 inhibitor; and
   c) administering a therapeutically effective amount of PRMT5 inhibitor (e.g., an MTA-uncompetitive, non-competitive, or mixed mode PRMT5 inhibitor or an MTA-cooperative binding agent, e.g., a compound of formula (I), crystalline forms (e.g., crystalline form A), pharmaceutical compositions (e.g., comprising a compound of formula (I) or a pharmaceutically acceptable salt thereof) or dosage forms thereof, as described herein) to the subject identified in step b).

In one aspect provided is a therapeutic method of treating a cancer (e.g., a cancer associated with MTAP deficiency and/or MTA accumulation) in a subject in need thereof comprising the steps of:
a) assessing the level of MTAP and/or MTA in a test sample obtained from said subject (e.g., by contacting the sample with a reagent capable of detecting human MTAP-deficient and/or MTA-accumulating cancer cells), wherein the MTA level can be assessed directly (e.g., by ELISA or LC-MS/MS) or indirectly (e.g., by SDMA-modified protein ELISA or IHC, or by RNA splicing);
b) comparing the test sample with a reference (e.g., a reference sample taken from a non cancerous or normal control subject), wherein MTAP deficiency and/or MTA accumulation in said test sample indicates said cancer will respond to therapeutic treatment with a PRMT5 inhibitor (e.g., an MTA-uncompetitive, non-competitive, or mixed mode PRMT5 inhibitor or an MTA-cooperative binding agent); and
c) administering a therapeutically effective amount of PRMT5 inhibitor (e.g., an MTA-uncompetitive, non-competitive, or mixed mode PRMT5 inhibitor or an MTA-cooperative binding agent, e.g., a compound of formula (I), crystalline forms (e.g., crystalline form A), pharmaceutical compositions (e.g., comprising a compound of formula (I) or a pharmaceutically acceptable salt thereof) or dosage forms thereof, as described herein) to the subject identified in step b).

In some embodiments, the cancer is glioma, glioblastoma, malignant peripheral nerve sheath tumors (MPNST, e.g., intracranial MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine, sarcoma or a CNS metastasis from a solid tumor.

In some embodiments, the cancer is a CNS malignancy. In some embodiments, the CNS malignancy is selected from glioma (e.g., low grade glioma, intermediate grade glioma), intracranial MPNST tumors, glioblastoma, glioblastoma multiforme, or CNS metastases from solid tumors. In some embodiments, the CNS malignancy is glioma. In some embodiments, the CNS malignancy is low grade glioma. In some embodiments, the CNS malignancy is intermediate grade glioma. In some embodiments, the CNS malignancy is glioblastoma or glioblastoma multiforme. In some embodiments, the CNS malignancy is glioblastoma. In some embodiments, the CNS malignancy is glioblastoma multiforme. In some embodiments, the CNS malignancy is a MTAP-deleted glioblastoma. In some embodiments, the CNS malignancy is an intracranial MPNST tumor. In some embodiments, the CNS malignancy is CNS metastases from solid tumors.

In some embodiments, the cancer is a cancer selected from the group of cholangiocarcinoma, NSCLC (adenocarcinoma), NSCLC (squamous), bladder cancer, and DLBCL. In some embodiments, the cancer is a cancer selected from the group consisting of glioma, glioblastoma, non-small cell lung cancer (adenocarcinoma and squamous), mesothelioma, cholangiocarcinoma, urothelial carcinoma, and malignant peripheral nerve sheath tumor. In some embodiments, the cancer is cholangiocarcinoma. In some embodiments, the cancer is NSCLC (adenocarcinoma). In some embodiments, the cancer is NSCLC (squamous). In some embodiments, the cancer is bladder cancer. In some embodiments, the cancer is DLBCL.

In some embodiments, the cancer is a cancer selected from the group consisting of glioma, glioblastoma, non-small cell lung cancer (adenocarcinoma and squamous), mesothelioma, cholangiocarcinoma, urothelial carcinoma, and malignant peripheral nerve sheath tumor.

In some embodiments, the method further comprises the step of determining the level of PRMT5 in the cancer cells.

In one aspect provided is a therapeutic method of treating a subject having or having been diagnosed with a cancer associated with MTAP deficiency and/or MTA accumulation comprising the steps of:
a) assessing the level of MTAP and/or MTA in a test sample obtained from said subject (e.g., by contacting the sample with a reagent capable of detecting human MTAP-deficient and/or MTA-accumulating cancer cells), wherein the MTA level can be assessed directly (e.g., by ELISA or LC-MS/MS) or indirectly (e.g., by SDMA-modified protein ELISA or IHC, or by RNA splicing);
b) comparing the test sample with a reference sample (e.g., a reference sample taken from a non-cancerous or normal control subject), wherein MTAP deficiency and/or MTA accumulation in said test sample indicates said cancer will respond to therapeutic treatment with a PRMT5 inhibitor (e.g., an MTA-uncompetitive, non-competitive, or mixed mode PRMT5 inhibitor or an MTA-cooperative binding agent); and
c) administering a therapeutically effective amount of a composition comprising a PRMT5 inhibitor (e.g., an MTA-uncompetitive, non-competitive, or mixed mode PRMT5 inhibitor or an MTA-cooperative binding agent, e.g., a compound of formula (I), crystalline forms (e.g., crystalline form A), pharmaceutical compositions (e.g., comprising a compound of formula (I) or a pharmaceutically acceptable salt thereof) or dosage forms thereof, as described herein) to the subject identified in step b).

In one aspect provided is a therapeutic method of treating cancer associated with MTAP deficiency and/or MTA accumulation in a subject in need thereof comprising the steps of:
a) assessing the level of MTAP and/or MTA in a test sample obtained from said subject (e.g., by contacting the sample with a reagent capable of detecting human MTAP-deficient and/or MTA-accumulating cancer cells), wherein the MTA level can be assessed directly (e.g., by ELISA or LC-MS/MS) or indirectly (e.g., by SDMA-modified protein ELISA or IHC, or by RNA splicing);
b) comparing the test sample with a reference sample (e.g., a reference sample taken from a non-cancerous or normal control subject), wherein MTAP deficiency and/or MTA accumulation in said test sample indicates said cancer will respond to therapeutic treatment with a PRMT5 inhibitor (e.g., an MTA-uncompetitive, non-competitive, or mixed mode PRMT5 inhibitor or an MTA-cooperative binding agent); and
c) administering a therapeutically effective amount of a composition comprising a PRMT5 inhibitor (e.g., an MTA-uncompetitive PRMT5 inhibitor e.g., a compound of formula (I), crystalline forms (e.g., crystalline form A), pharmaceutical compositions (e.g., comprising a compound of formula (I) or a pharmaceutically acceptable salt thereof) or dosage forms thereof, as described herein) to the subject identified in step b).

In some embodiments, the cancer is glioma, glioblastoma, malignant peripheral nerve sheath tumors (MPNST, e.g., intracranial MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine, sarcoma or a CNS metastasis from a solid tumor.

In some embodiments, the cancer is a CNS malignancy. In some embodiments, the CNS malignancy is selected from glioma (e.g., low grade glioma, intermediate grade glioma), intracranial MPNST tumors, glioblastoma, glioblastoma multiforme, or CNS metastases from solid tumors. In some embodiments, the CNS malignancy is glioma. In some embodiments, the CNS malignancy is low grade glioma. In some embodiments, the CNS malignancy is intermediate grade glioma. In some embodiments, the CNS malignancy is glioblastoma or glioblastoma multiforme. In some embodiments, the CNS malignancy is glioblastoma. In some embodiments, the CNS malignancy is glioblastoma multiforme. In some embodiments, the CNS malignancy is a MTAP-deleted glioblastoma. In some embodiments, the CNS malignancy is an intracranial MPNST tumor. In some embodiments, the CNS malignancy is CNS metastases from solid tumors.

In some embodiments, the cancer is a cancer selected from the group of cholangiocarcinoma, NSCLC (adenocarcinoma), NSCLC (squamous), bladder cancer, and DLBCL. In some embodiments, the cancer is a cancer selected from the group consisting of glioma, glioblastoma, non-small cell lung cancer (adenocarcinoma and squamous), mesothelioma, cholangiocarcinoma, urothelial carcinoma, and malignant peripheral nerve sheath tumor. In some embodiments, the cancer is cholangiocarcinoma. In some embodiments, the cancer is NSCLC (adenocarcinoma). In some embodiments, the cancer is NSCLC (squamous). In some embodiments, the cancer is bladder cancer. In some embodiments, the cancer is DLBCL.

In some embodiments, the cancer is a cancer selected from the group consisting of glioma, glioblastoma, non-small cell lung cancer (adenocarcinoma and squamous), mesothelioma, cholangiocarcinoma, urothelial carcinoma, and malignant peripheral nerve sheath tumor.

In some embodiments, the method further comprises the step of determining the level of PRMT5 in the cancer cells.

In some embodiments provided is a method of determining if a subject having or having been diagnosed with a cancer associated with MTAP deficiency and/or MTA accumulation will respond to treatment with a PRMT5 inhibitor (e.g., an MTA-uncompetitive, non-competitive, or mixed mode PRMT5 inhibitor or an MTA-cooperative binding agent, e.g., a compound of formula (I), crystalline forms (e.g., crystalline form A), pharmaceutical compositions (e.g., comprising a compound of formula (I) or a pharmaceutically acceptable salt thereof) or dosage forms thereof, as described herein) comprising the steps of:
a) assessing the level of MTAP and/or MTA in a test sample obtained from said subject (e.g., by contacting the sample with a reagent capable of detecting human MTAP-deficient and/or MTA-accumulating cancer cells), wherein the MTA level can be assessed directly (e.g., by ELISA or LC-MS/MS) or indirectly (e.g., by SDMA-modified protein ELISA or IHC, or by RNA splicing);
b) comparing the test sample with a reference (e.g., a reference sample taken from a non cancerous or normal control subject), wherein MTAP deficiency and/or MTA accumulation in said test sample indicates said subject will respond to therapeutic treatment with a PRMT5 inhibitor (e.g., an MTA-uncompetitive, non-competitive, or mixed mode PRMT5 inhibitor or an MTA-cooperative binding agent).

In some embodiments provided is a method of determining if a cancer associated with MTAP deficiency and/or MTA accumulation will respond to treatment with a PRMT5 inhibitor (e.g., an MTA-uncompetitive PRMT5 inhibitor, e.g., a compound of formula (I), crystalline forms (e.g., crystalline form A), pharmaceutical compositions (e.g., comprising a compound of formula (I) or a pharmaceutically acceptable salt thereof) or dosage forms thereof, as described herein) comprising the steps of:
a) assessing the level of MTAP and/or MTA in a test sample obtained from a subject having or having been diagnosed with said cancer (e.g., by contacting the sample with a reagent capable of detecting human MTAP-deficient and/or MTA-accumulating cancer cells), wherein the MTA level can be assessed directly (e.g., by ELISA or LC-MS/MS) or indirectly (e.g., by SDMA-modified protein ELISA or IHC, or by RNA splicing);
b) comparing the test sample with a reference (e.g., a reference sample taken from a non cancerous or normal control subject), wherein MTAP deficiency and/or MTA accumulation in said test sample indicates said cancer will respond to therapeutic treatment with a PRMT5 inhibitor (e.g., an MTA-uncompetitive, non-competitive, or mixed mode PRMT5 inhibitor or an MTA-cooperative binding agent).

In some embodiments, the cancer is glioma, glioblastoma, malignant peripheral nerve sheath tumors (MPNST, e.g., intracranial MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine, sarcoma or a CNS metastasis from a solid tumor.

In some embodiments, the cancer is a CNS malignancy. In some embodiments, the CNS malignancy is selected from glioma (e.g., low grade glioma, intermediate grade glioma), intracranial MPNST tumors, glioblastoma, glioblastoma multiforme, or CNS metastases from solid tumors. In some embodiments, the CNS malignancy is glioma. In some embodiments, the CNS malignancy is low grade glioma. In some embodiments, the CNS malignancy is intermediate grade glioma. In some embodiments, the CNS malignancy is glioblastoma or glioblastoma multiforme. In some embodiments, the CNS malignancy is glioblastoma. In some embodiments, the CNS malignancy is glioblastoma multiforme. In some embodiments, the CNS malignancy is a MTAP-deleted glioblastoma. In some embodiments, the CNS malignancy is an intracranial MPNST tumor. In some embodiments, the CNS malignancy is CNS metastases from solid tumors.

In some embodiments, the cancer is a cancer selected from the group of cholangiocarcinoma, NSCLC (adenocarcinoma), NSCLC (squamous), bladder cancer, and DLBCL. In some embodiments, the cancer is a cancer selected from the group consisting of glioma, glioblastoma, non-small cell lung cancer (adenocarcinoma and squamous), mesothelioma, cholangiocarcinoma, urothelial carcinoma, and malignant peripheral nerve sheath tumor. In some embodiments, the cancer is cholangiocarcinoma. In some embodiments, the cancer is NSCLC (adenocarcinoma). In some embodiments, the cancer is NSCLC (squamous). In some embodiments, the cancer is bladder cancer. In some embodiments, the cancer is DLBCL.

In some embodiments, the cancer is a cancer selected from the group consisting of glioma, glioblastoma, non-small cell lung cancer (adenocarcinoma and squamous), mesothelioma, cholangiocarcinoma, urothelial carcinoma, and malignant peripheral nerve sheath tumor.

In some embodiments, the method further comprises the step of determining the level of PRMT5 in the cancer cells.

### Sample Preparation

Further provided are assays for the detection of MTAP deficiency and/or MTA accumulation. They can include detecting a mutation related to MTAP deficiency and/or MTA accumulation, e.g., in a body fluid such as blood (e.g., serum or plasma) bone marrow, cerebral spinal fluid, peritoneal/pleural fluid, lymph fluid, ascites, serous fluid, sputum, lacrimal fluid, stool, and urine, or in a tissue such as a tumor tissue. The tumor tissue can be fresh tissue or preserved tissue (e.g., formalin fixed tissue, e.g., paraffin-embedded tissue).

Body fluid samples can be obtained from a subject using any of the methods known in the art. Methods for extracting cellular DNA from body fluid samples are well known in the art. Typically, cells are lysed with detergents. After cell lysis, proteins are removed from DNA using various proteases. DNA is then extracted with phenol, precipitated in alcohol, and dissolved in an aqueous solution. Methods for extracting acellular DNA from body fluid samples are also known in the art. Commonly, a cellular DNA in a body fluid sample is separated from cells, precipitated in alcohol, and dissolved in an aqueous solution.

### Detection of PRMT5 Selectivity

Samples, once prepared, can be tested for MTAP deficiency and/or MTA accumulation, either or both of which indicates that the sample is sensitive to treatment with a PRMT5 inhibitor. Cells can be determined to be MTA accumulating by techniques known in the art; methods for detecting MTA include, as a non-limiting example, liquid chromatography-electrospray ionization-tandem mass spectrometry (LC-ESI-MS/MS), as described in Stevens et al. 2010. J. Chromatogr. A. 1217: 3282-3288; and Kirovski et al. 2011 Am. J. Pathol. 178: 1145-1152; and references cited therein. The detection of MTAP deficiency can be done by any number of ways, for example: DNA sequencing, PCR based methods, including RT-PCR, microarray analysis, Southern blotting, Northern blotting, Next Generation Sequencing, and dip stick analysis. In some embodiments, MTAP deficiency is evaluated by any technique known in the art, for example, immunohistochemistry utilizing an anti-MTAP antibody or derivative thereof, and/or genomic sequencing, or nucleic acid hybridization, or amplification utilizing at least one probe or primer comprising a sequence of at least 12 contiguous nucleotides (nt) of the sequence of MTAP wherein the primer is no longer than about 30 nt.

The polymerase chain reaction (PCR) can be used to amplify and identify MTAP deficiency from either genomic DNA or RNA extracted from tumor tissue. PCR is well known in the art and is described in detail in Saiki et al., Science 1988, 239:487.

Methods of detecting MTAP deficiency by hybridization are provided. The method comprises identifying MTAP deficiency in a sample by its inability to hybridize to MTAP nucleic acid. The nucleic acid probe is detectably labeled with a label such as a radioisotope, a fluorescent agent or a chromogenic agent. Radioisotopes can include without limitation; 3H, 32P, 33P and 35S etc. Fluorescent agents can include without limitation: FITC, texas red, rhodamine, etc.

The probe used in detection that is capable of hybridizing to MTAP nucleic acid can be from about 8 nucleotides to about 100 nucleotides, from about 10 nucleotides to about 75 nucleotides, from about 15 nucleotides to about 50 nucleotides, or about 20 to about 30 nucleotides. The kit can also provide instructions for analysis of patient cancer samples, wherein the presence or absence of MTAP deficiency indicates if the subject is sensitive or insensitive to treatment with a PRMT5 inhibitor.

Single stranded conformational polymorphism (SSCP) can also be used to detect MTAP deficiency. This technique is well described in Orita et al., PNAS 1989, 86:2766-2770.

### Measurement of Gene Expression

Evaluation of MTAP deficiency and measurement of MTAP gene expression, and measurement of PRMT5 gene expression can be performed using any method or reagent known in the art.

Detection of gene expression can be by any appropriate method, including for example, detecting the quantity of mRNA transcribed from the gene or the quantity of cDNA produced from the reverse transcription of the mRNA transcribed from the gene or the quantity of the polypeptide or protein encoded by the gene. These methods can be performed on a sample by sample basis or modified for high throughput analysis. For example, using Affymetrix™ U133 microarray chips.

In one aspect, gene expression is detected and quantitated by hybridization to a probe that specifically hybridizes to the appropriate probe for that biomarker. The probes also can be attached to a solid support for use in high throughput screening assays using methods known in the art.

In one aspect, the expression level of a gene is determined through exposure of a nucleic acid sample to the probe-modified chip. Extracted nucleic acid is labeled, for example, with a fluorescent tag, preferably during an amplification step.

Hybridization of the labeled sample is performed at an appropriate stringency level. The degree of probe-nucleic acid hybridization is quantitatively measured using a detection device.

Alternatively, any one of gene copy number, transcription, or translation can be determined using known techniques. For example, an amplification method such as PCR may be useful. General procedures for PCR are taught in MacPherson et al., PCR: A Practical Approach, (IRL Press at Oxford University Press (1991)). However, PCR conditions used for each application reaction are empirically determined. A number of parameters influence the success of a reaction. Among them are annealing temperature and time, extension time, Mg 2+ and/or ATP concentration, pH, and the relative concentration of primers, templates, and deoxyribonucleotides. After amplification, the resulting DNA fragments can be detected by agarose gel electrophoresis followed by visualization with ethidium bromide staining and ultraviolet illumination. In one embodiment, the hybridized nucleic acids are detected by detecting one or more labels attached to the sample nucleic acids. The labels can be incorporated by any of a number of means well known to those of skill in the art. However, in one aspect, the label is simultaneously incorporated during the amplification step in the preparation of the sample nucleic acid. Thus, for example, polymerase chain reaction (PCR) with labeled primers or labeled nucleotides will provide a labeled amplification product. In a separate embodiment, transcription amplification, as described above, using a labeled nucleotide (e.g., fluorescein-labeled UTP and/or CTP) incorporates a label in to the transcribed nucleic acids.

Alternatively, a label may be added directly to the original nucleic acid sample (e.g., mRNA, polyA, mRNA, cDNA, etc.) or to the amplification product after the amplification is completed. Means of attaching labels to nucleic acids are well known to those of skill in the art and include, for example nick translation or end-labeling (e.g., with a labeled RNA) by kinasing of the nucleic acid and subsequent attachment (ligation) of a nucleic acid linker joining the sample nucleic acid to a label (e.g., a fluorophore).

In one example, the gene expression can be measured through an in-situ hybridization protocol that can detect RNA molecules on a slide containing tissue sections or cells (e.g., through RNAscope®).

Detectable labels suitable for use in the methods disclosed herein include any composition detectable by spectroscopic, photochemical, biochemical, immunochemical, electrical, optical or chemical means. Useful labels include biotin for staining with labeled streptavidin conjugate, magnetic beads (e.g., Dynabeads™), fluorescent dyes (e.g., fluorescein, texas red, rhodamine, green fluorescent protein, and the like), radiolabels (e.g., 3H, 125I, 35S, 14C, or 32P) enzymes (e.g., horse radish peroxidase, alkaline phosphatase and others commonly used in an ELISA), and calorimetric labels such as colloidal gold or colored glass or plastic (e.g., polystyrene, polypropylene, latex, etc.) beads.

Detection of labels is well known to those of skill in the art. Thus, for example, radiolabels may be detected using photographic film or scintillation counters, fluorescent markers may be detected using a photodetector to detect emitted light. Enzymatic labels are typically detected by providing the enzyme with a substrate and detecting the reaction product produced by the action of the enzyme on the substrate, and calorimetric labels are detected by simply visualizing the colored label. The detectable label may be added to the target (sample) nucleic acid(s) prior to, or after the hybridization, such as described in WO 97/10365. These detectable labels are directly attached to or incorporated into the target (sample) nucleic acid prior to hybridization. In contrast, “indirect labels” are joined to the hybrid duplex after hybridization. Generally, the indirect label is attached to a binding moiety that has been attached to the target nucleic acid prior to the hybridization. For example, the target nucleic acid may be biotinylated before the hybridization. After hybridization, an avidin-conjugated fluorophore will bind the biotin bearing hybrid duplexes providing a label that is easily detected. For a detailed review of methods of labeling nucleic acids and detecting labeled hybridized nucleic acids see Laboratory Techniques in Biochemistry and Molecular Biology, Vol. 24: Hybridization with Nucleic Acid Probes, P. Tijssen, ed. Elsevier, N.Y. (1993).

### Detection of Polypeptides

Protein levels of MTAP can be determined by examining protein expression or the protein product. Determining the protein level involves measuring the amount of any immunospecific binding that occurs between an antibody that selectively recognizes and binds to the polypeptide of the biomarker in a sample obtained from a subject and comparing this to the amount of immunospecific binding of at least one biomarker in a control sample.

A variety of techniques are available in the art for protein analysis. They include but are not limited to radioimmunoassays, ELISA (enzyme linked immunosorbent assays), “sandwich” immunoassays, immunoradiometric assays, in situ immunoassays (using e.g., colloidal gold, enzyme or radioisotope labels), Western blot analysis, immunoprecipitation assays, immunofluorescent assays, flow cytometry, immunohistochemistry, HPLC, mass spectrometry, confocal microscopy, enzymatic assays, surface plasmon resonance and PAGE-SDS.

### Adjacent Biomarkers

Near or adjacent to MTAP on chromosome 9 are several other biomarkers. CDKN2A is often, if not usually, deleted along with MTAP. Additional genes or pseudogenes in this region include: C9orf53, ERVFRD-3, TUBB8P1, KHSRPP1, MIR31, and MIR31HG.

In some embodiments of the methods, the cell that is MTAP-deficient is also deficient in CDKN2A. In some embodiments, the cell that is MTAP-deficient is also deficient in one or more of: CDKN2A, C9orf53, ERVFRD-3, TUBB8P1, KHSRPP1, MIR31, and MIR31HG.

Thus, in various methods involving a step of evaluating a cell for MTAP deficiency or determining if a cell is MTAP-deficient, this step can comprise the step of determining if the cell is deficient for one or more of these markers: CDKN2A, C9orf53, ERVFRD-3, TUBB8P1, KHSRPP1, MIR31, and MIR31HG.

Thus, in some embodiments, the disclosure encompasses: A method of determining if a subject having or having been diagnosed with a cancer will respond to therapeutic treatment with a PRMT5 inhibitor (e.g., an MTA-uncompetitive, non-competitive, or mixed mode PRMT5 inhibitor or an MTA-cooperative binding agent), comprising the steps of:
a) evaluating a test sample obtained from said subject for MTAP deficiency, and evaluating a reference sample from a non-cancerous or normal control subject for MTAP deficiency, wherein MTAP deficiency in the test sample relative to the reference sample indicates that the subject will respond to therapeutic treatment with a PRMT5 inhibitor (e.g., an MTA-uncompetitive PRMT5 inhibitor, e.g., a crystalline form of a compound of formula (I)); wherein MTAP deficiency is evaluated by evaluating the deficiency of one or more of the following biomarkers: CDKN2A, C9orf53, ERVFRD-3, TUBB8P1, KHSRPP1, MIR31, and MIR31HG, and wherein the method can further comprise the following steps:
b) determining the level of MTAP in the subject, wherein steps a) and b) can be performed in any order;
c) administering a therapeutically effective amount of a PRMT5 inhibitor (e.g., an MTA-uncompetitive, non-competitive, or mixed mode PRMT5 inhibitor or an MTA-cooperative binding agent, e.g., a compound of formula (I), crystalline forms (e.g., crystalline form A), pharmaceutical compositions (e.g., comprising a compound of formula (I) or a pharmaceutically acceptable salt thereof) or dosage forms thereof, as described herein) to the subject; and
d) determining the level of PRMT5 activity in the subject following step c), wherein a decrease in the level of PRMT5 activity is correlated with the inhibition of the proliferation of the cancer, and wherein steps c) and d) are performed after steps a) and b).

In some embodiments, the disclosure encompasses: A method of determining if a cancer will respond to therapeutic treatment with a PRMT5 inhibitor (e.g., an MTA-uncompetitive, non competitive, or mixed mode PRMT5 inhibitor or an MTA-cooperative binding agent), comprising the steps of:
a) evaluating a test sample obtained from a subject having or having been diagnosed with said cancer for MTAP deficiency, and evaluating a reference sample from a non cancerous or normal control subject for MTAP deficiency, wherein MTAP deficiency in the test sample relative to the reference sample indicates that the cancer will respond to therapeutic treatment with a PRMT5 inhibitor (e.g., an MTA-uncompetitive, non competitive, or mixed mode PRMT5 inhibitor or an MTA-cooperative binding agent, e.g., a compound of formula (I), crystalline forms (e.g., crystalline form A), pharmaceutical compositions (e.g., comprising a compound of formula (I) or a pharmaceutically acceptable salt thereof) or dosage forms thereof, as described herein); wherein MTAP deficiency is evaluated by evaluating the deficiency of one or more of the following biomarkers: CDKN2A, C9orf53, ERVFRD-3, TUBB8P1, KHSRPP1, MIR31, and MIR31HG, and wherein the method can further comprise the following steps:
b) determining the level of MTAP in the subject, wherein steps a) and b) can be performed in any order;
c) administering a therapeutically effective amount of a PRMT5 inhibitor (e.g., an MTA-uncompetitive PRMT5 inhibitor, e.g., a compound of formula (I), crystalline forms (e.g., crystalline form A), pharmaceutical compositions (e.g., comprising a compound of formula (I) or a pharmaceutically acceptable salt thereof) or dosage forms thereof, as described herein) to the subject; and
d) determining the level of PRMT5 activity in the subject following step c), wherein a decrease in the level of PRMT5 activity is correlated with the inhibition of the proliferation of the cancer, and wherein steps c) and d) are performed after steps a) and b).

### Assaying for Biomarkers and PRMT5 Inhibitor Treatment

A number of patient stratification strategies could be employed to find patients likely to be sensitive to PRMT5 inhibition with an MTA-uncompetitive, non-competitive, or mixed mode PRMT5 inhibitor or an MTA-cooperative binding agent (e.g., a compound of formula (I), crystalline forms (e.g., crystalline form A), pharmaceutical compositions (e.g., comprising a compound of formula (I) or a pharmaceutically acceptable salt thereof) or dosage forms thereof, as described herein), including but not limited to, testing for MTAP deficiency and/or MTA accumulation.

Once a patient has been assayed for MTAP deficiency and/or MTA accumulation and predicted to be sensitive to treatment with a PRMT5 inhibitor, administration of any PRMT5 inhibitor (e.g., an MTA-uncompetitive, non-competitive, or mixed mode PRMT5 inhibitor or an MTA-cooperative binding agent, e.g., a crystalline form of a compound of formula (I)) to a patient can be effected in one dose, continuously or intermittently throughout the course of treatment. Methods of determining the most effective means and dosage of administration are well known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents may be empirically adjusted.

### Dosage

The dose ranges and recitations below refer to the dose of the compound of formula (I) contained in the dosage forms and pharmaceutical compositions described herein that are administered to the subject in need thereof as part of the methods described herein.

In some embodiments, the method comprises administering to the subject a dose of about 8 mg to about 17 mg of the compound of formula (I) once or twice daily. In some embodiments, the method comprises administering to the subject a dose of about 10 mg to about 15 mg of the compound of formula (I) once or twice daily. In some embodiments, the method comprises administering to the subject a dose of about 12 mg to about 13 mg of the compound of formula (I) once or twice daily.

In some embodiments, the method comprises administering to the subject a dose of about 8 mg, about 9 mg, about 10 mg, about 11 mg, about 12 mg, about 12.5 mg, about 13 mg, about 14 mg, about 15 mg, about 16 mg or about 17 mg of the compound of formula (I) once or twice daily.

In further embodiments, the method comprises administering to the subject a dose of about 12.5 mg of the compound of formula (I) once or twice daily.

In some embodiments, the method comprises administering to the subject a dose of about 40 mg to about 60 mg of the compound of formula (I) once or twice daily. In some embodiments, the method comprises administering to the subject a dose of about 45 mg to about 55 mg of the compound of formula (I) once or twice daily. In some embodiments, the method comprises administering to the subject a dose of about 47 mg to about 53 mg of the compound of formula (I) once or twice daily. In some embodiments, the method comprises administering to the subject a dose of about 49 mg to about 51 mg of the compound of formula (I) once or twice daily.

In some embodiments, the method comprises administering to the subject a dose of about 45 mg, about 46 mg, about 47 mg, about 48 mg, about 49 mg, about 50 mg, about 51 mg, about 52 mg, about 53 mg, about 54 mg or about 55 mg of the compound of formula (I) once or twice daily.

In some embodiments, the method comprises administering to the subject a dose of about 50 mg of the compound of formula (I) once or twice daily.

In some embodiments, the method comprises administering to the subject a dose of about 80 mg to about 120 mg of the compound of formula (I) once or twice daily. In some embodiments, the method comprises administering to the subject a dose of about 90 mg to about 110 mg of the compound of formula (I) once or twice daily. In some embodiments, the method comprises administering to the subject a dose of about 95 mg to about 105 mg of the compound of formula (I) once or twice daily. In some embodiments, the method comprises administering to the subject a dose of about 98 mg to about 102 mg of the compound of formula (I) once or twice daily.

In some embodiments, the method comprises administering to the subject a dose of about 90 mg, about 91 mg, about 92 mg, about 93 mg, about 94 mg, about 95 mg, about 96 mg, about 97 mg, about 98 mg, about 99 mg, about 100 mg, about 101 mg, about 102 mg, about 103 mg, about 104 mg, about 105 mg about 106 mg, about 107 mg, about 108 mg, about 109 mg or about 110 mg of the compound of formula (I) once or twice daily.

In some embodiments, the method comprises administering to the subject a dose of about 100 mg of the compound of formula (I) once or twice daily.

In some embodiments, the method comprises administering to the subject a dose of about 250 to about 350 mg of the compound of formula (I) once or twice daily. In some embodiments, the method comprises administering to the subject a dose of about 260 to about 340 mg of the compound of formula (I) once or twice daily. In some embodiments, the method comprises administering to the subject a dose of about 270 to about 330 mg of the compound of formula (I) once or twice daily. In some embodiments, the method comprises administering to the subject a dose of about 280 to about 320 mg of the compound of formula (I) once or twice daily. In some embodiments, the method comprises administering to the subject a dose of about 290 to about 310 mg of the compound of formula (I) once or twice daily. In some embodiments, the method comprises administering to the subject a dose of about 295 to about 305 mg of the compound of formula (I) once or twice daily.

In some embodiments, the method comprises administering to the subject a dose of about 290 mg, about 291 mg, about 292 mg, about 293 mg, about 294 mg, about 295 mg, about 296 mg, about 297 mg, about 298 mg, about 299 mg, about 300 mg, about 301 mg, about 302 mg, about 303 mg, about 304 mg, about 305 mg about 306 mg, about 307 mg, about 308 mg, about 309 mg or about 310 mg of the compound of formula (I) once or twice daily.

In some embodiments, the method comprises administering to the subject a dose of about 300 mg of the compound of formula (I) once or twice daily.

In some embodiments, the method comprises administering to the subject a dose of about 8 mg, about 9 mg, about 10 mg, about 11 mg, about 12 mg, about 12.5 mg, about 13 mg, about 14 mg, about 15 mg, about 16 mg, about 17 mg, about 45 mg, about 46 mg, about 47 mg, about 48 mg, about 49 mg, about 50 mg, about 51 mg, about 52 mg, about 53 mg, about 54 mg, about 55 mg, about 90 mg, about 91 mg, about 92 mg, about 93 mg, about 94 mg, about 95 mg, about 96 mg, about 97 mg, about 98 mg, about 99 mg, about 100 mg, about 101 mg, about 102 mg, about 103 mg, about 104 mg, about 105 mg about 106 mg, about 107 mg, about 108 mg, about 109 mg, about 110 mg, about 290 mg, about 291 mg, about 292 mg, about 293 mg, about 294 mg, about 295 mg, about 296 mg, about 297 mg, about 298 mg, about 299 mg, about 300 mg, about 301 mg, about 302 mg, about 303 mg, about 304 mg, about 305 mg about 306 mg, about 307 mg, about 308 mg, about 309 mg or about 310 mg of the compound of formula (I) once or twice daily.

In some embodiments, the method comprises administering to the subject a dose of about 10 mg, about 11 mg, about 12 mg, about 12.5 mg, about 13 mg, about 14 mg, about 15 mg, about 48 mg, about 49 mg, about 50 mg, about 51 mg, about 52 mg, about 53 mg, about 96 mg, about 97 mg, about 98 mg, about 99 mg, about 100 mg, about 101 mg, about 102 mg, about 103 mg, about 104 mg, about 295 mg, about 296 mg, about 297 mg, about 298 mg, about 299 mg, about 300 mg, about 301 mg, about 302 mg, about 303 mg, about 304 mg or about 305 mg of the compound of formula (I) once or twice daily.

In some embodiments, the method comprises administering to the subject a dose of about 12.5 mg, about 50 mg, about 100 mg or about 300 mg of the compound of formula (I) once or twice daily.

In some embodiments, the method comprises administering the dose of the compound of formula (I) once daily (e.g., every 24 hours).

In some embodiments, the method comprises administering to the subject a dose of about 8 mg to about 17 mg of the compound of formula (I) once daily (e.g., every 24 hours). In some embodiments, the method comprises administering to the subject a dose of about 10 mg to about mg of the compound of formula (I) once daily (e.g., every 24 hours). In some embodiments, the method comprises administering to the subject a dose of about 12 mg to about 13 mg of the compound of formula (I) once daily (e.g., every 24 hours).

In some embodiments, the method comprises administering to the subject a dose of about 8 mg, about 9 mg, about 10 mg, about 11 mg, about 12 mg, about 12.5 mg, about 13 mg, about 14 mg, about 15 mg, about 16 mg or about 17 mg of the compound of formula (I) once daily (e.g., every 24 hours).

In further embodiments, the method comprises administering to the subject a dose of about 12.5 mg of the compound of formula (I) once daily (e.g., every 24 hours).

In some embodiments, the method comprises administering to the subject a dose of about 40 mg to about 60 mg of the compound of formula (I) once daily (e.g., every 24 hours). In some embodiments, the method comprises administering to the subject a dose of about 45 mg to about 55 mg of the compound of formula (I) once daily (e.g., every 24 hours). In some embodiments, the method comprises administering to the subject a dose of about 47 mg to about 53 mg of the compound of formula (I) once daily (e.g., every 24 hours). In some embodiments, the method comprises administering to the subject a dose of about 49 mg to about 51 mg of the compound of formula (I) once daily (e.g., every 24 hours).

In some embodiments, the method comprises administering to the subject a dose of about 45 mg, about 46 mg, about 47 mg, about 48 mg, about 49 mg, about 50 mg, about 51 mg, about 52 mg, about 53 mg, about 54 mg or about 55 mg of the compound of formula (I) once daily (e.g., every 24 hours).

In some embodiments, the method comprises administering to the subject a dose of about 50 mg of the compound of formula (I) once daily (e.g., every 24 hours).

In some embodiments, the method comprises administering to the subject a dose of about 80 mg to about 120 mg of the compound of formula (I) once daily (e.g., every 24 hours). In some embodiments, the method comprises administering to the subject a dose of about 90 mg to about 110 mg of the compound of formula (I) once daily (e.g., every 24 hours). In some embodiments, the method comprises administering to the subject a dose of about 95 mg to about 105 mg of the compound of formula (I) once daily (e.g., every 24 hours). In some embodiments, the method comprises administering to the subject a dose of about 98 mg to about 102 mg of the compound of formula (I) once daily (e.g., every 24 hours).

In some embodiments, the method comprises administering to the subject a dose of about 90 mg, about 91 mg, about 92 mg, about 93 mg, about 94 mg, about 95 mg, about 96 mg, about 97 mg, about 98 mg, about 99 mg, about 100 mg, about 101 mg, about 102 mg, about 103 mg, about 104 mg, about 105 mg about 106 mg, about 107 mg, about 108 mg, about 109 mg or about 110 mg of the compound of formula (I) once daily (e.g., every 24 hours).

In some embodiments, the method comprises administering to the subject a dose of about 100 mg of the compound of formula (I) once daily (e.g., every 24 hours).

In some embodiments, the method comprises administering to the subject a dose of about 250 to about 350 mg of the compound of formula (I) once daily (e.g., every 24 hours). In some embodiments, the method comprises administering to the subject a dose of about 260 to about 340 mg of the compound of formula (I) once daily (e.g., every 24 hours). In some embodiments, the method comprises administering to the subject a dose of about 270 to about 330 mg of the compound of formula (I) once daily (e.g., every 24 hours). In some embodiments, the method comprises administering to the subject a dose of about 280 to about 320 mg of the compound of formula (I) once daily (e.g., every 24 hours). In some embodiments, the method comprises administering to the subject a dose of about 290 to about 310 mg of the compound of formula (I) once daily (e.g., every 24 hours). In some embodiments, the method comprises administering to the subject a dose of about 295 to about 305 mg of the compound of formula (I) once daily (e.g., every 24 hours).

In some embodiments, the method comprises administering to the subject a dose of about 290 mg, about 291 mg, about 292 mg, about 293 mg, about 294 mg, about 295 mg, about 296 mg, about 297 mg, about 298 mg, about 299 mg, about 300 mg, about 301 mg, about 302 mg, about 303 mg, about 304 mg, about 305 mg about 306 mg, about 307 mg, about 308 mg, about 309 mg or about 310 mg of the compound of formula (I) once daily (e.g., every 24 hours).

In some embodiments, the method comprises administering to the subject a dose of about 300 mg of the compound of formula (I) once daily (e.g., every 24 hours).

In some embodiments, the method comprises administering to the subject a dose of about 8 mg, about 9 mg, about 10 mg, about 11 mg, about 12 mg, about 12.5 mg, about 13 mg, about 14 mg, about 15 mg, about 16 mg, about 17 mg, about 45 mg, about 46 mg, about 47 mg, about 48 mg, about 49 mg, about 50 mg, about 51 mg, about 52 mg, about 53 mg, about 54 mg, about 55 mg, about 90 mg, about 91 mg, about 92 mg, about 93 mg, about 94 mg, about 95 mg, about 96 mg, about 97 mg, about 98 mg, about 99 mg, about 100 mg, about 101 mg, about 102 mg, about 103 mg, about 104 mg, about 105 mg about 106 mg, about 107 mg, about 108 mg, about 109 mg, about 110 mg, about 290 mg, about 291 mg, about 292 mg, about 293 mg, about 294 mg, about 295 mg, about 296 mg, about 297 mg, about 298 mg, about 299 mg, about 300 mg, about 301 mg, about 302 mg, about 303 mg, about 304 mg, about 305 mg about 306 mg, about 307 mg, about 308 mg, about 309 mg or about 310 mg of the compound of formula (I) once daily (e.g., every 24 hours).

In some embodiments, the method comprises administering to the subject a dose of about 10 mg, about 11 mg, about 12 mg, about 12.5 mg, about 13 mg, about 14 mg, about 15 mg, about 48 mg, about 49 mg, about 50 mg, about 51 mg, about 52 mg, about 53 mg, about 96 mg, about 97 mg, about 98 mg, about 99 mg, about 100 mg, about 101 mg, about 102 mg, about 103 mg, about 104 mg, about 295 mg, about 296 mg, about 297 mg, about 298 mg, about 299 mg, about 300 mg, about 301 mg, about 302 mg, about 303 mg, about 304 mg or about 305 mg of the compound of formula (I) once daily (e.g., every 24 hours).

In some embodiments, the method comprises administering the dose of the compound of formula (I) twice daily.

In some embodiments, the method comprises administering to the subject a dose of about 8 mg to about 17 mg of the compound of formula (I) twice daily (e.g., every 12 hours). In some embodiments, the method comprises administering to the subject a dose of about 10 mg to about 15 mg of the compound of formula (I) twice daily (e.g., every 12 hours). In some embodiments, the method comprises administering to the subject a dose of about 12 mg to about 13 mg of the compound of formula (I) twice daily (e.g., every 12 hours).

In some embodiments, the method comprises administering to the subject a dose of about 8 mg, about 9 mg, about 10 mg, about 11 mg, about 12 mg, about 12.5 mg, about 13 mg, about 14 mg, about 15 mg, about 16 mg or about 17 mg of the compound of formula (I) twice daily (e.g., every 12 hours).

In further embodiments, the method comprises administering to the subject a dose of about 12.5 mg of the compound of formula (I) twice daily (e.g., every 12 hours).

In some embodiments, the method comprises administering to the subject a dose of about 40 mg to about 60 mg of the compound of formula (I) twice daily (e.g., every 12 hours). In some embodiments, the method comprises administering to the subject a dose of about 45 mg to about 55 mg of the compound of formula (I) twice daily (e.g., every 12 hours). In some embodiments, the method comprises administering to the subject a dose of about 47 mg to about 53 mg of the compound of formula (I) twice daily (e.g., every 12 hours). In some embodiments, the method comprises administering to the subject a dose of about 49 mg to about 51 mg of the compound of formula (I) twice daily (e.g., every 12 hours).

In some embodiments, the method comprises administering to the subject a dose of about 45 mg, about 46 mg, about 47 mg, about 48 mg, about 49 mg, about 50 mg, about 51 mg, about 52 mg, about 53 mg, about 54 mg or about 55 mg of the compound of formula (I) twice daily (e.g., every 12 hours).

In some embodiments, the method comprises administering to the subject a dose of about 50 mg of the compound of formula (I) twice daily (e.g., every 12 hours).

In some embodiments, the method comprises administering to the subject a dose of about 80 mg to about 120 mg of the compound of formula (I) twice daily (e.g., every 12 hours). In some embodiments, the method comprises administering to the subject a dose of about 90 mg to about 110 mg of the compound of formula (I) twice daily (e.g., every 12 hours). In some embodiments, the method comprises administering to the subject a dose of about 95 mg to about 105 mg of the compound of formula (I) twice daily (e.g., every 12 hours). In some embodiments, the method comprises administering to the subject a dose of about 98 mg to about 102 mg of the compound of formula (I) twice daily (e.g., every 12 hours).

In some embodiments, the method comprises administering to the subject a dose of about 90 mg, about 91 mg, about 92 mg, about 93 mg, about 94 mg, about 95 mg, about 96 mg, about 97 mg, about 98 mg, about 99 mg, about 100 mg, about 101 mg, about 102 mg, about 103 mg, about 104 mg, about 105 mg about 106 mg, about 107 mg, about 108 mg, about 109 mg or about 110 mg of the compound of formula (I) twice daily (e.g., every 12 hours).

In some embodiments, the method comprises administering to the subject a dose of about 100 mg of the compound of formula (I) twice daily (e.g., every 12 hours).

In some embodiments, the method comprises administering to the subject a dose of about 250 to about 350 mg of the compound of formula (I) twice daily (e.g., every 12 hours). In some embodiments, the method comprises administering to the subject a dose of about 260 to about 340 mg of the compound of formula (I) twice daily (e.g., every 12 hours). In some embodiments, the method comprises administering to the subject a dose of about 270 to about 330 mg of the compound of formula (I) twice daily (e.g., every 12 hours). In some embodiments, the method comprises administering to the subject a dose of about 280 to about 320 mg of the compound of formula (I) twice daily (e.g., every 12 hours). In some embodiments, the method comprises administering to the subject a dose of about 290 to about 310 mg of the compound of formula (I) twice daily (e.g., every 12 hours). In some embodiments, the method comprises administering to the subject a dose of about 295 to about 305 mg of the compound of formula (I) twice daily (e.g., every 12 hours).

In some embodiments, the method comprises administering to the subject a dose of about 290 mg, about 291 mg, about 292 mg, about 293 mg, about 294 mg, about 295 mg, about 296 mg, about 297 mg, about 298 mg, about 299 mg, about 300 mg, about 301 mg, about 302 mg, about 303 mg, about 304 mg, about 305 mg about 306 mg, about 307 mg, about 308 mg, about 309 mg or about 310 mg of the compound of formula (I) twice daily (e.g., every 12 hours).

In some embodiments, the method comprises administering to the subject a dose of about 300 mg of the compound of formula (I) twice daily (e.g., every 12 hours).

In some embodiments, the method comprises administering to the subject a dose of about 8 mg, about 9 mg, about 10 mg, about 11 mg, about 12 mg, about 12.5 mg, about 13 mg, about 14 mg, about 15 mg, about 16 mg, about 17 mg, about 45 mg, about 46 mg, about 47 mg, about 48 mg, about 49 mg, about 50 mg, about 51 mg, about 52 mg, about 53 mg, about 54 mg, about 55 mg, about 90 mg, about 91 mg, about 92 mg, about 93 mg, about 94 mg, about 95 mg, about 96 mg, about 97 mg, about 98 mg, about 99 mg, about 100 mg, about 101 mg, about 102 mg, about 103 mg, about 104 mg, about 105 mg about 106 mg, about 107 mg, about 108 mg, about 109 mg, about 110 mg, about 290 mg, about 291 mg, about 292 mg, about 293 mg, about 294 mg, about 295 mg, about 296 mg, about 297 mg, about 298 mg, about 299 mg, about 300 mg, about 301 mg, about 302 mg, about 303 mg, about 304 mg, about 305 mg about 306 mg, about 307 mg, about 308 mg, about 309 mg or about 310 mg of the compound of formula (I) twice daily (e.g., every 12 hours).

In some embodiments, the method comprises administering to the subject a dose of about 10 mg, about 11 mg, about 12 mg, about 12.5 mg, about 13 mg, about 14 mg, about 15 mg, about 48 mg, about 49 mg, about 50 mg, about 51 mg, about 52 mg, about 53 mg, about 96 mg, about 97 mg, about 98 mg, about 99 mg, about 100 mg, about 101 mg, about 102 mg, about 103 mg, about 104 mg, about 295 mg, about 296 mg, about 297 mg, about 298 mg, about 299 mg, about 300 mg, about 301 mg, about 302 mg, about 303 mg, about 304 mg or about 305 mg of the compound of formula (I) twice daily (e.g., every 12 hours).

### Kits

In some embodiments provided are kits related to methods of use described herein.

In one embodiment, provided is a kit for predicting the sensitivity of a subject having or having been diagnosed with an MTAP-deficiency-related cancer for treatment with a PRMT5 inhibitor is provided. The kit comprises: i) reagents capable of detecting human MTAP-deficient and/or MTA-accumulating cancer cells; and ii) instructions for how to use said kit.

### Selected Embodiments

Embodiment 1. A crystalline form of N-(6-amino-5-methylpyridin-3-yl)-2-((2R,5S)-2-(benzo[d]thiazol-5-yl)-5-methylpiperidin-1-yl)-2-oxoacetamide (a compound of formula (I))

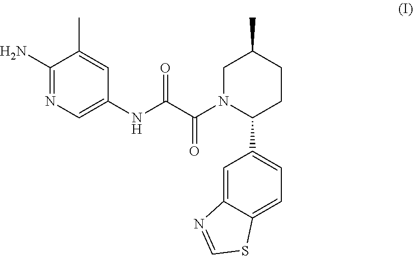 [SMILES: Cc1cc(NC(=O)C(=O)N2C[C@@H](C)CC[C@@H]2c2ccc3scnc3c2)cnc1N]

wherein the X-ray powder diffraction (XRPD) pattern of the crystalline form comprises one or more peaks at 2θ angles selected from 6.4±0.2, 8.9±0.2, 12.7±0.2, 14.0±0.2, 19.1±0.2, 19.9±0.2, 22.6±0.2 degrees.
Embodiment 2. The crystalline form of embodiment 1, wherein the XRPD pattern of the crystalline form comprises one or more peaks at 2θ angles selected from 6.4±0.2, 8.9±0.2, 12.7±0.2, 13.8±0.2, 14.0±0.2, 18.3±0.2, 19.1±0.2, 19.9±0.2, 22.6±0.2, 24.3±0.2, 26.2±0.2, 26.7±0.2 and 28.2±0.2 degrees.
Embodiment 3. The crystalline form of embodiment 1 wherein the crystalline form has an XRPD diffraction pattern substantially corresponding to the XRPD diffraction pattern shown in FIG. 1A.
Embodiment 4. The crystalline form of any one of embodiments 1 to 3 wherein crystalline form is substantially pure.
Embodiment 5. A pharmaceutical composition comprising a compound of formula (I) and at least one pharmaceutically acceptable carrier, diluent, excipient or adjuvant.
Embodiment 6. The pharmaceutical composition of embodiment 5 wherein the compound of formula (I) is a crystalline form of any one of embodiments 1 to 4.
Embodiment 7. The pharmaceutical composition of embodiment 5 or 6, wherein the composition comprises about 5% (w/w) to about 50% (w/w) of the compound of formula (I). Embodiment 8. The pharmaceutical composition of embodiment 5 or 6, wherein the composition comprises about 10% (w/w) to about 45% (w/w) of a compound of formula (I).
Embodiment 9. The pharmaceutical composition of embodiment 5 or 6, wherein the composition comprises about 8% (w/w) to about 17% (w/w), about 25% (w/w) to about 35% (w/w) or about 35% (w/w) to about 45% (w/w) of a compound of formula (I).
Embodiment 10. The pharmaceutical composition of embodiment 5 or 6, wherein the composition comprises about 8% (w/w) to about 17% (w/w) of a compound of formula (I).
Embodiment 11. The pharmaceutical composition of embodiment 5 or 6, wherein the composition comprises about 10% (w/w) to about 15% (w/w) of a compound of formula (I).
Embodiment 12. The pharmaceutical composition of embodiment 5 or 6, wherein the composition comprises about 12% (w/w) to about 13% (w/w) of a compound of formula (I).
Embodiment 13. The pharmaceutical composition of embodiment 5 or 6, wherein the composition comprises about 12.5% (w/w) of a compound of formula (I).
Embodiment 14. The pharmaceutical composition of embodiment 5 or 6, wherein the composition comprises about 25% (w/w) to about 35% (w/w) of a compound of formula (I).
Embodiment 15. The pharmaceutical composition of embodiment 5 or 6, wherein the composition comprises about 27% (w/w) to about 33% (w/w) of a compound of formula (I).
Embodiment 16. The pharmaceutical composition of embodiment 5 or 6, wherein the composition comprises about 29% (w/w) to about 31% (w/w) of a compound of formula (I).
Embodiment 17. The pharmaceutical composition of embodiment 5 or 6, wherein the composition comprises about 29.2% (w/w) to about 29.7% (w/w) of a compound of formula (I).
Embodiment 18. The pharmaceutical composition of embodiment 5 or 6, wherein the composition comprises about 29.4% (w/w) of a compound of formula (I).
Embodiment 19. The pharmaceutical composition of embodiment 5 or 6, wherein the composition comprises about 35% (w/w) to about 45% (w/w) of a compound of formula (I).
Embodiment 20. The pharmaceutical composition of embodiment 5 or 6, wherein the composition comprises about 37% (w/w) to about 43% (w/w) of a compound of formula (I).
Embodiment 21. The pharmaceutical composition of embodiment 5 or 6, wherein the composition comprises about 39% (w/w) to about 31% (w/w) of a compound of formula (I).
Embodiment 22. The pharmaceutical composition of embodiment 5 or 6, wherein the composition comprises about 40% (w/w) of a compound of formula (I).
Embodiment 23. The pharmaceutical composition of embodiment 5 or 6, wherein the composition comprises about 12.5% (w/w), about 29.4% (w/w) or about 40% (w/w) of a compound of formula (I).
Embodiment 24. The pharmaceutical composition of any one of embodiments 5 to 23, wherein the pharmaceutical composition comprises a filler (e.g., microcrystalline cellulose).
Embodiment 25. The pharmaceutical composition of any one of embodiments 5 to 24, wherein the pharmaceutical composition comprises a glidant (e.g., colloidal silicon dioxide).
Embodiment 26. The pharmaceutical composition of any one of embodiments 5 to 25, wherein the pharmaceutical composition comprises a disintegrant (e.g., croscarmellose sodium).
Embodiment 27. The pharmaceutical composition of any one of embodiments 5 to 26, wherein the pharmaceutical composition comprises a lubricant (e.g., magnesium stearate).
Embodiment 28. A pharmaceutical composition comprising:
(a) a compound of formula (I)

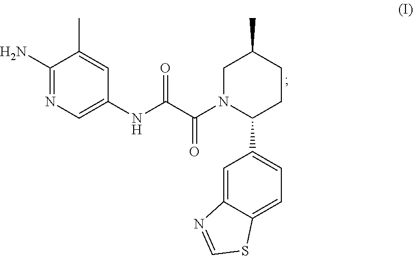 [SMILES: Cc1cc(NC(=O)C(=O)N2C[C@@H](C)CC[C@@H]2c2ccc3scnc3c2)cnc1N]

(b) a filler (e.g., microcrystalline cellulose);
(c) a glidant (e.g., colloidal silicon dioxide);
(d) a disintegrant (e.g., croscarmellose sodium); and
(e) a lubricant (e.g., magnesium stearate).
Embodiment 29. The pharmaceutical composition of embodiment 29, wherein the composition comprises a crystalline form of the compound of formula (I) of any one of embodiments 1 to 4.
Embodiment 30. The pharmaceutical composition of any one of embodiments 5 to 29, wherein the pharmaceutical composition comprises about 50% (w/w) to about 90% (w/w) filler.
Embodiment 31. The pharmaceutical composition of any one of embodiments 5 to 29, wherein the pharmaceutical composition comprises about 50% (w/w) to about 60% (w/w) filler.
Embodiment 32. The pharmaceutical composition of any one of embodiments 5 to 29, wherein the pharmaceutical composition comprises about 52% (w/w) to about 58% (w/w) filler.
Embodiment 33. The pharmaceutical composition of any one of embodiments 5 to 29, wherein the pharmaceutical composition comprises about 52% (w/w) to about 56% (w/w) filler.
Embodiment 34. The pharmaceutical composition of any one of embodiments 5 to 29, wherein the pharmaceutical composition comprises about 50% (w/w), about 51% (w/w), about 52% (w/w), about 53% (w/w), about 54% (w/w), about 55% (w/w), about 56% (w/w), about 57% (w/w) or about 58% (w/w) filler.
Embodiment 35. The pharmaceutical composition of any one of embodiments 5 to 29, wherein the pharmaceutical composition comprises about 54% (w/w) filler.
Embodiment 36. The pharmaceutical composition of any one of embodiments 5 to 29, wherein the pharmaceutical composition comprises about 61% (w/w) to about 70% (w/w) filler.
Embodiment 37. The pharmaceutical composition of any one of embodiments 5 to 29, wherein the pharmaceutical composition comprises about 63% (w/w) to about 68% (w/w) filler.
Embodiment 38. The pharmaceutical composition of any one of embodiments 5 to 29, wherein the pharmaceutical composition comprises about 65% (w/w) to about 66% (w/w) filler.
Embodiment 39. The pharmaceutical composition of any one of embodiments 5 to 29, wherein the pharmaceutical composition comprises about 61% (w/w), about 62% (w/w), about 63% (w/w), about 64% (w/w), about 65% (w/w), about 65.4% (w/w), about 66% (w/w), about 67% (w/w), about 68% (w/w), about 69% (w/w) or about 70% (w/w) filler.
Embodiment 40. The pharmaceutical composition of any one of embodiments 5 to 29, wherein the pharmaceutical composition comprises about 64% (w/w), about 65% (w/w), about 65.4% (w/w), about 66% (w/w) or about 67% (w/w) filler.
Embodiment 41. The pharmaceutical composition of any one of embodiments 5 to 29, wherein the pharmaceutical composition comprises about 65.4% (w/w) filler.
Embodiment 42. The pharmaceutical composition of any one of embodiments 5 to 29, wherein the pharmaceutical composition comprises about 75% (w/w) to about 85% (w/w) filler.
Embodiment 43. The pharmaceutical composition of any one of embodiments 5 to 29, wherein the pharmaceutical composition comprises about 77% (w/w) to about 83% (w/w) filler.
Embodiment 44. The pharmaceutical composition of any one of embodiments 5 to 29, wherein the pharmaceutical composition comprises about 79% (w/w) to about 82% (w/w) filler.
Embodiment 45. The pharmaceutical composition of any one of embodiments 5 to 29, wherein the pharmaceutical composition comprises about 80% (w/w) to about 82% (w/w) filler.
Embodiment 46. The pharmaceutical composition of any one of embodiments 5 to 29, wherein the pharmaceutical composition comprises about 75% (w/w), about 76% (w/w), about 77% (w/w), about 78% (w/w), about 79% (w/w), about 80% (w/w), about 81% (w/w), about 81.5% (w/w), about 82% (w/w), about 83% (w/w), about 84% (w/w) or about 85% (w/w) filler.
Embodiment 47. The pharmaceutical composition of any one of embodiments 5 to 29, wherein the pharmaceutical composition comprises about 80% (w/w), about 81% (w/w), about 81.5% (w/w), about 82% (w/w) or about 83% (w/w) filler.
Embodiment 48. The pharmaceutical composition of any one of embodiments 5 to 29, wherein the pharmaceutical composition comprises about 81.5% (w/w) filler.
Embodiment 49. The pharmaceutical composition of any one of embodiments 5 to 48, wherein the pharmaceutical composition comprises about 0.5% (w/w) to about 1.5% (w/w) glidant.
Embodiment 50. The pharmaceutical composition of any one of embodiments 5 to 48, wherein the pharmaceutical composition comprises about 0.75% (w/w) to about 1.25% (w/w) glidant.
Embodiment 51. The pharmaceutical composition of any one of embodiments 5 to 48, wherein the pharmaceutical composition comprises about 0.8% (w/w) to about 1% (w/w) glidant.
Embodiment 52. The pharmaceutical composition of any one of embodiments 5 to 48, wherein the pharmaceutical composition comprises about 0.75% (w/w) to about 0.95% (w/w) glidant.
Embodiment 53. The pharmaceutical composition of any one of embodiments 5 to 48, wherein the pharmaceutical composition comprises about 0.85% (w/w) to about 0.9% (w/w) glidant.
Embodiment 54. The pharmaceutical composition of any one of embodiments 5 to 48, wherein the pharmaceutical composition comprises about 0.9% (w/w) to about 1.1% (w/w) glidant.
Embodiment 55. The pharmaceutical composition of any one of embodiments 5 to 48, wherein the pharmaceutical composition comprises about 0.95% (w/w) to about 1.05% (w/w) glidant.
Embodiment 56. The pharmaceutical composition of any one of embodiments 5 to 48, wherein the pharmaceutical composition comprises about 0.8% (w/w), about 0.82% (w/w), about 0.84% (w/w), about 0.86% (w/w), about 0.87% (w/w), about 0.88% (w/w), about 0.9% (w/w), about 0.92% (w/w), about 0.94% (w/w), about 0.96% (w/w), about 0.98% (w/w), about 1.0% (w/w), about 1.02% (w/w), about 1.04% (w/w), about 1.06% (w/w), about 1.08% (w/w) or about 1.1% (w/w) glidant.
Embodiment 57. The pharmaceutical composition of any one of embodiments 5 to 48, wherein the pharmaceutical composition comprises about 0.86% (w/w), about 0.87% (w/w), about 0.88% (w/w), about 0.98% (w/w), about 1.00% (w/w) or about 1.02% (w/w) glidant.
Embodiment 58. The pharmaceutical composition of any one of embodiments 5 to 48, wherein the pharmaceutical composition comprises about 0.87% (w/w) glidant.
Embodiment 59. The pharmaceutical composition of any one of embodiments 5 to 48, wherein the pharmaceutical composition comprises about 1% (w/w) glidant.
Embodiment 60. The pharmaceutical composition of any one of embodiments 5 to 59, wherein the pharmaceutical composition comprises about 2% (w/w) to about 6% (w/w) disintegrant.
Embodiment 61. The pharmaceutical composition of any one of embodiments 5 to 59, wherein the pharmaceutical composition comprises about 3% (w/w) to about 5% (w/w) disintegrant.
Embodiment 62. The pharmaceutical composition of any one of embodiments 5 to 59, wherein the pharmaceutical composition comprises about 3.2% (w/w) to about 4% (w/w) disintegrant.
Embodiment 63. The pharmaceutical composition of any one of embodiments 5 to 59, wherein the pharmaceutical composition comprises about 3% (w/w) to about 3.8% (w/w) disintegrant.
Embodiment 64. The pharmaceutical composition of any one of embodiments 5 to 59, wherein the pharmaceutical composition comprises about 3.4% (w/w) to about 3.6% (w/w) disintegrant.
Embodiment 65. The pharmaceutical composition of any one of embodiments 5 to 59, wherein the pharmaceutical composition comprises about 3.6% (w/w) to about 4.4% (w/w) disintegrant.
Embodiment 66. The pharmaceutical composition of any one of embodiments 5 to 59, wherein the pharmaceutical composition comprises about 3.8% (w/w) to about 4.2% (w/w) disintegrant.
Embodiment 67. The pharmaceutical composition of any one of embodiments 5 to 59, wherein the pharmaceutical composition comprises about 3.2% (w/w), about 3.28% (w/w), about 3.36% (w/w), about 3.44% (w/w), about 3.47% (w/w), about 3.52% (w/w), about 3.6% (w/w), about 3.68% (w/w), about 3.76% (w/w), about 3.84% (w/w), about 3.92% (w/w), about 4% (w/w), about 4.08% (w/w), about 4.16% (w/w), about 4.24% (w/w), about 4.32% (w/w) or about 4.4% (w/w) disintegrant.
Embodiment 68. The pharmaceutical composition of any one of embodiments 5 to 59, wherein the pharmaceutical composition comprises about 3.44% (w/w), about 3.47% (w/w), about 3.52% (w/w), about 3.92% (w/w), about 4.00% (w/w) or about 4.08% (w/w) disintegrant.
Embodiment 69. The pharmaceutical composition of any one of embodiments 5 to 59, wherein the pharmaceutical composition comprises about 3.47% (w/w) disintegrant.
Embodiment 70. The pharmaceutical composition of any one of embodiments 5 to 59, wherein the pharmaceutical composition comprises about 4% (w/w) disintegrant.
Embodiment 71. The pharmaceutical composition of any one of embodiments 5 to 70, wherein the pharmaceutical composition comprises about 0.5% (w/w) to about 1.5% (w/w) lubricant.
Embodiment 72. The pharmaceutical composition of any one of embodiments 5 to 70, wherein the pharmaceutical composition comprises about 0.75% (w/w) to about 1.25% (w/w) lubricant.
Embodiment 73. The pharmaceutical composition of any one of embodiments 5 to 70, wherein the pharmaceutical composition comprises about 0.8% (w/w) to about 1% (w/w) lubricant.
Embodiment 74. The pharmaceutical composition of any one of embodiments 5 to 70, wherein the pharmaceutical composition comprises about 0.75% (w/w) to about 0.95% (w/w) lubricant.
Embodiment 75. The pharmaceutical composition of any one of embodiments 5 to 70, wherein the pharmaceutical composition comprises about 0.85% (w/w) to about 0.9% (w/w) lubricant.
Embodiment 76. The pharmaceutical composition of any one of embodiments 5 to 70, wherein the pharmaceutical composition comprises about 0.9% (w/w) to about 1.1% (w/w) lubricant.
Embodiment 77. The pharmaceutical composition of any one of embodiments 5 to 70, wherein the pharmaceutical composition comprises about 0.95% (w/w) to about 1.05% (w/w) lubricant.
Embodiment 78. The pharmaceutical composition of any one of embodiments 5 to 70, wherein the pharmaceutical composition comprises about 0.8% (w/w), about 0.82% (w/w), about 0.84% (w/w), about 0.86% (w/w), about 0.87% (w/w), about 0.88% (w/w), about 0.9% (w/w), about 0.92% (w/w), about 0.94% (w/w), about 0.96% (w/w), about 0.98% (w/w), about 1.0% (w/w), about 1.02% (w/w), about 1.04% (w/w), about 1.06% (w/w), about 1.08% (w/w) or about 1.1% (w/w) lubricant.
Embodiment 79. The pharmaceutical composition of any one of embodiments 5 to 70, wherein the pharmaceutical composition comprises about 0.86% (w/w), about 0.87% (w/w), about 0.88% (w/w), about 0.98% (w/w), about 1.00% (w/w) or about 1.02% (w/w) lubricant.
Embodiment 80. The pharmaceutical composition of any one of embodiments 5 to 70, wherein the pharmaceutical composition comprises about 0.87% (w/w) lubricant.
Embodiment 81. The pharmaceutical composition of any one of embodiments 5 to 70, wherein the pharmaceutical composition comprises about 1% (w/w) lubricant.
Embodiment 82. A pharmaceutical composition of embodiment 28 or 29, wherein the composition comprises:
(a) about 5% (w/w) to about 50% (w/w) of the compound of formula (I);
(b) about 50% (w/w) to about 90% (w/w) of a filler (e.g., microcrystalline cellulose);
(c) about 0.5% (w/w) to about 1.5% (w/w) of a glidant (e.g., colloidal silicon dioxide);
(d) about 2% (w/w) to about 6% (w/w) of a disintegrant (e.g., croscarmellose sodium); and
(e) about 0.5% (w/w) to about 1.5% (w/w) of a lubricant (e.g., magnesium stearate);
thereby totaling 100% (w/w) of the composition.
Embodiment 83. A pharmaceutical composition of embodiment 28 or 29, wherein the composition comprises:
(a) about 10% (w/w) to about 45% (w/w) of the compound of formula (I);
(b) about 50% (w/w) to about 90% (w/w) of a filler (e.g., microcrystalline cellulose);
(c) about 0.8% (w/w) to about 1% (w/w) of a glidant (e.g., colloidal silicon dioxide);
(d) about 3.2% (w/w) to about 4% (w/w) of a disintegrant (e.g., croscarmellose sodium); and
(e) about 0.8% (w/w) to about 1% (w/w) of a lubricant (e.g., magnesium stearate);
thereby totaling 100% (w/w) of the composition.
Embodiment 84. A pharmaceutical composition of embodiment 28 or 29, wherein the composition comprises:
(a) about 10% (w/w) to about 15% (w/w) of the compound of formula (I);
(b) about 77% (w/w) to about 83% (w/w) of a filler (e.g., microcrystalline cellulose);
(c) about 0.95% (w/w) to about 1.05% (w/w) of a glidant (e.g., colloidal silicon dioxide);
(d) about 3.8% (w/w) to about 4.2% (w/w) of a disintegrant (e.g., croscarmellose sodium); and
(e) about 0.95% (w/w) to about 1.05% (w/w) of a lubricant (e.g., magnesium stearate);
thereby totaling 100% (w/w) of the composition.
Embodiment 85. A pharmaceutical composition of embodiment 28 or 29, wherein the composition comprises:
(a) about 12.5% (w/w) of the compound of formula (I);
(b) about 81.5% (w/w) of a filler (e.g., microcrystalline cellulose);
(c) about 1% (w/w) of a glidant (e.g., colloidal silicon dioxide);
(d) about 4% (w/w) of a disintegrant (e.g., croscarmellose sodium); and
(e) about 1% (w/w) of a lubricant (e.g., magnesium stearate);
thereby totaling 100% (w/w) of the composition.
Embodiment 86. A pharmaceutical composition of embodiment 28 or 29, wherein the composition comprises:
(a) about 27% (w/w) to about 33% (w/w) of the compound of formula (I);
(b) about 63% (w/w) to about 68% (w/w) of a filler (e.g., microcrystalline cellulose);
(c) about 0.85% (w/w) to about 0.9% (w/w) of a glidant (e.g., colloidal silicon dioxide);
(d) about 3.4% (w/w) to about 3.6% (w/w) of a disintegrant (e.g., croscarmellose sodium); and
(e) about 0.85% (w/w) to about 0.9% (w/w) of a lubricant (e.g., magnesium stearate);
thereby totaling 100% (w/w) of the composition.
Embodiment 87. A pharmaceutical composition of embodiment 28 or 29, wherein the composition comprises:
(a) about 29.4% (w/w) of the compound of formula (I);
(b) about 65.4% (w/w) of a filler (e.g., microcrystalline cellulose);
(c) about 0.87% (w/w) of a glidant (e.g., colloidal silicon dioxide);
(d) about 3.47% (w/w) of a disintegrant (e.g., croscarmellose sodium); and
(e) about 0.87% (w/w) of a lubricant (e.g., magnesium stearate);
thereby totaling 100% (w/w) of the composition.
Embodiment 88. A pharmaceutical composition of embodiment 28 or 29, wherein the composition comprises:
(a) about 37% (w/w) to about 43% (w/w) of the compound of formula (I);
(b) about 52% (w/w) to about 58% (w/w) of a filler (e.g., microcrystalline cellulose);
(c) about 0.95% (w/w) to about 1.05% (w/w) of a glidant (e.g., colloidal silicon dioxide);
(d) about 3.8% (w/w) to about 4.2% (w/w) of a disintegrant (e.g., croscarmellose sodium); and
(e) about 0.95% (w/w) to about 1.05% (w/w) of a lubricant (e.g., magnesium stearate);
thereby totaling 100% (w/w) of the composition.
Embodiment 89. A pharmaceutical composition of embodiment 28 or 29, wherein the composition comprises:
(a) about 40% (w/w) of the compound of formula (I);
(b) about 54% (w/w) of a filler (e.g., microcrystalline cellulose);
(c) about 1% (w/w) of a glidant (e.g., colloidal silicon dioxide);
(d) about 4% (w/w) of a disintegrant (e.g., croscarmellose sodium); and
(e) about 1% (w/w) of a lubricant (e.g., magnesium stearate);
thereby totaling 100% (w/w) of the composition.
Embodiment 90. The pharmaceutical composition of any one of embodiments 5 to 89 wherein the filler is microcrystalline cellulose.
Embodiment 91. The pharmaceutical composition of any one of embodiments 5 to 89 wherein the filler is microcrystalline cellulose PH 102, PH 200 or a mixture thereof.
Embodiment 92. The pharmaceutical composition of any one of embodiments 5 to 91 wherein the glidant is colloidal silicon dioxide.
Embodiment 93. The pharmaceutical composition of any one of embodiments 5 to 92 wherein the disintegrant is croscarmellose sodium.
Embodiment 94. The pharmaceutical composition of any one of embodiments 5 to 93 wherein the lubricant is magnesium stearate.
Embodiment 95. The pharmaceutical composition of any one of embodiments 5 to 94, wherein the pharmaceutical composition comprises about 30% (w/w) to about 70% (w/w) intragranular filler.
Embodiment 96. The pharmaceutical composition of any one of embodiments 5 to 94, wherein the pharmaceutical composition comprises about 35% (w/w) to about 60% (w/w) intragranular filler.
Embodiment 97. The pharmaceutical composition of any one of embodiments 5 to 94, wherein the pharmaceutical composition comprises about 50% (w/w) to about 60% (w/w) intragranular filler.
Embodiment 98. The pharmaceutical composition of any one of embodiments 5 to 94, wherein the pharmaceutical composition comprises about 52% (w/w) to about 58% (w/w) intragranular filler.
Embodiment 99. The pharmaceutical composition of any one of embodiments 5 to 94, wherein the pharmaceutical composition comprises about 54% (w/w) to about 57% (w/w) intragranular filler.
Embodiment 100. The pharmaceutical composition of any one of embodiments 5 to 94, wherein the pharmaceutical composition comprises about 50% (w/w), about 51% (w/w), about 52% (w/w), about 53% (w/w), about 54% (w/w), about 55% (w/w), about 56% (w/w), about 56.5% (w/w), about 57% (w/w), about 58% (w/w), about 59% (w/w), or about 60% (w/w) intragranular filler.
Embodiment 101. The pharmaceutical composition of any one of embodiments 5 to 94, wherein the pharmaceutical composition comprises about 53% (w/w), about 54% (w/w), about 55% (w/w), about 56% (w/w), about 56.5% (w/w), about 57% (w/w) or about 58% (w/w) intragranular filler.
Embodiment 102. The pharmaceutical composition of any one of embodiments 5 to 94, wherein the pharmaceutical composition comprises about 54% (w/w) intragranular filler.
Embodiment 103. The pharmaceutical composition of any one of embodiments 5 to 94, wherein the pharmaceutical composition comprises about 56.5% (w/w) intragranular filler.
Embodiment 104. The pharmaceutical composition of any one of embodiments 5 to 94, wherein the pharmaceutical composition comprises about 35% (w/w) to about 45% (w/w) intragranular filler.
Embodiment 105. The pharmaceutical composition of any one of embodiments 5 to 94, wherein the pharmaceutical composition comprises about 37% (w/w) to about 43% (w/w) intragranular filler.
Embodiment 106. The pharmaceutical composition of any one of embodiments 5 to 94, wherein the pharmaceutical composition comprises about 37% (w/w) to about 41% (w/w) intragranular filler.
Embodiment 107. The pharmaceutical composition of any one of embodiments 5 to 94, wherein the pharmaceutical composition comprises about 35% (w/w), about 36% (w/w), about 37% (w/w), about 38% (w/w), about 39% (w/w), about 39.7% (w/w), about 40% (w/w), about 41% (w/w), about 42% (w/w), or about 43% (w/w) intragranular filler.
Embodiment 108. The pharmaceutical composition of any one of embodiments 5 to 94, wherein the pharmaceutical composition comprises about 38% (w/w), about 39% (w/w), about 39.7% (w/w), about 40% (w/w) or about 41% (w/w) intragranular filler.
Embodiment 109. The pharmaceutical composition of any one of embodiments 5 to 94, wherein the pharmaceutical composition comprises about 39.7% (w/w) intragranular filler.
Embodiment 110. The pharmaceutical composition of any one of embodiments 5 to 109, wherein the pharmaceutical composition comprises about 0% (w/w) to about 40% (w/w) extragranular filler.
Embodiment 111. The pharmaceutical composition of any one of embodiments 5 to 109, wherein the pharmaceutical composition comprises about 0% (w/w) to about 30% (w/w) extragranular filler.
Embodiment 112. The pharmaceutical composition of any one of embodiments 5 to 109, wherein the pharmaceutical composition comprises about 0% (w/w) to about 25% (w/w) extragranular filler.
Embodiment 113. The pharmaceutical composition of any one of embodiments 5 to 109, wherein the pharmaceutical composition comprises 0% (w/w) extragranular filler.
Embodiment 114. The pharmaceutical composition of any one of embodiments 5 to 109, wherein the pharmaceutical composition comprises about 15% (w/w) to about 35% (w/w) extragranular filler.
Embodiment 115. The pharmaceutical composition of any one of embodiments 5 to 109, wherein the pharmaceutical composition comprises about 20% (w/w) to about 30% (w/w) extragranular filler.
Embodiment 116. The pharmaceutical composition of any one of embodiments 5 to 109, wherein the pharmaceutical composition comprises about 22% (w/w) to about 27% (w/w) extragranular filler.
Embodiment 117. The pharmaceutical composition of any one of embodiments 5 to 109, wherein the pharmaceutical composition comprises about 20% (w/w), about 21% (w/w), about 22% (w/w), about 23% (w/w), about 24% (w/w), about 25% (w/w), about 25.7% (w/w), about 26% (w/w), about 27% (w/w), about 28% (w/w), about 29% (w/w) or about 30% (w/w) extragranular filler.
Embodiment 118. The pharmaceutical composition of any one of embodiments 5 to 109, wherein the pharmaceutical composition comprises about 25% (w/w) extragranular filler.
Embodiment 119. The pharmaceutical composition of any one of embodiments 5 to 109, wherein the pharmaceutical composition comprises about 25.7% (w/w) extragranular filler.
Embodiment 120. The pharmaceutical composition of any one of embodiments 5 to 119, wherein the pharmaceutical composition comprises about 0.25% (w/w) to about 0.75% (w/w) intragranular glidant.
Embodiment 121. The pharmaceutical composition of any one of embodiments 5 to 119, wherein the pharmaceutical composition comprises about 0.3% (w/w) to about 0.6% (w/w) intragranular glidant.
Embodiment 122. The pharmaceutical composition of any one of embodiments 5 to 119, wherein the pharmaceutical composition comprises about 0.35% (w/w) to about 0.55% (w/w) intragranular glidant.
Embodiment 123. The pharmaceutical composition of any one of embodiments 5 to 119, wherein the pharmaceutical composition comprises about 0.3% (w/w) to about 0.45% (w/w) intragranular glidant.
Embodiment 124. The pharmaceutical composition of any one of embodiments 5 to 119, wherein the pharmaceutical composition comprises about 0.35% (w/w) to about 0.4% (w/w) intragranular glidant.
Embodiment 125. The pharmaceutical composition of any one of embodiments 5 to 119, wherein the pharmaceutical composition comprises about 0.45% (w/w) to about 0.55% (w/w) intragranular glidant.
Embodiment 126. The pharmaceutical composition of any one of embodiments 5 to 119, wherein the pharmaceutical composition comprises about 0.48% (w/w) to about 0.52% (w/w) intragranular glidant.
Embodiment 127. The pharmaceutical composition of any one of embodiments 5 to 119, wherein the pharmaceutical composition comprises about 0.30% (w/w), about 0.31% (w/w), about 0.32% (w/w), about 0.33% (w/w), about 0.34% (w/w), about 0.35% (w/w), about 0.36% (w/w), about 0.37% (w/w), about 0.38% (w/w), about 0.39% (w/w), about 0.4% (w/w), about 0.41% (w/w), about 0.42% (w/w), about 0.43% (w/w), about 0.44% (w/w), about 0.45% (w/w), about 0.46% (w/w), about 0.47% (w/w), about 0.48% (w/w), about 0.49% (w/w), about 0.5% (w/w), about 0.51% (w/w), about 0.52% (w/w), about 0.53% (w/w), about 0.54% (w/w) or about 0.55% (w/w) intragranular glidant.
Embodiment 128. The pharmaceutical composition of any one of embodiments 5 to 119, wherein the pharmaceutical composition comprises about 0.35% (w/w), about 0.36% (w/w), about 0.37% (w/w), about 0.38% (w/w), about 0.39% (w/w), about 0.48% (w/w), about 0.49% (w/w), about 0.5% (w/w), about 0.51% (w/w) or about 0.52% (w/w) intragranular glidant.
Embodiment 129. The pharmaceutical composition of any one of embodiments 5 to 119, wherein the pharmaceutical composition comprises about 0.37% (w/w) intragranular glidant.
Embodiment 130. The pharmaceutical composition of any one of embodiments 5 to 119, wherein the pharmaceutical composition comprises about 0.5% (w/w) intragranular glidant.
Embodiment 131. The pharmaceutical composition of any one of embodiments 5 to 130, wherein the pharmaceutical composition comprises about 0.25% (w/w) to about 0.75% (w/w) extragranular glidant.
Embodiment 132. The pharmaceutical composition of any one of embodiments 5 to 130, wherein the pharmaceutical composition comprises about 0.3% (w/w) to about 0.7% (w/w) extragranular glidant.
Embodiment 133. The pharmaceutical composition of any one of embodiments 5 to 130, wherein the pharmaceutical composition comprises about 0.4% (w/w) to about 0.6% (w/w) extragranular glidant.
Embodiment 134. The pharmaceutical composition of any one of embodiments 5 to 130, wherein the pharmaceutical composition comprises about 0.45% (w/w) to about 0.55% (w/w) extragranular glidant.
Embodiment 135. The pharmaceutical composition of any one of embodiments 5 to 130, wherein the pharmaceutical composition comprises about 0.45% (w/w), about 0.46% (w/w), about 0.47% (w/w), about 0.48% (w/w), about 0.49% (w/w), about 0.5% (w/w), about 0.51% (w/w), about 0.52% (w/w), about 0.53% (w/w), about 0.54% (w/w) or about 0.55% (w/w) extragranular glidant.
Embodiment 136. The pharmaceutical composition of any one of embodiments 5 to 130, wherein the pharmaceutical composition comprises about 0.48% (w/w), about 0.49% (w/w), about 0.5% (w/w), about 0.51% (w/w) or about 0.52% (w/w) extragranular glidant.
Embodiment 137. The pharmaceutical composition of any one of embodiments 5 to 130, wherein the pharmaceutical composition comprises about 0.5% (w/w) extragranular glidant.
Embodiment 138. The pharmaceutical composition of any one of embodiments 5 to 137, wherein the pharmaceutical composition comprises about 1% (w/w) to about 3% (w/w) intragranular disintegrant.
Embodiment 139. The pharmaceutical composition of any one of embodiments 5 to 137, wherein the pharmaceutical composition comprises about 1.2% (w/w) to about 2.4% (w/w) intragranular disintegrant.
Embodiment 140. The pharmaceutical composition of any one of embodiments 5 to 137, wherein the pharmaceutical composition comprises about 1.4% (w/w) to about 2.2% (w/w) intragranular disintegrant.
Embodiment 141. The pharmaceutical composition of any one of embodiments 5 to 137, wherein the pharmaceutical composition comprises about 1.2% (w/w) to about 1.8% (w/w) intragranular disintegrant.
Embodiment 142. The pharmaceutical composition of any one of embodiments 5 to 137, wherein the pharmaceutical composition comprises about 1.4% (w/w) to about 1.6% (w/w) intragranular disintegrant.
Embodiment 143. The pharmaceutical composition of any one of embodiments 5 to 137, wherein the pharmaceutical composition comprises about 1.8% (w/w) to about 2.2% (w/w) intragranular disintegrant.
Embodiment 144. The pharmaceutical composition of any one of embodiments 5 to 137, wherein the pharmaceutical composition comprises about 1.92% (w/w) to about 2.08% (w/w) intragranular disintegrant.
Embodiment 145. The pharmaceutical composition of any one of embodiments 5 to 137, wherein the pharmaceutical composition comprises about 1.2% (w/w), about 1.24% (w/w), about 1.28% (w/w), about 1.32% (w/w), about 1.36% (w/w), about 1.4% (w/w), about 1.44% (w/w), about 1.47% (w/w), about 1.52% (w/w), about 1.56% (w/w), about 1.6% (w/w), about 1.64% (w/w), about 1.68% (w/w), about 1.72% (w/w), about 1.76% (w/w), about 1.8% (w/w), about 1.84% (w/w), about 1.88% (w/w), about 1.92% (w/w), about 1.96% (w/w), about 2% (w/w), about 2.04% (w/w), about 2.08% (w/w), about 2.12% (w/w), about 2.16% (w/w) or about 2.2% (w/w) intragranular disintegrant.
Embodiment 146. The pharmaceutical composition of any one of embodiments 5 to 137, wherein the pharmaceutical composition comprises about 1.4% (w/w), about 1.44% (w/w), about 1.47% (w/w), about 1.52% (w/w), about 1.56% (w/w), about 1.92% (w/w), about 1.96% (w/w), about 2% (w/w), about 2.04% (w/w) or about 2.08% (w/w) intragranular disintegrant.
Embodiment 147. The pharmaceutical composition of any one of embodiments 5 to 137, wherein the pharmaceutical composition comprises about 1.47% (w/w) intragranular disintegrant.
Embodiment 148. The pharmaceutical composition of any one of embodiments 5 to 137, wherein the pharmaceutical composition comprises about 2% (w/w) intragranular disintegrant.
Embodiment 149. The pharmaceutical composition of any one of embodiments 5 to 148, wherein the pharmaceutical composition comprises about 2% (w/w) to about 3% (w/w) extragranular disintegrant.
Embodiment 150. The pharmaceutical composition of any one of embodiments 5 to 148, wherein the pharmaceutical composition comprises about 1.2% (w/w) to about 2.8% (w/w) extragranular disintegrant.
Embodiment 151. The pharmaceutical composition of any one of embodiments 5 to 148, wherein the pharmaceutical composition comprises about 1.6% (w/w) to about 2.4% (w/w) extragranular disintegrant.
Embodiment 152. The pharmaceutical composition of any one of embodiments 5 to 148, wherein the pharmaceutical composition comprises about 1.8% (w/w) to about 2.2% (w/w) extragranular disintegrant.
Embodiment 153. The pharmaceutical composition of any one of embodiments 5 to 148, wherein the pharmaceutical composition comprises about 1.8% (w/w), about 1.84% (w/w), about 1.88% (w/w), about 1.92% (w/w), about 1.96% (w/w), about 2% (w/w), about 2.04% (w/w), about 2.08% (w/w), about 2.12% (w/w), about 2.16% (w/w) or about 2.2% (w/w) extragranular disintegrant.
Embodiment 154. The pharmaceutical composition of any one of embodiments 5 to 148, wherein the pharmaceutical composition comprises about 1.92% (w/w), about 1.96% (w/w), about 2% (w/w), about 2.04% (w/w) or about 2.08% (w/w) extragranular disintegrant.
Embodiment 155. The pharmaceutical composition of any one of embodiments 5 to 148, wherein the pharmaceutical composition comprises about 2% (w/w) extragranular disintegrant.
Embodiment 156. The pharmaceutical composition of any one of embodiments 5 to 155, wherein the pharmaceutical composition comprises about 0.25% (w/w) to about 0.75% (w/w) intragranular lubricant.
Embodiment 157. The pharmaceutical composition of any one of embodiments 5 to 155, wherein the pharmaceutical composition comprises about 0.3% (w/w) to about 0.6% (w/w) intragranular lubricant.
Embodiment 158. The pharmaceutical composition of any one of embodiments 5 to 155, wherein the pharmaceutical composition comprises about 0.35% (w/w) to about 0.55% (w/w) intragranular lubricant.
Embodiment 159. The pharmaceutical composition of any one of embodiments 5 to 155, wherein the pharmaceutical composition comprises about 0.3% (w/w) to about 0.45% (w/w) intragranular lubricant.
Embodiment 160. The pharmaceutical composition of any one of embodiments 5 to 155, wherein the pharmaceutical composition comprises about 0.35% (w/w) to about 0.4% (w/w) intragranular lubricant.
Embodiment 161. The pharmaceutical composition of any one of embodiments 5 to 155, wherein the pharmaceutical composition comprises about 0.45% (w/w) to about 0.55% (w/w) intragranular lubricant.
Embodiment 162. The pharmaceutical composition of any one of embodiments 5 to 155, wherein the pharmaceutical composition comprises about 0.48% (w/w) to about 0.52% (w/w) intragranular lubricant.
Embodiment 163. The pharmaceutical composition of any one of embodiments 5 to 155, wherein the pharmaceutical composition comprises about 0.30% (w/w), about 0.31% (w/w), about 0.32% (w/w), about 0.33% (w/w), about 0.34% (w/w), about 0.35% (w/w), about 0.36% (w/w), about 0.37% (w/w), about 0.38% (w/w), about 0.39% (w/w), about 0.4% (w/w), about 0.41% (w/w), about 0.42% (w/w), about 0.43% (w/w), about 0.44% (w/w), about 0.45% (w/w), about 0.46% (w/w), about 0.47% (w/w), about 0.48% (w/w), about 0.49% (w/w), about 0.5% (w/w), about 0.51% (w/w), about 0.52% (w/w), about 0.53% (w/w), about 0.54% (w/w) or about 0.55% (w/w) intragranular lubricant.
Embodiment 164. The pharmaceutical composition of any one of embodiments 5 to 155, wherein the pharmaceutical composition comprises about 0.35% (w/w), about 0.36% (w/w), about 0.37% (w/w), about 0.38% (w/w), about 0.39% (w/w), about 0.48% (w/w), about 0.49% (w/w), about 0.5% (w/w), about 0.51% (w/w) or about 0.52% (w/w) intragranular lubricant.
Embodiment 165. The pharmaceutical composition of any one of embodiments 5 to 155, wherein the pharmaceutical composition comprises about 0.37% (w/w) intragranular lubricant.
Embodiment 166. The pharmaceutical composition of any one of embodiments 5 to 155, wherein the pharmaceutical composition comprises about 0.5% (w/w) intragranular lubricant.
Embodiment 167. The pharmaceutical composition of any one of embodiments 5 to 166, wherein the pharmaceutical composition comprises about 0.25% (w/w) to about 0.75% (w/w) extragranular lubricant.
Embodiment 168. The pharmaceutical composition of any one of embodiments 5 to 166, wherein the pharmaceutical composition comprises about 0.3% (w/w) to about 0.7% (w/w) extragranular lubricant.
Embodiment 169. The pharmaceutical composition of any one of embodiments 5 to 166, wherein the pharmaceutical composition comprises about 0.4% (w/w) to about 0.6% (w/w) extragranular lubricant.
Embodiment 170. The pharmaceutical composition of any one of embodiments 5 to 166, wherein the pharmaceutical composition comprises about 0.45% (w/w) to about 0.55% (w/w) extragranular lubricant.
Embodiment 171. The pharmaceutical composition of any one of embodiments 5 to 166, wherein the pharmaceutical composition comprises about 0.45% (w/w), about 0.46% (w/w), about 0.47% (w/w), about 0.48% (w/w), about 0.49% (w/w), about 0.5% (w/w), about 0.51% (w/w), about 0.52% (w/w), about 0.53% (w/w), about 0.54% (w/w) or about 0.55% (w/w) extragranular lubricant.
Embodiment 172. The pharmaceutical composition of any one of embodiments 5 to 166, wherein the pharmaceutical composition comprises about 0.48% (w/w), about 0.49% (w/w), about 0.5% (w/w), about 0.51% (w/w) or about 0.52% (w/w) extragranular lubricant.
Embodiment 173. The pharmaceutical composition of any one of embodiments 5 to 166, wherein the pharmaceutical composition comprises about 0.5% (w/w) extragranular lubricant.
Embodiment 174. A pharmaceutical composition comprising:
(a) a compound of formula (I)

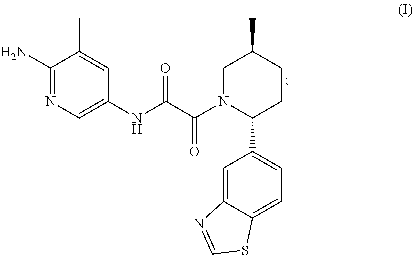 [SMILES: Cc1cc(NC(=O)C(=O)N2C[C@@H](C)CC[C@@H]2c2ccc3scnc3c2)cnc1N]

(b) an intragranular filler (e.g., microcrystalline cellulose);
(c) an intragranular glidant (e.g., colloidal silicon dioxide);
(d) an intragranular disintegrant (e.g., croscarmellose sodium);
(e) an extragranular lubricant (e.g., magnesium stearate);
(f) an extragranular filler (e.g., microcrystalline cellulose);
(g) an extragranular glidant (e.g., colloidal silicon dioxide);
(h) an extragranular disintegrant (e.g., croscarmellose sodium); and
(i) an extragranular lubricant (e.g., magnesium stearate).
Embodiment 175. The pharmaceutical composition of embodiment 174, wherein the composition comprises a crystalline form of the compound of formula (I) of any one of embodiments 1 to 4.
Embodiment 176. A pharmaceutical composition of embodiment 174 or 175, wherein the composition comprises:
(a) about 5% (w/w) to about 50% (w/w) of the compound of formula (I);
(b) about 30% (w/w) to about 70% (w/w) of an intragranular filler (e.g., microcrystalline cellulose);
(c) about 0.25% (w/w) to about 0.75% (w/w) of an intragranular glidant (e.g., colloidal silicon dioxide);
(d) about 1% (w/w) to about 3% (w/w) of an intragranular disintegrant (e.g., croscarmellose sodium);
(e) about 0.25% (w/w) to about 0.75% (w/w) of an intragranular lubricant (e.g., magnesium stearate);
(f) about 0% (w/w) to about 40% (w/w) of an extragranular filler (e.g., microcrystalline cellulose);
(g) about 0.25% (w/w) to about 0.75% (w/w) of an extragranular glidant (e.g., colloidal silicon dioxide);
(h) about 1% (w/w) to about 3% (w/w) of an extragranular disintegrant (e.g., croscarmellose sodium); and
(i) about 0.25% (w/w) to about 0.75% (w/w) of an extragranular lubricant (e.g., magnesium stearate);
thereby totaling 100% (w/w) of the composition.
Embodiment 177. A pharmaceutical composition of embodiment 174 or 175, wherein the composition comprises:
(a) about 10% (w/w) to about 45% (w/w) of the compound of formula (I);
(b) about 35% (w/w) to about 60% (w/w) of an intragranular filler (e.g., microcrystalline cellulose);
(c) about 0.35% (w/w) to about 0.55% (w/w) of an intragranular glidant (e.g., colloidal silicon dioxide);
(d) about 1.4% (w/w) to about 2.2% (w/w) of an intragranular disintegrant (e.g., croscarmellose sodium);
(e) about 0.35% (w/w) to about 0.55% (w/w) of an intragranular lubricant (e.g., magnesium stearate);
(f) about 0% (w/w) to about 30% (w/w) of an extragranular filler (e.g., microcrystalline cellulose);
(g) about 0.35% (w/w) to about 0.55% (w/w) of an extragranular glidant (e.g., colloidal silicon dioxide);
(h) about 1.8% (w/w) to about 2.2% (w/w) of an extragranular disintegrant (e.g., croscarmellose sodium); and
(i) about 0.35% (w/w) to about 0.55% (w/w) of an extragranular lubricant (e.g., magnesium stearate);
thereby totaling 100% (w/w) of the composition.
Embodiment 178. A pharmaceutical composition of embodiment 174 or 175, wherein the composition comprises:
(a) about 10% (w/w) to about 15% (w/w) of the compound of formula (I);
(b) about 52% (w/w) to about 58% (w/w) of an intragranular filler (e.g., microcrystalline cellulose);
(c) about 0.95% (w/w) to about 1.05% (w/w) of an intragranular glidant (e.g., colloidal silicon dioxide);
(d) about 1.92% (w/w) to about 2.08% (w/w) of an intragranular disintegrant (e.g., croscarmellose sodium);
(e) about 0.95% (w/w) to about 1.05% (w/w) of an intragranular lubricant (e.g., magnesium stearate);
(f) about 22% (w/w) to about 27% (w/w) of an extragranular filler (e.g., microcrystalline cellulose);
(g) about 0.45% (w/w) to about 0.55% (w/w) of an extragranular glidant (e.g., colloidal silicon dioxide);
(h) about 1.8% (w/w) to about 2.2% (w/w) of an extragranular disintegrant (e.g., croscarmellose sodium); an;
(i) about 0.45% (w/w) to about 0.55% (w/w) of an extragranular lubricant (e.g., magnesium stearate);
thereby totaling 100% (w/w) of the composition.
Embodiment 179. A pharmaceutical composition of embodiment 174 or 175, wherein the composition comprises:
(a) about 12.5% (w/w) of the compound of formula (I);
(b) about 56.5% (w/w) of an intragranular filler (e.g., microcrystalline cellulose);
(c) about 0.5% (w/w) of an intragranular glidant (e.g., colloidal silicon dioxide);
(d) about 2% (w/w) of an intragranular disintegrant (e.g., croscarmellose sodium);
(e) about 0.5% (w/w) of an intragranular lubricant (e.g., magnesium stearate);
(f) about 25% (w/w) of an extragranular filler (e.g., microcrystalline cellulose);
(g) about 0.5% (w/w) of an extragranular glidant (e.g., colloidal silicon dioxide);
(h) about 2% (w/w) of an extragranular disintegrant (e.g., croscarmellose sodium); and
(i) about 0.5% (w/w) of an extragranular lubricant (e.g., magnesium stearate);
thereby totaling 100% (w/w) of the composition.
Embodiment 180. A pharmaceutical composition of embodiment 174 or 175, wherein the composition comprises:
(a) about 27% (w/w) to about 33% (w/w) of the compound of formula (I);
(b) about 37% (w/w) to about 43% (w/w) of an intragranular filler (e.g., microcrystalline cellulose);
(c) about 0.85% (w/w) to about 0.9% (w/w) of an intragranular glidant (e.g., colloidal silicon dioxide);
(d) about 1.4% (w/w) to about 1.6% (w/w) of an intragranular disintegrant (e.g., croscarmellose sodium);
(e) about 0.85% (w/w) to about 0.9% (w/w) of an intragranular lubricant (e.g., magnesium stearate);
(f) about 0% (w/w) to about 30% (w/w) of an extragranular filler (e.g., microcrystalline cellulose);
(g) about 0.45% (w/w) to about 0.55% (w/w) of an extragranular glidant (e.g., colloidal silicon dioxide);
(h) about 1.8% (w/w) to about 2.2% (w/w) of an extragranular disintegrant (e.g., croscarmellose sodium); and
(i) about 0.45% (w/w) to about 0.55% (w/w) of an extragranular lubricant (e.g., magnesium stearate);
thereby totaling 100% (w/w) of the composition.
Embodiment 181. A pharmaceutical composition of embodiment 174 or 175, wherein the composition comprises:
(a) about 29.4% (w/w) of the compound of formula (I);
(b) about 39.7% (w/w) of an intragranular filler (e.g., microcrystalline cellulose);
(c) about 0.37% (w/w) of an intragranular glidant (e.g., colloidal silicon dioxide);
(d) about 1.47% (w/w) of an intragranular disintegrant (e.g., croscarmellose sodium);
(e) about 0.37% (w/w) of an intragranular lubricant (e.g., magnesium stearate);
(f) about 25.7% (w/w) of an extragranular filler (e.g., microcrystalline cellulose);
(g) about 0.5% (w/w) of an extragranular glidant (e.g., colloidal silicon dioxide);
(h) about 2% (w/w) of an extragranular disintegrant (e.g., croscarmellose sodium); and
(i) about 0.5% (w/w) of an extragranular lubricant (e.g., magnesium stearate);
thereby totaling 100% (w/w) of the composition.
Embodiment 182. A pharmaceutical composition of embodiment 174 or 175, wherein the composition comprises:
(a) about 37% (w/w) to about 43% (w/w) of the compound of formula (I);
(b) about 52% (w/w) to about 58% (w/w) of an intragranular filler (e.g., microcrystalline cellulose);
(c) about 0.95% (w/w) to about 1.05% (w/w) of an intragranular glidant (e.g., colloidal silicon dioxide);
(d) about 1.92% (w/w) to about 2.08% (w/w) of an intragranular disintegrant (e.g., croscarmellose sodium);
(e) about 0.95% (w/w) to about 1.05% (w/w) of an intragranular lubricant (e.g., magnesium stearate);
(f) about 0% (w/w) to about 30% (w/w) of an extragranular filler (e.g., microcrystalline cellulose);
(g) about 0.45% (w/w) to about 0.55% (w/w) of an extragranular glidant (e.g., colloidal silicon dioxide);
(h) about 1.8% (w/w) to about 2.2% (w/w) of an extragranular disintegrant (e.g., croscarmellose sodium); and
(i) about 0.45% (w/w) to about 0.55% (w/w) of an extragranular lubricant (e.g., magnesium stearate);
thereby totaling 100% (w/w) of the composition.
Embodiment 183. A pharmaceutical composition of embodiment 174 or 175, wherein the composition comprises:
(a) about 40% (w/w) of the compound of formula (I);
(b) about 54% (w/w) of an intragranular filler (e.g., microcrystalline cellulose);
(c) about 0.5% (w/w) of an intragranular glidant (e.g., colloidal silicon dioxide);
(d) about 2% (w/w) of an intragranular disintegrant (e.g., croscarmellose sodium);
(e) about 0.5% (w/w) of an intragranular lubricant (e.g., magnesium stearate);
(f) about 0% (w/w) of an extragranular filler (e.g., microcrystalline cellulose);
(g) about 0.5% (w/w) of an extragranular glidant (e.g., colloidal silicon dioxide);
(h) about 2% (w/w) of an extragranular disintegrant (e.g., croscarmellose sodium); and
(i) about 0.5% (w/w) of an extragranular lubricant (e.g., magnesium stearate);
thereby totaling 100% (w/w) of the composition.
Embodiment 184. The pharmaceutical composition of any one of embodiments 5 to 183 wherein the intragranular filler is microcrystalline cellulose.
Embodiment 185. The pharmaceutical composition of any one of embodiments 5 to 183 wherein the intragranular filler is microcrystalline cellulose PH 102.
Embodiment 186. The pharmaceutical composition of any one of embodiments 5 to 185 wherein the extragranular filler is microcrystalline cellulose.
Embodiment 187. The pharmaceutical composition of any one of embodiments 5 to 186 wherein the extragranular filler is microcrystalline cellulose PH 102, PH 200 or a mixture thereof.
Embodiment 188. The pharmaceutical composition of any one of embodiments 5 to 186 wherein the extragranular filler is a single grade of microcrystalline cellulose.
Embodiment 189. The pharmaceutical composition of embodiment 187 wherein the extragranular filler is microcrystalline cellulose PH 200.
Embodiment 190. The pharmaceutical composition of embodiment 187 wherein the extragranular filler is microcrystalline cellulose PH 120.
Embodiment 191. The pharmaceutical composition of any one of embodiments 5 to 186 wherein the extragranular filler is a mixture of grades of microcrystalline cellulose.
Embodiment 192. The pharmaceutical composition of any one of embodiments 5 to 191 wherein the extragranular filler is a mixture of microcrystalline cellulose PH 200 and PH102.
Embodiment 193. The pharmaceutical composition of embodiment 192 wherein the mixture contains equal amounts of microcrystalline cellulose PH 200 and PH102.
Embodiment 194. The pharmaceutical composition of any one of embodiments 5 to 193 wherein the intragranular glidant is colloidal silicon dioxide.
Embodiment 195. The pharmaceutical composition of any one of embodiments 5 to 194 wherein the extragranular glidant is colloidal silicon dioxide.
Embodiment 196. The pharmaceutical composition of any one of embodiments 5 to 195 wherein the intragranular disintegrant is croscarmellose sodium.
Embodiment 197. The pharmaceutical composition of any one of embodiments 5 to 196 wherein the extragranular disintegrant is croscarmellose sodium.
Embodiment 198. The pharmaceutical composition of any one of embodiments 5 to 197 wherein the intragranular lubricant is magnesium stearate.
Embodiment 199. The pharmaceutical composition of any one of embodiments 5 to 198 wherein the extragranular lubricant is magnesium stearate.
Embodiment 200. A dosage form comprising a pharmaceutical composition of any one of embodiments 5 to 199.
Embodiment 201. The dosage form of embodiment 200, wherein the total weight of the pharmaceutical composition in the dosage form is about 50 mg to 1000 mg.
Embodiment 202. The dosage form of embodiment 200, wherein the total weight of the pharmaceutical composition in the dosage form is about 100 mg to 750 mg.
Embodiment 203. The dosage form of embodiment 200, wherein the total weight of the pharmaceutical composition in the dosage form is about 50 mg to 150 mg.
Embodiment 204. The dosage form of embodiment 200, wherein the total weight of the pharmaceutical composition in the dosage form is about 50 mg, about 60 mg, about 70 mg, about 80 mg, about 90 mg, about 100 mg, about 110 mg, about 120 mg, about 130 mg, about 140 mg or about 150 mg.
Embodiment 205. The dosage form of embodiment 200, wherein the total weight of the pharmaceutical composition in the dosage form is about 80 mg, about 90 mg, about 100 mg, about 110 mg, or about 120 mg.
Embodiment 206. The dosage form of embodiment 200, wherein the total weight of the pharmaceutical composition in the dosage form is about 100 mg.
Embodiment 207. The dosage form of embodiment 200, wherein the total weight of the pharmaceutical composition in the dosage form is about 300 mg to 500 mg.
Embodiment 208. The dosage form of embodiment 200, wherein the total weight of the pharmaceutical composition in the dosage form is about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg or about 500 mg.
Embodiment 209. The dosage form of embodiment 200, wherein the total weight of the pharmaceutical composition in the dosage form is about 320 mg, about 330 mg, about 340 mg, about 350 mg or about 360 mg.
Embodiment 210. The dosage form of embodiment 200, wherein the total weight of the pharmaceutical composition in the dosage form is about 340 mg.
Embodiment 211. The dosage form of embodiment 200, wherein the total weight of the pharmaceutical composition in the dosage form is about 380 mg, about 390 mg, about 400 mg, about 410 mg or about 420 mg.
Embodiment 212. The dosage form of embodiment 200, wherein the total weight of the pharmaceutical composition in the dosage form is about 400 mg.
Embodiment 213. The dosage form of embodiment 200, wherein the total weight of the pharmaceutical composition in the dosage form is about 600 mg to 900 mg.
Embodiment 214. The dosage form of embodiment 200, wherein the total weight of the pharmaceutical composition in the dosage form is about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, about 800 mg, about 810 mg, about 820 mg, about 830 mg, about 840 mg, about 850 mg, about 860 mg, about 870 mg, about 880 mg, about 890 mg or about 900 mg.
Embodiment 215. The dosage form of embodiment 200, wherein the total weight of the pharmaceutical composition in the dosage form is about 700 mg, about 710 mg, about 720 mg, about 730 mg, about 740 mg, about 750 mg, about 760 mg, about 770 mg, about 780 mg, about 790 mg or about 800 mg.
Embodiment 216. The dosage form of embodiment 200, wherein the total weight of the pharmaceutical composition in the dosage form is about 750 mg.
Embodiment 217. The dosage form of embodiment 200, wherein the total weight of the pharmaceutical composition in the dosage form is about 100 mg, about 340 mg, about 400 mg or about 750 mg.
Embodiment 218. The dosage form of any one of embodiments 200 to 217, wherein the composition comprises about 5 mg to about 400 mg of a compound of formula (I).
Embodiment 219. The dosage form of any one of embodiments 200 to 217, wherein the composition comprises about 10 mg to about 350 mg of the compound of formula (I).
Embodiment 220. The dosage form of any one of embodiments 200 to 217, wherein the composition comprises about 8 mg to about 17 mg, about 40 mg to about 60 mg, about 80 mg to about 120 mg or about 250 to about 350 mg of the compound of formula (I).
Embodiment 221. The dosage form of any one of embodiments 200 to 217, wherein the composition comprises about 8 mg to about 17 mg of the compound of formula (I).
Embodiment 222. The dosage form of any one of embodiments 200 to 217, wherein the composition comprises about 10 mg to about 15 mg of the compound of formula (I).
Embodiment 223. The dosage form of any one of embodiments 200 to 217, wherein the composition comprises about 12 mg to about 13 mg of the compound of formula (I).
Embodiment 224. The dosage form of any one of embodiments 200 to 217, wherein the composition comprises about 8 mg, about 9 mg, about 10 mg, about 11 mg, about 12 mg, about 12.5 mg, about 13 mg, about 14 mg, about 15 mg, about 16 mg or about 17 mg of the compound of formula (I).
Embodiment 225. The dosage form of any one of embodiments 200 to 217, wherein the composition comprises about 12.5 mg of the compound of formula (I).
Embodiment 226. The dosage form of any one of embodiments 200 to 217, wherein the composition comprises about 40 mg to about 60 mg of the compound of formula (I).
Embodiment 227. The dosage form of any one of embodiments 200 to 217, wherein the composition comprises about 45 mg to about 55 mg of the compound of formula (I).
Embodiment 228. The dosage form of any one of embodiments 200 to 217, wherein the composition comprises about 47 mg to about 53 mg of the compound of formula (I).
Embodiment 229. The dosage form of any one of embodiments 200 to 217, wherein the composition comprises about 49 mg to about 51 mg of the compound of formula (I).
Embodiment 230. The dosage form of any one of embodiments 200 to 217, wherein the composition comprises about 45 mg, about 46 mg, about 47 mg, about 48 mg, about 49 mg, about 50 mg, about 51 mg, about 52 mg, about 53 mg, about 54 mg or about 55 mg of the compound of formula (I).
Embodiment 231. The dosage form of any one of embodiments 200 to 217, wherein the composition comprises about 50 mg of the compound of formula (I).
Embodiment 232. The dosage form of any one of embodiments 200 to 217, wherein the composition comprises about 80 mg to about 120 mg of the compound of formula (I).
Embodiment 233. The dosage form of any one of embodiments 200 to 217, wherein the composition comprises about 90 mg to about 110 mg of the compound of formula (I).
Embodiment 234. The dosage form of any one of embodiments 200 to 217, wherein the composition comprises about 95 mg to about 105 mg of the compound of formula (I).
Embodiment 235. The dosage form of any one of embodiments 200 to 217, wherein the composition comprises about 98 mg to about 102 mg of the compound of formula (I).
Embodiment 236. The dosage form of any one of embodiments 200 to 217, wherein the composition comprises about 90 mg, about 91 mg, about 92 mg, about 93 mg, about 94 mg, about 95 mg, about 96 mg, about 97 mg, about 98 mg, about 99 mg, about 100 mg, about 101 mg, about 102 mg, about 103 mg, about 104 mg, about 105 mg about 106 mg, about 107 mg, about 108 mg, about 109 mg or about 110 mg of the compound of formula (I).
Embodiment 237. The dosage form of any one of embodiments 200 to 217, wherein the composition comprises about 100 mg of the compound of formula (I).
Embodiment 238. The dosage form of any one of embodiments 200 to 217, wherein the composition comprises about 250 to about 350 mg of the compound of formula (I).
Embodiment 239. The dosage form of any one of embodiments 200 to 217, wherein the composition comprises about 260 to about 340 mg of the compound of formula (I).
Embodiment 240. The dosage form of any one of embodiments 200 to 217, wherein the composition comprises about 270 to about 330 mg of the compound of formula (I).
Embodiment 241. The dosage form of any one of embodiments 200 to 217, wherein the composition comprises about 280 to about 320 mg of the compound of formula (I).
Embodiment 242. The dosage form of any one of embodiments 200 to 217, wherein the composition comprises about 290 to about 310 mg of the compound of formula (I).
Embodiment 243. The dosage form of any one of embodiments 200 to 217, wherein the composition comprises about 295 to about 305 mg of the compound of formula (I).
Embodiment 244. The dosage form of any one of embodiments 200 to 217, wherein the composition comprises about 290 mg, about 291 mg, about 292 mg, about 293 mg, about 294 mg, about 295 mg, about 296 mg, about 297 mg, about 298 mg, about 299 mg, about 300 mg, about 301 mg, about 302 mg, about 303 mg, about 304 mg, about 305 mg about 306 mg, about 307 mg, about 308 mg, about 309 mg or about 310 mg of the compound of formula (I).
Embodiment 245. The dosage form of any one of embodiments 200 to 217, wherein the composition comprises about 300 mg of the compound of formula (I).
Embodiment 246. The dosage form of any one of embodiments 200 to 217, wherein the composition comprises about 8 mg, about 9 mg, about 10 mg, about 11 mg, about 12 mg, about 12.5 mg, about 13 mg, about 14 mg, about 15 mg, about 16 mg, about 17 mg, about 45 mg, about 46 mg, about 47 mg, about 48 mg, about 49 mg, about 50 mg, about 51 mg, about 52 mg, about 53 mg, about 54 mg, about 55 mg, about 90 mg, about 91 mg, about 92 mg, about 93 mg, about 94 mg, about 95 mg, about 96 mg, about 97 mg, about 98 mg, about 99 mg, about 100 mg, about 101 mg, about 102 mg, about 103 mg, about 104 mg, about 105 mg about 106 mg, about 107 mg, about 108 mg, about 109 mg, about 110 mg, about 290 mg, about 291 mg, about 292 mg, about 293 mg, about 294 mg, about 295 mg, about 296 mg, about 297 mg, about 298 mg, about 299 mg, about 300 mg, about 301 mg, about 302 mg, about 303 mg, about 304 mg, about 305 mg about 306 mg, about 307 mg, about 308 mg, about 309 mg or about 310 mg of the compound of formula (I).
Embodiment 247. The dosage form of any one of embodiments 200 to 217, wherein the composition comprises about 10 mg, about 11 mg, about 12 mg, about 12.5 mg, about 13 mg, about 14 mg, about 15 mg, about 48 mg, about 49 mg, about 50 mg, about 51 mg, about 52 mg, about 53 mg, about 96 mg, about 97 mg, about 98 mg, about 99 mg, about 100 mg, about 101 mg, about 102 mg, about 103 mg, about 104 mg, about 295 mg, about 296 mg, about 297 mg, about 298 mg, about 299 mg, about 300 mg, about 301 mg, about 302 mg, about 303 mg, about 304 mg or about 305 mg of the compound of formula (I).
Embodiment 248. The dosage form of any one of embodiments 200 to 217, wherein the composition comprises about 12.5 mg, about 50 mg, about 100 mg or about 300 mg of the compound of formula (I).
Embodiment 249. The dosage form of any one of embodiments 200 to 217 wherein the composition comprises 12.5 mg Compound of formula (I), 56.5 mg intragranular microcrystalline cellulose, 0.5 mg intragranular colloidal silicon dioxide, 2 mg intragranular croscarmellose sodium, 0.5 mg intragranular magnesium stearate, 25 mg extragranular microcrystalline cellulose, 0.5 mg extragranular colloidal silicon dioxide, 2 mg extragranular croscarmellose sodium and 0.5 mg extragranular magnesium stearate.
Embodiment 250. The dosage form of any one of embodiments 200 to 217 wherein the composition comprises 50 mg Compound of formula (I), 226 mg intragranular microcrystalline cellulose, 2 mg intragranular colloidal silicon dioxide, 8 mg intragranular croscarmellose sodium, 2 mg intragranular magnesium stearate, 100 mg extragranular microcrystalline cellulose, 2 mg extragranular colloidal silicon dioxide, 8 mg extragranular croscarmellose sodium and 2 mg extragranular magnesium stearate.
Embodiment 251. The dosage form of any one of embodiments 200 to 217 wherein the composition comprises 100 mg Compound of formula (I), 135 mg intragranular microcrystalline cellulose, 1.25 mg intragranular colloidal silicon dioxide, 5 mg intragranular croscarmellose sodium, 1.25 mg intragranular magnesium stearate, 87.3 mg extragranular microcrystalline cellulose (e.g., 43.65 mg PH 200 and 43.65 mg PH 102), 1.7 mg extragranular colloidal silicon dioxide, 6.8 mg extragranular croscarmellose sodium and 1.7 mg extragranular magnesium stearate.
Embodiment 252. The dosage form of any one of embodiments 200 to 217 wherein the composition comprises 300 mg Compound of formula (I), 405 mg intragranular microcrystalline cellulose, 3.75 mg intragranular colloidal silicon dioxide, 15 mg intragranular croscarmellose sodium, 3.75 mg intragranular magnesium stearate, 3.75 mg extragranular colloidal silicon dioxide, 15 mg extragranular croscarmellose sodium and 3.75 mg extragranular magnesium stearate.
Embodiment 253. The dosage form of any one of embodiments 200 to 252, wherein the dosage form is a solid dosage form.
Embodiment 254. The dosage form of any one of embodiments 200 to 253, wherein the dosage form is an oral dosage form.
Embodiment 255. The dosage form of any one of embodiments 200 to 254 wherein the dosage form is selected from the group consisting of a powder, a sachet, a stick pack, a capsule, a minitab, and a tablet.
Embodiment 256. The dosage form of any one of embodiments 200 to 254 wherein the dosage form is a tablet.
Embodiment 257. The dosage form of embodiment 256, wherein the tablet comprises a coating.
Embodiment 258. The dosage form of embodiment 257 wherein the coating comprises a polyvinyl alcohol.
Embodiment 259. A method for treating an MTAP-deficient and/or an MTA-accumulating disease in a subject in need thereof comprising administering to the subject a therapeutically effective amount of a crystalline form of any one of embodiments 1 to 4.
Embodiment 260. A method for treating an MTAP-deficient and/or an MTA-accumulating disease in a subject in need thereof comprising administering to the subject a pharmaceutical composition of any one of embodiments 5 to 199 containing a therapeutically effective amount of the compound of formula (I).
Embodiment 261. A method for treating an MTAP-deficient and/or an MTA-accumulating disease in a subject in need thereof comprising administering to the subject a dosage form of any one of embodiments 200 to 258 containing a therapeutically effective amount of the compound of formula (I).
Embodiment 262. The method of any one of embodiments 259 to 261 wherein the disease is a proliferating disease.
Embodiment 263. The method of any one of embodiments 259 to 262 wherein the disease is an MTAP-deficient and/or MTA-accumulating cancer.
Embodiment 264. A method of treating a cancer in a subject in need thereof comprising the steps of:
a) assessing the level of MTAP and/or MTA in a test sample obtained from said subject, wherein the MTA level can be assessed directly (e.g., by ELISA or LC-MS/MS) or indirectly (e.g., by SDMA-modified protein ELISA or IHC, or by RNA splicing);
b) comparing the test sample with a reference, wherein MTAP deficiency and/or MTA accumulation in said test sample compared to the reference indicates the cancer in said subject will respond to therapeutic treatment with a PRMT5 inhibitor; and
c) administering an effective amount (e.g., a therapeutically effective amount) of a crystalline form of any one of embodiments 1 to 4 to the subject identified in step b).
   Embodiment 265. A method of treating a cancer in a subject in need thereof comprising the steps of:
a) assessing the level of MTAP and/or MTA in a test sample obtained from said subject, wherein the MTA level can be assessed directly (e.g., by ELISA or LC-MS/MS) or indirectly (e.g., by SDMA-modified protein ELISA or IHC, or by RNA splicing);
b) comparing the test sample with a reference, wherein MTAP deficiency and/or MTA accumulation in said test sample compared to the reference indicates the cancer in said subject will respond to therapeutic treatment with a PRMT5 inhibitor; and
c) administering the pharmaceutical composition of any one of embodiments 5 to 199 containing an effective amount (e.g., a therapeutically effective amount) of the compound of formula a (I) to the subject identified in step b).
   Embodiment 266. A method of treating a cancer in a subject in need thereof comprising the steps of:
a) assessing the level of MTAP and/or MTA in a test sample obtained from said subject, wherein the MTA level can be assessed directly (e.g., by ELISA or LC-MS/MS) or indirectly (e.g., by SDMA-modified protein ELISA or IHC, or by RNA splicing);
b) comparing the test sample with a reference, wherein MTAP deficiency and/or MTA accumulation in said test sample compared to the reference indicates the cancer in said subject will respond to therapeutic treatment with a PRMT5 inhibitor; and
c) administering the dosage form of any one of embodiments 200 to 259 containing an effective amount (e.g., a therapeutically effective amount) of the compound of formula (I) to the subject identified in step b).
   Embodiment 267. The method of any one of embodiments 263 to 266 wherein the cancer is glioma, glioblastoma, malignant peripheral nerve sheath tumors (MPNST, e.g., intracranial MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine, sarcoma or a CNS metastasis from a solid tumor.
   Embodiment 268. The method of any one of embodiments 263 to 266 wherein the cancer is a CNS malignancy.
   Embodiment 269. The method of embodiment 268, wherein the CNS malignancy is glioma (e.g., low grade glioma, intermediate grade glioma), intracranial MPNST tumors, glioblastoma, glioblastoma multiforme, or CNS metastases from solid tumors.
   Embodiment 270. The method of embodiment 268, wherein the CNS malignancy is glioblastoma or glioblastoma multiforme.
   Embodiment 271. The method of embodiment 268, wherein the CNS malignancy is glioma.
   Embodiment 272. The method of embodiment 271, wherein the glioma is low grade glioma or intermediate grade glioma.
   Embodiment 273. The method of embodiment 271, wherein the glioma is low grade glioma.
   Embodiment 274. The method of embodiment 271, wherein the glioma is intermediate grade glioma.
   Embodiment 275. The method of embodiment 268, wherein the CNS malignancy is glioblastoma.
   Embodiment 276. The method of embodiment 268, wherein the CNS malignancy is glioblastoma multiforme.
   Embodiment 277. The method of embodiment 268, wherein the CNS malignancy is a MTAP-deleted glioblastoma.
   Embodiment 278. The method of embodiment 268, wherein the CNS malignancy is an intracranial MPNST tumor.
   Embodiment 279. The method of embodiment 268, wherein the CNS malignancy is solid tumor CNS metastases.
   Embodiment 280. The method of any one of embodiments 263 to 266 wherein the cancer is a cancer selected from the group of cholangiocarcinoma, NSCLC (adenocarcinoma), NSCLC (nonsquamous), bladder cancer, and DLBCL.
   Embodiment 281. The method of any one of embodiments 263 to 266 wherein the cancer is cholangiocarcinoma.
   Embodiment 282. The method of any one of embodiments 263 to 266 wherein the cancer is NSCLC (adenocarcinoma).
   Embodiment 283. The method of any one of embodiments 263 to 266 wherein the cancer is NSCLC (nonsquamous).
   Embodiment 284. The method of any one of embodiments 263 to 266 wherein the cancer is bladder cancer.
   Embodiment 285. The method of any one of embodiments 263 to 266 wherein the cancer is DLBCL.
   Embodiment 286. The method of any one of embodiments 263 to 266 wherein the cancer is a cancer selected from the group consisting of glioma, glioblastoma, non-small cell lung cancer (adenocarcinoma and squamous), mesothelioma, cholangiocarcinoma, urothelial carcinoma, and malignant peripheral nerve sheath tumor.
   Embodiment 287. The method of any one of embodiments 259 to 286, wherein the method comprises administering to the subject a dose of about 8 mg to about 17 mg of the compound of formula (I) once or twice daily.
   Embodiment 288. The method of any one of embodiments 259 to 286, wherein the method comprises administering to the subject a dose of about 10 mg to about 15 mg of the compound of formula (I) once or twice daily.
   Embodiment 289. The method of any one of embodiments 259 to 286, wherein the method comprises administering to the subject a dose of about 12 mg to about 13 mg of the compound of formula (I) once or twice daily.
   Embodiment 290. The method of any one of embodiments 259 to 286, wherein the method comprises administering to the subject a dose of about 8 mg, about 9 mg, about 10 mg, about 11 mg, about 12 mg, about 12.5 mg, about 13 mg, about 14 mg, about 15 mg, about 16 mg or about 17 mg of the compound of formula (I) once or twice daily.
   Embodiment 291. The method of any one of embodiments 259 to 286, wherein the method comprises administering to the subject a dose of about 12.5 mg of the compound of formula (I) once or twice daily.
   Embodiment 292. The method of any one of embodiments 259 to 286, wherein the method comprises administering to the subject a dose of about 40 mg to about 60 mg of the compound of formula (I) once or twice daily.
   Embodiment 293. The method of any one of embodiments 259 to 286, wherein the method comprises administering to the subject a dose of about 45 mg to about 55 mg of the compound of formula (I) once or twice daily.
   Embodiment 294. The method of any one of embodiments 259 to 286, wherein the method comprises administering to the subject a dose of about 47 mg to about 53 mg of the compound of formula (I) once or twice daily.
   Embodiment 295. The method of any one of embodiments 259 to 286, wherein the method comprises administering to the subject a dose of about 49 mg to about 51 mg of the compound of formula (I) once or twice daily.
   Embodiment 296. The method of any one of embodiments 259 to 286, wherein the method comprises administering to the subject a dose of about 45 mg, about 46 mg, about 47 mg, about 48 mg, about 49 mg, about 50 mg, about 51 mg, about 52 mg, about 53 mg, about 54 mg or about 55 mg of the compound of formula (I) once or twice daily.
   Embodiment 297. The method of any one of embodiments 259 to 286, wherein the method comprises administering to the subject a dose of about 50 mg of the compound of formula (I) once or twice daily.
   Embodiment 298. The method of any one of embodiments 259 to 286, wherein the method comprises administering to the subject a dose of about 80 mg to about 120 mg of the compound of formula (I) once or twice daily.
   Embodiment 299. The method of any one of embodiments 259 to 286, wherein the method comprises administering to the subject a dose of about 90 mg to about 110 mg of the compound of formula (I) once or twice daily.
   Embodiment 300. The method of any one of embodiments 259 to 286, wherein the method comprises administering to the subject a dose of about 95 mg to about 105 mg of the compound of formula (I) once or twice daily.
   Embodiment 301. The method of any one of embodiments 259 to 286, wherein the method comprises administering to the subject a dose of about 98 mg to about 102 mg of the compound of formula (I) once or twice daily.
   Embodiment 302. The method of any one of embodiments 259 to 286, wherein the method comprises administering to the subject a dose of about 90 mg, about 91 mg, about 92 mg, about 93 mg, about 94 mg, about 95 mg, about 96 mg, about 97 mg, about 98 mg, about 99 mg, about 100 mg, about 101 mg, about 102 mg, about 103 mg, about 104 mg, about 105 mg about 106 mg, about 107 mg, about 108 mg, about 109 mg or about 110 mg of the compound of formula (I) once or twice daily.
   Embodiment 303. The method of any one of embodiments 259 to 286, wherein the method comprises administering to the subject a dose of about 100 mg of the compound of formula (I) once or twice daily.
   Embodiment 304. The method of any one of embodiments 259 to 286, wherein the method comprises administering to the subject a dose of about 250 to about 350 mg of the compound of formula (I) once or twice daily.
   Embodiment 305. The method of any one of embodiments 259 to 286, wherein the method comprises administering to the subject a dose of about 260 to about 340 mg of the compound of formula (I) once or twice daily.
   Embodiment 306. The method of any one of embodiments 259 to 286, wherein the method comprises administering to the subject a dose of about 270 to about 330 mg of the compound of formula (I) once or twice daily.
   Embodiment 307. The method of any one of embodiments 259 to 286, wherein the method comprises administering to the subject a dose of about 280 to about 320 mg of the compound of formula (I) once or twice daily.
   Embodiment 308. The method of any one of embodiments 259 to 286, wherein the method comprises administering to the subject a dose of about 290 to about 310 mg of the compound of formula (I) once or twice daily.
   Embodiment 309. The method of any one of embodiments 259 to 286, wherein the method comprises administering to the subject a dose of about 295 to about 305 mg of the compound of formula (I) once or twice daily.
   Embodiment 310. The method of any one of embodiments 259 to 286, wherein the method comprises administering to the subject a dose of about 290 mg, about 291 mg, about 292 mg, about 293 mg, about 294 mg, about 295 mg, about 296 mg, about 297 mg, about 298 mg, about 299 mg, about 300 mg, about 301 mg, about 302 mg, about 303 mg, about 304 mg, about 305 mg about 306 mg, about 307 mg, about 308 mg, about 309 mg or about 310 mg of the compound of formula (I) once or twice daily.
   Embodiment 311. The method of any one of embodiments 259 to 286, wherein the method comprises administering to the subject a dose of about 300 mg of the compound of formula (I) once or twice daily.
   Embodiment 312. The method of any one of embodiments 259 to 286, wherein the method comprises administering to the subject a dose of about 8 mg, about 9 mg, about 10 mg, about 11 mg, about 12 mg, about 12.5 mg, about 13 mg, about 14 mg, about 15 mg, about 16 mg, about 17 mg, about 45 mg, about 46 mg, about 47 mg, about 48 mg, about 49 mg, about 50 mg, about 51 mg, about 52 mg, about 53 mg, about 54 mg, about 55 mg, about 90 mg, about 91 mg, about 92 mg, about 93 mg, about 94 mg, about 95 mg, about 96 mg, about 97 mg, about 98 mg, about 99 mg, about 100 mg, about 101 mg, about 102 mg, about 103 mg, about 104 mg, about 105 mg about 106 mg, about 107 mg, about 108 mg, about 109 mg, about 110 mg, about 290 mg, about 291 mg, about 292 mg, about 293 mg, about 294 mg, about 295 mg, about 296 mg, about 297 mg, about 298 mg, about 299 mg, about 300 mg, about 301 mg, about 302 mg, about 303 mg, about 304 mg, about 305 mg about 306 mg, about 307 mg, about 308 mg, about 309 mg or about 310 mg of the compound of formula (I) once or twice daily.
   Embodiment 313. The method of any one of embodiments 259 to 286, wherein the method comprises administering to the subject a dose of about 10 mg, about 11 mg, about 12 mg, about 12.5 mg, about 13 mg, about 14 mg, about 15 mg, about 48 mg, about 49 mg, about 50 mg, about 51 mg, about 52 mg, about 53 mg, about 96 mg, about 97 mg, about 98 mg, about 99 mg, about 100 mg, about 101 mg, about 102 mg, about 103 mg, about 104 mg, about 295 mg, about 296 mg, about 297 mg, about 298 mg, about 299 mg, about 300 mg, about 301 mg, about 302 mg, about 303 mg, about 304 mg or about 305 mg of the compound of formula (I) once or twice daily.
   Embodiment 314. The method of any one of embodiments 259 to 286, wherein the method comprises administering to the subject a dose of about 12.5 mg, about 50 mg, about 100 mg or about 300 mg of the compound of formula (I) once or twice daily.
   Embodiment 315. The method of any one of embodiments 259 to 314 wherein the method comprises administering the dose of the compound of formula (I) once daily.
   Embodiment 316. The method of any one of embodiments 259 to 314 wherein the method comprises administering the dose of the compound of formula (I) twice daily.
   Embodiment 317. The method of any one of embodiments 259 to 316, wherein the method further comprises administration of a second therapeutic agent.
   Embodiment 318. A process for preparing N-(6-amino-5-methylpyridin-3-yl)-2-((2R,5S)-2-(benzo[d]thiazol-5-yl)-5-methylpiperidin-1-yl)-2-oxoacetamide (a compound of formula (I)) or a salt thereof:

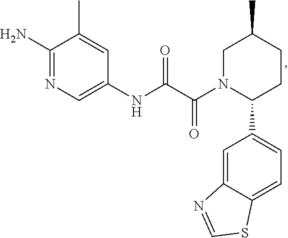 [SMILES: Cc1cc(NC(=O)C(=O)N2C[C@@H](C)CC[C@@H]2c2ccc3scnc3c2)cnc1N]

comprising:

hydrogenating a compound of formula (II):

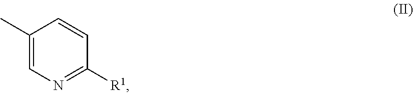

thereby producing a compound of formula (III-a):

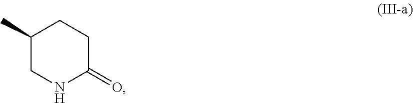

wherein R¹ is a chiral auxiliary.

Embodiment 319. The process of embodiment 318, wherein the process further comprises:

protecting the nitrogen group of the compound of formula (III-a), thereby forming a compound of formula (III):

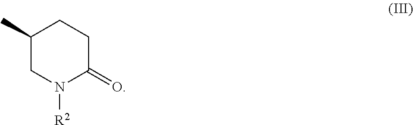

wherein R² is a nitrogen protecting group.
Embodiment 320. The process of embodiment 318 or 319, wherein the process further comprises:

cross-coupling a compound of formula (III) with a compound of formula (IV):

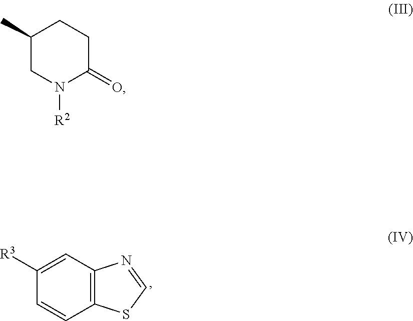

thereby producing a compound of formula (V):

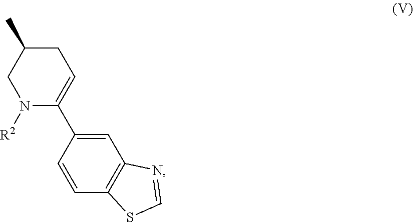

wherein R² is a nitrogen protecting group; and

R³ is a boronic acid or a boronic ester.

Embodiment 321. The process of embodiment 320, wherein the cross-coupling the compound of formula (III) with the compound of formula (IV) comprises:

(a) converting the compound of formula (III):

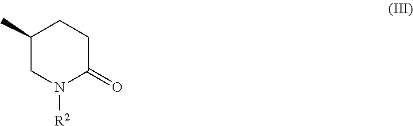

to a compound of formula (III-b):

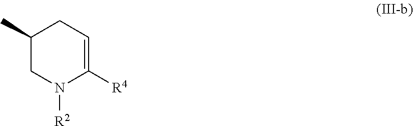

by contacting the compound of formula (III) with a sulfonylating/dehydrating agent, thereby providing the compound of formula (III-b),

wherein R² is a nitrogen protecting group as defined herein and R⁴ is an alkyl, haloalkyl or aryl sulfonate (e.g., methanesulfonate (—OS(═O)₂CH₃, trifluoromethanesulfonate (—OS(═O)₂CF₃), phenyl sulfonate (—OS(═O)₂Ph), toluenesulfonate (—OS(═O)₂C₆H₄—CH₃); and

(b) contacting the compound of formula (III-b) with a compound of formula (IV):

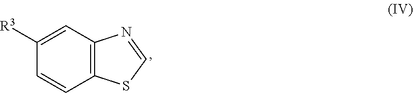

thereby producing a compound of formula (V):

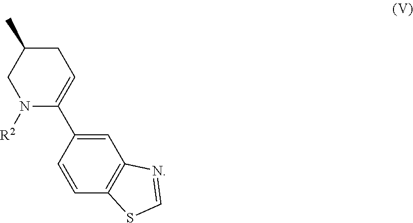

Embodiment 322. The process of any one of embodiments 318-321, wherein the process further comprises removing the nitrogen protective group from the compound of formula (V):

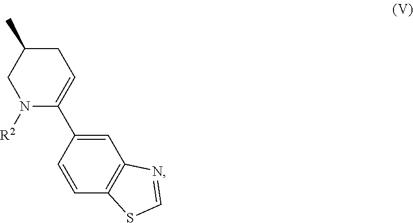

thereby forming a compound of formula (V-a):

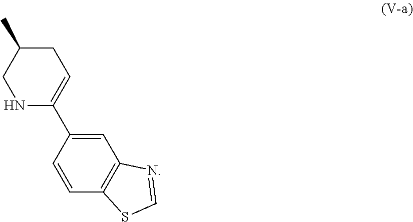 [SMILES: C[C@H]1CC=C(c2ccc3scnc3c2)NC1]

Embodiment 323. The process of any one of embodiments 318-322, wherein the process further comprises:

reducing the compound of formula (V-a):

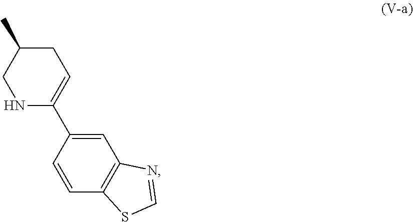 [SMILES: C[C@H]1CC=C(c2ccc3scnc3c2)NC1]

thereby producing a compound of formula (VI):

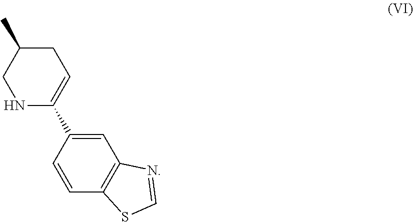

Embodiment 324. The process of any one of embodiments 318 to 323, wherein the process further comprises:

coupling the compound of formula (VI) with a compound of formula (VII):

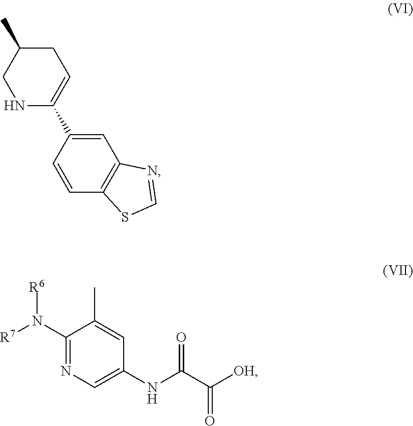

thereby producing a compound of formula (I-a):

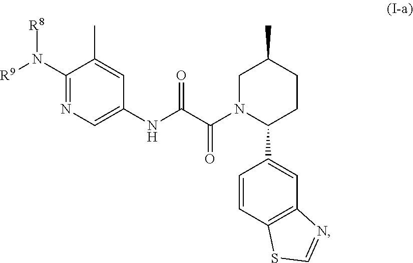

wherein each of R⁶, R⁷, R⁸, and R⁹ is, independently, H or a nitrogen protecting group.

Embodiment 325. The process of any one of embodiments 318 to 324, wherein, if R⁸, R⁹ or both R⁸ and R⁹ are a nitrogen protecting group, the process further comprises a deprotection step to remove the nitrogen protecting group from the compound of formula (I-a), thereby producing the compound of formula (I) or a salt thereof.
Embodiment 326. The process of any one of embodiments 318 to 325, wherein the process further comprises producing a crystalline form of the compound of formula (I) by subjecting a solution of the compound of formula (I) to conditions that result in crystallization of the compound of formula (I).
Embodiment 327. A process for preparing N-(6-amino-5-methylpyridin-3-yl)-2-((2R,5S)-2-(benzo[d]thiazol-5-yl)-5-methylpiperidin-1-yl)-2-oxoacetamide (a compound of formula (I)) or a salt thereof:

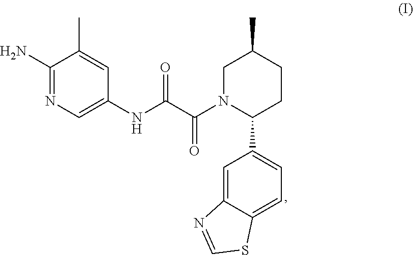 [SMILES: Cc1cc(NC(=O)C(=O)N2C[C@@H](C)CC[C@@H]2c2ccc3scnc3c2)cnc1N]

comprising:

(a) hydrogenating a compound of formula (II):

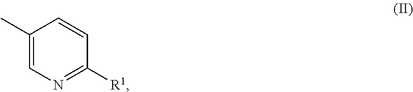

thereby producing a compound of formula (III-a):

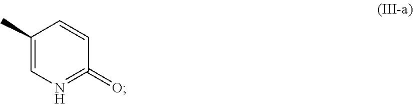

(b) protecting the nitrogen of the compound of formula (III-a) with a nitrogen protecting group, thereby providing a compound of formula (III)

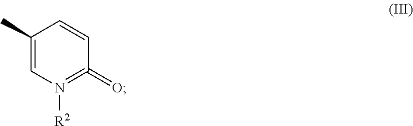

(c) cross-coupling the compound of formula (III) with a compound of formula (IV):

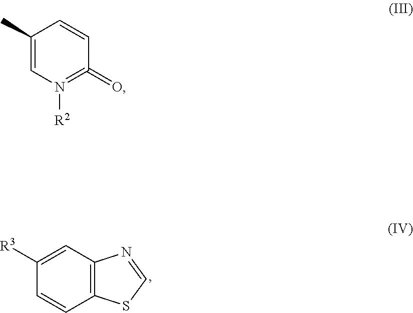

thereby producing a compound of formula (V):

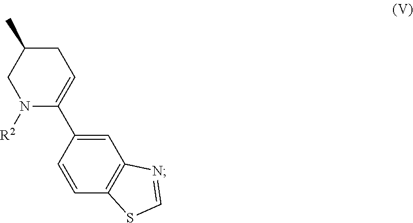

(d) removing nitrogen protecting group from the compound of formula (V), thereby producing a compound of formula (V-a);

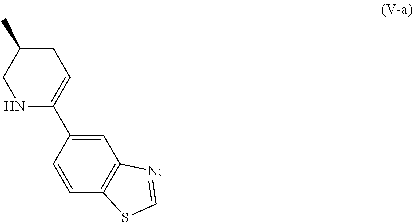 [SMILES: C[C@H]1CC=C(c2ccc3scnc3c2)NC1]

(e) reducing the compound of formula (V-a), thereby producing a compound of formula (VI):

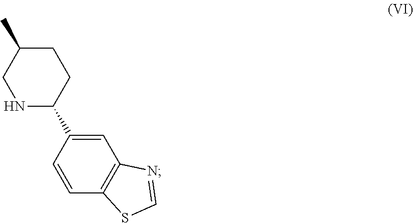 [SMILES: C[C@H]1CC[C@H](c2ccc3scnc3c2)NC1]

(f) coupling the compound of formula (VI) with a compound of formula (VII):

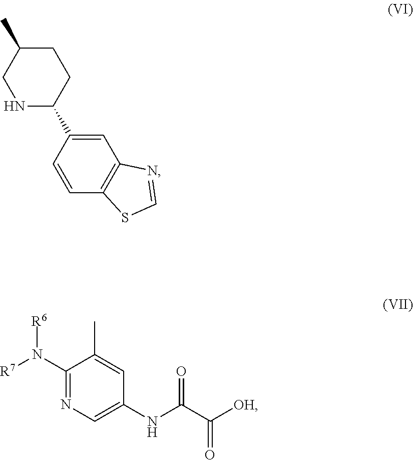

thereby producing a compound of formula (I-a):

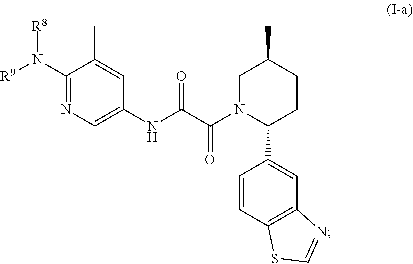

and

(g) optionally, if R⁸, R⁹, or both R⁸ and R⁹ are nitrogen protecting groups, deprotecting the compound of formula (I-a) by removing each nitrogen protecting group from the compound of formula (I-a), thereby providing the compound of formula (I) or a salt thereof;

wherein R¹ is a chiral auxiliary;

R² is a nitrogen protecting group;

each of R⁶, R⁷, R⁸, and R⁹ is, independently, H or a nitrogen protecting group; and

R³ is a boronic acid group or a boronic ester group.

Embodiment 328. The process of embodiment 327, wherein cross-coupling the compound of formula (III) with the compound of formula (IV) comprises:

(a) converting the compound of formula (III):

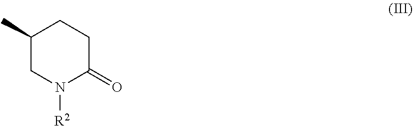

to a compound of formula (III-b):

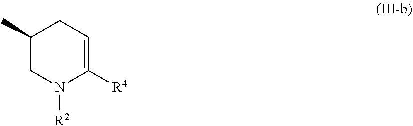

by contacting the compound of formula (III) with a sulfonylating/dehydrating agent, thereby providing the compound of formula (III-b),

wherein R² is a nitrogen protecting group as defined herein and R⁴ is an alkyl, haloalkyl or aryl sulfonate (e.g., methanesulfonate (—OS(═O)₂CH₃, trifluoromethanesulfonate (—OS(═O)₂CF₃), phenyl sulfonate (—OS(═O)₂Ph), toluenesulfonate (—OS(═O)₂C₆H₄—CH₃); and

(b) contacting the compound of formula (III-b) with a compound of formula (IV):

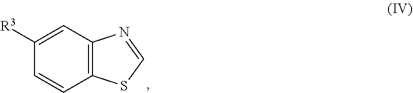

thereby producing a compound of formula (V):

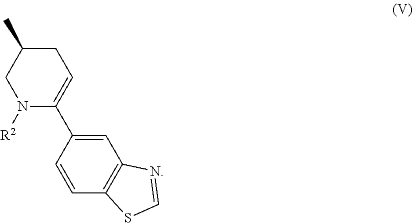

Embodiment 329. A process for preparing N-(6-amino-5-methylpyridin-3-yl)-2-((2R,5S)-2-(benzo[d]thiazol-5-yl)-5-methylpiperidin-1-yl)-2-oxoacetamide (a compound of formula (I)) or a salt thereof:

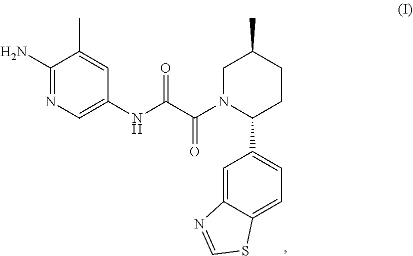 [SMILES: Cc1cc(NC(=O)C(=O)N2C[C@@H](C)CC[C@@H]2c2ccc3scnc3c2)cnc1N]

comprising:

coupling a compound of formula (VI) with a compound of formula (VII):

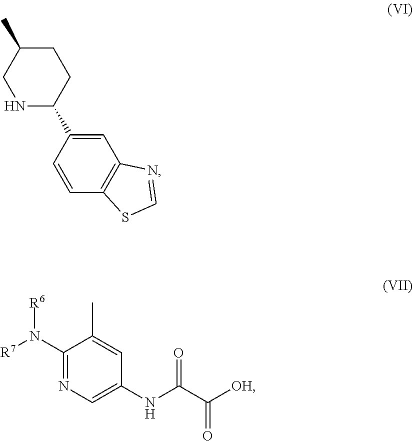

thereby producing a compound of formula (I-a):

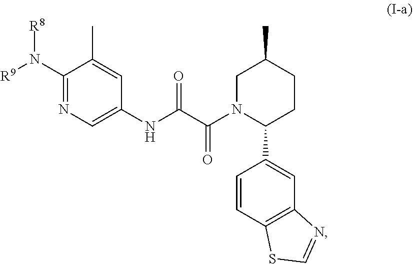

wherein each of R⁶, R⁷, R⁸, and R⁹ is, independently, H or a nitrogen protecting group; and

optionally, if R⁸, R⁹ or both R⁸ and R⁹ are nitrogen protecting groups, deprotecting the compound of formula (I-a) by removing each nitrogen protecting group from the compound of formula (I-a), thereby providing the compound of formula (I) or a salt thereof.

Embodiment 330. The process of embodiment 329, wherein the process further comprises:

reducing a compound of formula (V-a):

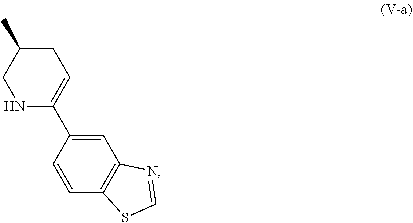 [SMILES: C[C@H]1CC=C(c2ccc3scnc3c2)NC1]

thereby producing the compound of formula (VI):

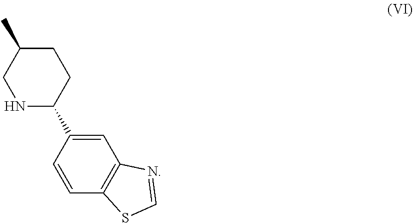 [SMILES: C[C@H]1CC[C@H](c2ccc3scnc3c2)NC1]

Embodiment 331. The process of embodiment 329 or 330, wherein the process further comprises removing the nitrogen protecting group from a compound of formula (V),

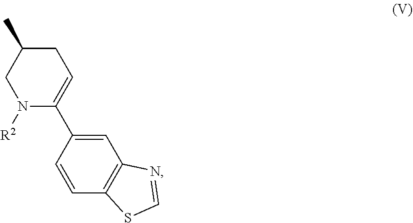

thereby producing the compound of formula (V-a):

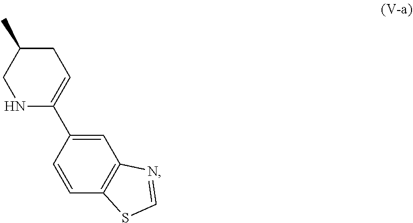

wherein R² is a nitrogen protecting group.

Embodiment 332. The process of any one of embodiments 329 to 331, wherein the process further comprises:

cross-coupling a compound of formula (III) with a compound of formula (IV):

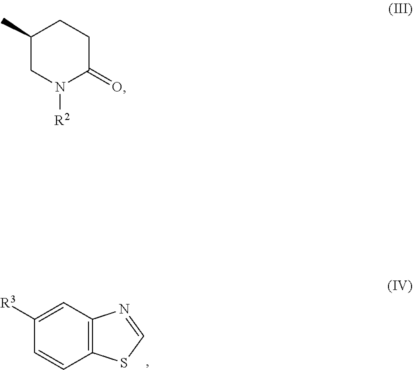

thereby producing the compound of formula (V):

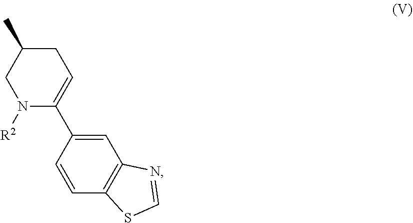

wherein R² is a nitrogen protecting group; and

R³ is a boronic acid group or a boronic ester group.

Embodiment 333. The process of embodiment 332, wherein cross-coupling the compound of formula (III) with the compound of formula (IV) comprises:

(a) converting a compound of formula (III):

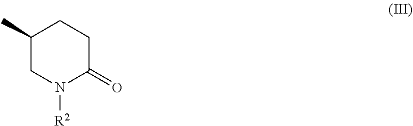

to a compound of formula (III-b):

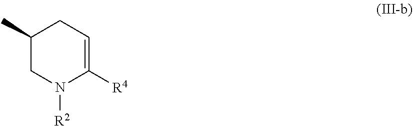

by contacting the compound of formula (III) with a sulfonylating/dehydrating agent, thereby providing the compound of formula (III-b),

wherein R² is a nitrogen protecting group as defined herein and R⁴ is an alkyl, haloalkyl or aryl sulfonate (e.g., methanesulfonate (—OS(═O)₂CH₃, trifluoromethanesulfonate (—OS(═O)₂CF₃), phenyl sulfonate (—OS(═O)₂Ph), toluenesulfonate (—OS(═O)₂C₆H₄—CH₃); and

(b) contacting the compound of formula (III-b) with a compound of formula (IV):

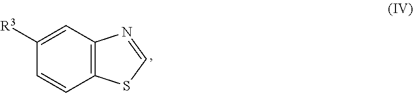

thereby producing the compound of formula (V):

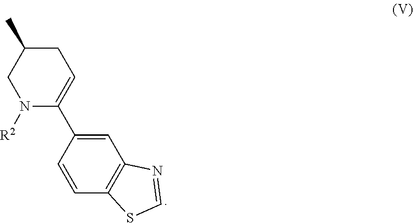

Embodiment 334. The process of any one of embodiments 329 to 333, wherein the process further comprises:

protecting the nitrogen group of a compound of formula (III-a)

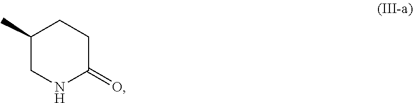

with a nitrogen protecting group, thereby producing the compound of formula (III):

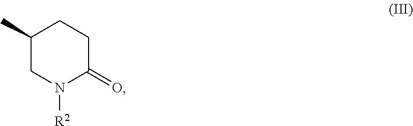

wherein R² is a nitrogen protecting group.

Embodiment 335. The process of any one of embodiments 329 to 334, wherein the process further comprises:

hydrogenating a compound of formula (II):

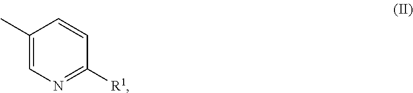

thereby producing the compound of formula (III-a):

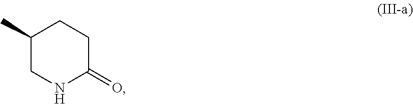

wherein R¹ is a chiral auxiliary.

Embodiment 336. The process of any one of embodiments 318-328 and 335, wherein the chiral auxiliary is an optionally substituted oxazolidinone.
Embodiment 337. The process of embodiment 336, wherein the chiral auxiliary is

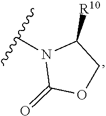

wherein R¹⁰ is C₁₋₆ alkyl, benzyl (Bn), or phenyl (Ph).
Embodiment 338. The process of embodiment 337, wherein the chiral auxiliary is

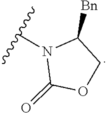 [SMILES: *N1C(=O)OC[C@@H]1Cc1ccccc1]

Embodiment 339. The process of any one of embodiments 319-328 and 331-338, wherein R² is a carbamate group.
Embodiment 340. The process of any one of embodiments 319-328 and 331-338, wherein R² is

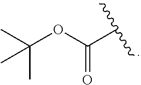 [SMILES: *C(=O)OC(C)(C)C]

Embodiment 341. The process of any one of embodiments 318-328 and 335-340, wherein hydrogenating the compound of formula (II) comprises contacting the compound of formula (II) with a first catalyst.
Embodiment 342. The process of any one of embodiments 318-328 and 335-340, wherein hydrogenating the compound of formula (II) comprises contacting the compound of formula (II) with less than about 10 mol % of a first catalyst.
Embodiment 343. The process of any one of embodiments 318-328 and 335-340, wherein hydrogenating the compound of formula (II) comprises contacting the compound of formula (II) with less than about 5 mol % of a first catalyst.
Embodiment 344. The process of any one of embodiments 318-328 and 335-340, wherein hydrogenating the compound of formula (II) comprises contacting the compound of formula (II) with less than about 4 mol % of a first catalyst.
Embodiment 345. The process of any one of embodiments 318-328 and 335-340, wherein hydrogenating the compound of formula (II) comprises contacting the compound of formula (II) with less than about 3 mol % of a first catalyst.
Embodiment 346. The process of any one of embodiments 318-328 and 335-340, wherein hydrogenating the compound of formula (II) comprises contacting the compound of formula (II) with less than about 2 mol % of a first catalyst.
Embodiment 347. The process of any one of embodiments 318-328 and 335-340, wherein hydrogenating the compound of formula (II) comprises contacting the compound of formula (II) with less than about 1.75 mol % of a first catalyst.
Embodiment 348. The process of any one of embodiments 318-328 and 335-340, wherein hydrogenating the compound of formula (II) comprises contacting the compound of formula (II) with about 1 mol % to about 2 mol % of a first catalyst.
Embodiment 349. The process of any one of embodiments 318-328 and 335-340, wherein hydrogenating the compound of formula (II) comprises contacting the compound of formula (II) with about 1.5 mol % to about 2 mol % of a first catalyst.
Embodiment 350. The process of any one of embodiments 318-328 and 335-340, wherein hydrogenating the compound of formula (II) comprises contacting the compound of formula (II) with about 1.7 mol % of a first catalyst.
Embodiment 351. The process of any one of embodiments 341-350, wherein the first catalyst is a palladium catalyst.
Embodiment 352. The process of any one of embodiments 341-350, wherein the first catalyst is a palladium (0) catalyst.
Embodiment 353. The process of any one of embodiments 341-350, wherein the first catalyst is a palladium on carbon.
Embodiment 354. The process of any one of embodiments 341-350, wherein the first catalyst is about 5% by weight palladium on carbon.
Embodiment 355. The process of any one of embodiments 341-350, wherein the first catalyst is about 10% by weight palladium on carbon.
Embodiment 356. The process of any one of embodiments 318-328 and 335-355, wherein hydrogenating the compound of formula (II) comprises the use of a flow system.
Embodiment 357. The process of any one of embodiments 318-328 and 335-355, wherein hydrogenating the compound of formula (II) comprises the use of a continuous flow system.
Embodiment 358. The process of any one of embodiments 318-328 and 335-355, wherein hydrogenating the compound of formula (II) comprises the use of a micropacked bed reactor.
Embodiment 359. The process of any one of embodiments 318-328 and 335-355 wherein hydrogenating the compound of formula (II) is performed under flow hydrogenation conditions.
Embodiment 360. The process of any one of embodiments 318-328 and 335-355, wherein hydrogenating the compound of formula (II) is performed under continuous flow hydrogenation conditions.
Embodiment 361. The process of any one of embodiments 318-328 and 335-360, wherein hydrogenating the compound of formula (II) comprises contacting the compound of formula (II) with hydrogen gas.
Embodiment 362. The process of any one of embodiments 318-328 and 335-360, wherein hydrogenating the compound of formula (II) comprises contacting the compound of formula (II) with hydrogen gas at a pressure of at least about 1.0 megapascal (MPa).
Embodiment 363. The process of any one of embodiments 318-328 and 335-360, wherein hydrogenating the compound of formula (II) comprises contacting the compound of formula (II) with hydrogen gas at a pressure of at least about 2.0 megapascal (MPa).
Embodiment 364. The process of any one of embodiments 318-328 and 335-360, wherein hydrogenating the compound of formula (II) comprises contacting the compound of formula (II) with hydrogen gas at a pressure of at least about 3.0 megapascal (MPa).
Embodiment 365. The process of any one of embodiments 318-328 and 335-360, wherein hydrogenating the compound of formula (II) comprises contacting the compound of formula (II) with hydrogen gas at a pressure of between about 2.0 megapascal (MPa) and 4.0 megapascal (MPa).
Embodiment 366. The process of any one of embodiments 318-328 and 335-360, wherein hydrogenating the compound of formula (II) comprises contacting the compound of formula (II) with hydrogen gas at a pressure of about 3.0 megapascal (MPa).
Embodiment 367. The process of any one of embodiments 320-328 and 332 to 366, wherein R³ is a boronic ester group.
Embodiment 368. The process of any one of embodiments 320-328 and 332 to 366, wherein R³ is

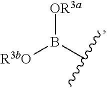

wherein each of R^{3a} and R^{3b} is, independently, H or C₁₋₆ alkyl, wherein R^{3a} and R^{3b} are optionally joined together with their intervening atoms to form a 5-10 membered ring that is optionally substituted with 0, 1, 2, 3, 4, 5, or 6 instances of R^{3c}, wherein each R^{3c} is, independently, C₁₋₆ alkyl.
Embodiment 369. The process of embodiment 368, wherein each of R^{3a} and R^{3b} is, independently, C₁₋₆ alkyl, wherein R^{3a} and R^{3b} are joined together with their intervening atoms to form a 5-10 membered ring that is optionally substituted with 0, 1, 2, 3, 4, 5, or 6 instances of R^{3c}, wherein each R^{3c} is, independently, C₁₋₆ alkyl.
Embodiment 370. The process of embodiment 368, wherein each of R^{3a} and R^{3b} is, independently, C₁₋₆ alkyl, wherein R^{3a} and R^{3b} are joined together with their intervening atoms to form a 5-6 membered ring that is optionally substituted with 0, 1, 2, 3, 4, 5, or 6 instances of R^{3c}, wherein each R^{3c} is -Me.
Embodiment 371. The process of embodiment 368, wherein R³ is

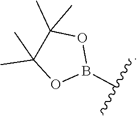 [SMILES: *B1OC(C)(C)C(C)(C)O1]

Embodiment 372. The process of any one of embodiments 321, 328, and 333-371, wherein contacting the compound of formula (III) with the sulfonylating/dehydrating agent takes place in the presence of a first base.
Embodiment 373. The process of any one of embodiments 321, 328 and 333-372, wherein the sulfonylating/dehydrating agent is a sulfonimide.
Embodiment 374. The process of any one of embodiments 321, 328 and 333-372, wherein the sulfonylating/dehydrating agent is 1,1,1-trifluoro-N-phenyl-N-(trifluoromethanesulfonyl)methanesulfonamide (PHNTf₂).
Embodiment 375. The process of any one of embodiments 372 to 374, wherein the first base is an inorganic base.
Embodiment 376. The process of any one of embodiments 372 to 374, wherein the first base is an organic base.
Embodiment 377. The process of any one of embodiments 372 to 374, wherein the first base is a lithium base.
Embodiment 378. The process of any one of embodiments 372 to 374, wherein the first base is an amine base.
Embodiment 379. The process of any one of embodiments 372 to 374, wherein the first base is lithium bis(trimethylsilyl)amide (LiHMDS).
Embodiment 380. The process of any one of embodiments 321, 328, and 333-379, wherein contacting the compound of formula (III-b) with the compound of formula (IV) takes place in the presence of a second catalyst.
Embodiment 381. The process of any one of embodiments 321, 328, and 333-379, wherein contacting the compound of formula (III-b) with the compound of formula (IV) takes place in the presence of less than about 10 mol % of a second catalyst.
Embodiment 382. The process of any one of embodiments 321, 328, and 333-379, wherein contacting the compound of formula (III-b) with the compound of formula (IV) takes place in the presence of less than about 5 mol % of a second catalyst.
Embodiment 383. The process of any one of embodiments 321, 328, and 333-379, wherein contacting the compound of formula (III-b) with the compound of formula (IV) takes place in the presence of less than about 4 mol % of a second catalyst.
Embodiment 384. The process of any one of embodiments 321, 328, and 333-379, wherein contacting the compound of formula (III-b) with the compound of formula (IV) takes place in the presence of about 1 mol % to about 5 mol % of a second catalyst.
Embodiment 385. The process of any one of embodiments 321, 328, and 333-379, wherein contacting the compound of formula (III-b) with the compound of formula (IV) takes place in the presence of about 1 mol % to about 4 mol % of a second catalyst.
Embodiment 386. The process of any one of embodiments 321, 328, and 333-379, wherein contacting the compound of formula (III-b) with the compound of formula (IV) takes place in the presence of about 2 mol % to about 4 mol % of a second catalyst.
Embodiment 387. The process of any one of embodiments 321, 328, and 333-379, wherein contacting the compound of formula (III-b) with the compound of formula (IV) takes place in the presence of about 3 mol % of a second catalyst.
Embodiment 388. The process of any one of embodiments 380 to 387, wherein the second catalyst is a palladium catalyst.
Embodiment 389. The process of any one of embodiments 380 to 387, wherein the second catalyst is a palladium (II) catalyst.
Embodiment 390. The process of any one of embodiments 380 to 387, wherein the second catalyst is bis(triphenylphosphine)palladium(II) dichloride.
Embodiment 391. The process of any one of embodiments 321, 328 and 333-390, wherein contacting the compound of formula (III-b) with the compound of formula (IV) takes place in the presence of a second base.
Embodiment 392. The process of embodiment 391, wherein the second base is an inorganic base.
Embodiment 393. The process of embodiment 391, wherein the second base is tripotassium phosphate (K₃PO₄).
Embodiment 394. The process any one of embodiments 322-328 and 331-393, wherein removing the nitrogen protecting group of the compound of formula (V) comprises contacting the compound of formula (V) with a first acid.
Embodiment 395. The process of embodiment 394, wherein the first acid is an inorganic acid.
Embodiment 396. The process of embodiment 394, wherein the first acid is hydrochloric acid (HCl) or phosphoric acid (H₃PO₄).
Embodiment 397. The process of embodiment 394, wherein the first acid is hydrochloric acid (HCl).
Embodiment 398. The process of embodiment 394, wherein the first acid is an organic acid.
Embodiment 399. The process of embodiment 394, wherein the first acid is trifluoromethanesulfonic acid (TfOH), trifluoroacetic acid (TFA), or p-toluenesulfonic acid (PTSA).
Embodiment 400. The process of any one of embodiments 323-328 and 330 to 399, wherein reducing the compound of formula (V-a) comprises contacting the compound of formula (V-a) with a reducing agent.
Embodiment 401. The process of embodiment 400, wherein the reducing agent is a hydride reducing agent.
Embodiment 402. The process of embodiment 400, wherein the reducing agent is a borohydride reducing agent.
Embodiment 403. The process of embodiment 400, wherein the reducing agent is sodium borohydride (NaBH₄).
Embodiment 404. The process of any one of embodiments 323-328 and 330 to 403, wherein reducing the compound of formula (V-a) comprises contacting the compound of formula (V-a) with a first solvent at a temperature below about 0° C.
Embodiment 405. The process of embodiment 404, wherein the first solvent is a protic solvent.
Embodiment 406. The process of embodiment 404, wherein the first solvent is methanol.
Embodiment 407. The process of any one of embodiments 324-406, wherein R⁶ is a nitrogen protecting group and R⁷ is a nitrogen protecting group.
Embodiment 408. The process of any one of embodiments 324-406, wherein R⁶ is a carbamate group and R⁷ is a carbamate group.

### Embodiment 409.

The process of any one of embodiments 324-406, wherein R⁶ is

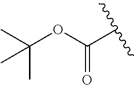 [SMILES: *C(=O)OC(C)(C)C]

### and R⁷ is

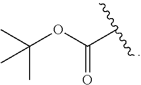 [SMILES: *C(=O)OC(C)(C)C]

Embodiment 410. The process of any one of embodiments 324-409, wherein coupling the compound of formula (VI) with the compound of formula (VII) comprises contacting the compound of formula (VI) with the compound of formula (VII) having a molar ratio of about 1.5:1 to about 1:1.5.
Embodiment 411. The process of any one of embodiments 324-409, wherein coupling the compound of formula (VI) with the compound of formula (VII) comprises contacting the compound of formula (VI) with the compound of formula (VII) having a molar ratio of about 1:1 to about 1:1.4.
Embodiment 412. The process of any one of embodiments 324-409, wherein coupling the compound of formula (VI) with the compound of formula (VII) comprises contacting the compound of formula (VI) with the compound of formula (VII) having a molar ratio of about 1:1.1 to about 1:1.4.
Embodiment 413. The process of any one of embodiments 324-409, wherein coupling the compound of formula (VI) with the compound of formula (VII) comprises contacting the compound of formula (VI) with the compound of formula (VII) having a molar ratio of about 1:1.3.
Embodiment 414. The process of any one of embodiments 324-413, wherein coupling the compound of formula (VI) with the compound of formula (VII) comprises contacting the compound of formula (VI) with the compound of formula (VII) in the presence of a coupling reagent.
Embodiment 415. The process of embodiment 414, wherein the coupling reagent is an anhydride coupling reagent; a triazole-based coupling reagent; a carbodiimide coupling reagent; an imidazolium coupling reagent; a phosphonium salt coupling reagent; or a pyridinium salt coupling reagent.
Embodiment 416. The process of embodiment 414, wherein the coupling reagent is a triazole-based coupling reagent.
Embodiment 417. The process of embodiment 414, wherein the coupling reagent is 1-[bis(dimethylamino)methylene]-1H-1,2,3-triazolo[4,5-b]pyridinium 3-oxide hexafluorophosphate (HATU); N,N,N′,N′-tetramethyl-O-(1H-benzotriazol-1-yl)uronium hexafluorophosphate (HBTU); O-(1H-6-chlorobenzotriazole-1-yl)-1,1,3,3-tetramethyluronium hexafluorophosphate (HCTU); or 2-(1H-benzotriazole-1-yl)-1,1,3,3-tetramethylaminium tetrafluoroborate (TBTU).
Embodiment 418. The process of embodiment 414, wherein the coupling reagent is 1-[bis(dimethylamino)methylene]-1H-1,2,3-triazolo[4,5-b]pyridinium 3-oxide hexafluorophosphate (HATU) or 2-(1H-benzotriazole-1-yl)-1,1,3,3-tetramethylaminium tetrafluoroborate (TBTU).
Embodiment 419. The process of embodiment 414, wherein the coupling reagent is 2-(1H-benzotriazole-1-yl)-1,1,3,3-tetramethylaminium tetrafluoroborate (TBTU).
Embodiment 420. The process of any one of embodiments 414 to 419, wherein the compound of formula (VI) and the coupling reagent have a molar ratio of about 1.5:1 to about 1:1.5.
Embodiment 421. The process of any one of embodiments 414 to 419, wherein the compound of formula (VI) and the coupling reagent have a molar ratio of about 1:1 to about 1:1.5.
Embodiment 422. The process of any one of embodiments 414 to 419, wherein the compound of formula (VI) and the coupling reagent have a molar ratio of about 1:1.1 to about 1:1.4.
Embodiment 423. The process of any one of embodiments 414 to 419, wherein the compound of formula (VI) and the coupling reagent have a molar ratio of about 1:1.2 to about 1:1.4.
Embodiment 424. The process of any one of embodiments 414 to 419, wherein the compound of formula (VI) and the coupling reagent have a molar ratio of about 1:1.3.
Embodiment 425. The process of any one of embodiments 324-424, wherein coupling the compound of formula (VI) with the compound of formula (VII) comprises contacting the compound of formula (VI) with the compound of formula (VII) in the presence of a third base.
Embodiment 426. The process of embodiment 425, wherein the third base is an organic base.
Embodiment 427. The process of embodiment 426, wherein the third base is an amine base.
Embodiment 428. The process of embodiment 427, wherein the third base is triethylamine (TEA), N,N-diisopropylethylamine (DIPEA), or pyridine.
Embodiment 429. The process of embodiment 427, wherein the third base is triethylamine (TEA) or N,N-diisopropylethylamine (DIPEA).
Embodiment 430. The process of embodiment 427, wherein the third base is N,N-diisopropylethylamine (DIPEA).
Embodiment 431. The process of any one of embodiments 324-430, wherein R⁸ is a nitrogen protecting group and R⁹ is a nitrogen protecting group.
Embodiment 432. The process of any one of embodiments 324-430, wherein R⁸ is a carbamate group and R⁹ is a carbamate group.
Embodiment 433. The process of any one of embodiments 324-430, wherein R⁸ is

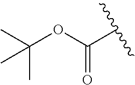 [SMILES: *C(=O)OC(C)(C)C]

### and R⁹ is

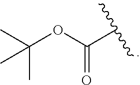 [SMILES: *C(=O)OC(C)(C)C]

Embodiment 434. The process any one of embodiments 425-433, wherein removing each nitrogen protecting group from the compound of formula (I-a) comprises contacting the compound of formula (I-a) with a second acid.
Embodiment 435. The process of embodiment 434, wherein removing each nitrogen protecting group from the compound of formula (I-a) comprises contacting the compound of formula (I-a) with a second acid at a temperature of about 0° C. to about 100° C.
Embodiment 446. The process of embodiment 434, wherein removing each nitrogen protecting group from the compound of formula (I-a) comprises contacting the compound of formula (I-a) with a second acid at a temperature of about 15° C. to about 65° C.
Embodiment 437. The process of embodiment 434, wherein removing each nitrogen protecting group from the compound of formula (I-a) comprises contacting the compound of formula (I-a) with a second acid at a temperature of about 20° C. to about 50° C.
Embodiment 438. The process of embodiment 434, wherein removing each nitrogen protecting group from the compound of formula (I-a) comprises contacting the compound of formula (I-a) with a second acid at a temperature of about 25° C. to about 45° C.
Embodiment 439. The process of embodiment 434, wherein removing each nitrogen protecting group from the compound of formula (I-a) comprises contacting the compound of formula (I-a) with a second acid at a temperature of about 35° C. to about 55° C.
Embodiment 440. The process of embodiment 434, wherein removing each nitrogen protecting group from the compound of formula (I-a) comprises contacting the compound of formula (I-a) with a second acid at a temperature of about 45° C.
Embodiment 441. The process of embodiment 434, the compound of formula (I-a) and the second acid have a molar ratio of about 1:1 to about 1:10.
Embodiment 442. The process of any one of embodiments 434 to 441, wherein the compound of formula (I-a) and the second acid have a molar ratio of about 1:2 to about 1:7.
Embodiment 443. The process of any one of embodiments 434 to 441, wherein the compound of formula (I-a) and the second acid have a molar ratio of about 1:3 to about 1:6.
Embodiment 444. The process of any one of embodiments 434 to 441, wherein the compound of formula (I-a) and the second acid have a molar ratio of about 1:3 to about 1:5.
Embodiment 445. The process of any one of embodiments 434 to 441, wherein the compound of formula (I-a) and the second acid have a molar ratio of about 1:4.
Embodiment 446. The process of any one of embodiments 434 to 445, wherein the second acid is an inorganic acid.
Embodiment 447. The process of embodiment 446, wherein the inorganic acid is hydrochloric acid (HCl) or phosphoric acid (H₃PO₄).
Embodiment 448. The process of any one of embodiments 434 to 445, wherein the second acid is an organic acid.
Embodiment 449. The process of embodiment 448, wherein the organic acid is trifluoromethanesulfonic acid (TfOH), trifluoroacetic acid (TFA), or p-toluenesulfonic acid (PTSA).
Embodiment 450. The process of embodiment 448, wherein the organic acid is trifluoromethanesulfonic acid (TfOH).
Embodiment 451. The process of any one of embodiments 318 to 450, wherein the compound of formula (I) is not purified by chromatography.
Embodiment 452. The process of any one of embodiments 318 to 450, wherein the the process does not comprise purification by chromatography.
Embodiment 453. The process of any one of embodiments 318 to 452, wherein the process further comprises converting a salt of the compound of formula (I) into the free base of the compound of formula (I).
Embodiment 454. The process of embodiment 453, wherein converting the salt of the compound of formula (I) (e.g., the trifluoromethanesulfonate salt) into the free base of the compound of formula (I) comprises contacting the salt of the compound of formula (I) with a fifth base in a in a tenth solvent.
Embodiment 455. The process of embodiment 454, wherein the fifth base is an inorganic base.
Embodiment 456. The process of embodiment 454, wherein the fifth base is a carbonate base.
Embodiment 457. The process of embodiment 454, wherein the fifth base is sodium carbonate.
Embodiment 458. The process of any one of embodiments 454 to 457, wherein the tenth solvent is a mixture of water and an organic solvent.
Embodiment 459. The process of any one of embodiments 454 to 457, wherein the tenth solvent is a mixture of water, ethanol and tetrahydrofuran.
Embodiment 460. The process of any one of embodiments 318 to 459, wherein the process further comprises producing a crystalline form of the compound of formula (I) (e.g., a crystalline form of the free base compound of formula (I).
Embodiment 461. The process of embodiment 460, wherein producing a crystalline form of the compound of formula (I) comprises subjecting a solution of the compound of formula (I) to conditions that result in crystallization of the compound of formula (I).
Embodiment 462. The process of embodiment 460 or 461, wherein producing a crystalline form of the compound of formula (I) comprises:
dissolving the compound of formula (I) in a first solvent; and
partially evaporating the first solvent to generate a first suspension.
   Embodiment 463. The process of any one of embodiments 460 to 462, wherein the process further comprises seeding a solution resulting from dissolving the compound of formula (I) in the first solvent with a small amount of crystalline compound of formula (I) (e.g., less than about 5% of the amount of compound of formula (I) present in the solution).
   Embodiment 464. The process of any one of embodiments 460 to 463, wherein the first solvent is a protic solvent.
   Embodiment 465. The process of any one of embodiments 460 to 463, wherein the first solvent is a polar solvent.
   Embodiment 466. The process of any one of embodiments 460 to 463, wherein the first solvent is MeCN, methanol, ethanol, isopropyl alcohol, n-propanol, n-BuOH, water, or a mixture thereof.
   Embodiment 467. The process of any one of embodiments 460 to 463, wherein the first solvent is MeCN, ethanol, methanol, water, or a mixture thereof.
   Embodiment 468. The process of any one of embodiments 460 to 463, wherein the first solvent is a mixture of water and methanol (e.g., a 9:1 v/v mixture).
   Embodiment 469. The process of any one of embodiments 460 to 468, wherein the process further comprises adding water to a mixture obtained after partially evaporating the first solvent.
   Embodiment 470. The process of any one of embodiments 460 to 469, wherein the process further comprises stirring the first suspension for at least 1 hr.
   Embodiment 471. The process of any one of embodiments 460 to 470, further comprising collecting a crude crystalline form of the compound of formula (I) by filtration of the first suspension (e.g., filtration by centrifugation).
   Embodiment 472. The process of any one of embodiments 460 to 471, further comprising:
slurrying the crude crystalline form of the compound of formula (I) in a second solvent; and
filtering by centrifugation.
   Embodiment 473. The process of embodiment 472, wherein the second solvent is a protic solvent.
   Embodiment 474. The process of embodiment 472, wherein the second solvent is a polar solvent.
   Embodiment 475. The process of embodiment 472, wherein the second solvent is MeCN, methanol, ethanol, isopropyl alcohol, n-propanol, n-BuOH, water, or a mixture thereof.
   Embodiment 476. The process of embodiment 472, wherein the second solvent is MeCN, ethanol, methanol, water, or a mixture thereof.
   Embodiment 477. The process of embodiment 472, wherein the second solvent is a mixture of methanol and water.

### Examples

In order that the disclosure described herein may be more fully understood, the following examples are set forth. The compound of formula (I) may be prepared a variety of ways (e.g., using various reactions, reagents, and/or conditions). The synthetic examples described in this application are offered to illustrate the compounds, pharmaceutical compositions, and methods provided herein and are not to be construed in any way as limiting their scope.

In order that the invention described herein may be more fully understood, the following examples are set forth. The examples described in this application are offered to illustrate the crystalline solid forms provided herein and are not to be construed in any way as limiting their scope.

### Abbreviations and Definitions

API active pharmaceutical ingredient
AUC area under the curve
BA bioavailability
Boc₂O di-tert-butyl dicarbonate
Cₘₐₓ maximum observed plasma concentration
DCM dichloromethane
DMAP 4-dimethylaminopyridine
DP drug product
DS drug substance
DSC differential scanning calorimetry
dm/dt change in mass per unit of time
DVS dynamic vapor sorption
EtOH ethanol
FaSSIF fasted state simulated intestinal fluid
FeSSIF fed state simulated intestinal fluid
GMP Good Manufacturing Practice
HATU 1-[bis(dimethylamino)methylene]-1H-1,2,3-triazolo[4,5-b]pyridinium 3-oxide hexafluorophosphate
HPLC high performance liquid chromatography
IPA isopropanol
IPAc isopropyl acetate
LiHMDS lithium bis(trimethylsilyl)amide
LOD limit of detection
max maximum
min minimum
MCC microcrystalline cellulose
McOH methanol
MeCN acetonitrile
NA not applicable
NMP N-methyl-2-pyrrolidone
PhNTf₂ bis(trifluoromethanesulfonyl)aniline
RSD relative standard deviation
RH relative humidity
RT room temperature or real time
sccm standard cubic centimeter per minute
SD Standard Deviation
TGA thermogravimetric analysis
THE tetrahydrofuran
USP United States Pharmacopoeia
w/w weight-to-weight
XRPD X-ray powder diffraction

### Example 1. The synthesis of N-(6-amino-5-methylpyridin-3-yl)-2-((2R,5S)-2-(benzo[d]thiazol-5-yl)-5-methylpiperidin-1-yl)-2-oxoacetamide (Compound (I)) and N-(6-amino-5-methylpyridin-3-yl)-2-((2S,5R)-2-(benzo [d]thiazol-5-yl)-5-methylpiperidin-1-yl)-2-oxoacetamide (Compound (Ia))

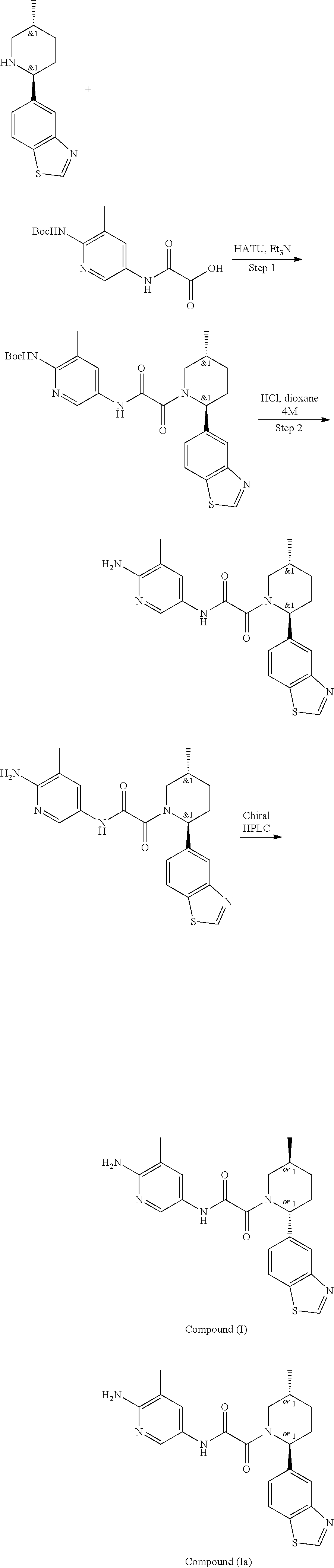

### Step 1: Synthesis of tert-butyl N-[5-[[2-[2-(1,3-benzothiazol-5-yl)-5-methyl-1 piperidyl]-2-oxo-acetyl]amino]-3-methyl-2 pyridyl]carbamate

HATU (490.95 mg, 1.29 mmol) was added portion wise at r.t. to a suspension of 2-[[6-(tert-butoxycarbonylamino)-5-methyl-3-pyridyl]amino]-2-oxo-acetic acid (381.28 mg, 1.29 mmol), 5-(5-methyl-2-piperidyl)-1,3-benzothiazole (300 mg, 1.29 mmol) and TEA (783.93 mg, 7.75 mmol, 1.08 mL) in DMF (10 mL). The clear solution was stirred at 25° C. for 18 hr and the solvents were evaporated in vacuo. The residue was dissolved in EtOAc (100 mL), washed with water (3×50 mL) and evaporated in vacuo to give tert-butyl N-[5-[[2-[2-(1,3-benzothiazol-5-yl)-5-methyl-1-piperidyl]-2-oxo-acetyl]amino]-3-methyl-2-pyridyl]carbamate (700 mg, crude).

¹H NMR (DMSO-d6, 400 MHz): δ 1.01 (d, 3H), 1.39 (m, 13H), 2.09 (m, 8H), 5.71 (m, 1H), 7.43 (m, 2H), 8.12 (m, 1H), 8.43 (s, 1H), 9.03 (s, 1H), 9.38 (m, 1H), 11.00 (s, 1H).

LCMS(ESI): [M+H]⁺ m/z: calcd 509.2; found 510.2; Rt=1.319 min.

### Step 2: Synthesis of N-(6-amino-5-methyl-3 pyridyl)-2-[2-(1,3-benzothiazol-5-yl)-5-methyl-1-piperidyl]-2-oxo-acetamide

Hydrogen chloride solution 4.0M in dioxane (3.58 g, 13.74 mmol, 3.41 mL, 14% purity) was carefully added at r.t. to a solution of tert-butyl N-[5-[[2-[2-(1,3-benzothiazol-5-yl)-5-methyl-1-piperidyl]-2-oxo-acetyl]amino]-3-methyl-2-pyridyl]carbamate (700 mg, 1.37 mmol) in DCM (10 mL). The reaction mixture was then stirred for 12 hr at r.t. and the solvents were evaporated in vacuo. The residue was subjected to RP-HPLC (column: YMC Triart C18 100×20 mm, Sum; 40-40-90% 0-1-5 min 0.1% NH₃-methanol as mobile phase) to give N-(6-amino-5-methyl-3-pyridyl)-2-[2-(1,3-benzothiazol-5-yl)-5-methyl-1-piperidyl]-2-oxo-acetamide (331 mg, 808.30 μmol, 58.85% yield).

LCMS(ESI): [M+H]⁺ m/z: calcd 409.2; found 410.2; Rt=2.176 min.

### Step 3: Synthesis of N-(6-amino-5-methylpyridin-3-yl)-2-((2R,5S)-2-(benzo[d]thiazol-5-yl)-5-methylpiperidin-1-yl)-2-oxoacetamide (Compound (I)) and N-(6-amino-5-methylpyridin-3-yl)-2-((2S,5R)-2-(benzo[d]thiazol-5-yl)-5-methylpiperidin-1-yl)-2-oxoacetamide (Compound (Ia))

The enantiomers were separated by chiral HPLC (column: IC II, Hexane-IPA-MeOH, 50-25-25, 12 ml/min as mobile phase) to give the two individual enantiomers Compound (Ia)N-(6-amino-5-methylpyridin-3-yl)-2-((2 S, 5R)-2-(benzo[d]thiazol-5-yl)-5-methylpiperidin-1-yl)-2-oxoacetamide (161 mg, 393.16 μmol, 97.28% yield) RetTime=32.4 min, [α]21D=−176.7°(c=0.1 g/100 mL, EtOH) and Compound (I)N-(6-amino-5-methylpyridin-3-yl)-2-((2R,5S)-2-(benzo[d]thiazol-5-yl)-5-methylpiperidin-1-yl)-2-oxoacetamide (160 mg, 390.72 μmol, 96.68% yield) RetTime=45.8 min, [α]21D=+191.5° (c=0.1 g/100 mL, EtOH).

Compound (I): RT (IC, Hexane-IPA-MeOH, 50-25-25, 0.6 ml/min)=47.098 min.

¹H NMR (600 MHz, DMSO-d6) δ 0.98-1.06 (m, 3H), 1.30-1.42 (m, 1H), 1.66-1.75 (m, 1H), 1.82-1.91 (m, 1H), 1.95-2.04 (m, 3H), 2.06-2.23 (m, 1H), 2.26-2.35 (m, 1H), 2.76-3.27 (m, 1H), 3.38-4.06 (m, 1H), 5.26-5.60 (m, 1H), 5.60-5.76 (m, 2H), 7.39-7.46 (m, 1H), 7.50 (s, 1H), 7.92-8.01 (m, 1H), 8.01-8.06 (m, 1H), 8.13-8.20 (m, 1H), 9.37-9.43 (m, 1H), 10.50-10.70 (m, 1H).

LCMS(ESI): [M+H]⁺ m/z: calcd 409.2; found 410.0; Rt=1.992 min.

Compound (Ia) RT (IC, Hexane-IPA-MeOH, 50-25-25, 0.6 ml/min)=35.176 min.

¹H NMR (600 MHz, DMSO-d6) δ 1.00-1.05 (m, 3H), 1.27-1.40 (m, 1H), 1.64-1.75 (m, 1H), 1.82-1.93 (m, 1H), 1.95-2.04 (m, 3H), 2.07-2.25 (m, 1H), 2.27-2.35 (m, 1H), 2.75-3.27 (m, 1H), 3.42-4.11 (m, 1H), 5.28-5.59 (m, 1H), 5.60-5.74 (m, 2H), 7.40-7.45 (m, 1H), 7.49 (s, 1H), 7.94-8.01 (m, 1H), 8.01-8.06 (m, 1H), 8.13-8.19 (m, 1H), 9.36-9.42 (m, 1H), 10.52-10.58 (m, 1H).

LCMS(ESI): [M+H]⁺ m/z: calcd 409.2; found 410.2; Rt=2.001 min.

### Example 2. The synthesis of tert-butyl (S)-5-methyl-2-oxopiperidine-1-carboxylate

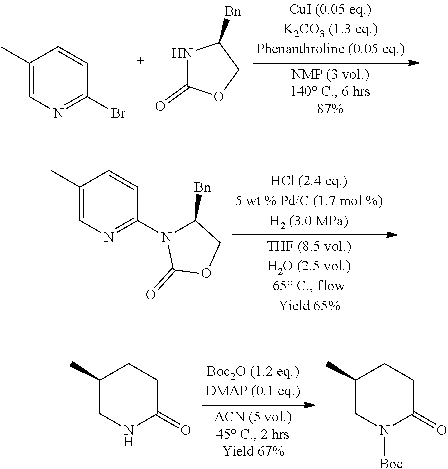

### Step 1: Synthesis of (S)-4-benzyl-3-(5-methylpyridin-2 yl)oxazolidin-2-one

NMP (14.1 L, 3 v), 2-Bromo-5-methylpyridine (4700 g, 27.32 mol, 1.0 eq), (S)-4-Benzyl-2-oxazolidinone (4987 g, 28.14 mol, 1.03 eq), K₂CO₃ (4908 g, 35.52 mol, 1.3 eq) and Phenanthroline (246.2 g, 1.37 mol, 0.05 eq) were charged to a 50 L reactor. Degassed with N₂ three times. Charged CuI (259.8 g, 1.37 mol, 0.05 eq) to the reactor. Degassed with N₂ three times. Heated up to 140° C. and stirred for 5 h. HPLC showed that the reaction was completed. The mixture was cooled to 20-30° C., then filtered through diatomite, and the filter cake was washed with NMP (9.4 L, 2 v). Combined the filtrate and charged the filtrate dropwise to the H₂O (47 L, 10 v) at 15-25° C. and stirred for 1-2 h. Filtered, then the cake was dissolved with EA (6 v) and washed with 5% NH₃·H₂O (14.1 L, 3 v), then washed with H₂O (14.1 L, 3 v). The organic phase was concentrated to ˜2 v (9.8 L) at 40-50° C. Charged n-Heptane (37.6 L, 8 v) to the concentrate and concentrated to 5 v (23.5 L) at 40-50° C. The mixture was stirred for 2 h at 15-25° C. Filtered and the cake was washed with n-Heptane (4.7 L, 1 v). The cake was dried at 40-50° C. for 12-24 h. Obtained (S)-4-benzyl-3-(5-methylpyridin-2-yl)oxazolidin-2-one (6731 g, 87% yield).

¹H NMR (400 MHz, Chloroform-d) δ 8.25-8.24 (q, 1H), 8.12-8.10 (d, J=8.56 Hz 1H), 7.59-7.56 (m, 1H), 7.37-7.23 (m, 5H), 5.13-5.08 (m, 1H), 4.34-4.23 (m, 2H), 3.44-3.40 (m, 1H), 2.87-2.82 (m, 1H), 2.35 (s, 3H).

MS=269.0 [M+H]⁺

### Step 2: Synthesis of (S)-5-methylpiperidin-2-one

To a 20 L reactor was charged with (S)-4-benzyl-3-(5-methylpyridin-2-yl)oxazolidin-2-one (5900 g), THF (50 L), water (14.75 L) and 37% hydrochloric acid (810 g). The mixture was stirred to dissolve and filtered through a pad of the Celite to afford clear solution. Confirm the 1.25 L Micropacked bed reactor was clean and dry. To the 1.25 L of Micropacked bed reactor was charged with PDC 1051 (5 wt % Pd/C, 810.00 g, 1.7 mol %). The jacket temp. was set as 65° C. and the preheater was set as 65° C. The pressure of hydrogen was set as 3.0 MPa. The flow rate of hydrogen was set as 1000 mL/min. The charging pump can be started after the hydrogen was stable and the flow of the solution was set as 30.0 mL/min. The reaction was started; during the reaction, IPC every 2-4 hours to check whether the (S)-4-benzyl-3-(5-methylpyridin-2-yl)oxazolidin-2-one was remained or not. If remained, reaction solution should be hydrogenated again.

For the workup, charged 40% NaOH (2.6 kg) aq. to the reaction at 5-15° C. and adjusted pH=10. The mixture was concentrated at 40-50° C. to removed THF. Then stirred for 2 h at 5-10° C., filtered and collected filtrate (Weight: 18.1 kg, took 1 kg filtrate reserved sample and continued work-up for the rest). The filtrate was extracted with 2-MeTHF (7.5 L, 1.35 v), separation, the bottom of aqueous phase was concentrated to no fraction at 50-55° C. Charged MeCN (9.7 L, 1.6 v) to the concentrate and stirred for 1 h at 15-25° C. Filtrated and the cake was washed with MeCN (1 L, 0.2 v). The filtrate was concentrated to no fraction at 40-50° C. Obtained 1622 g yellow oil with 70% yield as (S)-5-methylpiperidin-2-one.

The current procedure including the flow hydrogenation allowed for a lower catalyst loading as compared to results published in Angew. Chem. Int. Ed., 2021, 60, 6425-6429, which is incorporated by reference.

1H NMR: (400 MHz, Chloroform-d) 7.42 (s, 1H), 3.20-3.17 (m, 1H), 2.93-2.76 (m, 1H), 2.31-2.22 (m, 2H), 1.82-1.70 (m, 2H), 1.39-1.33 (m, 1H), 0.91-0.89 (d, J=6.6 Hz, 3H).

MS=114.1 [M+H]⁺.

### Step 3: Synthesis of tert-butyl (S)-5-methyl-2-oxopiperidine-1-carboxylate

Charged (S)-5-methylpiperidin-2-one (1600 g, 14.14 mol, 1.0 eq, QNMR: 86.3%), DMAP (172.7 g, 1.41 mol, 0.1 eq) and MeCN (8 L, 5 v) to a reactor. Heated up to 40-50° C. Charged (Boc)₂O (3703 g, 16.97 mol, 1.2 eq) dropwise to the reactor. Stirred for 2 h at 40-50° C. HPLC showed the reaction was completed. The reaction was concentrated to no fraction at 40-50° C. Charged MTBE (8 L, 5 v) to the concentrate and stirred for 1 h, then washed with 10% citric acid aq. (4800 g X2, 3 v) twice and washed with 10% NaCl aq. (4800 g, 3 v). The organic phase was concentrated to no fraction at 40-50° C., obtained crude: 2560 g yellow oil.

The oil was charged with MTBE (7 L) and 200300 mesh silica (2300 g) to the reactor. Stirred for 2 h and filtered. The wet cake was washed with MTBE (2.3 L). Combined the filtrate and concentrated to no fraction at 40-50° C., obtained 2388 g crude as light-yellow oil. The oil was charged with n-Heptane (7.2 L, 3 v) to the reactor. Stirred for 4 h at −15 to −13° C., then filtered. The cake was dried with N₂ at 15-25° C. Obtained 1791 g off-white solid of tert-butyl (S)-5-methyl-2-oxopiperidine-1-carboxylate (59.4% yield, HPLC: 99.0%; ee: 98.6%, Daicel CHIRALPAK AD-RH 150 mm×4.6 mm, 5 μm, wavelength, 220 nm).

¹H NMR: (400 MHz, Chloroform-d) δ 3.82-3.77 (m, 1H), 3.15-3.09 (m, 1H), 2.60-2.43 (m, 2H), 2.00-1.85 (m, 2H), 1.51-1.43 (m, 10H), 1.05-1.04 (d, J=6.6 Hz, 3H).

MS=449.2 [2M+Na]⁺.

### Example 3. The synthesis of 2-((6-(bis(tert-butoxycarbonyl)amino)-5-methylpyridin-3-yl)amino)-2-oxoacetic acid

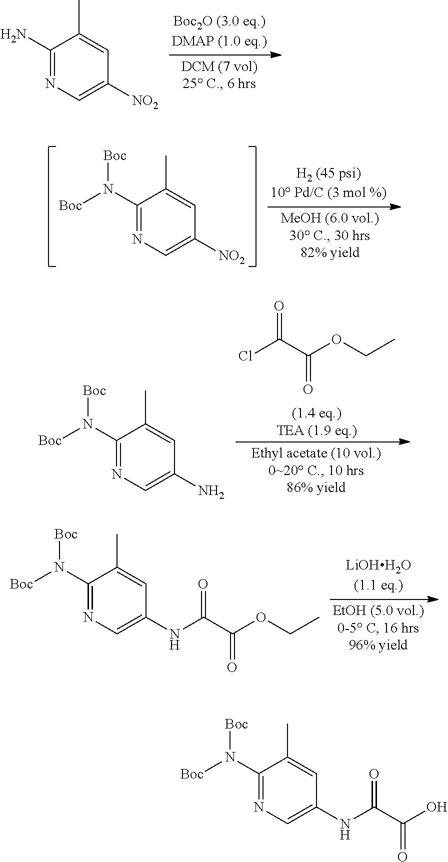

### Step 4: Synthesis of tert-butyl (5-amino-3-methylpyridin-2 yl)(tert-butoxycarbonyl)carbamate

To a solution of 3-methyl-5-nitropyridin-2-amine (11.4 kg, 74.44 mol, 1 eq) in DCM (80 L, 7 vol) was added DMAP (9.1 kg, 74.49 mol, 1 eq). The reactor was stirred for 0.5 h, then was added Boc₂O (48 kg, 219.93 mol, 3 eq) portion wisely. The reaction mixture was stirred for 6 h at 25° C., then charged with 10% citric acid monohydrate aqueous solution (10 kg) at 25° C. The mixture was stirred for 2 h at rt, and layers were separated. The top aqueous layer was back washed with DCM (40 L), and combined organic layers were washed with water (60 L). The organic layer was separated, concentrated to 3 vol (around 34 L), and charged THF (87 L). The mixture was concentrated to 3 vol (around 34 L), then added THF (72 L), followed by McOH (121 L). The mixture was stirred at rt for 1 h, then charged 10% Pd/C (2.3 kg). The reactor was purged with N₂ for three times, then charged with H₂ (45 psi). The mixture was heated to 30° C. and stirred under 45 psi H₂ for 30 h, then filtered through 15 kg celite. The celite cake was rinsed with 60 kg McOH twice. The filtrate was transferred back to reactor, and concentrated to 37 L. Charged THF (121 kg) to the mixture, and concentrated to 37 L. The mixture was charged with THF (22 kg) and heated to 65° C. Stirred at 65° C. for 2.5 h, then cooled down to 50° C. At 50° C., charged n-heptane (90 kg) drop wisely over 3 h, then stirred at 50° C. for 1.5 h. The mixture was cooled down to 15° C. and stirred for 4 h. The mixture was filtered, washed with n-heptane (38 kg) twice, cake was dried in over at 45° C. for 24 h, afforded 19.8 kg solid of tert-butyl (5-amino-3-methylpyridin-2-yl)(tert-butoxycarbonyl)carbamate (82% yield).

¹H NMR: (400 MHz, Chloroform-d) δ 7.49-7.68 (m, 1H), 6.82 (d, J=2.50 Hz, 1H), 5.34 (s, 2H), 1.98 (s, 3H), 1.36 (s, 18H).

MS=324.05 [M+1]⁺.

### Step 2: Synthesis of ethyl 2-((6-(bis(tert-butoxycarbonyl)amino)-5-methylpyridin-3 yl)amino)-2-oxoacetate

To a solution of tert-butyl (5-amino-3-methylpyridin-2-yl)(tert-butoxycarbonyl)carbamate (21.7 g, 65.4 mmol, 1 eq) in ethyl acetate (220 mL, 10 vol) was charged triethylamine (12.2 g, 120.7 mmol, 1.9 eq). The mixture was cooled to 0-5° C. and stirred for 0.5 h, then charged with ethyl oxalyl chloride (12.5 g, 91.6 mmol, 1.4 eq) dropwise at 0-5° C. The mixture was stirred at 25° C. for 16 h and monitored by HPLC. The mixture was cooled to 20° C. and charged with water (80 mL) drop wisely over 1 h, then stirred for another 2 h. The two layers were separated and organic layer was back washed with water (80 mL). The organic layer was separated, and concentrated down to 87 mL below 45° C. under vacuum. Kept the mixture temp at 40° C. and charged n-heptane (170 mL), and stirred at 45° C. for 4 h, then cooled down to 20° C. Stirred the mixture for another 8 h. The mixture was filtered, and washed with ethyl acetate/heptane (1:3 ratio, 40 mL). The cake was dried at 45° C. for 30 h, affording off-white solid 24.5 g (86% yield).

¹H NMR: (400 MHz, Chloroform-d) δ 11.08 (s, 1H), 8.64 (d, J=2.38 Hz, 1H), 8.13 (d, J=2.38 Hz, 1H), 4.33 (q, J=7.13 Hz, 2H), 2.11-2.18 (m, 3H), 1.35-1.42 (m, 1H), 1.33 (t, J=7.07 Hz, 3H).

MS=424.40 [M+1]⁺.

### Step 3: Synthesis of 2-((6-(bis(tert-butoxycarbonyl)amino)-5-methylpyridin-3 yl)amino)-2-oxoacetic acid

To a solution of ethyl 2-((6-(bis(tert-butoxycarbonyl)amino)-5-methylpyridin-3-yl)amino)-2-oxoacetate (19.7 g, 45.7 mmol, 1 eq) in water (40 mL) was added EtOH (100 mL). The mixture was stirred and cooled down to 0-5° C. The solution of LiOH—H₂O (2.1 g, 50 mmol, 1.1 eq) in water (60 mL) was added to previous mixture at 0-5° C. over 1 h. The resulting mixture was stirred at 0-5° C. for 16 h and monitored by HPLC. Charged 15% citric acid aqueous solution (200 g) into above mixture over 4-6 h until pH=3-4 at 0-5° C. Stirred for 16 h at 0-5° C., and got suspension. The suspension was filtered, washed the cake with EtOH/H₂O (1:3 ratio, 20 mL), then the cake was dried in oven at 45° C. for 24 h, afforded off-white solid 17.7 g, 96% yield.

¹H NMR: (400 MHz, Chloroform-d) δ (ppm) δ 10.86-11.13 (m, 1H), 8.65 (d, J=2.25 Hz, 1H), 8.15 (d, J=2.00 Hz, 1H), 2.37-2.64 (m, 6H), 1.36 (s, 19H).

MS=396.10 [M+1]⁺.

### Example 4. The synthesis of 5-((2R,5S)-5-methylpiperidin-2-yl)benzo[d]thiazole

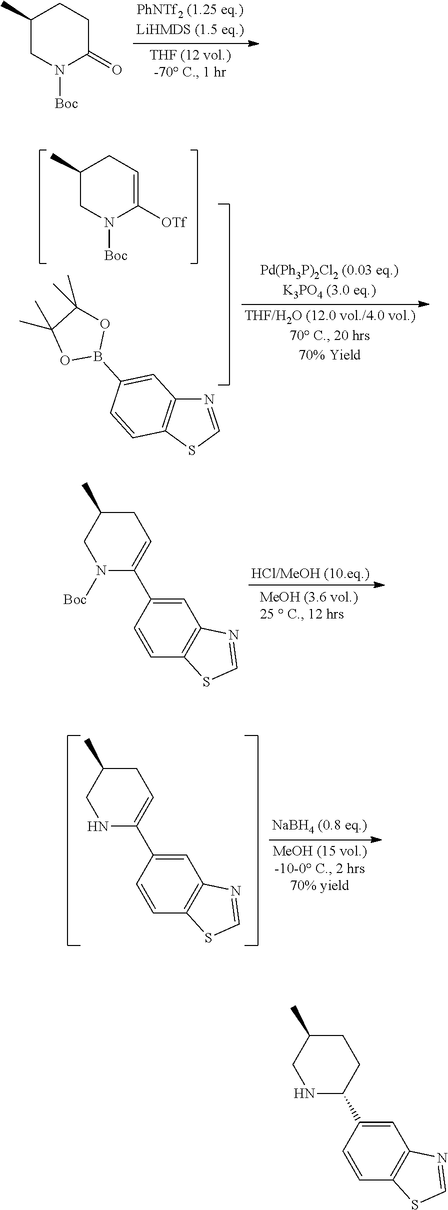

### Step 1: Synthesis of tert-butyl (S)-6-(benzo[d]thiazol-5-yl)-3-methyl-3,4-dihydropyridine-1(2H)-carboxylate

The solution tert-butyl (S)-5-methyl-2-oxopiperidine-1-carboxylate (20.58 g, 96.5 mmol) in dry THF (110 mL) was cooled to −70° C., then added LiHDMS THF solution (1M, 145 mL, 145 mmol) drop wisely over 1 h while keeping temperature below −70° C. The mixture was stirred at −70° C. for 1 h, then the solution of PhNTf₂ (43.1 g, 120.6 mmol) in dry THF (150 mL) was added drop wisely. Stirred at −70° C. for 1 h, then warmed to 25° C. and stirred for 6 h. The mixture was washed by 7% NaHCO3 solution (300 mL), and organic layer was dried, and added IPAc (250 mL). The IPAc solution was washed with 1N NaOH (100 mL) twice, then water (100 mL) three times. The organic layer was dried, added 5-(4,4,5,5-tetramethyl-1,3,2-dioxaborolan-2-yl)benzo[d]thiazole (20 g, 76.6 mmol) and THF (200 mL), water (80 mL), then purged with N₂ for 30 min. Charged K₃PO₄ (48.8 g, 223 mmol) and Pd(PPh₃)₂Cl₂ (806 mg, 1.15 mmol) and purged with N₂ for 30 min. The mixture was heated to 70° C. for 20 h, and HPLC showed reaction is completed. Cooled down to rt, and filtered, washed the cake with THF (40 mL) twice. The filtrate was separated two layers, and organic layer wad dried. Then 200 mL of toluene was added, washed with water (100 mL). The organic layer was added 10 g of 2-mercaptonicotinic acid, heated to 50° C. for 6 h, then cooled down, filtered. This process was repeated another two times to remove Pd. The filtrate was dried, added IPA (60 mL). The solution was heated to 50° C., and water (80 mL) was added over 3 hrs. Stirred at 50° C. for 6 h, then cooled down to 10° C. and stirred for 12 h. The suspension was filtered, and washed with IPA/water (20 mL, 4:3). The cake was dried at 45° C. for 24 h, affording 17.6 g (70% yield) of tert-butyl (S)-6-(benzo[d]thiazol-5-yl)-3-methyl-3,4-dihydropyridine-1(2H)-carboxylate.

¹H NMR: (400 MHz, Chloroform-d) δ 9.01 (d, J=1.88 Hz, 1H), 8.10 (s, 1H), 7.88 (br d, J=8.38 Hz, 1H), 7.38-7.56 (m, 1H), 7.44 (br d, J=8.38 Hz, 1H), 5.44 (br d, J=1.75 Hz, 1H), 4.13 (br d, J=12.26 Hz, 1H), 3.00-3.09 (m, 1H), 2.33-2.57 (m, 1H), 0.88-1.27 (m, 13H).

MS=331.20 [M+1]⁺.

### Step 2: Synthesis of 5-((2R,5S)-5-methylpiperidin-2 yl)benzo[d]thiazole

To solution of tert-butyl (S)-6-(benzo[d]thiazol-5-yl)-3-methyl-3,4-dihydropyridine-1(2H)-carboxylate (50 g, 151.3 mmol, 1 eq) in McOH (180 mL) was added 4 M HCl solution in McOH (375.2 g, 1501.7 mmol, 10 eq). The mixture was stirred at 25° C. for 12 h, and monitored by HPLC. Concentrated the mixture to around 75 mL and charged DCM (150 mL). The mixture was basified to pH=9-10 by adding 10% Na₂CO₃ solution over 1 h. After stirring for 1 h, the two layers were separated. The aqueous layer was back washed by DCM (100 mL) and separated. Combined organic layers were concentrated to 75 mL and charged with McOH (250 mL). The resulting mixture was concentrated to 180 mL and charged with 550 mL of McOH. Then cooled down to −10-0° C., added NaBH₄ (4.75 g, 0.8 eq), stirred at −10-0° C. for 2 h. HPLC showed reaction was done, and charged water (100 mL) at −10-0° C. over 1 h. After addition, the mixture was concentrated to 270 mL and charged with DCM (290 mL), water (100 mL). After stirring for 1 h, two layers were separated. The aqueous layer was back washed with DCM (90 mL). The combined organic layers were passed through CUNO for decoloring. The resulting solution was switched from DCM to ACN with 75 mL total volume. Water (30 mL) was added at 25° C. over 60 min at 30° C., and charged with crystal seeds. After stirring for 2 h at 30° C., charged water (270 mL) dropwise over 4 h, and stirred at 30° C. for 5 h. The mixture was cooled down to −5-5° C., and filtered. The cake was washed with ACN/H₂O (1:2 ratio). The wet cake was transferred back to flask and added ACN (40 mL) and water (120 mL). Stirred at 30° C. for 3 h, then cooled down to −5-5° C. and stirred for another 12 h. The suspension was filtered and washed the cake with ACN/water (50 mL, 1:2 ratio). The cake was dried at 30° C. for 17 h, affording desired product as bis-hydrate (29.8 g, 70% yield).

¹H NMR: (400 MHz, Chloroform-d) δ 9.38-9.59 (m, 1H), 8.27 (br d, J=11.57 Hz, 2H), 7.60 (d, J=8.41 Hz, 1H), 4.40 (br d, J=9.72 Hz, 1H), 3.34 (br d, J=10.63 Hz, 1H), 2.70-2.92 (m, 1H), 1.76-2.19 (m, 4H), 1.25-1.55 (m, 1H), 0.98 (d, J=6.35 Hz, 3H).

MS=233.10 [M+1]⁺.

### Example 5. The synthesis of N-(6-amino-5-methylpyridin-3-yl)-2-((2R,5S)-2-benzo d thiazol-5-yl)-5-methylpiperidin-1-yl)-2-oxoacetamide

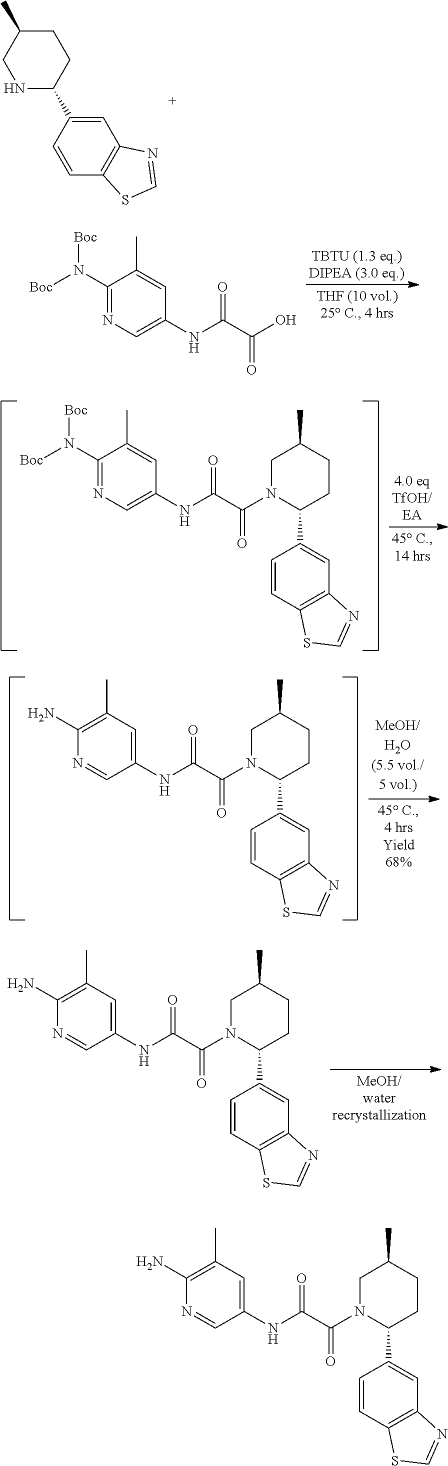

### Step 1: Synthesis of purified N-(6-amino-5-methylpyridin-3 yl)-2-((2R,5S)-2-(benzo[d]thiazol-5-yl)-5-methylpiperidin-1-yl)-2-oxoacetamide

5-((2R,5S)-5-methylpiperidin-2-yl)benzo[d]thiazole (bis-hydrate, 100 g, 372.6 mmol) in THE (500 mL) was concentrated to 200 mL, and recharged THE (500 mL) twice to azeotrope water until KF below 0.5%. 300 mL of THE was added, followed by 2-((6-(bis(tert-butoxycarbonyl)amino)-5-methylpyridin-3-yl)amino)-2-oxoacetic acid (187.2 g, 473.4 mmol), N-diisopropylethylamine (167.1 g, 1.29 mol), and 2-(1H-benzotriazole-1-yl)-1,1,3,3-tetramethyluronium tetrafluoroborate (207.6 g, 646.6 mmol). Stirred at 25° C. for 10 h, HPLC showed reaction was completed. The reaction mixture was added water (350 mL), and concentrated down to 700 mL of volume. Ethyl acetate (600 mL) was added and concentrated to 700 mL of volume again. Added K₂CO₃ aqueous solution (16%, 500 mL) and stirred for 1 h, with pH around 9. The mixture was filtered, and cake was washed with 200 mL of ethyl acetate. The filtrate was separated two layers, and organic layer was washed with 5% Na₂CO₃ solution (500 mL), water (500 mL). All aqueous layers were combined and washed with ethyl acetate (250 mL) once. The organic layers were combined, added N-acetyl-L-cysteine (50 g) and stirred at 50° C. for 5 h, then filtered. The cake was washed with ethyl acetate (200 mL). The filtrate was concentrated to dryness, then added ethyl acetate (1 L), then trifluoromethanesulfonic acid (TfOH, 260 g, 1.73 moles) was added. The mixture was stirred at 25° C. for 4 h, and HPLC showed that reaction was completed, suspension was obtained. The mixture was filtered, and cake (TfOH salt) was washed with ethyl acetate (200 mL). As an alternative to TfOH, hydrochloric acid (HCl) also removes the Boc groups. However, the corresponding HCl salt is amorphous, whereas the TfOH salt is crystalline and gives better impurity purging effect.

In another flask, Na₂CO₃ (100 g, 943 mmol) in water (1.9 L) was added EtOH (550 g) and THE (1.25 kg) and stirred for 1 h until a clear solution. Charged the wet cake (TfOH salt) to the solution slowly below 35° C., and added extra Na₂CO₃ to adjust pH=9.5 after addition of wet cake. After stirring for 2 h, the mixture was concentrated to 3 L of volume and kept at around 45° C. Seeds was added, stirred at 45° C. for 2 h, then concentrated to 2 L volume. The mixture was cooled down to 20° C., then filtered. The cake was washed with 200 mL of water, then wet cake was transferred back to reactor and 800 mL of water was added. The suspension was stirred for 3 h, then filtered. The cake was washed with water, then dried in oven under vacuum at 45° C. for 24 h, affording 122 g off-white solid, 80% yield.

### Step 2: Recrystallization of N-(6-amino-5-methylpyridin-3 yl)-2-((2R,5S)-2-(benzo[d]thiazol-5-yl)-5-methylpiperidin-1-yl)-2-oxoacetamide

Crude N-(6-amino-5-methylpyridin-3-yl)-2-((2R,5S)-2-(benzo[d]thiazol-5-yl)-5-methylpiperidin-1-yl)-2-oxoacetamide (122 g) was dissolved in 1.15 L of McOH/H₂O (v/v, 9/1) and heated to 60° C. to a clear solution. Stirred for 2 h, then cooled down to 40° C., and seeded with crystalline compound. The mixture was concentrated to 880 mL and water (500 mL) was added. The mixture was stirred at 40° C. for 2 h, then cooled down to 5° C., stirred for 5 h. The mixture was filtered and washed with McOH/water (1:1), then dried in oven at 50° C. for 24 h, affording 116 g off white solid, 95% yield.

¹H NMR: (400 MHz, DMSO-d6) δ 10.53-10.66 (m, 1H), 9.43 (s, 1H), 8.14-8.25 (m, 1H), 7.95-8.13 (m, 2H), 7.42-7.60 (m, 2H), 5.57-5.71 (m, 2H), 5.51-5.83 (m, 1H), 5.25-5.39 (m, 1H), 4.02-4.15 (m, 1H), 3.44-3.59 (m, 1H), 2.81 (br d, J=11.13 Hz, 1H), 2.28-2.45 (m, 1H), 1.97-2.03 (m, 1H), 2.06 (s, 2H), 1.82-1.97 (m, 1H), 1.73 (br d, J=2.38 Hz, 1H), 1.29-1.52 (m, 1H), 1.05 (br d, J=6.50 Hz, 3H).

MS=410.11 [M+1]⁺.

### Example 6. Exemplary Preparation of Crystalline Form A

Slow evaporation at about room temperature of a solution obtained from about 9.8 mg Compound (I) dissolved in 4.0 mL MeCN resulted in the formation of crystalline Form A.

### Example 7. Additional Exemplary Preparation of Crystalline Form A

A sample of Compound of formula (I) was slurried in ethanol at 60° C. for two days, and the resulting materials were filtered by centrifugation and determined to correspond to crystalline form A.

### Example 8. Exemplary Thermogravimetric Characterization of Crystalline Form A

TG-FTIR: Thermogravimetric measurements were carried out with a Netzsch Thermo-Microbalance TG 209 coupled to a Bruker FTIR Spectrometer Vector 22 (sample pans with a pinhole, N₂ atmosphere, heating rate 10 K/min). The TG-FTIR spectrum of the compound of formula (I) is shown in FIG. 2A.

### Example 9. XRPD Measurements for Crystalline Form A

X-ray powder diffraction was carried out as a two theta theta coupled measurement, using a Bruker D8 Advance XRPD diffractometer equipped with a LYNXEYE (1D mode) detector (with a PSD opening of 2.1°), operating with Cu-Kα1 radiation. The measurements with this instrument were performed at a tube voltage of 40 kV and tube current of 40 mA. The following parameters were set for the coupled 2θ/θ scan: continuous PSD fast mode; 0.02° 2θ step size; 0.12 s step time; 4-40° 2θ scanning range. An appropriate amount of sample (e.g., 20-50 mg) was placed in the central area of a monocrystalline silicone wafer. If necessary, a thin layer of petroleum jelly or silicone oil was applied to the surface of the single silicon wafer to attach the sample, and the excess sample was removed by gentle tapping. The sample plate was loaded into the XRPD sample holder. The sample was rotated during the measurement. All sample preparation and measurement were done in an ambient air atmosphere. The XRPD pattern obtained for crystalline form A is shown in FIG. 1A.

### Example 10. Exemplary Dynamic Vapor Sorption Measurements

Dynamic vapor sorption: DVS measurements were performed with an SMS Dynamic Vapor Sorption Advantage System with a total gas flow of 200 sccm, at an oven temperature of 25° C. Humidity changes were performed in steps of 10%, in a sequence of 40-0-95-0-40% humidity, with equilibrium being 0.01 dm/dt (%/min), minimum dm/dt stability duration of 60 minutes and maximum dm/dt stability duration of 180 minutes. The results from the DVS test are presented in FIG. 3A and FIG. 3B, showing an uptake of ˜0.1% water on the sorption curve between 40% RH and 80% RH, at 25° C. Based on these results, the material is nonhygroscopic below 80% RH at 25° C.

### Example 0.1.1. Exemplary Single Crystal X-Ray Diffraction Studies

The single crystal X-ray diffraction studies were carried out on a Bruker Kappa Photon II CPAD diffractometer equipped with Cu Kα radiation (λ=1.54178). Crystals of the subject compound were grown by dissolving approximately 1 mg of sample in 350 μL of heated Acetonitrile, that was allowed to slowly cool and sit undisturbed over several weeks. A 0.472×0.074×0.060 mm colorless block was mounted on a Cryoloop with Paratone oil. Data were collected in a nitrogen gas stream at 100(2) K using φ and ω̅ scans. Crystal-to-detector distance was 60 mm using variable exposure time (10 s-60 s) depending on θ with a scan width of 1.0°. Data collection was 99.8% complete to 68.00° in θ. A total of 21266 reflections were collected covering the indices, −6<=h<=5, −17<=k<=17, −17<=1<=17. 7481 reflections were found to be symmetry independent, with a Rᵢₙₜ of 0.0237. Indexing and unit cell refinement indicated a primitive, triclinic lattice. The space group was found to be P1. The data were integrated using the Bruker SAINT software program and scaled using the SADABS software program. Solution by direct methods (SHELXT) produced a complete phasing model for refinement.

All nonhydrogen atoms were refined anisotropically by full-matrix least-squares (SHELXL-2014). All carbon bonded hydrogen atoms were placed using a riding model. Their positions were constrained relative to their parent atom using the appropriate HFIX command in SHELXL-2014. All other hydrogen atoms (H-bonding) were located in the difference map. Their relative positions were restrained using DFIX commands and their thermals freely refined. The absolute stereochemistry of the molecule was established by anomalous dispersion using the Parson's method with a Flack parameter of −0.004(8). The crystal system was identified as a triclinic system with a P1 space group. Crystallographic data are summarized in Table 2.

TABLE 2
Single crystal X-ray Data for Form A of a compound of formula (I)

| | | |
|---|---|---|
| Distance (Å) | a | 5.0705 |
| | b | 13.9249 |
| | c | 13.9877 |
| Angle (°) | α | 87.522 |
| | β | 85.730 |
| | γ | 81.474 |

| | |
|---|---|
| Volume (Å³) | 973.47 |
| Z | 2 |
| Density (Mg/m³) | 1.397 |

### Example 12. Exemplary Pharmaceutical Compositions and Dosage Forms

The pharmaceutical compositions and dosage forms examples described in this application are offered to illustrate embodiments of the compounds, pharmaceutical compositions, and methods provided herein and are not to be construed in any way as limiting their scope. For example, an excipient may act with a one or more similar or various functions (e.g., filler, glidant, disintegrant, etc.) and identification of a particular function herein is not to be construed in any way as limiting the scope of the respective component.

TABLE 3

| | | | Dosage Strength, mg | | | |
|---|---|---|---|---|---|---|
| | | | | 12.5 | 50 | |
| | | | 12.5/50 | Unit | Unit | |
| | | | Formulation | Weight, | Weight, | |
| Component | | Function | Composition | mg | mg | |
| Intragranular | Compound of formula | API | 12.5% | 12.5 | 50.0 | |
| | (I) | | | | | |
| | microcrystalline | Filler | 56.5% | 56.5 | 226.0 | |
| | cellulose PH 102 | | | | | |
| | (Avicel ® PH102) | | | | | |
| | Colloidal Silicon | Glidant | 0.5% | 0.5 | 2.0 | |
| | Dioxide (CAB-O- | | | | | |
| | SIL ® M-5P) | | | | | |
| | croscarmellose | Disintegrant | 2.0% | 2.0 | 8.0 | |
| | sodium (Ac-Di-Sol ®) | | | | | |
| | Magnesium Stearate | Lubricant | 0.5% | 0.5 | 2.0 | |
| Extragranular | microcrystalline | Extragranular | 25.0% | 25.0 | 100.0 | |
| | cellulose PH 200 | filler | | | | |
| | (Avicel ® PH 200) | | | | | |
| | microcrystalline | Extragranular | NA | NA | NA | |
| | cellulose PH 102 | filler | | | | |
| | (Avicel ® PH102) | | | | | |
| | Colloidal Silicon | Glidant | 0.5% | 0.5 | 2.0 | |
| | Dioxide (CAB-O- | | | | | |
| | SIL ® M-5P) | | | | | |
| | croscarmellose | Disintegrant | 2.0% | 2.0 | 8.0 | |
| | sodium (Ac-Di-Sol ®) | | | | | |
| | Magnesium Stearate | Lubricant | 0.5% | 0.5 | 2.0 | |
| Total Tablet Weight | | | 100.0% | 100.0 | 400.0 | |
| Coating | Opadry ® II White | Coating | 3.5% of Core | 3.5 | 14.00 | |
| | 85F18422 | | | | | |
| | | | Dosage Strength, mg | | | |
| | | | 100 | | 300 | |
| | | | | Unit | | Unit |
| | | | Formulation | Weight, | Formulation | Weight, |
| Component | | Function | Composition | mg | Composition | mg |
| Intragranular | Compound of | API | 29.41% | 100.00 | 40.00% | 300.00 |
| | formula (I) | | | | | |
| | Avicel ® PH102 | Filler | 39.70% | 135.0 | 54.00% | 405.00 |
| | Colloidal Silicon | Glidant | 0.37% | 1.25 | 0.50% | 3.75 |
| | Dioxide (CAB-O- | | | | | |
| | SIL ® M-5P) | | | | | |
| | croscarmellose | Disintegrant | 1.47% | 5.00 | 2.00% | 15.00 |
| | sodium (Ac-Di- | | | | | |
| | Sol ®) | | | | | |
| | Magnesium | Lubricant | 0.37% | 1.25 | 0.50% | 3.75 |
| | Stearate | | | | | |
| Extragranular | microcrystalline | Extragranular | 12.84% | 43.65 | NA | NA |
| | cellulose PH 200 | filler | | | | |
| | (Avicel ® PH 200 | | | | | |
| | microcrystalline | Extragranular | 12.84% | 43.65 | NA | NA |
| | cellulose PH 102 | filler | | | | |
| | (Avicel ® PH102 | | | | | |
| | Colloidal Silicon | Glidant | 0.50% | 1.70 | 0.50% | 3.75 |
| | Dioxide (CAB-O- | | | | | |
| | SIL ® M-5P) | | | | | |
| | croscarmellose | Disintegrant | 2.00% | 6.80 | 2.00% | 15.00 |
| | sodium (Ac-Di- | | | | | |
| | Sol ®) | | | | | |
| | Magnesium | Lubricant | 0.50% | 1.70 | 0.50% | 3.75 |
| | Stearate | | | | | |
| Total Tablet Weight | | | 100.00% | 340.00 | 100.00% | 750.00 |
| Coating | Opadry ® II White | Coating | 3.5% of Core | 11.90 | 3.5% of Core | 26.25 |
| | 85F18422 | | | | | |

### Example 13. PRMT5 Inhibitor Selectivity In Vitro and In Vivo

MTAP-null-selectivity of the PRMT5 inhibitor of formula (I) was determined in a cell based assay in vitro. Briefly, MTAP-isogenic cell lines were engineered by either CRISPR-mediated MTAP gene knockout (HAP1, CML cancer model)) or by reconstituting exogenous MTAP in an endogenous MTAP-deleted cell line (SW1573, NSCLC cancer model).

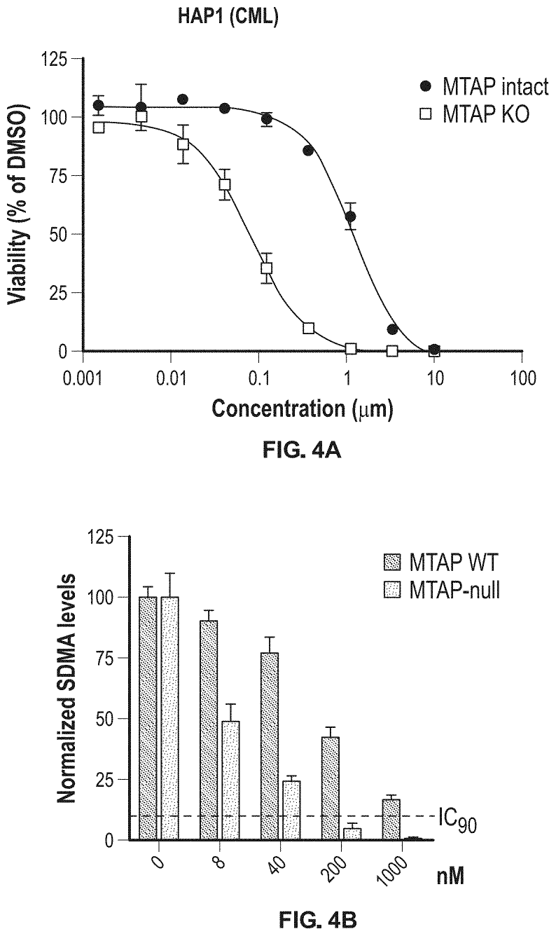

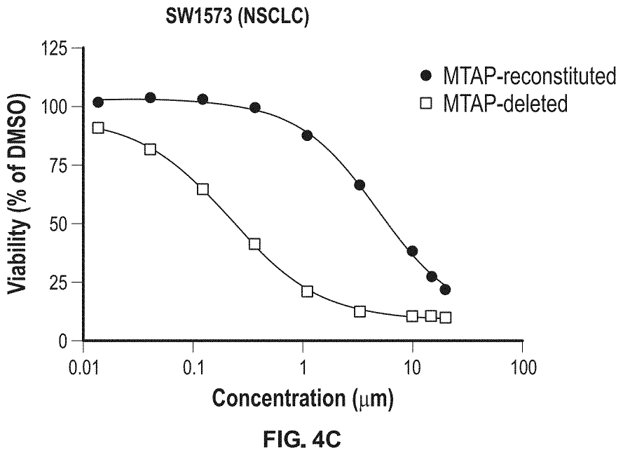

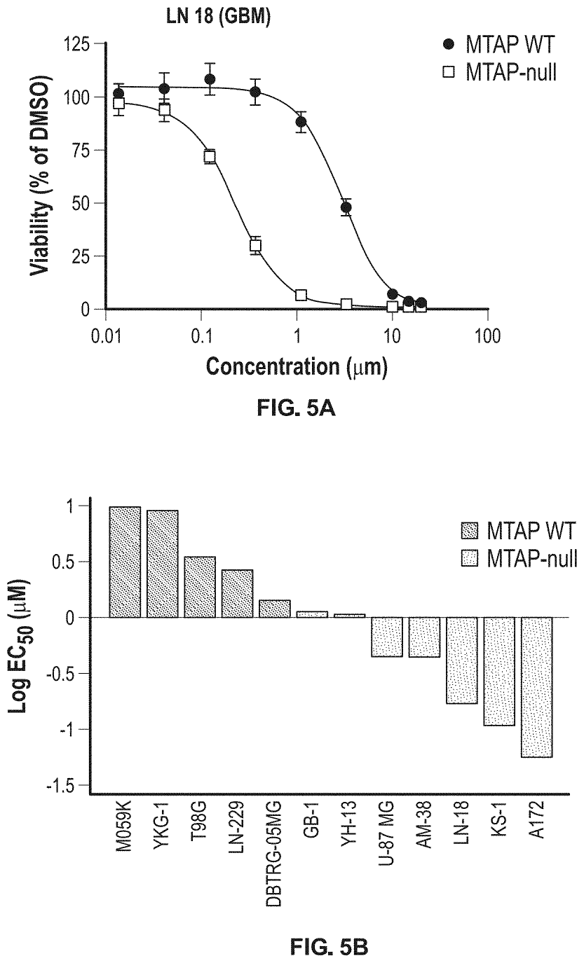

A similar experiment was performed in a pair of MTAP-isogenic LN18 (GBM cancer model) cell lines, where the pair was engineered by reconstituting exogenous MTAP in an endogenous MTAP-deleted LN18 cell line. Cells were treated with various amounts of PRMT5 inhibitor and live cells were determined at day 7 with the CellTiter-Glo viability assay. The results demonstrate 15×selectivity in MTAP-null cells in MTAP-isogenic cell pairs derived from multiple cancer lineages (FIGS. 4A, 4C, 5A) in vitro. Next, the pharmacodynamic activity of PRMT5 inhibitor of formula (I) to inhibit PRMT5 in the HAP1 MTAP-isogenic cell line pair was determined. Symmetric dimethylarginine (SDMA) levels at several concentrations of PRMT5 inhibitor of formula (I) (0, 8, 40, 200, and 1000 nM) were determined in MTAP WT and MTAP-null HAP1 cell lines. The results show that the PRMT5 inhibitor of formula (I) selectively inhibits PRMT5 (FIG. 4B) in a dose dependent manner in vitro.

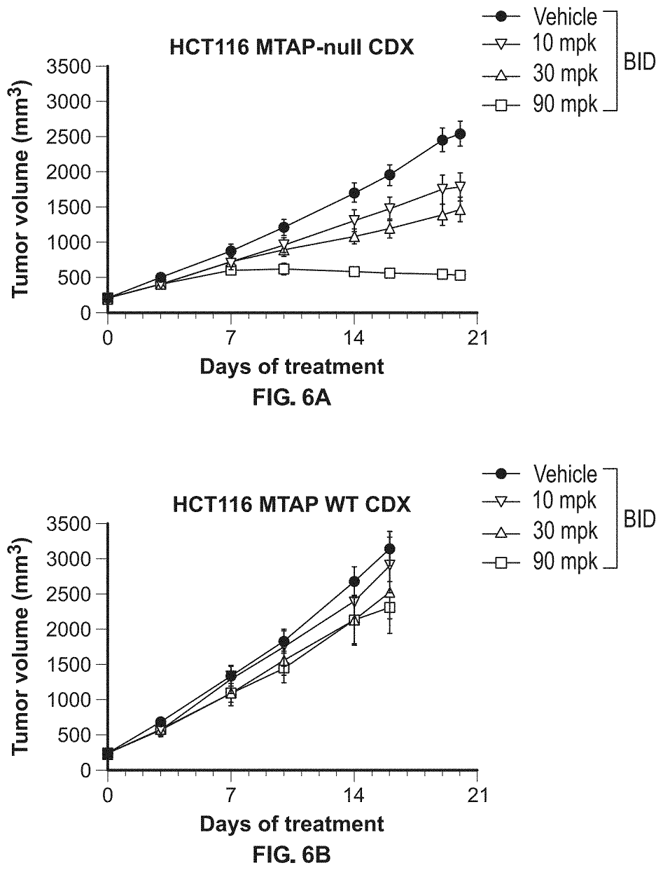

MTAP-null-selectivity of the PRMT5 inhibitor of formula (I) was determined in an in vivo mouse model. The colorectal carcinoma HCT116 MTAP-isogenic xenograft models that are either MTAP WT or MTAP-null were dosed with PRMT5 inhibitor of formula (I) at various concentrations (10 mpk, 30 mpk, and 90 mpk and vehicle as control) with n=8 mice per group and were dosed twice per day for the indicated time period. Tumor volume was measured on day 3, 7, 10, 14, 16, 19, and 20. The results show that the tumors in mice with the MTAP-null genotype grew slower, and shrunk at the highest PRMT5 inhibitor dose (FIGS. 6A and 6B). The treatment of tumors in mice with the MTAP WT genotype had a very small effect. The results show that PRMT5 inhibitor of formula (I) drives dose-dependent, MTAP-null-selective antitumor activity in vivo.

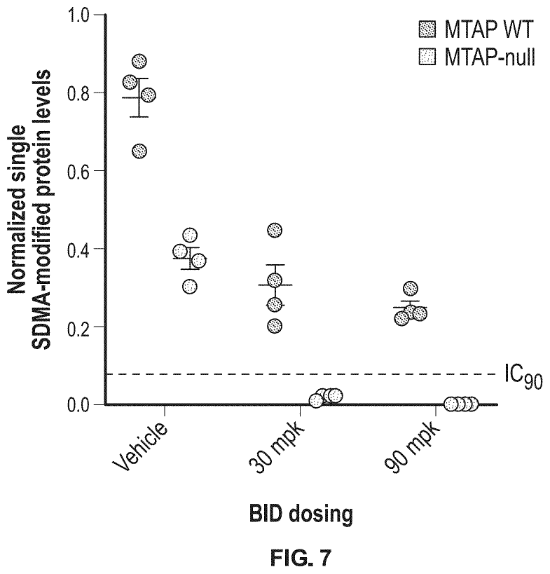

Tumors were further subjected to terminal pharmacodynamic analysis. A single SDMA-modified substrate was quantified by immunoblot in tumors from the HCT116 MTAP-isogenic xenograft models dosed with PRMT5 inhibitor of formula (I) and normalized to a loading control from tumors processed 8 hrs post-last dose (n=4 tumors per group). The results show that the PRMT5 inhibitor of formula (I) leads to a dose dependent inhibition of PRMT5 activity in vivo (FIG. 7).

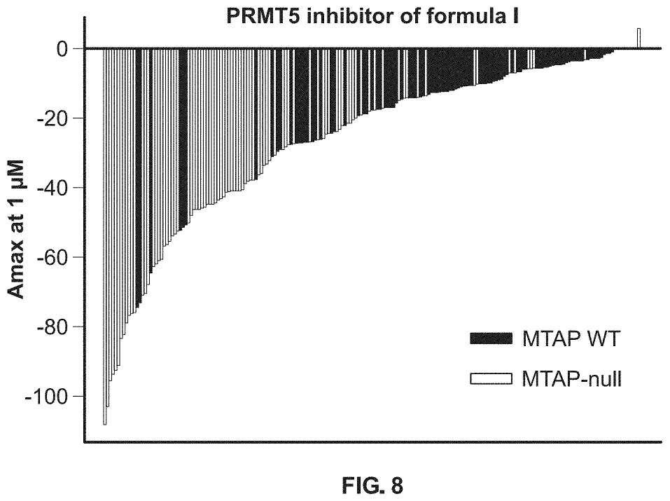

Additionally, 199 cancer cell lines representing multiple cancer lineages including NSCLC, pancreatic ductal cancer (PDAC), bladder cancer, CNS cancer, and heme malignancies were profiled with the PRMT5 inhibitor of formula (I) in a 7-day CellTiter-Glo assay. The maximum effect (Amax) at 1 μM (10× the GI₅₀) for each cell line was reported, and the cell lines were colored by MTAP status (MTAP-null in white, MTAP WT in black). MTAP-null cell lines were selectively targeted by the PRMT5 inhibitor of formula (I) (FIG. 8).

### Example 14. PRMT5 Inhibitor of Formula (I) in MTAP Deleted Xenograft Model In Vivo

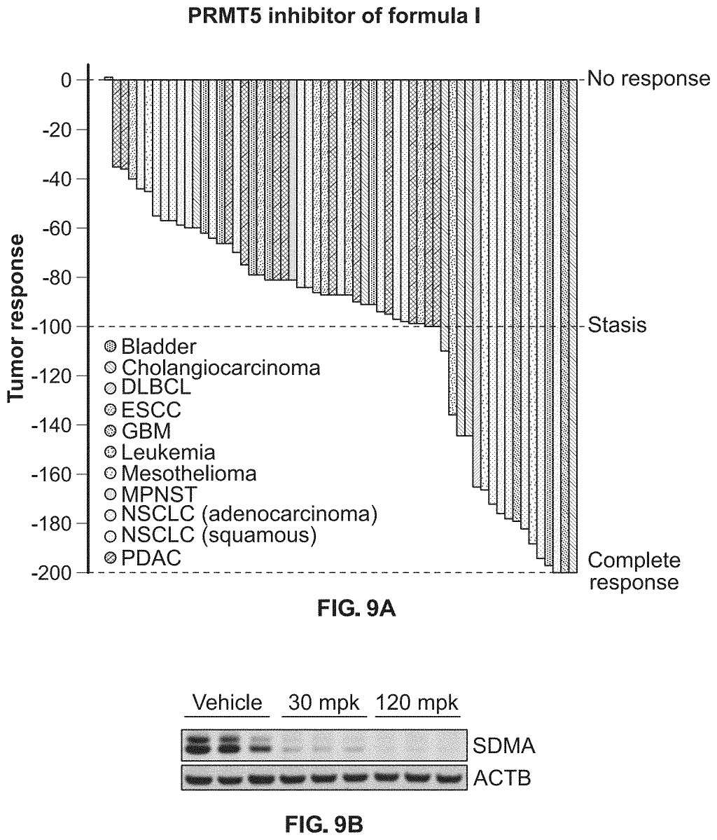

The efficacy of PRMT5 inhibitor of formula (I) was determined in MTAP-deleted CDX and PDX xenograft mouse models across clinically relevant lineages such as bladder cancer, cholangiocarcinoma, DLBCL, esophageal squamous cell carcinoma (ESCC), GBM, leukemia, mesothelioma, NSCLC (adeno) NSCLC (squamous), and PDAC. Mice were dosed at 30 and 120 mpk BID with PRMT5 inhibitor of formula (I) and vehicle as control. Tumor volume was measured and plotted for % growth relative to vehicle. The waterfall diagram in FIG. 9A shows the activity of PRMT5 inhibitor of formula (I) at 120 mpk in MTAP-deleted CDX and PDX models representing the indicated tumor histologies. The results show that PRMT5 inhibitor of formula (I) drives a strong, lineage agnostic antitumor response in vivo.

Tumors were further subjected to terminal pharmacodynamic (PD) analysis. Tumor tissue was harvested 8 hours post-last dose and analyzed by western blot for SDMA accumulation. A representative terminal PD analysis of a PRMT5 inhibitor of formula (I)-treated PDX tumor dosed at the 30 and 120 mpk BID are shown in FIG. 9B.

### Example 15. Brain-Penetration of PRMT5 Inhibitor of Formula (I)

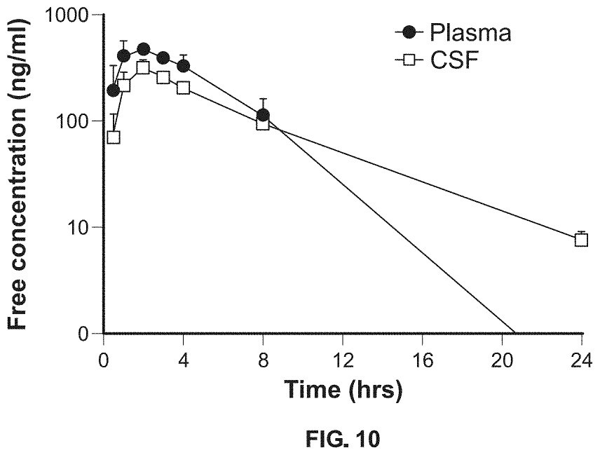

Brain penetration for PRMT5 inhibitor of formula (I) was determined in non-human primates. Following an oral administration of 10 mg/kg PRMT5 inhibitor of formula (I) to cisterna magna ported cynomolgus monkeys (N=3/group), serial samples of cerebrospinal fluid (CSF, a surrogate for free brain concentration) and plasma were collected. The results show that PRMT5 inhibitor of formula (I) CSF concentration closely approximated free PRMT5 inhibitor of formula (I) plasma concentration (Table 4 and FIG. 10). Thus, the results show that PRMT5 inhibitor of formula (I) is brain-penetrant in non-human primates in vivo.

TABLE 4

| | PRMT5 inhibitor of formula (I) |
|---|---|
| Plasma unbound AUC₀₋ₗₐₛₜ (ng*h/mL) | 1846 |
| CSF AUC₀₋ₗₐₛₜ (ng*h/mL) | 1649 |
| Kpuu_{brain} | 0.9 |

### Example 16. PRMT5 Inhibitor of Formula (I) Selectivity for MTAP^{null} GBM Cell Lines in Vitro

The antiproliferative activity of PRMT5 inhibitor of formula (I) in glioblastoma MTAP-isogenic cell line was determined. The MTAP-isogenic cell line was engineered by reconstituting exogenous MTAP in an endogenous MTAP-deleted cell line (LN18, GBM model). The antiproliferative activity was determined by CellTiter-Glo assay. The results show that PRMT5 inhibitor of formula (I) is efficacious and selective for the MTAP-deleted glioblastoma cell lines (FIG. 5A).

The efficacy and selectivity of PRMT5 inhibitor of formula (I) for MTAP-null cell lines representing glioblastoma was determined in vitro. Briefly, 12 glioblastoma cancer cell lines (5 MTAP WT and 7 MTAP-null) were treated for 7-days with a 9-point dose titration of PRMT5 inhibitor of formula (I) and the antiproliferative activity was determined by CellTiter-Glo assay. For each cell line a variable-slope (four parameter) curve was fit and the concentration at which half-maximal efficacy was determined and plotted on the y-axis in. The results show that PRMT5 inhibitor of formula (I) is efficacious and selective for the MTAP-null cell lines (FIG. 5B).

### Example 17. MTAP-Deletion and MGMT Methylation In Vitro

The influence of MTAP-deletion and MGMT methylation levels on the response of cancer cells to PRMT5 inhibitor of formula (I) was determined.

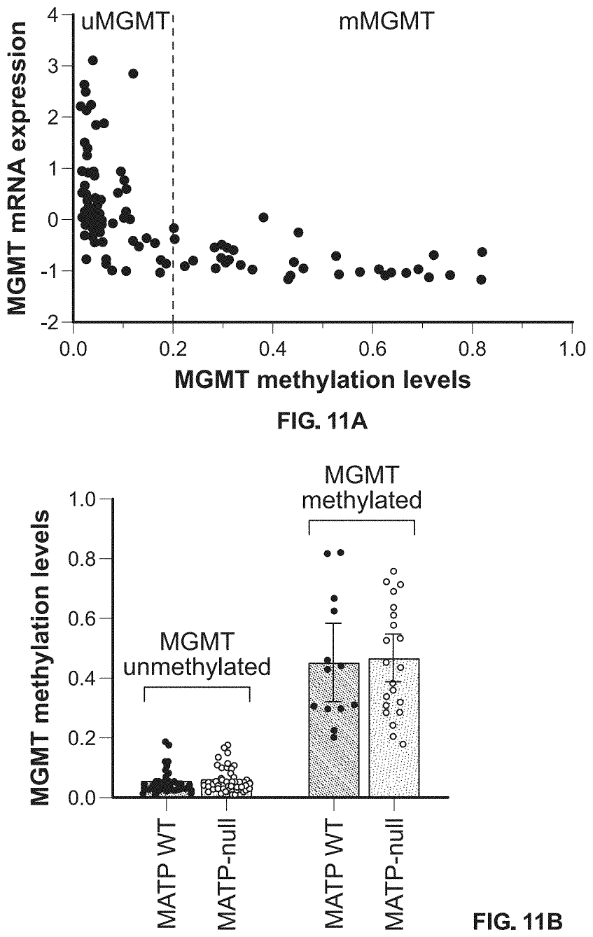

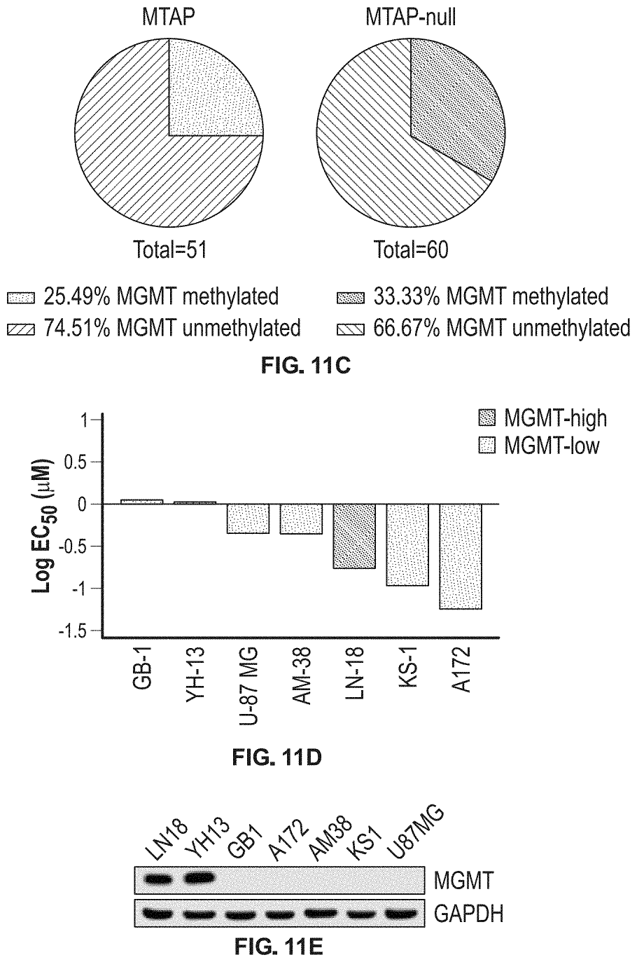

111 glioblastoma patient samples from TCGA Firehose Legacy dataset were profiled for MGMT methylation (HM27 and HM450) and expression status (z-scores relative to diploid samples; RNA Seq V2 RSEM). MGMT methylation threshold was defined as >0.2 for further analyses (FIG. 11A).

The MGMT methylation levels in GBM samples were then segregated by MTAP-status (FIG. 11B). The results show that the degree of MGMT methylation was not influenced by MTAP status.

The MGMT methylation status from GBM samples were then segregated by MTAP-status. MGMT methylation status did not significantly correlate with MTAP-status (FIG. 11C).

A 7-day antiproliferative assay data from MTAP-deleted GBM cell lines from Example 16 were further analyzed and color-coded by MGMT status (FIG. 11D) according to an MGMT immunoblot (FIG. 11E, LN18 and YH13 were positive for MGMT expression). The results show that MGMT methylation status does not significantly correlate with MTAP-status in GBM patient samples, nor predict response to PRMT5 inhibitor of formula (I) in MTAP-deleted GBM cell lines (FIGS. 11D and 11E).

### Example 18. PRMT5 Inhibitor of Formula (I) Antitumor Response in Subcutaneous and Orthotopic MTAPnull GBM Xenograft Models

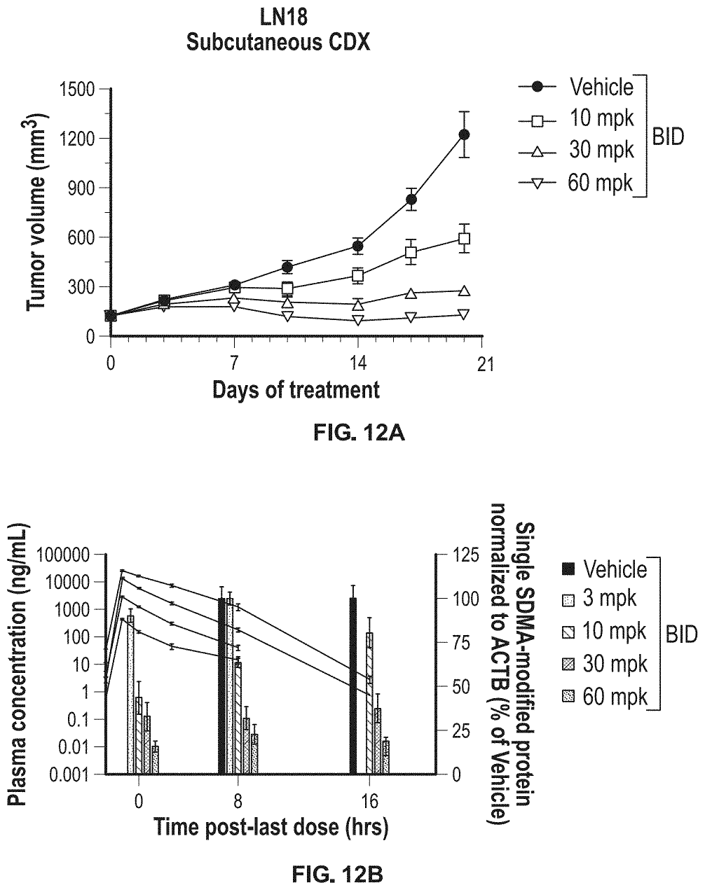

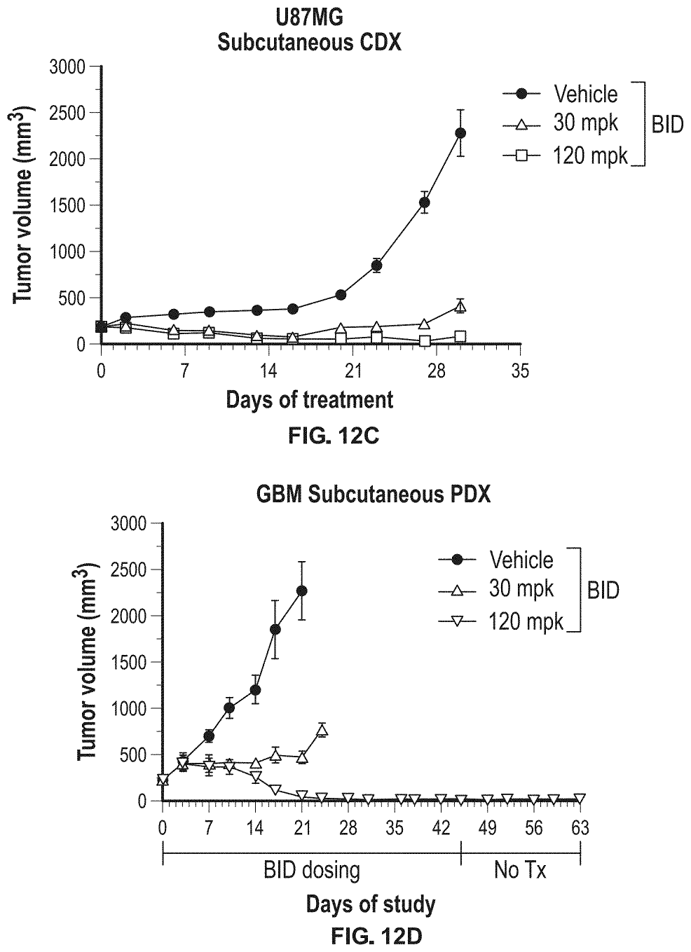

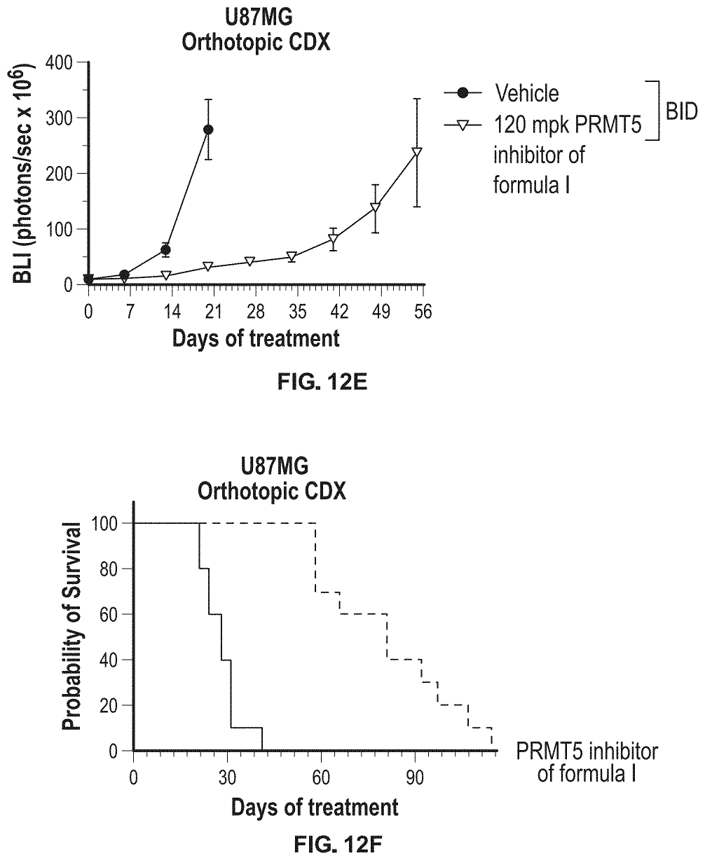

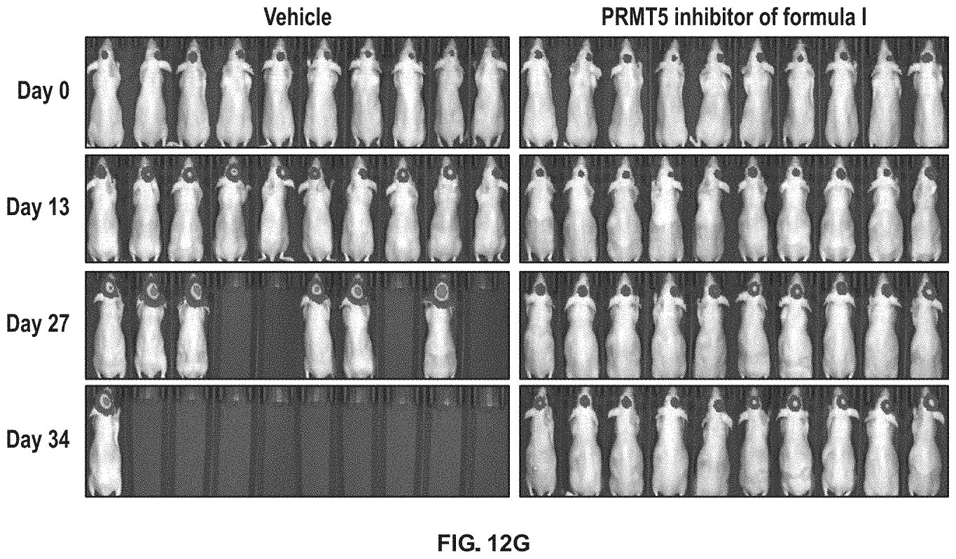

The efficacy of PRMT5 inhibitor of formula (I) in subcutaneous and orthotopic MTAP-null glioblastoma xenograft mouse models was determined. Briefly, subcutaneous LN18 MTAP-null GBM CDX mice were dosed with 10 mpk, 30 mpk, or 60 mpk PRMT5 inhibitor of formula (I) or vehicle BID for 20 days. n=8 mice per group. Additionally, activity was determined in a 7-day PK/PD study of PRMT5 inhibitor of formula (I) in the subcutaneous LN18 MTAP-null GBM CDX model. n=8 mice per group. Tumor volumes, plasma concentration of PRMT5 inhibitor of formula (I), and a single-SDMA modified protein were monitored and plotted. The results show that PRMT5 inhibitor of formula (I) drives strong antitumor responses in subcutaneous LN18 MTAP-null GBM xenograft model in vivo (FIGS. 12A and 12B).

Subcutaneous U87MG MTAP-null GBM CDX mice were dosed with 30 mpk or 60 mpk PRMT5 inhibitor of formula (I) or vehicle BID for 30 days. n=5 mice per group. Tumor volumes were monitored and plotted. The results show that PRMT5 inhibitor of formula (I) drives strong antitumor responses in subcutaneous U87MG MTAP-null GBM xenograft model in vivo (FIG. 12C).

Subcutaneous GBM PDX mouse model was dosed with 30 mpk or 60 mpk PRMT5 inhibitor of formula (I) or vehicle a maximum of 45 days BID. Tumor volumes were monitored for the indicated time period post-cessation of dosing. The results show that PRMT5 inhibitor of formula (I) was efficacious against the GBM PDX mouse model, and 4/5 mice were cured (FIG. 12D).

Orthotopic U87MG MTAP-null GBM CDX mouse model was either treated with vehicle or 120 mpk BID PRMT5 inhibitor of formula (I) (n=10 mice per group). Tumor volumes were monitored by bioluminescence and plotted (FIG. 12E). Mice were dosed and monitored for survival for a maximum of 110 days. The results show a 53-day median survival benefit for PRMT5 inhibitor of formula (I) dosed mice relative to vehicle indicated (FIG. 12F). The rodent PRMT5 inhibitor of formula (I) brain Kpuu was ˜0.15. Weekly bioluminescent data was collected until the first mouse from the PRMT5 inhibitor of formula (I) group was lost due to tumor burden. The data are shown in FIG. 12G. Where applicable, data are plotted as mean±SEM.

### Example 19. PRMT5 Inhibitor of Formula (I) Antitumor Response in Xenograft Tumor Models

The efficacy of PRMT5 inhibitor of formula (I) in PDX mouse models for cholangiocarcinoma, NSCLC (adeno), NSCLC (squamous), bladder cancer, and a CDX DLBCL (OCI-Ly19) mouse models was determined.

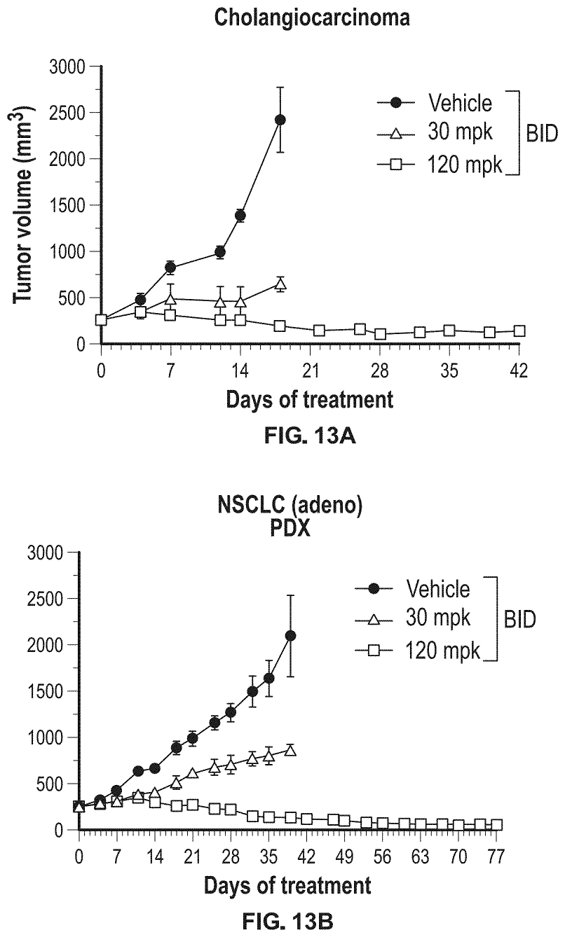

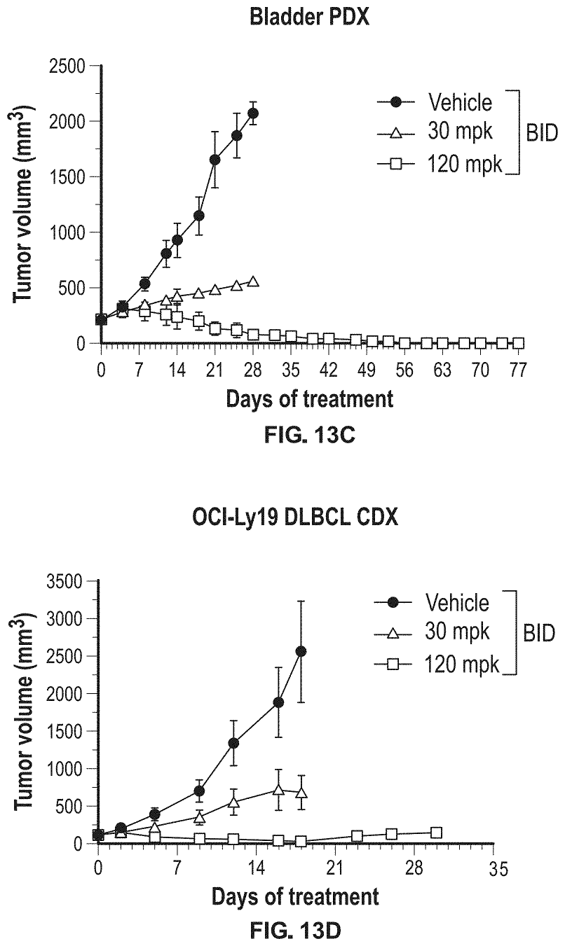

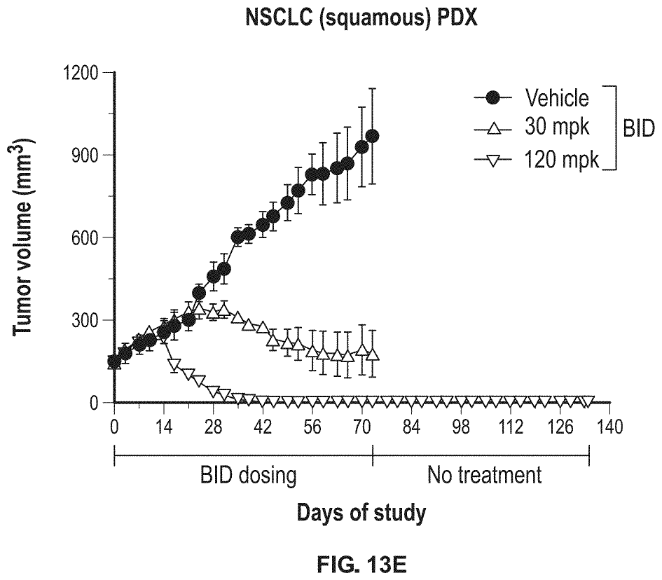

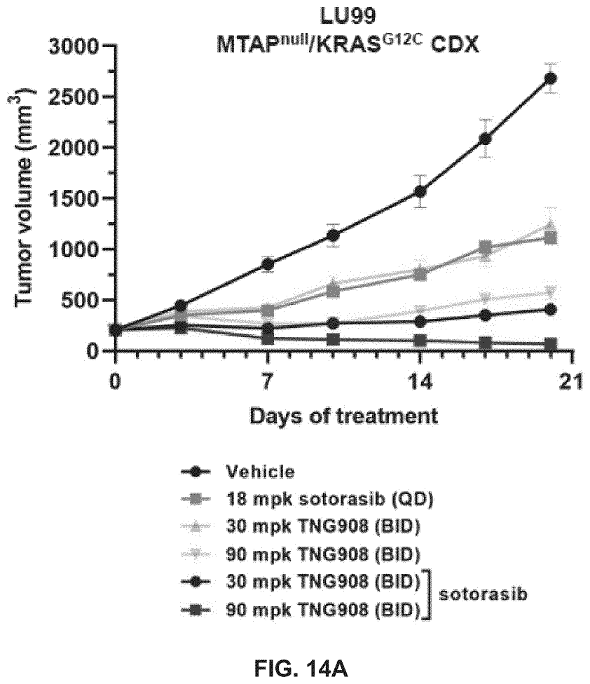

Briefly, the tumor implanted mice (n=3 mice per group) for the cholangiocarcinoma, NSCLC (adeno), bladder cancer, and a DLBCL models were dosed with 30 mpk or 120 mpk PRMT5 inhibitor of formula (I) or vehicle BID for a minimum of 20 days. Tumor volumes were monitored for the indicated time period post-cessation of dosing. The results show that PRMT5 inhibitor of formula (I) was efficacious against the cholangiocarcinoma, NSCLC (adeno), bladder cancer, and DLBCL xenograft mouse models (FIGS. 13A-13D).

In the NSCLC (squamous) PDX model, tumor-bearing mice were dosed with PRMT5 inhibitor of formula (I) for up to 70 days with 30 mpk or 120 mpk or vehicle control. The tumor volume of the 120 mpk dosed mice was then monitored for an additional 60 days after dosing was ended. The results show that in these mice, the tumors did not regrow after the cessation of dosing (FIG. 13E).

### Example 20. In Vivo Combinations of the Compound of Formula (I) and KRAS Inhibitors

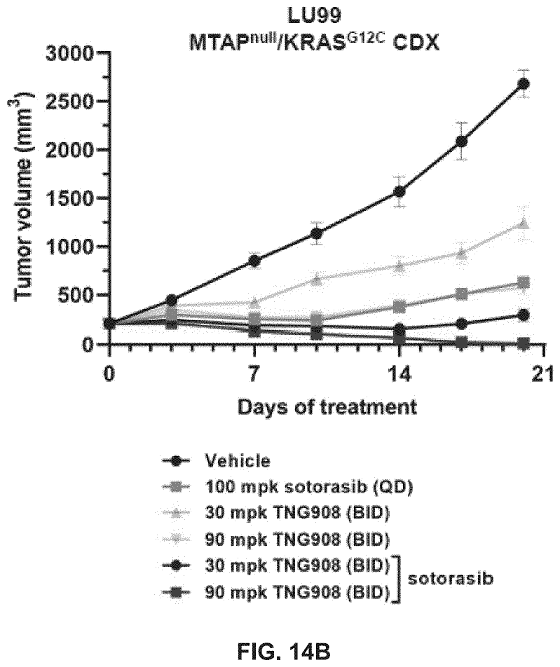

The efficacy of PRMT5 inhibitor of formula (I) in combination with KRAS inhibitor sotorasib was determined in a mouse xenograft model. Briefly, mice bearing MTAP-null/KRAS G12C lung cancer LU99 CDX tumors (8 mice/group) were dosed with 18 mpk sotorasib QD, 30 mpk compound of formula (I) BID, 90 mpk compound of formula (I) BID, a combination of 30 mpk compound of formula (I) BID and sotorasib 18 mpk QD, a combination of 90 mpk compound of formula (I) BID and sotorasib 18 mpk QD, 100 mpk sotorasib QD, a combination of 30 mpk compound of formula (I) BID and sotorasib 100 mpk QD, a combination of 90 mpk compound of formula (I) BID and sotorasib 100 mpk QD, and the tumor volumes were recorded for a minimum of 20 days. The compound of formula (I) has strong single agent and combination activity in vivo (FIGS. 14A and 14B).

### Example 21. In Vivo Combination of the Compound of Formula (I) and CDK4/6 Inhibitors

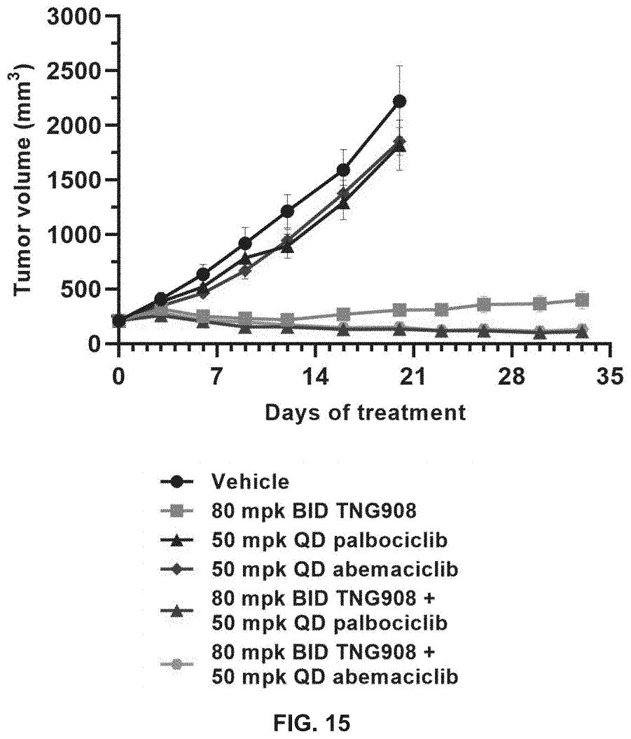

The efficacy of PRMT5 inhibitor of formula (I) in combination with CDK 4/6 inhibitors palbociclib and abemaciclib was determined in a mouse xenograft model. Briefly, mice bearing subcutaneous MTAP-null lung cancer LU99 CDX tumors (8 mice/group) were dosed with 50 mpk palbociclib QD, 50 mpk abemaciclib QD, 80 mpk compound of formula (I) BID, a combination of 80 mpk compound of formula (I) BID and palbociclib 50 mpk QD, a combination of 80 mpk compound of formula (I) BID and abemaciclib 50 mpk QD and the tumor volumes were recorded for a minimum of 20 days. The compound of formula (I) has strong single agent and combination activity in vivo (FIG. 15).

### Example 22. Combination Viability Assay Protocol with MAT2A Inhibitors

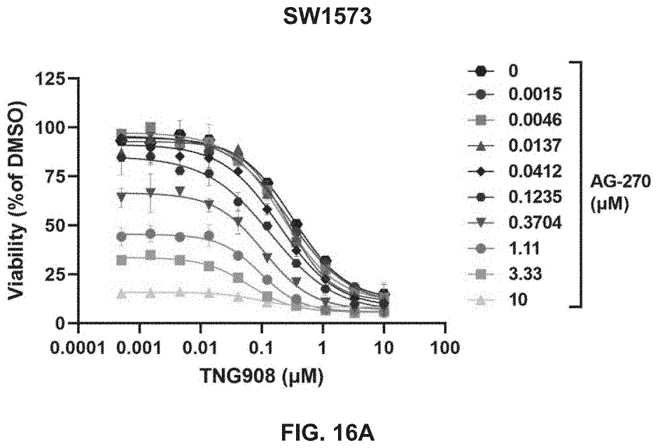

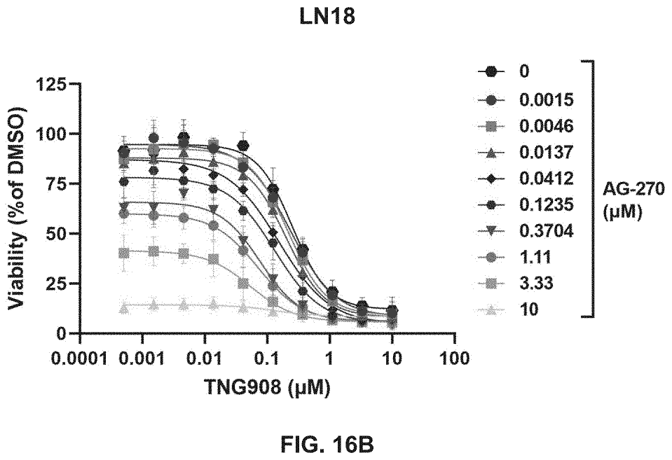

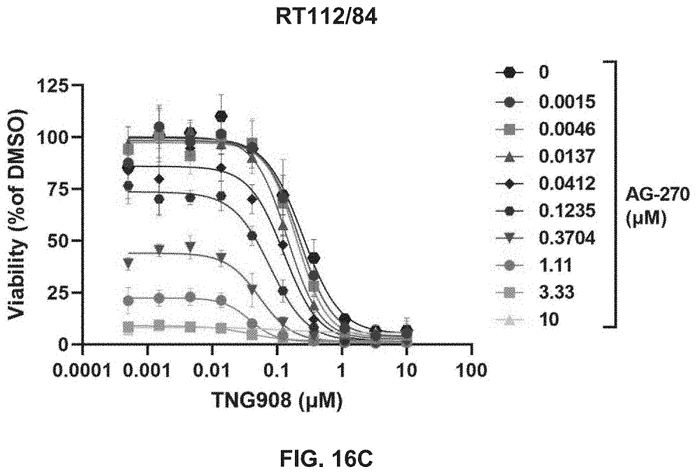

Marjon et al (Cell Reports 2016) and Kalev et al (Cancer Cell 2021) identify MAT2A as a therapeutic target in MTAP-deleted tumors. A combination of AG-270, a MAT2A inhibitor, with the compound of formula (I) in a 7-day viability assay in MTAP-null SW1573 (NSCLC), LN18 (GBM) and RT112/84 (bladder) cancer cell lines, which represent multiple cancer histologies, demonstrates enhanced cellular viability defects regardless of histology. (FIG. 16A (SW1573), FIG. 16B (LN18) and FIG. 16C (RT112/84).

### Incorporation by Reference

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

### Equivalents

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A crystalline form of N-(6-amino-5-methylpyridin-3-yl)-2-((2R,5S)-2-(benzo[d]thiazol-5-yl)-5-methylpiperidin-1-yl)-2-oxoacetamide (a compound of formula (I)) 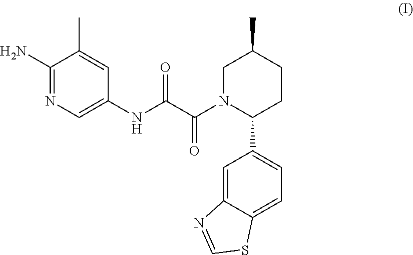 [SMILES: Cc1cc(NC(=O)C(=O)N2C[C@@H](C)CC[C@@H]2c2ccc3scnc3c2)cnc1N]
wherein the X-ray powder diffraction (XRPD) pattern of the crystalline form comprises one or more peaks at 2θ angles selected from 6.4±0.2, 8.9±0.2, 12.7±0.2, 14.0±0.2, 19.1±0.2, 19.9±0.2, 22.6±0.2 degrees.

2. A pharmaceutical composition comprising a compound of formula (I) or a pharmaceutically acceptable salt thereof and at least one pharmaceutically acceptable carrier, diluent, excipient or adjuvant.

3. The pharmaceutical composition of claim 2 wherein the compound of formula (I) is a crystalline form of claim 1.

4. The pharmaceutical composition of claim 2 or 3, wherein the composition comprises about 5% (w/w) to about 50% (w/w) of the compound of formula (I).

5. A pharmaceutical composition comprising:
(a) a compound of formula (I) or a pharmaceutically acceptable salt thereof 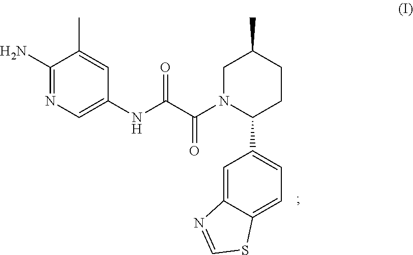 [SMILES: Cc1cc(NC(=O)C(=O)N2C[C@@H](C)CC[C@@H]2c2ccc3scnc3c2)cnc1N]
(b) a filler (e.g., microcrystalline cellulose);
(c) a glidant (e.g., colloidal silicon dioxide);
(d) a disintegrant (e.g., croscarmellose sodium); and
(e) a lubricant (e.g., magnesium stearate).

6. The pharmaceutical composition of claim 5, wherein the composition comprises a crystalline form of the compound of formula (I) of claim 1.

7. A pharmaceutical composition of claim 5 or 6, wherein the composition comprises:
(a) about 5% (w/w) to about 50% (w/w) of the compound of formula (I);
(b) about 50% (w/w) to about 90% (w/w) of a filler (e.g., microcrystalline cellulose);
(c) about 0.5% (w/w) to about 1.5% (w/w) of a glidant (e.g., colloidal silicon dioxide);
(d) about 2% (w/w) to about 6% (w/w) of a disintegrant (e.g., croscarmellose sodium); and
(e) about 0.5% (w/w) to about 1.5% (w/w) of a lubricant (e.g., magnesium stearate);
thereby totaling 100% (w/w) of the composition.

8. A pharmaceutical composition comprising:
(a) a compound of formula (I) 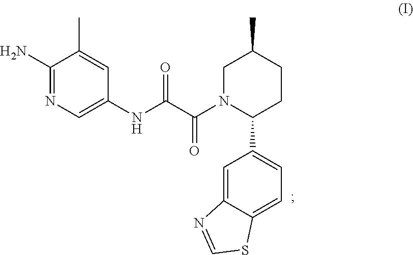 [SMILES: Cc1cc(NC(=O)C(=O)N2C[C@@H](C)CC[C@@H]2c2ccc3scnc3c2)cnc1N]
(b) an intragranular filler (e.g., microcrystalline cellulose);
(c) an intragranular glidant (e.g., colloidal silicon dioxide;
(d) an intragranular disintegrant (e.g., croscarmellose sodium);
(e) an extragranular lubricant (e.g., magnesium stearate);
(f) an extragranular filler (e.g., microcrystalline cellulose);
(g) an extragranular glidant (e.g., colloidal silicon dioxide);
(h) an extragranular disintegrant (e.g., croscarmellose sodium); and
(i) an extragranular lubricant (e.g., magnesium stearate).

9. The pharmaceutical composition of claim 8, wherein the composition comprises a crystalline form of the compound of formula (I) of any one of claims 1 to 4.

10. A pharmaceutical composition of claim 8 or 9, wherein the composition comprises:
(a) about 5% (w/w) to about 50% (w/w) of the compound of formula (I);
(b) about 30% (w/w) to about 70% (w/w) of an intragranular filler (e.g., microcrystalline cellulose);
(c) about 0.25% (w/w) to about 0.75% (w/w) of an intragranular glidant (e.g., colloidal silicon dioxide);
(d) about 1% (w/w) to about 3% (w/w) of an intragranular disintegrant (e.g., croscarmellose sodium);
(e) about 0.25% (w/w) to about 0.75% (w/w) of an intragranular lubricant (e.g., magnesium stearate);
(f) about 0% (w/w) to about 40% (w/w) of an extragranular filler (e.g., microcrystalline cellulose);
(g) about 0.25% (w/w) to about 0.75% (w/w) of an extragranular glidant (e.g., colloidal silicon dioxide);
(h) about 1% (w/w) to about 3% (w/w) of an extragranular disintegrant (e.g., croscarmellose sodium); and
(i) about 0.25% (w/w) to about 0.75% (w/w) of an extragranular lubricant (e.g., magnesium stearate);
thereby totaling 100% (w/w) of the composition.

11. A pharmaceutical composition of claim 8 or 9, wherein the composition comprises:
(a) about 12.5% (w/w) of the compound of formula (I);
(b) about 56.5% (w/w) of an intragranular filler (e.g., microcrystalline cellulose);
(c) about 0.5% (w/w) of an intragranular glidant (e.g., colloidal silicon dioxide);
(d) about 2% (w/w) of an intragranular disintegrant (e.g., croscarmellose sodium);
(e) about 0.5% (w/w) of an intragranular lubricant (e.g., magnesium stearate);
(f) about 25% (w/w) of an extragranular filler (e.g., microcrystalline cellulose);
(g) about 0.5% (w/w) of an extragranular glidant (e.g., colloidal silicon dioxide);
(h) about 2% (w/w) of an extragranular disintegrant (e.g., croscarmellose sodium);
(i) about 0.5% (w/w) of an extragranular lubricant (e.g., magnesium stearate);
thereby totaling 100% (w/w) of the composition.

12. A pharmaceutical composition of claim 8 or 9, wherein the composition comprises:
(a) about 29.4% (w/w) of the compound of formula (I);
(b) about 39.7% (w/w) of an intragranular filler (e.g., microcrystalline cellulose);
(c) about 0.37% (w/w) of an intragranular glidant (e.g., colloidal silicon dioxide);
(d) about 1.47% (w/w) of an intragranular disintegrant (e.g., croscarmellose sodium);
(e) about 0.37% (w/w) of an intragranular lubricant (e.g., magnesium stearate);
(f) about 25.7% (w/w) of an extragranular filler (e.g., microcrystalline cellulose);
(g) about 0.5% (w/w) of an extragranular glidant (e.g., colloidal silicon dioxide);
(h) about 2% (w/w) of an extragranular disintegrant (e.g., croscarmellose sodium);
(i) about 0.5% (w/w) of an extragranular lubricant (e.g., magnesium stearate);
thereby totaling 100% (w/w) of the composition.

13. A pharmaceutical composition of claim 8 or 9, wherein the composition comprises:
(a) about 40% (w/w) of the compound of formula (I);
(b) about 54% (w/w) of an intragranular filler (e.g., microcrystalline cellulose);
(c) about 0.5% (w/w) of an intragranular glidant (e.g., colloidal silicon dioxide);
(d) about 2% (w/w) of an intragranular disintegrant (e.g., croscarmellose sodium);
(e) about 0.5% (w/w) of an intragranular lubricant (e.g., magnesium stearate);
(f) about 0% (w/w) of an extragranular filler (e.g., microcrystalline cellulose);
(g) about 0.5% (w/w) of an extragranular glidant (e.g., colloidal silicon dioxide);
(h) about 2% (w/w) of an extragranular disintegrant (e.g., croscarmellose sodium);
(i) about 0.5% (w/w) of an extragranular lubricant (e.g., magnesium stearate);
thereby totaling 100% (w/w) of the composition.

14. A dosage form comprising a pharmaceutical composition of any one of claims 2 to 13.

15. The dosage form of claim 14, wherein the total weight of the pharmaceutical composition in the dosage form is about 50 mg to 1000 mg.

16. The dosage form of claim 14 or 15, wherein the composition comprises about 5 mg to about 400 mg of a compound of formula (I).

17. The dosage form of any one of claims 14 to 16, wherein the composition comprises about 12.5 mg, about 50 mg, about 100 mg or about 300 mg of the compound of formula (I).

18. A method for treating an MTAP-deficient and/or an MTA-accumulating disease in a subject in need thereof comprising administering to the subject a pharmaceutical composition of any one of claims 2 to 13 containing a therapeutically effective amount of the compound of formula (I).

19. The method of claim 18 wherein the disease is an MTAP-deficient and/or MTA-accumulating cancer.

20. A method of treating a cancer in a subject in need thereof comprising the steps of:
a) assessing the level of MTAP and/or MTA in a test sample obtained from said subject, wherein the MTA level can be assessed directly (e.g., by ELISA or LC-MS/MS) or indirectly (e.g., by SDMA-modified protein ELISA or IHC, or by RNA splicing);
b) comparing the test sample with a reference, wherein MTAP deficiency and/or MTA accumulation in said test sample compared to the reference indicates the cancer in said subject will respond to therapeutic treatment with a PRMT5 inhibitor; and
c) administering the pharmaceutical composition of any one of claims 2 to 13 containing an effective amount (e.g., a therapeutically effective amount) of the compound of formula a (I) to the subject identified in step b).

21. The method of claim 19 or 20 wherein the cancer is glioma, glioblastoma, malignant peripheral nerve sheath tumors (MPNST, e.g., intracranial MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine, sarcoma or a CNS metastasis from a solid tumor.

22. The method of claim 19 or 20, wherein the cancer is a central nervous system (CNS) malignancy.

23. The method of claim 22, wherein the CNS malignancy is selected from glioma (e.g., low grade glioma, intermediate grade glioma), intracranial MPNST tumors, glioblastoma, glioblastoma multiforme, or CNS metastases from solid tumors.

24. The method of claim 19 or 20 wherein the cancer is glioblastoma.

25. The method of any one of claims 19 to 24, wherein the method further comprises administration of a second therapeutic agent.

26. A process for preparing N-(6-amino-5-methylpyridin-3-yl)-2-((2R,5S)-2-(benzo[d]thiazol-5-yl)-5-methylpiperidin-1-yl)-2-oxoacetamide (a compound of formula (I)): 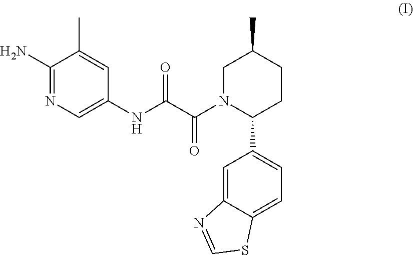 [SMILES: Cc1cc(NC(=O)C(=O)N2C[C@@H](C)CC[C@@H]2c2ccc3scnc3c2)cnc1N]
or a salt thereof,
comprising:
hydrogenating a compound of formula (II): 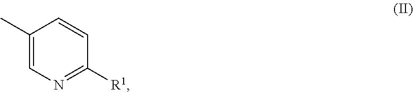
thereby producing a compound of formula (III-a): 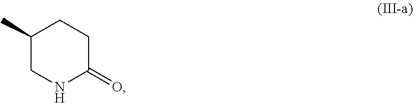
wherein R¹ is a chiral auxiliary.

27. The process of claim 26, wherein the process further comprises:
protecting the nitrogen group of the compound of formula (III-a): 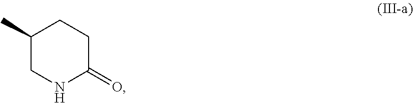
thereby producing a compound of formula (III):

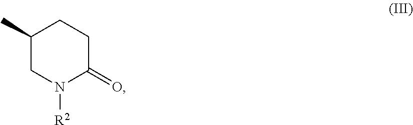

28. The process of claim 26 or 27, wherein the process further comprises:
cross-coupling a compound of formula (III) with a compound of formula (IV): 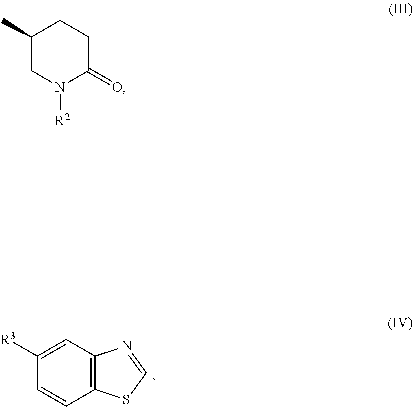
thereby producing a compound of formula (V): 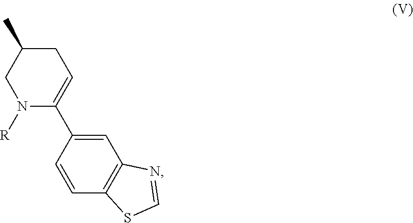
wherein R² is a nitrogen protecting group; and
R³ is a boronic acid or a boronic ester.

29. The process of any one of claims 26 to 28, wherein the process further comprises removing the nitrogen protecting group from the compound of formula (V) 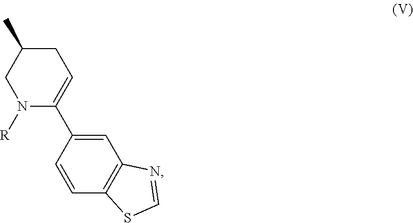
thereby producing a compound of formula (V-a):

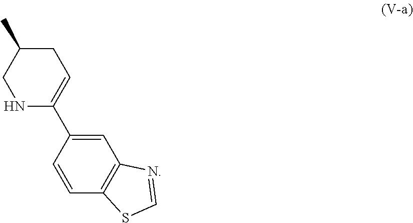 [SMILES: C[C@H]1CC=C(c2ccc3scnc3c2)NC1]

30. The process of any one of claims 26 to 29, wherein the process further comprises:
reducing the compound of formula (V-a): 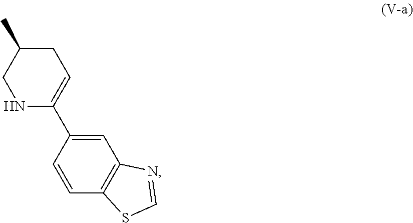 [SMILES: C[C@H]1CC=C(c2ccc3scnc3c2)NC1]
thereby producing a compound of formula (VI):

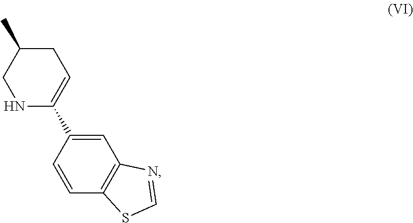

31. The process of any one of claims 26 to 30, wherein the process further comprises:
coupling the compound of formula (VI) with a compound of formula (VII): 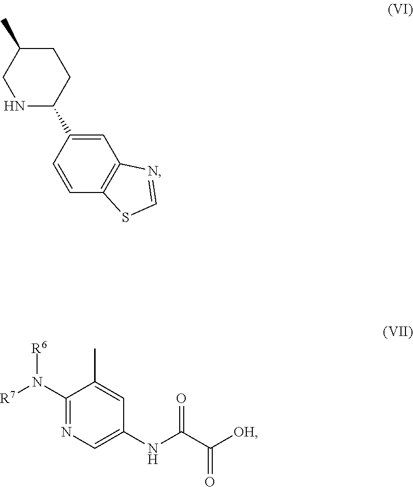
thereby producing a compound of formula (I-a): 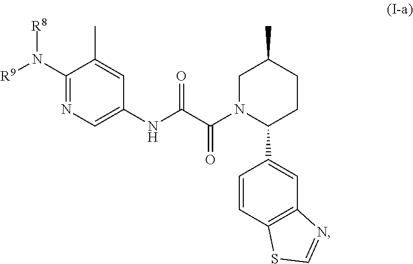
wherein each of R⁶, R⁷, R⁸, and R⁹ is, independently, H or a nitrogen protecting group.

32. The process of any one of claims 26 to 31, wherein the process further comprises removing the nitrogen protecting groups from the compound of formula (I-a): 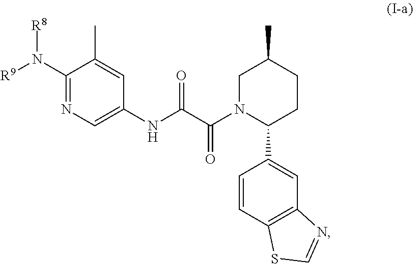
thereby producing the compound of formula (I) or a salt thereof:

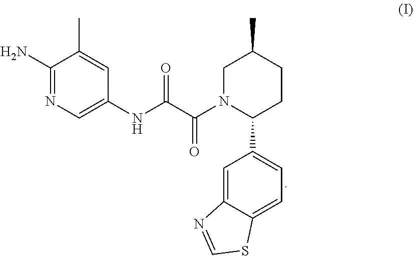 [SMILES: Cc1cc(NC(=O)C(=O)N2C[C@@H](C)CC[C@@H]2c2ccc3scnc3c2)cnc1N]

33. A process for preparing N-(6-amino-5-methylpyridin-3-yl)-2-((2R,5S) (benzo[d]thiazol-5-yl)-5-methylpiperidin-1-yl)-2-oxoacetamide (a compound of formula (I)) or a salt thereof: 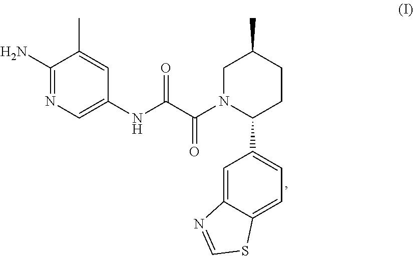 [SMILES: Cc1cc(NC(=O)C(=O)N2C[C@@H](C)CC[C@@H]2c2ccc3scnc3c2)cnc1N]
comprising:
(a) hydrogenating a compound of formula (II): 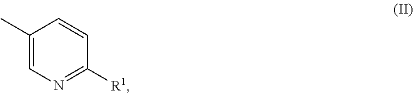
thereby producing a compound of formula (III-a): 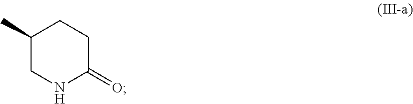
(b) protecting the nitrogen group of the compound of formula (III-a): 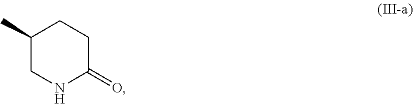
thereby producing a compound of formula (III): 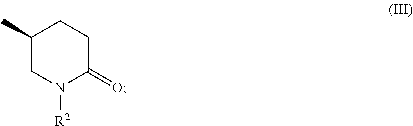
(c) cross-coupling a compound of formula (III) with a compound of formula (IV): 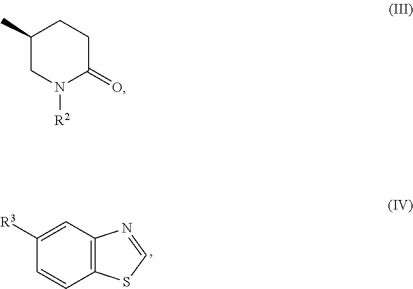
thereby producing a compound of formula (V): 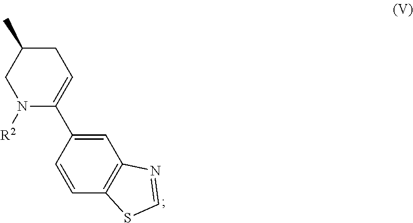
(d) removing the nitrogen protecting group from the compound of formula (V) 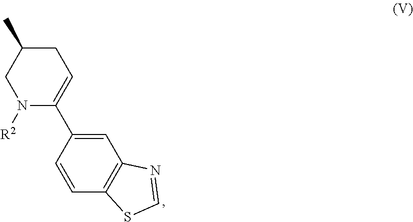
thereby producing a compound of formula (V-a): 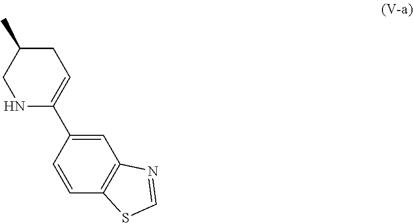 [SMILES: C[C@H]1CC=C(c2ccc3scnc3c2)NC1]
(e) reducing the compound of formula (V-a), thereby producing a compound of formula (VI): 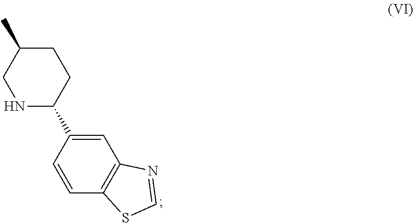 [SMILES: C[C@H]1CC[C@H](c2ccc3scnc3c2)NC1]
and
(f) coupling a compound of formula (VI) with a compound of formula (VII): 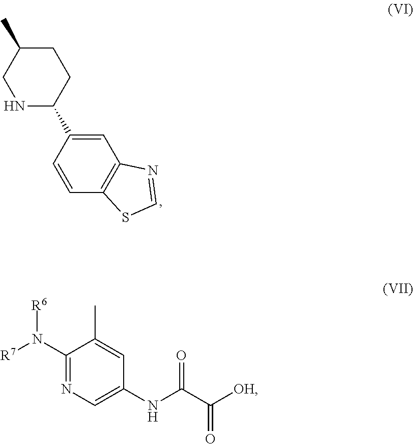
thereby producing a compound of formula (I-a): 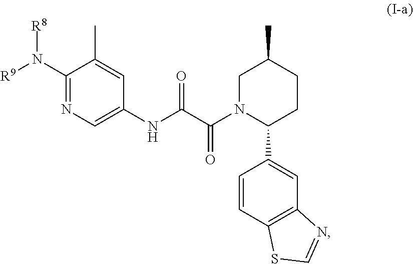
wherein R¹ is a chiral auxiliary;
R² is a nitrogen protecting group;
each of R⁶, R⁷, R⁸, and R⁹ is, independently, H or a nitrogen protecting group; and
R³ is a boronic acid group or a boronic ester group.

34. The process of claim 33, wherein R⁸, R⁹, or both are a nitrogen protecting group, the process further comprises removing the nitrogen protecting group from the compound of formula (I-a): 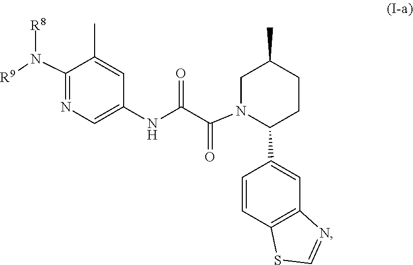
thereby producing the compound of formula (I) or a salt thereof:

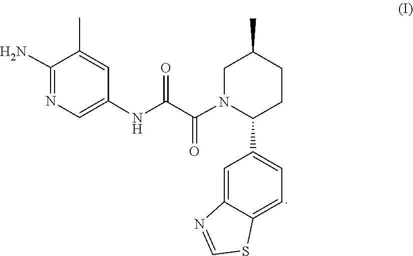 [SMILES: Cc1cc(NC(=O)C(=O)N2C[C@@H](C)CC[C@@H]2c2ccc3scnc3c2)cnc1N]

35. A process for preparing N-(6-amino-5-methylpyridin-3-yl)-2-((2R,5S) (benzo[d]thiazol-5-yl)-5-methylpiperidin-1-yl)-2-oxoacetamide (a compound of formula (I)) or a salt thereof: 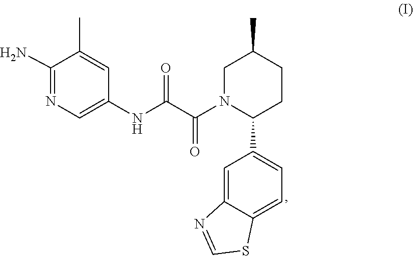 [SMILES: Cc1cc(NC(=O)C(=O)N2C[C@@H](C)CC[C@@H]2c2ccc3scnc3c2)cnc1N]
comprising:
coupling the compound of formula (VI) with a compound of formula (VII): 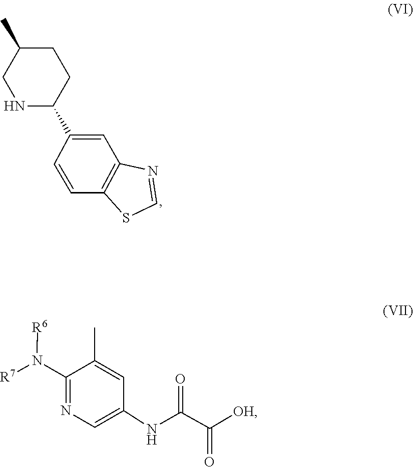
thereby producing a compound of formula (I-a): 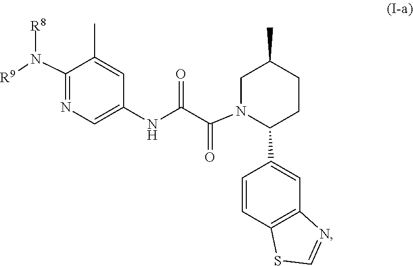
wherein each of R⁶, R⁷, R⁸, and R⁹ is, independently, H or a nitrogen protecting group;
optionally, if R⁸, R⁹, or both are a nitrogen protecting group, removing the nitrogen protecting group from the compound of formula (I-a), thereby producing the compound of formula (I) or a salt thereof.

36. The process of claim 35, wherein the process further comprises:
reducing the compound of formula (V-a): 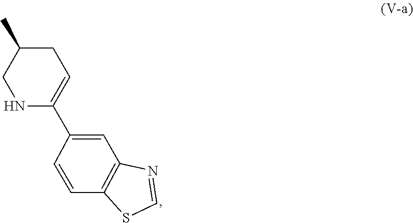 [SMILES: C[C@H]1CC=C(c2ccc3scnc3c2)NC1]
thereby producing a compound of formula (VI):

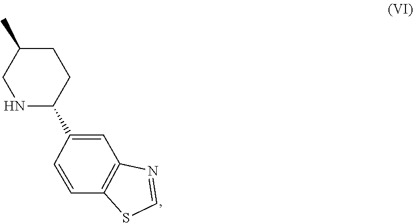 [SMILES: C[C@H]1CC[C@H](c2ccc3scnc3c2)NC1]

37. The process of claim 35 or 36, wherein the process further comprises removing the nitrogen protecting group from a compound of formula (V) 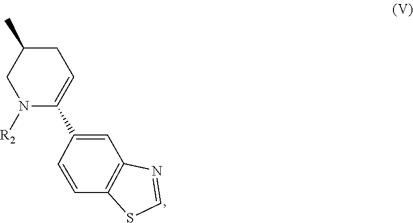
thereby producing the compound of formula (V-a):

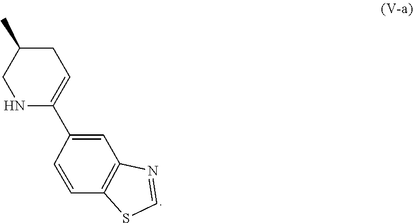 [SMILES: C[C@H]1CC=C(c2ccc3scnc3c2)NC1]

38. The process of any one of claims 35 to 37, wherein the process further comprises:
cross-coupling a compound of formula (III) with a compound of formula (IV): 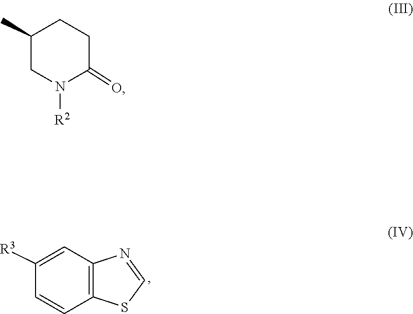
thereby producing a compound of formula (V): 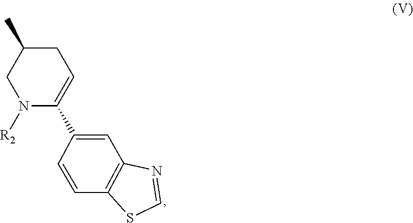
wherein R² is a nitrogen protecting group; and
R³ is a boronic acid group or a boronic ester group.

39. The process of any one of claims 35 to 38, wherein the process further comprises:
protecting the nitrogen group of a compound of formula (III-a): 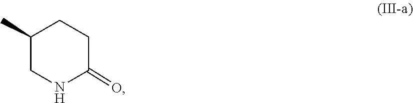
thereby producing the compound of formula (III):

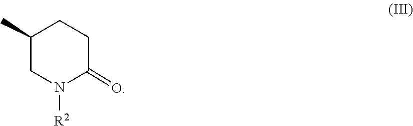

40. The process of any one of claims 35 to 39, wherein the process further comprises:
hydrogenating a compound of formula (II): 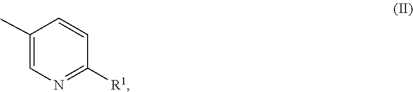
thereby producing a compound of formula (III-a): 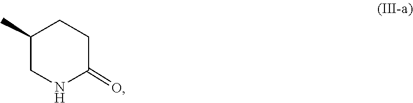
wherein R¹ is a chiral auxiliary.
